(19) United States
(12) Patent Application Publication
(10) Pub. No.: US 2024/0269143 A1
(43) Pub. Date: Aug. 15, 2024

(54) COMBINATION THERAPY COMPRISING A MAT2A INHIBITOR AND TYPE I PRMT INHIBITOR

(71) Applicants: IDEAYA BIOSCIENCES, INC., South San Francisco, CA (US); GlaxoSmithKline Intellectual Property (No.4) Limited, Brentford, Middlesex (GB)

(72) Inventors: Michael Patrick DILLON, South San Francisco, CA (US); Marcus Michael FISCHER, South San Francisco, CA (US); Kimberline Yang GERRICK, South San Francisco, CA (US); Jenny Laraio, Collegeville, PA (US); Helai Mohammad, Collegeville, PA (US); Kimberly Smitheman, Collegeville, PA (US)

(21) Appl. No.: 18/565,392

(22) PCT Filed: Jun. 1, 2022
(86) PCT No.: PCT/US2022/072697

§ 371 Date: Nov. 29, 2023

Related U.S. Application Data
(60) Provisional application No. 63/196,025, filed on Jun. 2, 2021.

(51) Int. Cl. A61K 31/519; A61K 31/415; A61P 35/00

(52) U.S. Cl. CPC A61K 31/519; A61K 31/415; A61P 35/00

(57) ABSTRACT

Provided herein is a combination therapy and methods of using such combination therapy to treat diseases or disorders associated with MAT2A and/or Type I PRMT.

### RELATED APPLICATIONS

This application claims priority to U.S. Provisional Application No. 63/196,025 filed on Jun. 2, 2021, the entire content of which is hereby incorporated by reference in its entirety.

### BACKGROUND

Cancer is a leading cause of death throughout the world. A limitation of prevailing therapeutic approaches, e.g., chemotherapy and immunotherapy, is that their cytotoxic effects are not restricted to cancer cells and adverse side effects can occur within normal tissues.

Arginine methylation occurs largely in the context of glycine-, arginine-rich (GAR) motifs through the activity of a family of Protein Arginine Methyltransferases (PRMTs) that transfer the methyl group from S-adenosyl-L-methionine (SAM) to the substrate arginine side chain producing S-adenosyl-homocysteine (SAH) and methylated arginine. Dysregulation and overexpression of PRMT1 have been associated with a number of solid and hematopoietic cancers (Yang, Y. & Bedford, M. T. Protein arginine methyltransferases and cancer (Nat Rev Cancer 13, 37-50, (2013); Yoshimatsu, M. et al.).

Dysregulation of PRMT1 and PRMT6, Type I arginine methyltransferases, is involved in various types of human cancers (Int J Cancer 128, 562-573, (2011)). The link between PRMT1 and cancer biology has largely been through regulation of methylation of arginine residues found on relevant substrates. In several tumor types, PRMT1 can drive expression of aberrant oncogenic programs through methylation of histone H4. In many of these experimental systems, disruption of the PRMT1-dependent ADMA modification of its substrates decreases the proliferative capacity of cancer cells (Yang, Y. & Bedford, M. T. Nat Rev Cancer 13, 37-50, (2013)).

As the primary methyl donor for arginine methylation in cells, SAM synthesis is crucial to the functionality of this process. The catalysis of methionine and ATP to form SAM by methionine adenosyltransferases is considered the rate-limiting step in this process. Two adenosyltransferase genes (MAT1A and MAT2A), which encode distinct catalytic isoforms, are known to be differentially impacted in cancer. While MAT1A is specifically expressed in the adult liver, MAT2A activity is widely distributed. Because of MAT2A's critical role in facilitating the growth of hepatoma cells, it is a target for antineoplastic therapy.

Despite many recent advances in cancer therapies, there remains a need for more effective and/or enhanced treatment for those individuals suffering the effects of cancer.

### SUMMARY

Provided herein is a combination product comprising a methionine adenosyltransferase II alpha (MAT2A) inhibitor and Type I protein arginine methyltransferase (Type I PRMT) inhibitor. The combination product is useful for the treatment of a variety of cancers, including solid tumors. The combination product is also useful for the treatment of any number of MAT2A-associated and/or PRMT-associated diseases. Thus, in one aspect, provided herein is a method of treating cancer in a subject in need thereof, comprising administering to the subject a combination therapy comprising a methionine adenosyltransferase II alpha (MAT2A) inhibitor and Type I protein arginine methyltransferase (Type I PRMT) inhibitor.

In an embodiment, provided herein is a first pharmaceutical composition comprising a therapeutically effective amount of a MAT2A inhibitor and a second pharmaceutical composition comprising a therapeutically effective amount of Type I PRMT inhibitor.

In an embodiment, provided herein are methods of treating cancer in a subject in need thereof, the methods comprising administering to the subject a combination comprising a MAT2A inhibitor and a Type I PRMT inhibitor, thereby treating the cancer in the subject.

In an embodiment, provided herein are methods of treating cancer in a subject in need thereof, the methods comprising administering to the subject a combination comprising a MAT2A inhibitor and a Type I PRMT inhibitor, together with at least a pharmaceutically acceptable carrier, thereby treating the cancer in the subject.

In an embodiment, provided herein are methods of treating cancer in a subject in need thereof, the methods comprising administering to the subject a therapeutically effective amount of a pharmaceutical composition comprising a MAT2A inhibitor and a therapeutically effective amount of a pharmaceutical composition comprising a Type I PRMT inhibitor, thereby treating the cancer in the subject.

In an embodiment, provided herein is a combination of a MAT2A inhibitor and a Type I PRMT inhibitor. In an embodiment, the Type I PRMT inhibitor is a PRMT1 inhibitor.

In an embodiment, provided herein are methods of treating a disease or disorder treatable by inhibiting MAT2A and/or Type I PRMT in a subject in need thereof, the methods comprising administering to the subject a combination comprising a MAT2A inhibitor and a Type I PRMT inhibitor, thereby treating the disease or disorder in the subject. In an embodiment, the Type I PRMT inhibitor is a PRMT1 inhibitor. In an embodiment, the disease or disorder is cancer.

In an embodiment, provided herein are methods of treating a disease or disorder treatable by inhibiting MAT2A and/or Type I PRMT in a subject in need thereof, the methods comprising administering to the subject a combination comprising a MAT2A inhibitor and a Type I PRMT inhibitor, together with at least a pharmaceutically acceptable carrier, thereby treating the disease or disorder in the subject. In an embodiment, the Type I PRMT inhibitor is a PRMT1 inhibitor. In an embodiment, the disease or disorder is cancer.

In an embodiment, the methionine adenosyltransferase II alpha (MAT2A) inhibitor is a compound of Formula I:

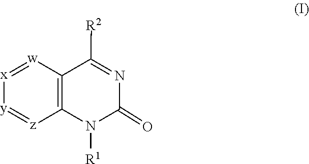

or a pharmaceutically acceptable salt thereof.

In yet another embodiment, the Type I PRMT inhibitor is a protein arginine methyltransferase 1 (PRMT1) inhibitor.

In still another embodiment, the Type I PRMT inhibitor is a compound of Formula II:

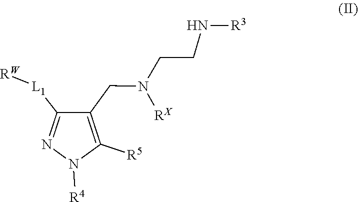

or a pharmaceutically acceptable salt thereof.

In another aspect, provided herein is a combination product comprising a methionine adenosyltransferase II alpha (MAT2A) inhibitor of Compound A:

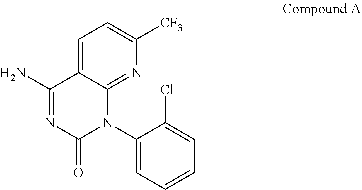 [SMILES: Nc1nc(=O)n(-c2ccccc2Cl)c2nc(C(F)(F)F)ccc12]

or a pharmaceutically acceptable salt thereof,

and a PRMT1 inhibitor of Compound B:

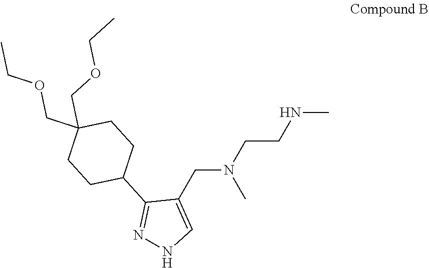 [SMILES: CCOCC1(COCC)CCC(c2n[nH]cc2CN(C)CCNC)CC1]

or a pharmaceutically acceptable salt thereof.

In still another embodiment, the cancer is characterized by a reduction or absence of MTAP gene expression, absence of the MTAP gene, reduced function of MTAP protein, reduced level of MTAP protein, absence of MTAP protein, MTA accumulation, or combination thereof.

### BRIEF DESCRIPTION OF THE DRAWINGS

FIG. 1 shows the efficacy of Compound A and Compound B in HCT-116 MTAP isogenic pair models.

FIG. 2 shows the efficacy of Compound A and Compound B in HCT-116 MTAP WT model.

FIG. 3 shows the efficacy of Compound A and Compound B in BxPC-3 model.

FIG. 4 is a BxPC3 cell line synergistic growth inhibition analysis. Synergy is measured at varying concentrations of MAT2A inhibitor (Compound A) and with the Type I PRMT inhibitor (Compound B) using HSA, Bliss and Loewe models.

FIG. 5 is a KP4 cell line synergistic growth inhibition analysis. Synergy is measured at varying concentrations of MAT2A inhibitor (Compound A) and with the Type I PRMT inhibitor (Compound B) using HSA, Bliss and Loewe models. No significant synergy was observed in the Bliss model.

FIG. 6 is a NCI-H838 cell line synergistic growth inhibition analysis. Synergy is measured at varying concentrations of MAT2A inhibitor (Compound A) and with the Type I PRMT inhibitor (Compound B) using HSA, Bliss and Loewe models. No significant synergy was observed in the Bliss model.

FIG. 7 is a HCT116 cell line synergistic growth inhibition analysis. Synergy is measured at varying concentrations of MAT2A inhibitor (Compound A) and with the Type I PRMT inhibitor (Compound B) using HSA, Bliss and Loewe models.

FIG. 8 is a HCT116-MTAP^{-/-} cell line synergistic growth inhibition analysis. Synergy is measured at varying concentrations of MAT2A inhibitor (Compound A) and with the Type I PRMT inhibitor (Compound B) using HSA, Bliss and Loewe models. No significant synergy was observed in the bliss model.

FIG. 9 shows a change in MTA levels in cell lines at baseline and following 48 hours of cell culture.

### DETAILED DESCRIPTION

Effective treatment of hyperproliferative disorders, such as cancer, is a continuing goal in the oncology field. Generally, cancer results from the deregulation of the normal processes that control cell division, differentiation and apoptotic cell death and is characterized by the proliferation of malignant cells which have the potential for unlimited growth, local expansion and systemic metastasis. Deregulation of normal processes includes abnormalities in signal transduction pathways and response to factors that differ from those found in normal cells.

Arginine methylation is an important post-translational modification on proteins involved in a diverse range of cellular processes such as gene regulation, RNA processing, DNA damage response, and signal transduction. Proteins containing methylated arginines are present in both nuclear and cytosolic fractions suggesting that the enzymes that catalyze the transfer of methyl groups on to arginines are also present throughout these subcellular compartments (Yang, Y. & Bedford, M. T. Nat Rev Cancer 13, 37-50, (2013); Lee, Y. H. & Stallcup, M. R. Mol Endocrinol 23, 425-433, (2009)). Each methylation state can affect protein-protein interactions in different ways and therefore has the potential to confer distinct functional consequences for the biological activity of the substrate (Yang, Y. & Bedford, M. T. Nat Rev Cancer 13, 37-50, (2013)).

Arginine methylation occurs largely in the context of glycine-, arginine-rich (GAR) motifs through the activity of a family of Protein Arginine Methyltransferases (PRMTs) that transfer the methyl group from S-adenosyl-L-methionine (SAM) to the substrate arginine side chain producing S-adenosyl-homocysteine (SAH) and methylated arginine. This family of proteins is comprised of ten members, of which nine have been shown to have enzymatic activity (Bedford, M. T. & Clarke, S. G. Mol Cell 33, 1-13, (2009)). The PRMT family is categorized into four sub-types (Type I-IV) depending on the product of the enzymatic reaction. Type IV enzymes methylate the internal guanidino nitrogen and have only been described in yeast (Fisk, J. C. & Read, L. K. Eukaryot Cell 10, 1013-1022, (2011)); types I-III enzymes generate monomethyl-arginine (MMA, Rmel) through a single methylation event. The MMA intermediate is considered a relatively low abundance intermediate, however, select substrates of the primarily Type III activity of PRMT7 can remain monomethylated, while Types I and II enzymes catalyze progression from MMA to either asymmetric dimethyl-arginine (ADMA, Rme2a) or symmetric dimethyl arginine (SDMA, Rme2s) respectively.

Dysregulation and overexpression of PRMT1 have been associated with a number of solid and hematopoietic cancers (Yang, Y. & Bedford, M. T. Nat Rev Cancer 13, 37-50, (2013); Yoshimatsu, M. et al. Int J Cancer 128, 562-573, (2011)). The link between PRMT1 and cancer biology has largely been through regulation of methylation of arginine residues found on relevant substrates. In several tumor types, PRMT1 can drive expression of aberrant oncogenic programs through methylation of histone H4 (Takai, H. et al Cell Rep 9, 48-60, (2014); Shia, W. J. et al. Blood 119, 4953-4962, (2012); Zhao, X. et al. Genes Dev 22, 640-653, (2008)), as well as through its activity on non-histone substrates (Wei, H., et al. Cell Cycle 13, 32-41, (2014)). In many of these experimental systems, disruption of the PRMT1-dependent ADMA modification of its substrates decreases the proliferative capacity of cancer cells (Yang and Bedford).

As the primary methyl donor for arginine methylation in cells, S-adenosyl methionine (SAM) synthesis is crucial to the functionality of this process. The catalysis of methionine and ATP to form SAM by methionine adenosyltransferases is considered the rate-limiting step in this process. Two adenosyltransferase genes (MAT1A and MAT2A), which encode distinct catalytic isoforms, as well as a third gene (MAT2B) that encodes a MAT2A regulatory subunit, are known to be differentially impacted in cancer. While MAT1A is specifically expressed in the adult liver, MAT2A activity is widely distributed. For example, in hepatocellular carcinoma (HCC), the downregulation of MAT1A and the upregulation of MAT2A occur, which is known as the MAT1A-MAT2A switch. This switch, accompanied with up regulation of MAT2B, results in lower SAM contents and provides a growth advantage to hepatoma cells. Because of MAT2A's critical role in facilitating the growth of hepatoma cells, it is a target for antineoplastic therapy.

Further, methylthioadenosine phosphorylase (MTAP), another component of the methionine cycle, also impacts the epigenetic dysregulation observed in some tumors. The locus encoding this enzyme that converts methylthioadenosine (MTA) into adenine and 5-melhylthioribose-1-phosphate is deleted in 15% of cancers (Tang et al., Cancer Res. 78(15), 4386-4395). Despite many recent advances in cancer therapies, there remains a need for more effective and/or enhanced treatment for those individuals suffering the effects of cancer.

Provided herein is a combination therapy comprising a methionine adenosyltransferase II alpha (MAT2A) inhibitor, or a pharmaceutically acceptable salt thereof, and Type I protein arginine methyltransferase (Type I PRMT) inhibitor, or a pharmaceutically acceptable salt thereof. The combination therapy is useful for the treatment of a variety of cancers, including solid tumors. The combination therapy can also be useful for treatment of cancer characterized by a reduction or absence of MTAP gene expression, absence of the MTAP gene, reduced function of MTAP protein, reduced level of MTAP protein, absence of MTAP protein, MTA accumulation, or combination thereof. In another aspect, the combination therapy is useful for the treatment of any number of MAT2A-associated and/or PRMT-associated diseases.

Administering a combination of a methionine adenosyltransferase II alpha (MAT2A) inhibitor and Type I protein arginine methyltransferase (Type I PRMT) inhibitor can provide beneficial effects for treating cancer, e.g., MTAP-null cancer, in a subject. Such an approach—combination or co-administration of the two types of agents—may offer an uninterrupted treatment to a subject in need over a clinically relevant treatment period.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

As used in the specification and in the claims, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically acceptable carriers, and excludes other compounds.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt. % to about 5 wt. %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, 2%, 3%, 4%, 5%, 6%, ±7%, 8%, 9%, or ±10%, of the numerical value(s) being modified. In addition, the phrase “about ‘x’ to ‘y’” includes “about ‘x’ to about ‘y’”.

The terms “combination,” “therapeutic combination,” “pharmaceutical combination,” or “combination product” as used herein refer to either a fixed combination in one dosage unit form, or non-fixed combination in separate dosage forms, or a kit of parts for the combined administration where two or more therapeutic agents may be administered independently, at the same time or separately within time intervals.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single formulation having a fixed ratio of active ingredients or in separate formulations (e.g., capsules and/or intravenous formulations) for each active ingredient. In addition, such administration also encompasses use of each type of therapeutic agent in a sequential or separate manner, either at approximately the same time or at different times. Regardless of whether the active ingredients are administered as a single formulation or in separate formulations, the drugs are administered to the same patient as part of the same course of therapy. In any case, the treatment regimen will provide beneficial effects in treating the conditions or disorders described herein.

As used herein, the term “treating” or “treatment” refers to inhibiting a disease; for example, inhibiting a disease, condition, or disorder in an individual who is experiencing or displaying the pathology or symptomology of the disease, condition, or disorder (i.e., arresting further development of the pathology and/or symptomology) or ameliorating the disease; for example, ameliorating a disease, condition, or disorder in an individual who is experiencing or displaying the pathology or symptomology of the disease, condition, or disorder (i.e., reversing the pathology and/or symptomology) such as decreasing the severity of the disease.

The term “prevent,” “preventing,” or “prevention” as used herein, comprises the prevention of at least one symptom associated with or caused by the state, disease or disorder being prevented.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein a parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts described herein include the conventional non-toxic salts of the parent compound formed, for example, from non-toxic inorganic or organic acids. The pharmaceutically acceptable salts discussed herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound with a pharmaceutically acceptable carrier. The pharmaceutical composition facilitates administration of the composition to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound disclosed herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of a compound disclosed herein, and are physiologically acceptable to the patient. Supplementary active compounds may also be incorporated into the compositions. The “pharmaceutically acceptable carrier” may further include a pharmaceutically acceptable salt of the compound(s) disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

The term “single formulation” as used herein refers to a single carrier or vehicle formulated to deliver effective amounts of both therapeutic agents to a patient. The single vehicle is designed to deliver an effective amount of each of the agents, along with any pharmaceutically acceptable carriers or excipients. In some embodiments, the vehicle is a tablet, capsule, pill, or a patch. In other embodiments, the vehicle is a solution or a suspension.

As used herein, “Type I protein arginine methyltransferase inhibitor” or “Type I PRMT inhibitor” means an agent that modulates Type I PRMT or inhibits any one or more of the following: protein arginine methyltransferase 1 (PRMT1), protein arginine methyltransferase 3 (PRMT3), protein arginine methyltransferase 4 (PRMT4), protein arginine methyltransferase 6 (PRMT6) inhibitor, and protein arginine methyltransferase 8 (PRMT8). In some embodiments, the Type I PRMT inhibitor is a small molecule compound. In some embodiments, the Type I PRMT inhibitor selectively inhibits any one or more of the following: protein arginine methyltransferase 1 (PRMT1), protein arginine methyltransferase 3 (PRMT3), protein arginine methyltransferase 4 (PRMT4), protein arginine methyltransferase 6 (PRMT6) inhibitor, and protein arginine methyltransferase 8 (PRMT8). In some embodiments, the Type I PRMT inhibitor is a selective inhibitor of PRMT1, PRMT3, PRMT4, PRMT6, and PRMT8.

As used herein “methionine adenosyltransferase II alpha inhibitor” or “MAT2A inhibitor” means an agent that modulates or inhibits the production of S-adenosylmethionine (SAM) by methionine adenosyltransferase 2A (MAT2A).

The term “unit dose” is used herein to mean simultaneous administration of both agents together, in one dosage form, to the patient being treated. In some embodiments, the unit dose is a single formulation. The term “a unit dose,” as used herein can also refer to the simultaneous administration of both agents separately, in two dosage forms, to the patient being treated. In certain embodiments, the unit dose includes one or more vehicles such that each vehicle includes an effective amount of at least one of the agents along with pharmaceutically acceptable carriers and excipients. In some embodiments, the unit dose is one or more tablets, capsules, pills, or patches administered to the patient at the same time.

The combination of agents described herein may display a synergistic effect. The term “synergistic effect” as used herein, refers to action of two agents such as, for example, a methionine adenosyltransferase II alpha (MAT2A) inhibitor and Type I protein arginine methyltransferase (Type I PRMT) inhibitor, producing an effect, for example, slowing the symptomatic progression of cancer or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

As used herein, the term “synergy” refers to the effect achieved when the active ingredients, i.e., a methionine adenosyltransferase II alpha (MAT2A) inhibitor and Type I protein arginine methyltransferase (Type I PRMT) inhibitor, used together is greater than the sum of the effects that results from using the compounds separately.

In an embodiment, provided herein is a combination therapy comprising an effective amount of a methionine adenosyltransferase II alpha (MAT2A) inhibitor and Type I protein arginine methyltransferase (Type I PRMT) inhibitor. An “effective amount” of a combination of agents (i.e., methionine adenosyltransferase II alpha (MAT2A) inhibitor and PRMT Type I inhibitor) is an amount sufficient to provide an observable improvement over the baseline clinically observable signs and symptoms of the disorders treated with the combination.

An “oral dosage form” includes a unit dosage form prescribed or intended for oral administration.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl, pentyl, and the like. It will be recognized by a person skilled in the art that the term “alkyl” may include “alkylene” groups.

“Alkylene” means a linear saturated divalent hydrocarbon radical of one to six carbon atoms or a branched saturated divalent hydrocarbon radical of three to six carbon atoms unless otherwise stated e.g., methylene, ethylene, propylene, 1-methylpropylene, 2-methylpropylene, butylene, pentylene, and the like.

“Alkenyl” means a linear monovalent hydrocarbon radical of two to six carbon atoms or a branched monovalent hydrocarbon radical of three to six carbon atoms containing a double bond, e.g., propenyl, butenyl, and the like.

“Alkynyl” means a linear monovalent hydrocarbon radical of two to six carbon atoms or a branched monovalent hydrocarbon radical of three to six carbon atoms containing a triple bond, e.g., ethynyl, propynyl, butynyl, and the like.

“Alkoxy” means a —OR radical where R is alkyl as defined above, e.g., methoxy, ethoxy, propoxy, or 2-propoxy, n-, iso-, or tert-butoxy, and the like.

“Alkoxyalkyl” means a linear monovalent hydrocarbon radical of one to six carbon atoms or a branched monovalent hydrocarbon radical of three to six carbons substituted with one alkoxy group, as defined above, e.g., 2-methoxyethyl, 1-, 2-, or 3-methoxypropyl, 2-ethoxyethyl, and the like.

“Alkoxyalkoxy” means a —OR radical where R is alkoxyalkyl as defined above e.g., methoxyethyloxy, ethyloxypropyloxy, and the like.

“Alkoxyalkylamino” means a —NRR′ radical where R is hydrogen or alkyl and R′ is alkoxyalkyl, each as defined above e.g., methoxyethylamino, methoxypropylamino, and the like.

“Alkylcarbonyl” means a —C(O)R radical where R is alkyl as defined herein, e.g., methylcarbonyl, ethylcarbonyl, and the like.

“Alkoxycarbonyl” means a —C(O)OR radical where R is alkyl as defined above, e.g., methoxycarbonyl, ethoxycarbonyl, and the like.

“Alkoxycarboxyalkyl” means an alkyl radical as defined above, that is substituted with an alkoxycarboxy group e.g., methylcarboxymethyl, ethylcarboxyethyl, and the like. 5“Alkylthio” means a —SR radical where R is alkyl as defined above, e.g., methylthio, ethylthio, and the like.

“Alkylsulfonyl” means a —SO₂R radical where R is alkyl as defined above, e.g., methylsulfonyl, ethylsulfonyl, and the like.

“Alkylsulfonylalkyl” means a -(alkylene)-SO2R radical where R is alkyl as defined above, e.g., methylsulfonylethyl, ethylsulfonylmethyl, and the like.

“Amino” means a —NH₂.

“Alkylamino” means a —NHR radical where R is alkyl as defined above, e.g., methylamino, ethylamino, propylamino, or 2-propylamino, and the like.

“Aminoalkyl” means a linear monovalent hydrocarbon radical of one to six carbon atoms or a branched monovalent hydrocarbon radical of three to six carbons substituted with —NR′R″ where R′ and R″ are independently hydrogen, alkyl, haloalkyl, hydroxyalkyl, alkoxyalkyl, or alkylcarbonyl, each as defined herein, e.g., aminomethyl, aminoethyl, methylaminomethyl, and the like.

“Aminoalkoxy” means a —OR radical where R is aminoalkyl as defined above e.g., aminoethyloxy, methylaminopropyloxy, dimethylaminoethyloxy, diethylaminopropyloxy, and the like.

“Aminoalkylamino” means a —NRR′ radical where R is hydrogen or alkyl and R′ is aminoalkyl, each as defined above e.g., aminoethylamino, methylaminopropylamino, dimethylaminoethylamino, diethylaminopropylamino, and the like.

“Aminocarbonyl” means a —CONH₂ radical.

“Alkylaminocarbonyl” means a —CONHR radical where R is alkyl as defined above, e.g., methylaminocarbonyl, ethylaminocarbonyl and the like.

“Aminosulfonyl” means a —SO₂NH₂ radical.

“Aminosulfonylalkyl” means a -(alkylene)SO₂NRR′ radical where R is hydrogen or alkyl and R′ is hydrogen, alkyl, or cycloalkyl, or R and R′ together with the nitrogen atom to which they are attached form heterocyclyl, as defined above, e.g., methylaminosulfonylethyl, dimethylsulfonylethyl, and the like.

“Alkylaminosulfonyl” means a —SO₂NHR radical where R is alkyl as defined above, e.g., methylaminosulfonyl, ethylaminosulfonyl and the like.

“Aminocarbonylalkyl” means a -(alkylene)-CONRR′ radical where R′ and R″ are independently hydrogen, alkyl, haloalkyl, hydroxyalkyl, or alkoxyalkyl, each as defined herein, e.g., aminocarbonylethyl, methylaminocarbonylethyl, dimethylaminocarbonylethyl, and the like.

“Aminosulfonylalkyl” means a -(alkylene)-SO₂NRR′ radical where R′ and R″ are independently hydrogen, alkyl, haloalkyl, hydroxyalkyl, or alkoxyalkyl, each as defined herein, e.g., aminosulfonylethyl, methylaminosulfonylethyl, dimethylaminosulfonylethyl, and the like.

“Aryl” means a monovalent monocyclic or bicyclic aromatic hydrocarbon radical of 6 to 10 ring atoms e.g., phenyl or naphthyl.

“Aralkyl” means a -(alkylene)-R radical where R is aryl as defined above e.g., benzyl, phenethyl, and the like.

“Bridged cycloalkyl” means a saturated monocyclic 5- to 7-membered hydrocarbon radical in which two non-adjacent ring atoms are linked by a (CRR′)ₙ group where n is 1 to 3 and each R is independently H or methyl (also referred to herein as the bridging group). The bridged cycloalkyl is optionally substituted with one or two substituents independently selected from alkyl, halo, alkoxy, hydroxy, or cyano. Examples of bridged cycloalkyl include but are not limited to bicyclo[2.2.1]heptane, bicyclo[2.2.2]octane, etc.

“Bridged cycloalkylalkyl” means -(alkylnene)-R radical where R is bridged cycloalkyl as defined above. Examples include, but are not limited to, bicyclo[2.2.1]heptylmethyl, and the like.

“Bridged heterocyclyl” means a saturated monocyclic ring having 5 to 7 ring carbon ring atoms in which two non-adjacent ring atoms are linked by a (CRR′)ₙ group where n is 1 to 3 and each R is independently H or methyl (also may be referred to herein as “bridging” group) and further wherein one or two ring carbon atoms, including an atom in the bridging group, is replaced by a heteroatom selected from N, O, or S(O)ₙ, where n is an integer from 0 to 2. Bridged heterocyclyl is optionally substituted with one or two substituents independently selected from alkyl, halo, alkoxy, hydroxy, or cyano. Examples include, but are not limited to, 2-azabicyclo[2.2.2]octane, quinuclidine, 7-oxabicyclo[2.2.1]heptane, and the like.

“Bridged heterocyclylalkyl” means -(alkylene)-R radical where R is bridged heterocyclyl (including specific bridged heterocyclyl rings) as defined above.

“Cycloalkyl” means a monocyclic monovalent hydrocarbon radical of three to six carbon atoms which may be saturated or contains one double bond. Cycloalkyl may be unsubstituted or substituted with one or two substituents independently selected from alkyl, halo, alkoxy, hydroxy, or cyano. Examples include, but are not limited to, cyclopropyl, cyclobutyl, cyclopentyl, cyclohexyl, 1-cyanocycloprop-1-yl, 1-cyanomethylcycloprop-1-yl, 3-fluorocyclohexyl, and the like. When cycloalkyl contains a double bond, it may be referred to herein as cycloalkenyl.

“Cycloalkylalkyl” means -(alkylene)-R radical where R is cycloalkyl as defined above. Examples include, but are not limited to, cyclopropylmethyl, cyclobutylmethyl, and the like.

“Cycloalkylalkyloxy” means —O—R radical where R is cycloalkylalkyl as defined above. Examples include, but are not limited to, cyclopropylmethyloxy, cyclobutylmethyloxy, and the like.

“Cycloalkyloxyalkyl” means -(alkylene)-OR radical where R is cycloalkyl as defined above. Examples include, but are not limited to, cyclopropyloxymethyl, cyclopropyloxyethyl, cyclobutyloxyethyl, and the like.

“Cycloalkylsulfonylamino” means —NRSO₂—R′ radical where R is hydrogen or alkyl and R′ is cycloalkyl, each as defined above. Examples include, but are not limited to, cyclopropylsulfonylamino, N-cyclopropylsulfonylN(CH₃), and the like.

“Cyanoalkyl” means an alkyl radical as defined above, that is substituted with a cyano group, e.g., cyanomethyl, cyanoethyl, and the like.

“Carboxy” means —COOH radical.

“Carboxyalkyl” means an alkyl radical as defined above, that is substituted with a carboxy group e.g., carboxymethyl, carboxyethyl, and the like.

“Deuteroalkyl” means alkyl radical, as defined above, wherein one to six hydrogen atoms in alkyl chain are replaced by deuterium atoms. Examples include, but are not limited to, -CD₃, —CH₂CHD₂, and the like.

“Dialkylamino” means a —NRR′ radical where R and R′ are alkyl as defined above, e.g., dimethylamino, methylethylamino, and the like.

“Dialkylaminocarbonyl” means a —CONRR′ radical where R and R′ are alkyl as defined above, e.g., dimethylaminocarbonyl, diethylaminocarbonyl and the like.

“Dialkylaminosulfonyl” means a —SO₂NRR′ radical where R and R′ are alkyl as defined above, e.g., dimethylaminosulfonyl, diethylaminosulfonyl and the like.

“Fused cycloalkyl” means a saturated monovalent hydrocarbon radical of three to six carbon atoms that is fused to phenyl or a five- or six-membered heteroaryl ring, as defined herein, and is optionally substituted with one, two, or three substituents independently selected from alkyl, halo, alkoxy, haloalkyl, haloalkoxy, hydroxy, and cyano. Examples include, but are not limited to, tetrahydronaphthyl, 4,5,6,7-tetrahydro-1H-indolyl, 4,5,6,7-tetrahydrobenzoxazolyl, and the like.

“Fused heterocyclyl” means heterocyclyl as defined herein that is fused to cycloalkyl, phenyl or a five- or six-membered heteroaryl ring, as defined herein. Fused heterocyclyl is optionally substituted with one or two substituents independently selected from alkyl, halo, alkoxy, hydroxy, or cyano. Examples include, but are not limited to, 4,5,6,7-tetrahydro-1H-pyrrolo[2,3-b]pyridinyl, 1,2,3,4-tetrahydroquinolinyl, 3,4-dihydroquinolin-2(1H)-one, and the like.

“Fused heterocyclylalkyl” means -(alkylene)-R radical where R is fused heterocyclyloxy (including specific fused heterocyclyl rings) as defined above.

“Halo” means fluoro, chloro, bromo, or iodo, preferably fluoro or chloro.

“Haloalkyl” means alkyl radical as defined above, which is substituted with one to five halogen atoms, such as fluorine or chlorine, including those substituted with different halogens, e.g., —CH₂Cl, —CF₃, —CHF₂, —CH₂CF₃, —CF₂CF₃, —CF(CH₃)₂, and the like. When the alkyl is substituted with only fluoro, it can be referred to as fluoroalkyl.

“Haloalkoxy” means a —OR radical where R is haloalkyl as defined above e.g., —OCF₃, —OCHF₂, and the like. When R is haloalkyl where the alkyl is substituted with only fluoro, it is referred to as fluoroalkoxy.

“Haloalkoxyalkyl” means an alkyl radical that is substituted with haloalkoxy, each as defined above, e.g., trifluoromethoxyethyl, and the like.

“Heteroalkylene” means a linear saturated divalent hydrocarbon radical of two to six carbon atoms or a branched saturated divalent hydrocarbon radical of three to six carbon atoms wherein one carbon atom are replaced with —O—, —NR—, —NR′CO—, —CONR′—, SO2NR′—, or —NR′SO2-, where R and R′ are independently H or alkyl as defined herein, unless stated otherwise, e.g., —CH₂O—, —OCH₂—, —(CH₂)₂O—, —O(CH₂)₂—, —(CH₂)₂NH—, —NH(CH₂)₂—, and the like.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxy-ethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Hydroxyalkoxy” means a —OR radical where R is hydroxyalkyl as defined above e.g., hydroxyethyloxy, hydroxypropyloxy, and the like.

“Hydroxyalkylamino” means a —NRR′ radical where R is hydrogen or alkyl and R′ is hydroxyalkyl, each as defined above e.g., hydroxyethylamino, hydroxypropylamino, and the like.

“Heteroaryl” means a monovalent monocyclic or bicyclic aromatic radical of 5 to 10 ring atoms, unless otherwise stated, where one or more, (in one embodiment, one, two, or three), ring atoms are heteroatom selected from N, O, or S, the remaining ring atoms being carbon. Non-limiting examples of heteroaryl groups include pyridyl, pyridazinyl, pyrazinyl, pyrimindinyl, triazinyl, quinolinyl, quinoxalinyl, quinazolinyl, cinnolinyl, phthalazinyl, benzotriazinyl, purinyl, benzimidazolyl, benzopyrazolyl, benzotriazolyl, benzisoxazolyl, isobenzofuryl, isoindolyl, indolizinyl, benzotriazinyl, thienopyridinyl, thienopyrimidinyl, pyrazolopyrimidinyl, imidazopyridines, benzothiaxolyl, benzofuranyl, benzothienyl, indolyl, quinolyl, isoquinolyl, isothiazolyl, pyrazolyl, indazolyl, pteridinyl, imidazolyl, triazolyl, tetrazolyl, oxazolyl, isoxazolyl, thiadiazolyl, pyrrolyl, thiazolyl, furyl, thienyl, and the like. As defined herein, the terms “heteroaryl” and “aryl” are mutually exclusive. When the heteroaryl ring contains 5- or 6 ring atoms it is also referred to herein as 5- or 6-membered heteroaryl.

“Heteroaralkyl” means a -(alkylene)-R radical where R is heteroaryl (including specific rings) as defined above.

“Heteroaryloxy” means —OR where R is heteroaryl (including specific rings) as defined above.

“Heteroaralkyloxy” means a —O-(alkylene)-R radical where R is heteroaryl (including specific rings) as defined above.

“Heteroarylcarbonyl” means —COR where R is heteroaryl (including specific rings) as defined above.

“Heteroarylamino” means —NRR′ where R is hydrogen or alkyl and R′ is heteroaryl (including specific rings) as defined above.

“Heterocyclyl” means a saturated or unsaturated monovalent monocyclic group of 4 to 8 ring atoms in which one or two ring atoms are heteroatom selected from N, O, or S(O)ₙ, where n is an integer from 0 to 2, the remaining ring atoms being C. Additionally, one or two ring carbon atoms in the heterocyclyl ring can optionally be replaced by a —CO— group. More specifically the term heterocyclyl includes, but is not limited to, azetidinyl, oxetanyl, pyrrolidino, piperidino, homopiperidino, 2-oxopyrrolidinyl, 2-oxopiperidinyl, morpholino, piperazino, tetrahydro-pyranyl, thiomorpholino, and the like. When the heterocyclyl ring is unsaturated it can contain one or two ring double bonds provided that the ring is not aromatic. When heterocyclyl contains at least one nitrogen atom, it may be referred to herein as heterocycloamino.

“Heterocyclylalkyl” means -(alkylene)- R radical where R is heterocyclyl (including specific heterocyclyl rings) as defined above. For example, oxetanylethyl, piperidinylethyl, and the like.

“Heterocyclyloxy” means —OR radical where R is heterocyclyl (including specific heterocyclyl rings) as defined above.

“Heterocyclylalkyloxy” means —O-(alkylene)-R radical where R is heterocyclyl (including specific heterocyclyl rings) as defined above. For example, oxetanylethyloxy, piperidinylethyloxy, and the like.

“Heterocyclylcarbonyl” means —COR where R is heterocyclyl (including specific rings) as defined above.

“Heterocyclylamino” means —NRR′ radical where R is hydrogen or alkyl and R′ is heterocyclyl (including specific heterocyclyl rings) as defined above.

“Heterocyclyloxyalkyl” means -(alkylene)-OR radical where R is heterocyclyl (including specific heterocyclyl rings) as defined above. For example, oxetanyloxyethyl, piperidinyloxyethyl, and the like.

“Heterocyclyloxyalkoxy” means —O-(alkylene)-R radical where R is heterocyclyloxy (including specific heterocyclyl rings) as defined above. For example, oxetanyloxyethyloxy, piperidinyloxyethyloxy, and the like.

“Heterocyclyloxyalkylamino” means —NR-(alkylene)-R′ radical where R is hydrogen or alkyl and R′ is heterocyclyloxy (including specific heterocyclyl rings) as defined above. For example, oxetanyloxyethylamino, piperidinyloxyethylamino, and the like.

“Oxo,” as used herein, alone or in combination, refers to ═(O).

“Optionally substituted aryl” means aryl that is optionally substituted with one, two, or three substituents independently selected from alkyl, cycloalkyl, carboxy, alkoxycarbonyl, hydroxy, hydroxyalkyl, alkoxy, alkylsulfonyl, amino, alkylamino, dialkylamino, halo, haloalkyl, haloalkoxy, and cyano.

“Optionally substituted heteroaryl” means heteroaryl as defined above that is optionally substituted with one, two, or three substituents independently selected from alkyl, alkylsulfonyl, cycloalkyl, carboxy, alkoxycarbonyl, hydroxy, alkoxy, halo, haloalkyl, haloalkoxy, amino, alkylamino, dialkylamino, and cyano.

“Optionally substituted heterocyclyl” means heterocyclyl as defined above that is optionally substituted with one, two, or three substituents independently selected from alkyl, alkylsulfonyl, cycloalkyl, carboxy, alkoxycarbonyl, hydroxy, hydroxyalkyl, alkoxy, alkoxyalkyl, aminoalkyl, halo, haloalkyl, haloalkoxy, and cyano, unless stated otherwise.

“Spirocycloalkyl” means a saturated bicyclic ring having 6 to 10 ring carbon atoms wherein the rings are connected through only one atom, the connecting atom is also called the spiroatom, most often a quaternary carbon (“spiro carbon”). The spirocycloalkyl ring is optionally substituted with one or two substituents independently selected from alkyl, halo, alkoxy, hydroxy, and cyano. Representative examples include, but are not limited to, spiro[3.3]heptane, spiro[3.4]octane, spiro[3.5]nonane, spiro[4.4]nonane (1:2:1:1), and the like.

“Spirocycloalkylalkyl” means -(alkylene)-R radical where R is spirocycloalkyl (including specific spirocycloalkyl) as defined above.

“Spiroheterocyclyl” means a saturated bicyclic ring having 6 to 10 ring atoms in which one, two, or three ring atoms are heteroatom selected from N, O, or S(O)ₙ, where n is an integer from 0 to 2, the remaining ring atoms being C and the rings are connected through only one atom, the connecting atom is also called the spiroatom, most often a quaternary carbon (“spiro carbon”). Spiroheterocyclyl is optionally substituted with one or two substituents independently selected from alkyl, halo, alkoxy, hydroxy, or cyano. Examples include, but are not limited to, Representative examples include, but are not limited to, 2,6-diazaspiro[3.3]heptane, 2,6-diazaspiro[3.4]octane, 2-azaspiro[3.4]octane, 2-azaspiro[3.5]-nonane, 2,7-diazaspiro[4.4]nonane, and the like.

“Spiroheterocyclylalkyl” means -(alkylene)-R radical where R is spiroheterocyclyl (including specific spiroheterocyclyl) as defined above.

“Sulfonylamino” means a —NRSO₂R′ radical where R is hydrogen or alkyl, and R′ is alkyl, optionally substituted aryl, optionally substituted heteroaryl, or optionally substituted heterocyclyl, each group as defined herein.

“Substituted cycloalkyl” means a saturated monocyclic monovalent hydrocarbon radical of three to six carbon atoms that is substituted with one, two or three substituents where two of the three substitutents are independently selected from alkyl, halo, alkoxy, hydroxy, haloalkyl, or haloalkoxy and the third substituent is alkyl, halo, hydroxyalkyl, haloalkyl, haloalkoxy, or cyano. Examples include, but are not limited to, 3-hydroxy-3-trifluorocyclobutyl, 2,2-dimethyl-3-hydroxycyclobutyl, and the like.

“Substituted cycloalkylalkyl” means -(alkylene)-substituted cycloalkyl, each term is defined herein. Examples include, but are not limited to, 1-hydroxymethylcycloprop-1-ylmethyl, and the like.

“Ureido” means a —NHCONRR′ radical where R and R′ are independently hydrogen or alkyl, as defined above, e.g., —NHCONHmethyl, —NHCON(CH₃)₂, and the like.

“Thioureidoalkyl” means a -(alkylene)-NHSO₂NRR′ radical where R and R′ are independently hydrogen or alkyl, as defined above, e.g., -ethylene-NHSO₂NHmethyl, -propylene-NHSO₂NH₂, and the like.

### Combination Product

Provided herein is a combination product comprising a methionine adenosyltransferase II alpha (MAT2A) inhibitor, or a pharmaceutically acceptable salt thereof, and Type I protein arginine methyltransferase (Type I PRMT) inhibitor, or a pharmaceutically acceptable salt thereof. The combination product is useful for the treatment of a variety of cancers, including MTAP-null cancers. In another aspect, the combination product is useful for the treatment of any number of MAT2A-associated and/or PRMT-associated diseases.

In an embodiment, the methionine adenosyltransferase II alpha (MAT2A) inhibitor is a compound of Formula I:

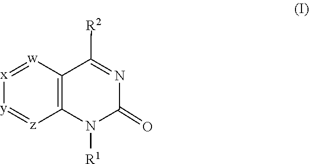

or a pharmaceutically acceptable salt thereof;

wherein
w is CR³ or N; x is CR⁴ or N; y is CR⁵ or N; and z is CR⁶ or N, wherein:
R³ is hydrogen, alkyl, alkenyl, alkynyl, alkoxy, alkylsulfonyl, halo, haloalkyl, haloalkoxy, cycloalkyl, cycloalkylalkyloxy, cyano, amino, alkylamino, dialkylamino, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroaralkyloxy, heteroarylamino, heterocyclyl, heterocyclyloxy, heterocyclylamino, heterocyclylalkyloxy, heterocyclyloxyalkoxy, or heterocyclyloxyalkylamino, wherein heterocyclyl or heteroaryl, by itself or as part of another group, is unsubstituted or substituted with R^{a}, R^{b}, or R^{c} independently selected from alkyl, cycloalkyl, haloalkyl, haloalkoxy, alkoxy, hydroxy, halo, cyano, alkoxycarbonyl, hydroxyalkyl, alkoxyalkyl, or aminoalkyl;
R⁵ is hydrogen, alkyl, alkenyl, alkynyl, alkoxy, alkylsulfonyl, halo, haloalkyl, haloalkoxy, cycloalkyl, cyano, amino, alkylamino, dialkylamino, alkoxycarbonyl, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroarylamino, heterocyclyl, heterocyclyloxy, heterocyclylamino, heterocyclyloxyalkoxy, or heterocyclyloxyalkylamino, wherein heterocyclyl or heteroaryl, by itself or as part of another group, is unsubstituted or substituted with R^{a}, R^{b}, or R^{c} independently selected from alkyl, cycloalkyl, haloalkyl, haloalkoxy, alkoxy, hydroxy, halo, cyano, hydroxyalkyl, alkoxyalkyl, or aminoalkyl;
R⁴ and R⁶ are independently hydrogen, alkyl, alkenyl, alkynyl, alkoxy, alkylthio, alkylsulfonyl, halo, haloalkyl, haloalkoxy, cycloalkyl, cyano, amino, alkylamino, dialkylamino, aminocarbonyl, alkylaminocarbonyl, or dialkylaminocarbonyl; provided that: (i) no more than two of w, x, y, and z can be N and (ii) at least one of R³, R⁴, R⁵, and R⁶ is other than hydrogen;
R¹ is R⁷ wherein R⁷ is cycloalkyl, bridged cycloalkyl, fused cycloalkyl, spirocycloalkyl, aryl, heteroaryl, heterocyclyl, bridged heterocyclyl, fused heterocyclyl, or spiroheterocyclyl, wherein aryl, heteroaryl, or heterocyclyl is unsubstituted or substituted with R^{d}, R^{e}, or R^{f};
R² is alkyl, haloalkyl, hydroxyalkyl, alkoxyalkyl, aminoalkyl, aminocarbonylalkyl, aminosulfonylalkyl, —O—R⁸, —NR⁹R¹⁰, or —X^{b}—R¹¹ wherein:
R⁸ is alkyl, haloalkyl, hydroxyalkyl, alkoxyalkyl, aminoalkyl, cycloalkyl, cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heterocyclyl, heterocyclylalkyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{g}, R^{h}, or Rⁱ;
R⁹ is hydrogen, alkyl, deuteroalkyl, or cycloalkyl; and
R¹⁰ is hydrogen, alkyl, deuteroalkyl, haloalkyl, hydroxyalkyl, alkoxyalkyl, haloalkoxyalkyl, aminoalkyl, aminosulfonylalkyl, thioureidoalkyl, alkylsulfonyl, alkylsulfonylalkyl, cyanoalkyl, alkylcarbonyl, alkoxycarbonyl, alkylaminocarbonyl, dialkylaminocarbonyl, aminocarbonylalkyl, cycloalkyl, cycloalkylalkyl, substituted cycloalkyl, substituted cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heteroarylcarbonyl, heterocyclyl, heterocyclylalkyl, heterocyclylcarbonyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{j}, R^{k}, or R^{l};
X^{b} is a bond or alkylene; and
R¹¹ is cycloalkyl, bridged cycloalkyl, fused cycloalkyl, spirocycloalkyl, heteroaryl, heterocyclyl, bridged heterocyclyl, fused heterocyclyl, or spiroheterocyclyl, wherein heteroaryl or heterocyclyl is unsubstituted or substituted with R^{m}, Rⁿ, or R^{o}; and
R^{d}, R^{e}, R^{g}, R^{h}, Rⁱ, R^{j}, R^{m}, and Rⁿ are independently selected from alkyl, haloalkyl, haloalkoxy, alkoxy, hydroxy, alkylsulfonyl, halo, cyano, carboxy, alkoxycarbonyl, hydroxyalkyl, alkoxyalkyl, aminoalkyl, aminosulfonyl, alkylaminosulfonyl, dialkylaminosulfonyl, sulfonylamino, aminocarbonyl, alkylaminocarbonyl, dialkylaminocarbonyl, heterocyclylcarbonyl, and ureido; and
R^{f}, Rⁱ, R^{l}, and R^{o} are independently selected from alkyl, cycloalkyl, haloalkyl, haloalkoxy, alkoxy, hydroxy, halo, amino, alkylamino, cycloalkylsulfonylamino, cyano, cyanoalkyl, alkoxycarbonylalkyl, carboxyalkyl, aminocarbonylalkyl, or —X^{c}—R¹² where X^{c} is bond, alkylene, or heteroalkylene and R¹² is optionally substituted aryl, optionally substituted heteroaryl, and optionally substituted heterocyclyl; provided that when R¹ is heterocyclyl then R^{f} is not hydroxy;
provided that:
(1) when

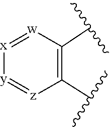

is

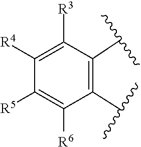

where: (a) when R² is piperazin-1-yl, 2-methylpiperazin-1-yl, or 1H-benzo[d][1,2,3]triazol-1-yl, R³ and R⁶ are hydrogen, R⁴ is chloro and R⁵ is bromo or 5-methylindazol-4-yl, then R¹ is not 2-isopropylphenyl; (b) when R² and R⁶ are methyl and R³, R⁴, and R⁵ are hydrogen; or R² and R³ are methyl and R⁴, R¹, and R⁶ are hydrogen, then R¹ is not 2,5-, 2,6- or 2,8-dimethylquinolin-4-yl or 2-methyl-5-methoxy-, 2-methyl-6-methoxy- or 2-methyl-8-methoxyquinolin-4-yl; (c) when R² is amino or acetylamino, R⁴ is dimethylamino, and R³, R⁵, and R⁶ are hydrogen, then R¹ is not 4-hydroxy-5-hydroxymethyl-tetrahydrofuran-2-yl; (d) when R⁵ is fluoro, R³, R⁴ and R⁶ are hydrogen, and R² is 4-aminocarbonylmethyl-2-methylphenylamino, then R¹ is not 4-fluoro-2-(2-thiazol-2-ylmethoxy)phenyl, 4-fluoro-2-(2-pyridin-2-ylmethoxy)phenyl, or 4-chloro-2-methoxyphenyl; (e) when R⁶ is fluoro, R³, R⁴ and R⁵ are hydrogen, and R² is 4-aminocarbonylmethyl-2-methylphenylamino, then R¹ is not 4-fluoro-2-methoxyphenyl; (f) when R¹ is 4-chloro-2-ethoxyphenyl, R⁵ is fluoro, and R³, R⁴ and R⁶ are hydrogen, then R² is not 3-(2-oxoimidazolidin-1-yl)-2-methylphenylamino;
(2) when

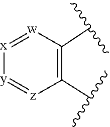

is

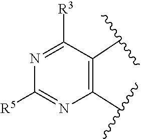

then when R¹ is 4-hydroxy-5-hydroxymethylfuran-1-yl, R⁵ is amino, and R³ is methoxy; then R² is not amino; and
(3) when

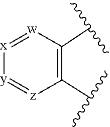

is

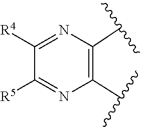

then when R¹ is 4-hydroxy-5-hydroxymethylfuran-1-yl, one of R⁴ and R⁵ is hydrogen, and the other of R⁴ and R⁵ is methyl or both of R⁴ and R⁵ are methyl, then R² is not amino.

In some embodiments, the compound of Formula (I), where:
w is CR³ or N; x is CR⁴ or N; y is CR⁵ or N; and z is CR⁶ or N, provided that no more than two of w, x, y, and z can be N, wherein:
R³ is hydrogen, alkyl, alkenyl, alkynyl, alkoxy, alkylsulfonyl, halo, haloalkyl, haloalkoxy, cycloalkyl, cycloalkylalkyloxy, cyano, amino, alkylamino, dialkylamino, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroaralkyloxy, heteroarylamino, heterocyclyl, heterocyclyloxy, heterocyclylamino, heterocyclylalkyloxy, heterocyclyloxyalkoxy, or heterocyclyloxyalkylamino, wherein heterocyclyl or heteroaryl, by itself or as part of another group, is unsubstituted or substituted with R^{a}, R^{b}, and/or R^{c} independently selected from alkyl, cycloalkyl, haloalkyl, haloalkoxy, alkoxy, hydroxy, halo, cyano, alkoxycarbonyl, hydroxyalkyl, alkoxyalkyl, or aminoalkyl;
R⁵ is alkyl, alkenyl, alkynyl, alkoxy, alkylsulfonyl, halo, haloalkyl, haloalkoxy, cycloalkyl, cyano, amino, alkylamino, dialkylamino, alkoxycarbonyl, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroarylamino, heterocyclyl, heterocyclyloxy, heterocyclylamino, heterocyclyloxyalkoxy, or heterocyclyloxyalkylamino, wherein heterocyclyl or heteroaryl, by itself or as part of another group, is unsubstituted or substituted with R^{a}, R^{b}, and/or R^{c} independently selected from alkyl, cycloalkyl, haloalkyl, haloalkoxy, alkoxy, hydroxy, halo, cyano, hydroxyalkyl, alkoxyalkyl, or aminoalkyl;
R⁴ and R⁶ are independently hydrogen, alkyl, alkenyl, alkynyl, alkoxy, alkylthio, alkylsulfonyl, halo, haloalkyl, haloalkoxy, cycloalkyl, cyano, amino, alkylamino, dialkylamino, aminocarbonyl, alkylaminocarbonyl, or dialkylaminocarbonyl;
R¹ is R⁷ wherein R⁷ is cycloalkyl, bridged cycloalkyl, fused cycloalkyl, spirocycloalkyl, aryl, heteroaryl, pyrrolidinyl, piperidinyl, piperazinyl, oxetanyl, morpholinyl, bridged heterocyclyl, fused heterocyclyl, or spiroheterocyclyl, wherein aryl, heteroaryl, or heterocyclyl is unsubstituted or substituted with R^{d}, R^{e}, and/or R^{f};
R² is alkyl, haloalkyl, hydroxyalkyl, alkoxyalkyl, aminoalkyl, aminocarbonylalkyl, aminosulfonylalkyl, —O—R⁸, —NR⁹R¹⁰, or —X^{b}—R¹¹ wherein:
R⁸ is alkyl, haloalkyl, hydroxyalkyl, alkoxyalkyl, aminoalkyl, cycloalkyl, cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heterocyclyl, heterocyclylalkyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{g}, R^{h}, and/or Rⁱ;
R⁹ is hydrogen, alkyl, deuteroalkyl, or cycloalkyl; and
R¹⁰ is hydrogen, alkyl, deuteroalkyl, haloalkyl, hydroxyalkyl, alkoxyalkyl, haloalkoxyalkyl, aminoalkyl, aminosulfonylalkyl, thioureidoalkyl, alkylsulfonyl, alkylsulfonylalkyl, cyanoalkyl, alkylcarbonyl, alkoxycarbonyl, alkylaminocarbonyl, dialkylaminocarbonyl, aminocarbonylalkyl, cycloalkyl, cycloalkylalkyl, substituted cycloalkyl, substituted cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heteroarylcarbonyl, heterocyclyl, heterocyclylalkyl, heterocyclylcarbonyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{j}, R^{k}, and/or R^{l};
X^{b} is a bond or alkylene; and
R¹¹ is cycloalkyl, bridged cycloalkyl, fused cycloalkyl, spirocycloalkyl, monocyclic heteroaryl, oxetanyl, azetidinyl, 2-oxoazetidinyl, pyrrolidinyl, 2-oxopyrrolidinyl, piperidinyl, or morpholinyl, bridged heterocyclyl, fused heterocyclyl, or spiroheterocyclyl, wherein heteroaryl or heterocyclyl is unsubstituted or substituted with R^{m}, Rⁿ, and/or R^{o}; and
R^{d}, R^{e}, R^{g}, R^{h}, Rⁱ, R^{k}, R^{m}, and Rⁿ are independently selected from alkyl, haloalkyl, haloalkoxy, alkoxy, hydroxy, alkylsulfonyl, halo, cyano, carboxy, alkoxycarbonyl, hydroxyalkyl, alkoxyalkyl, aminoalkyl, aminosulfonyl, alkylaminosulfonyl, dialkylaminosulfonyl, sulfonylamino, aminocarbonyl, alkylaminocarbonyl, dialkylaminocarbonyl, heterocyclylcarbonyl, and ureido; and
R^{f}, Rⁱ, R^{j}, and R^{o} are independently selected from alkyl, cycloalkyl, haloalkyl, haloalkoxy, alkoxy, hydroxy, halo, amino, alkylamino, cycloalkylsulfonylamino, cyano, cyanoalkyl, alkoxycarbonylalkyl, carboxyalkyl, or —X^{c}—R¹² where X^{c} is bond, alkylene, or heteroalkylene and R¹² is optionally substituted aryl, or optionally substituted heteroaryl; provided that when R¹ is pyrrolidinyl, piperidinyl, piperazinyl, oxetanyl, or morpholinyl then R^{f} is not hydroxy; or
a pharmaceutically acceptable salt thereof.

In another embodiment,
R⁴ and R⁶ are independently hydrogen, alkyl, alkenyl, alkynyl, alkoxy, alkylsulfonyl, halo, haloalkyl, haloalkoxy, cycloalkyl, cyano, amino, alkylamino, dialkylamino, aminocarbonyl, alkylaminocarbonyl, or dialkylaminocarbonyl; provided that: (i) no more than two of w, x, y, and z can be N and (ii) at least one of R³, R⁴, R⁵, and R⁶ is other than hydrogen;
R¹⁰ is hydrogen, alkyl, deuteroalkyl, haloalkyl, hydroxyalkyl, alkoxyalkyl, aminoalkyl, alkylsulfonyl, alkylsulfonylalkyl, cyanoalkyl, alkylcarbonyl, alkoxycarbonyl, alkylaminocarbonyl, dialkylaminocarbonyl, aminocarbonylalkyl, cycloalkyl, cycloalkylalkyl, substituted cycloalkyl, substituted cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heteroarylcarbonyl, heterocyclyl, heterocyclylalkyl, heterocyclylcarbonyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{j}, R^{k}, or R^{l}; and
R^{f}, Rⁱ, R^{l}, and R^{o} are independently selected from alkyl, cycloalkyl, haloalkyl, haloalkoxy, alkoxy, hydroxy, halo, amino, cycloalkylsulfonylamino, cyano, cyanoalkyl, alkoxycarbonylalkyl, carboxyalkyl, aminocarbonylalkyl, or —X^{c}—R¹² where X^{c} is bond, alkylene, or heteroalkylene and R¹² is optionally substituted aryl, optionally substituted heteroaryl, and optionally substituted heterocyclyl; provided that when R¹ is heterocyclyl then R^{f} is not hydroxy.

In another embodiment,
R³ is hydrogen, alkyl, alkenyl, alkynyl, alkoxy, alkylsulfonyl, halo, haloalkyl, haloalkoxy, cycloalkyl, cycloalkylalkyloxy, cyano, amino, alkylamino, dialkylamino, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroaralkyloxy, heteroarylamino, heterocyclyl, heterocyclyloxy, heterocyclylamino, heterocyclylalkyloxy, heterocyclyloxyalkoxy, or heterocyclyloxyalkylamino, wherein heterocyclyl or heteroaryl, by itself or as part of another group, is unsubstituted or substituted with R^{a}, R^{b}, or R^{c} independently selected from alkyl, cycloalkyl, haloalkyl, haloalkoxy, alkoxy, hydroxy, halo, cyano, alkoxycarbonyl, hydroxyalkyl, alkoxyalkyl, or aminoalkyl;
R⁵ is alkyl, alkenyl, alkynyl, alkoxy, alkylsulfonyl, halo, haloalkyl, haloalkoxy, cycloalkyl, cyano, amino, alkylamino, dialkylamino, alkoxycarbonyl, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroarylamino, heterocyclyl, heterocyclyloxy, heterocyclylamino, heterocyclyloxyalkoxy, or heterocyclyloxyalkylamino, wherein heterocyclyl or heteroaryl, by itself or as part of another group, is unsubstituted or substituted with R^{a}, R^{b}, and/or R^{c} independently selected from alkyl, cycloalkyl, haloalkyl, haloalkoxy, alkoxy, hydroxy, halo, cyano, hydroxyalkyl, alkoxyalkyl, or aminoalkyl;
R⁴ is hydrogen, alkyl, alkenyl, alkynyl, alkoxy, alkylthio, alkylsulfonyl, halo, haloalkyl, haloalkoxy, cycloalkyl, cyano, amino, alkylamino, dialkylamino, aminocarbonyl, alkylaminocarbonyl, or dialkylaminocarbonyl;
R¹ is R⁷ wherein R⁷ is cycloalkyl, bridged cycloalkyl, fused cycloalkyl, spirocycloalkyl, aryl, heteroaryl, pyrrolidinyl, piperidinyl, piperazinyl, oxetanyl, morpholinyl, bridged heterocyclyl, fused heterocyclyl, or spiroheterocyclyl, wherein aryl, heteroaryl, or heterocyclyl is unsubstituted or substituted with R^{d}, R^{e}, and/or R^{f};
R² is alkyl, haloalkyl, hydroxyalkyl, alkoxyalkyl, aminoalkyl, aminocarbonylalkyl, aminosulfonylalkyl, —O—R⁸, —NR⁹R¹⁰, wherein:
R⁸ is alkyl, haloalkyl, hydroxyalkyl, alkoxyalkyl, aminoalkyl, cycloalkyl, cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heterocyclyl, heterocyclylalkyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{g}, R^{h}, or Rⁱ;
R⁹ is hydrogen, alkyl, deuteroalkyl, or cycloalkyl; and
R¹⁰ is hydrogen, alkyl, deuteroalkyl, haloalkyl, hydroxyalkyl, alkoxyalkyl, haloalkoxyalkyl, aminoalkyl, aminosulfonylalkyl, thioureidoalkyl, alkylsulfonyl, alkylsulfonylalkyl, cyanoalkyl, alkylcarbonyl, alkoxycarbonyl, alkylaminocarbonyl, dialkylaminocarbonyl, aminocarbonylalkyl, cycloalkyl, cycloalkylalkyl, substituted cycloalkyl, substituted cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heteroarylcarbonyl, heterocyclyl, heterocyclylalkyl, heterocyclylcarbonyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{j}, R^{k}, or R^{l};
R^{d}, R^{e}, R^{g}, R^{h}, R^{j}, R^{k}, R^{m}, and Rⁿ are independently selected from alkyl, haloalkyl, haloalkoxy, alkoxy, hydroxy, alkylsulfonyl, halo, cyano, carboxy, alkoxycarbonyl, hydroxyalkyl, alkoxyalkyl, aminoalkyl, aminosulfonyl, alkylaminosulfonyl, dialkylaminosulfonyl, sulfonylamino, aminocarbonyl, alkylaminocarbonyl, dialkylaminocarbonyl, heterocyclylcarbonyl, and ureido; and
R^{f}, Rⁱ, R^{l}, and R^{o} are independently selected from alkyl, cycloalkyl, haloalkyl, haloalkoxy, alkoxy, hydroxy, halo, amino, alkylamino, cycloalkylsulfonylamino, cyano, cyanoalkyl, alkoxycarbonylalkyl, carboxyalkyl, or —X^{c}—R¹² where X^{c} is bond, alkylene, or heteroalkylene and R¹² is optionally substituted aryl, or optionally substituted heteroaryl; provided that when R¹ is pyrrolidinyl, piperidinyl, piperazinyl, oxetanyl, or morpholinyl then R^{f} is not hydroxy; or a pharmaceutically acceptable salt thereof.

In yet another embodiment,
R³ and R⁵ are independently hydrogen, alkyl, alkenyl, alkynyl, alkoxy, alkylsulfonyl, halo, haloalkyl, haloalkoxy, cycloalkyl, cyano, amino, alkylamino, dialkylamino, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroarylamino, heterocyclyl, heterocyclyloxy, heterocyclylamino, heterocyclyloxyalkoxy, or heterocyclyloxyalkylamino, wherein heterocyclyl or heteroaryl, by itself or as part of another group, is unsubstituted or substituted with R^{a}, R^{b}, or R^{c} independently selected from alkyl, cycloalkyl, haloalkyl, haloalkoxy, alkoxy, hydroxy, halo, cyano, hydroxyalkyl, alkoxyalkyl, or aminoalkyl;
R¹ is R⁷ wherein R⁷ is cycloalkyl, bridged cycloalkyl, fused cycloalkyl, spirocycloalkyl, phenyl, heteroaryl, heterocyclyl, bridged heterocyclyl, fused heterocyclyl, or spiroheterocyclyl, wherein phenyl, heteroaryl, or heterocyclyl is unsubstituted or substituted with R^{d}, R^{e}, or R^{f};
R⁹ is hydrogen, alkyl or cycloalkyl;
R¹⁰ is hydrogen, alkyl, haloalkyl, hydroxyalkyl, alkoxyalkyl, aminoalkyl, alkylcarbonyl, alkoxycarbonyl, alkylaminocarbonyl, dialkylaminocarbonyl, aminocarbonylalkyl, cycloalkyl, cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heteroarylcarbonyl, heterocyclyl, heterocyclylalkyl, heterocyclylcarbonyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{j}, R^{k}, or R^{l};
R^{d}, R^{e}, R^{g}, R^{h}, R^{j}, R^{k}, R^{m}, and Rⁿ are independently selected from alkyl, haloalkyl, haloalkoxy, alkoxy, alkylsulfonyl, halo, cyano, carboxy, alkoxycarbonyl, hydroxyalkyl, alkoxyalkyl, aminoalkyl, aminosulfonyl, alkylaminosulfonyl, dialkylaminosulfonyl, sulfonylamino, aminocarbonyl, alkylaminocarbonyl, dialkylaminocarbonyl, heterocyclylcarbonyl, and ureido; and
R^{f}, Rⁱ, R^{l}, and R^{o} are independently selected from alkyl, haloalkyl, haloalkoxy, alkoxy, hydroxy, halo, cyano, or —X^{c}—R¹² where X^{c} is bond, alkylene or heteroalkylene and R¹² is optionally substituted aryl, optionally substituted heteroaryl, or optionally substituted heterocyclyl.

In still another embodiment, the methionine adenosyltransferase II alpha (MAT2A) inhibitor is a compound of Formula (IIIa), (IIIb), (IIIc), (IIId), (IIIe), or (IIIg):

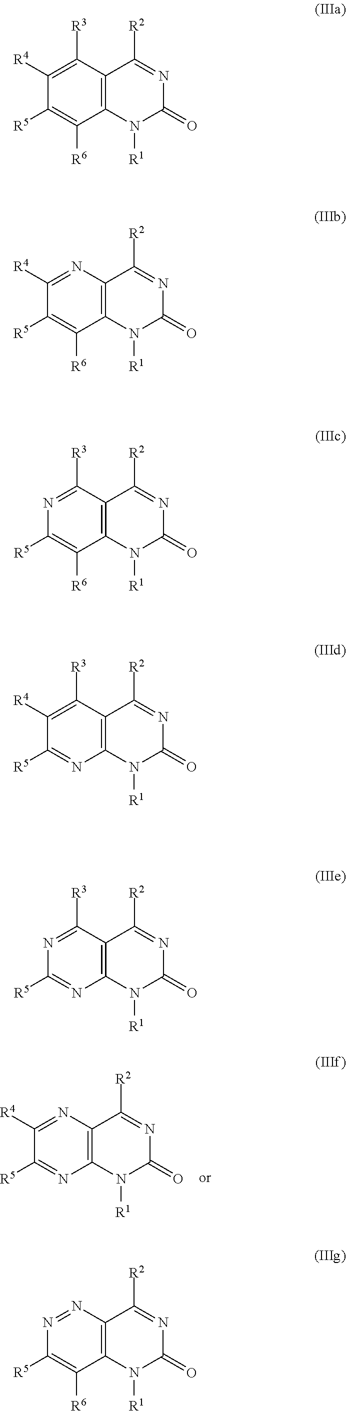

or a pharmaceutically acceptable salt thereof.

In an embodiment, the methionine adenosyltransferase II alpha (MAT2A) inhibitor is a compound of Formula IIIa, or a pharmaceutically acceptable salt thereof. In another embodiment, the methionine adenosyltransferase II alpha (MAT2A) inhibitor is a compound of Formula IIIb, or a pharmaceutically acceptable salt thereof. In yet another embodiment, the methionine adenosyltransferase II alpha (MAT2A) inhibitor is a compound of Formula IIIc, or a pharmaceutically acceptable salt thereof. In still another embodiment, the methionine adenosyltransferase II alpha (MAT2A) inhibitor is a compound of Formula IIId, or a pharmaceutically acceptable salt thereof. In an embodiment, the methionine adenosyltransferase II alpha (MAT2A) inhibitor is a compound of Formula IIIe, or a pharmaceutically acceptable salt thereof. In another embodiment, the methionine adenosyltransferase II alpha (MAT2A) inhibitor is a compound of Formula IIIf, or a pharmaceutically acceptable salt thereof. In yet another embodiment, the methionine adenosyltransferase II alpha (MAT2A) inhibitor is a compound of Formula IIIg, or a pharmaceutically acceptable salt thereof.

In an embodiment, the methionine adenosyltransferase II alpha (MAT2A) inhibitor is a compound of Formula IIId, or a pharmaceutically acceptable salt thereof, wherein
R³ is hydrogen, alkyl, alkenyl, alkynyl, alkoxy, alkylsulfonyl, halo, haloalkyl, haloalkoxy, cycloalkyl, cycloalkylalkyloxy, cyano, amino, alkylamino, dialkylamino, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroaralkyloxy, heteroarylamino, heterocyclyl, heterocyclyloxy, heterocyclylamino, heterocyclylalkyloxy, heterocyclyloxyalkoxy, or heterocyclyloxyalkylamino, wherein heterocyclyl or heteroaryl, by itself or as part of another group, is unsubstituted or substituted with R^{a}, R^{b}, or R^{c} independently selected from alkyl, cycloalkyl, haloalkyl, haloalkoxy, alkoxy, hydroxy, halo, cyano, alkoxycarbonyl, hydroxyalkyl, alkoxyalkyl, or aminoalkyl;
R⁵ is hydrogen, alkyl, alkenyl, alkynyl, alkoxy, alkylsulfonyl, halo, haloalkyl, haloalkoxy, cycloalkyl, cyano, amino, alkylamino, dialkylamino, alkoxycarbonyl, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroarylamino, heterocyclyl, heterocyclyloxy, heterocyclylamino, heterocyclyloxyalkoxy, or heterocyclyloxyalkylamino, wherein heterocyclyl or heteroaryl, by itself or as part of another group, is unsubstituted or substituted with R^{a}, R^{b}, or R^{c} independently selected from alkyl, cycloalkyl, haloalkyl, haloalkoxy, alkoxy, hydroxy, halo, cyano, hydroxyalkyl, alkoxyalkyl, or aminoalkyl;
R⁴ is hydrogen, alkyl, alkenyl, alkynyl, alkoxy, alkylthio, alkylsulfonyl, halo, haloalkyl, haloalkoxy, cycloalkyl, cyano, amino, alkylamino, dialkylamino, aminocarbonyl, alkylaminocarbonyl, or dialkylaminocarbonyl; provided that at least one of R³, R⁴, and R⁵ is other than hydrogen;
R¹ is R⁷ wherein R⁷ is cycloalkyl, bridged cycloalkyl, fused cycloalkyl, spirocycloalkyl, aryl, heteroaryl, heterocyclyl, bridged heterocyclyl, fused heterocyclyl, or spiroheterocyclyl, wherein aryl, heteroaryl, or heterocyclyl is unsubstituted or substituted with R^{d}, R^{e}, or R^{f};
R² is alkyl, haloalkyl, hydroxyalkyl, alkoxyalkyl, aminoalkyl, aminocarbonylalkyl, aminosulfonylalkyl, —O—R⁸, or —NR⁹R¹⁰ wherein:
R⁸ is alkyl, haloalkyl, hydroxyalkyl, alkoxyalkyl, aminoalkyl, cycloalkyl, cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heterocyclyl, heterocyclylalkyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{g}, R^{h}, or Rⁱ;
R⁹ is hydrogen, alkyl, deuteroalkyl, or cycloalkyl; and
R¹⁰ is hydrogen, alkyl, deuteroalkyl, haloalkyl, hydroxyalkyl, alkoxyalkyl, haloalkoxyalkyl, aminoalkyl, aminosulfonylalkyl, thioureidoalkyl, alkylsulfonyl, alkylsulfonylalkyl, cyanoalkyl, alkylcarbonyl, alkoxycarbonyl, alkylaminocarbonyl, dialkylaminocarbonyl, aminocarbonylalkyl, cycloalkyl, cycloalkylalkyl, substituted cycloalkyl, substituted cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heteroarylcarbonyl, heterocyclyl, heterocyclylalkyl, heterocyclylcarbonyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with Rⁱ, R^{k}, or R^{l};
R^{d}, R^{e}, R^{g}, R^{h}, R^{j}, R^{k}, R^{m}, and Rⁿ are independently selected from alkyl, haloalkyl, haloalkoxy, alkoxy, hydroxy, alkylsulfonyl, halo, cyano, carboxy, alkoxycarbonyl, hydroxyalkyl, alkoxyalkyl, aminoalkyl, aminosulfonyl, alkylaminosulfonyl, dialkylaminosulfonyl, sulfonylamino, aminocarbonyl, alkylaminocarbonyl, dialkylaminocarbonyl, heterocyclylcarbonyl, and ureido; and
R^{f}, Rⁱ, R^{l}, and R^{o} are independently selected from alkyl, cycloalkyl, haloalkyl, haloalkoxy, alkoxy, hydroxy, halo, amino, alkylamino, cycloalkylsulfonylamino, cyano, cyanoalkyl, alkoxycarbonylalkyl, carboxyalkyl, aminocarbonylalkyl, or —X^{c}—R¹² where X^{c} is bond, alkylene, or heteroalkylene and R¹² is optionally substituted aryl, optionally substituted heteroaryl, and optionally substituted heterocyclyl; provided that when R¹ is heterocyclyl then R^{f} is not hydroxy.

In an embodiment of Formula IIId, or a pharmaceutically acceptable salt thereof,
R¹ is R⁷ wherein R⁷ is aryl optionally substituted with R^{d}, R^{e}, or R^{f};
R² is —NR⁹R¹⁰, wherein
R⁹ is hydrogen or alkyl; and
R¹⁰ is hydrogen or alkyl;
R³ is hydrogen;
R⁴ is hydrogen;
R⁵ is haloalkyl; and
R^{d}, R^{e}, and R^{f} are each, independently, halo.

In still another embodiment, R² is —NR⁹R¹⁰. In an embodiment, R² is —OR⁸. In another embodiment, R² is R¹¹.

In yet another embodiment, R⁹ is deuteroalkyl. In still another embodiment, R⁹ is hydrogen. In an embodiment, R⁹ is alkyl. In another embodiment, R⁹ is methyl or ethyl.

In an embodiment, R¹⁰ is hydrogen, alkyl, haloalkyl, hydroxyalkyl, alkoxyalkyl, aminoalkyl, alkylcarbonyl, alkoxycarbonyl, alkylaminocarbonyl, dialkylaminocarbonyl, alkylaminocarbonylalkyl, or dialkylaminocarbonylalkyl. In another embodiment, R¹⁰ is hydrogen.

In yet another embodiment, R⁸ and R¹⁰ are alkyl. In still another embodiment, R⁸ and R¹⁰ are methyl.

In an embodiment, R⁸ and R¹⁰ are independently cycloalkyl or cycloalkylalkyl, each ring may independently be unsubstituted or substituted with one or two substituents independently selected from alkyl, halo, or cyano.

In another embodiment, R⁸ and R¹⁰ are independently cyclopropyl, cyclobutyl, 1-methylcyclopropyl, (cis)-3-hydroxy-3-methylcyclobutyl, (cis)-3-hydroxy-2,2-dimethylcyclobutyl, 1-cyanocyclobutyl, cyclopropylmethyl, 1-hydroxycyclopropmethyl, 1-fluorocyclopropmethyl, (trans)-3-hydroxy-1-methylcyclobutyl, (cis)-3-cyanocyclobutyl, 1-methylcyclobutyl, (cis)-3-hydroxycyclobutyl, (trans)-3-hydroxycyclobutyl, (trans)-3-cyanocyclobutyl, (2S,1R)-2-hydroxycyclobutyl, (1S,2S)-2-hydroxycyclobutyl, (1S,2R)-2-hydroxycyclobutyl, (1R,2R)-2-hydroxycyclobutyl, (1R,2R)-2-fluorocyclopropyl, 1-fluorocyclopropylmethyl, (1S,2R)-2-fluorocyclopropyl, (1R,2S)-2-fluorocyclopropyl, (1S,2S)-2-fluorocyclopropyl, 2,2-difluorocyclopropyl, (R)-1-cyclopropylethyl, or 2,2-difluorocyclopropylmethyl.

In yet another embodiment, R⁸ and R¹⁰ are independently cyclopropyl, cyclobutyl, 1-methylcyclopropyl, (cis)-3-hydroxy-3-methylcyclobutyl, (cis)-3-hydroxy-2,2-dimethylcyclobutyl, 1-cyanocyclobutyl, (trans)-3-hydroxy-1-methylcyclobutyl (cis)-3-cyanocyclobutyl, 1-methylcyclobutyl, (cis)-3-hydroxycyclobutyl, (trans)-3-hydroxycyclobutyl, (trans)-3-cyanocyclobutyl, (2S,1R)-2-hydroxycyclobutyl, (1S,2S)-2-hydroxycyclobutyl, (1S,2R)-2-hydroxycyclobutyl, (1R,2R)-2-hydroxycyclobutyl, (1R,2R)-2-fluorocyclopropyl, (1S,2R)-2-fluorocyclopropyl, (1R,2S)-2-fluorocyclopropyl, (1S,2S)-2-fluorocyclopropyl, or 2,2-difluorocyclopropyl.

In still another embodiment, R⁸ and R¹⁰ are independently cyclopropylmethyl, 1-hydroxycyclopropmethyl, 1-fluorocyclopropmethyl, 1-fluorocyclopropylmethyl, (R)-1-cyclopropylethyl, or 2,2-difluorocyclopropylmethyl.

In an embodiment, R⁸ and R¹⁰ are independently heteroaryl or heteroaralkyl wherein heteroaryl, by itself or as part heteroaralkyl, is unsubstituted or substituted with R^{j}, R^{k}, or R^{l}.

In another embodiment, R⁸ and R¹⁰ are heteroaryl independently selected from pyrazolyl, oxazolyl, isoxazolyl, imidazolyl, thienyl, pyrrolyl, pyridinyl, pyrimidinyl, pyrazinyl, pyridazinyl, quinolinyl, isoquinolinyl, indolyl, and indazolyl, each ring is either unsubstituted or substituted with Rⁱ, R^{k}, or R^{l}.

In yet another embodiment, R⁸ and R¹⁰ are heteroaryl independently selected from pyrazolyl, imidazolyl, thienyl, pyrrolyl, pyridinyl, pyrimidinyl, pyrazinyl, pyridazinyl, quinolinyl, isoquinolinyl, indolyl, and indazolyl, each ring is either unsubstituted or substituted with R^{j}, R^{k}, or R^{l}.

In still another embodiment, R⁸ and R¹⁰ are heteroaralkyl independently selected from pyrazolylmethyl, pyrazolylethyl, oxazolylmethyl, isoxazolylmethyl, imidazolylmethyl, imidazolylethyl, thienylmethyl, thienylethyl, pyrrolylmethyl, pyrrolylethyl, pyridinylmethyl, pyridinylethyl, pyrimidinylmethyl, pyrimidinylethyl, pyrazinylmethyl, pyrazinylethyl, pyridazinylmethyl, pyridazinylethyl, quinolinylmethyl, quinolinylethyl, isoquinolinylmethyl, isoquinolinylethyl, indolylmethyl, indolylethyl, indazolylmethyl and indazolylethyl, each ring is either unsubstituted or substituted with R^{j}, R^{k}, or R^{l}.

In an embodiment, R⁸ and R¹⁰ are heteroaralkyl independently selected from pyrazolylmethyl, pyrazolylethyl, imidazolylmethyl, imidazolylethyl, thienylmethyl, thienylethyl, pyrrolylmethyl, pyrrolylethyl, pyridinylmethyl, pyridinylethyl, pyrimidinylmethyl, pyrimidinylethyl, pyrazinylmethyl, pyrazinylethyl, pyridazinylmethyl, pyridazinylethyl, quinolinylmethyl, quinolinylethyl, isoquinolinylmethyl, isoquinolinylethyl, indolylmethyl, indolylethyl, indazolylmethyl and indazolylethyl, each ring is either unsubstituted or substituted with R^{j}, R^{k}, or R^{l}.

In another embodiment, R⁸ and R¹⁰ are 1-methyl-1H-pyrazol-5-yl, isoxazol-4-yl, 3-methyl-1,2,4-oxadiazol-5-yl, 5-methylisoxazol-3-yl, 5-methylisoxazol-4-yl, 3-methoxyisoxazol-5-yl, 3,5-dimethylisoxazol-4-yl, 3-methylisoxazol-4-yl, thiazol-4-yl, thiazol-5-yl, isothiazol-4-yl, pyridin-2-yl, pyridin-3-yl, pyridin-4-yl, 2-(difluoromethyl)pyridin-4-yl, 2-(difluoromethoxy)pyridin-4-yl, 5-methoxypyridin-3-yl, 6-methylpyridin-3-yl, 6-methoxypyridin-3-yl, 3-cyanopyridin-4-yl, 3-methoxypyridin-4-yl, 3-fluoropyridin-4-yl, 3-chloropyridin-4-yl, 2-(trifluoromethyl)pyridin-4-yl, 2-methylpyridin-4-yl, pyrimidin-5-yl, 1-methyl-1H-imidazol-4-yl, 1-methylpyrazol-3-ylmethyl, 3-methoxyisoxazol-5-ylmethyl, oxazol-2-ylmethyl, oxazol-4-ylmethyl, oxazol-5-ylmethyl, isoxazol-3-ylmethyl, isoxazol-4-ylmethyl, isoxazol-5-ylmethyl, 1-methyl-1H-pyrazol-3-ylmethyl, 1-methyl-1H-pyrazol-4-ylmethyl, 1-methyl-1H-pyrazol-5-ylmethyl, pyridin-4-ylmethyl, pyridin-3-ylmethyl, or pyridin-2-ylmethyl.

In yet another embodiment,
R² is R¹¹; and
R¹¹ is heterocyclyl which is unsubstituted or substituted with R^{m}, Rⁿ, or R^{o}.

In still another embodiment, R¹¹ is oxetanyl, azetidinyl, 2-oxoazetidinyl, pyrrolidinyl, 2-oxopyrrolidinyl, piperidinyl, piperazinyl, or morpholinyl, each ring is unsubstituted or substituted with R^{m}, Rⁿ, or R^{o}.

In an embodiment, R¹¹ is azetidin-1-yl, 4-hydroxyazetidin-1-yl, 4-methylaminocarbonylazetidin-1-yl, 4-dimethylaminocarbonylazetidin-1-yl, 2-hydromethyl-azetidin-1-yl, 2-methylazetidin-1-yl, 2-oxoazetidin-1-yl, pyrrolidin-1-yl, 2-oxopyrrolidin-1-yl, 3-hydroxypyrrolidin-1-yl, 3,3-dimethylpyrrolidin-1-yl, 3-methoxypyrrolidin-1-yl, 3-hydroxy-3-methylpyrrolidin-1-yl, piperidin-1-yl, 2-carboxypiperidin-1-yl, 2-aminocarbonylpiperidin-1-yl, piperazin-1-yl, 4-methylpiperazin-1-yl, or morpholin-4-yl.

In another embodiment,
R⁵ is alkyl, alkoxy, halo, haloalkyl, haloalkoxy, cycloalkyl, cyano, aminocarbonyl, heteroaryl, heterocyclyl, wherein heterocyclyl or heteroaryl is unsubstituted or substituted with R^{a}, R^{b}, or R^{c}; and
R^{a}, R^{b}, or R^{c} are each independently selected from alkyl, cycloalkyl, haloalkyl, haloalkoxy, alkoxy, hydroxy, halo, cyano, hydroxyalkyl, alkoxyalkyl, or aminoalkyl.

In yet another embodiment,
R⁵ is methyl, ethyl, isopropyl, tert-butyl, methoxy, ethoxy, fluoro, chloro, bromo, trifluoromethyl, difluoromethyl, trifluoromethoxy, difluoromethoxy, cyclopropyl, cyclopentyl, cyano, pyrazolyl, imidazolyl, thienyl, thiazolyl, oxazolyl, isoxazolyl, pyridinyl, pyrimidinyl, oxetan-3-yl, pyrrolidin-1-yl, tetrahydrofuranyl, 2-oxoazetidin-1-yl, or 2-oxopyrrolidin-1-yl, wherein heterocyclyl or heteroaryl rings are unsubstituted or substituted with R^{a}, R^{b}, or R^{c}; and
R^{a}, R^{b}, and R^{c} are each independently selected from alkyl, cycloalkyl, haloalkyl, haloalkoxy, alkoxy, hydroxy, halo, cyano, hydroxyalkyl, alkoxyalkyl, or aminoalkyl.

In still another embodiment, R⁵ is chloro, methyl, ethyl, trifluoromethyl, 1,1-difluoroethyl, or cyclopropyl. In an embodiment, R⁵ is chloro, ethyl, or trifluoromethyl.

In another embodiment, R⁴ and R⁶ are independently selected from hydrogen, methyl, chloro, fluoro, bromo, methoxy, methylthio, methylsulfonyl, trifluoromethyl, trifluoromethoxy, cyano, amino, methylamino, dimethylamino, methylaminocarbonyl, or dimethylaminocarbonyl.

In yet another embodiment,
R⁴ is hydrogen, fluoro, bromo, methyl, methoxy, or cyano; and
R⁶ is hydrogen.

In still another embodiment, R⁴ and R⁶ are hydrogen.

In an embodiment, R³ is hydrogen, alkyl, alkoxy, alkylsulfonyl, halo, haloalkyl, haloalkoxy, cycloalkyl, cyano, amino, alkylamino, dialkylamino, aminocarbonyl, alkylaminocarbonyl, or dialkylaminocarbonyl. In another embodiment, R³ is hydrogen or methoxy. In yet another embodiment, R³ is hydrogen.

In still another embodiment, R³ is methyl, ethyl, methoxy, ethoxy, fluoro, chloro, bromo, trifluoromethyl, difluoromethyl, trifluoromethoxy, difluoromethoxy, cyclopropyl, cyano, methylsulfonyl, aminocarbonyl, methylamino, or dimethylamino.

In an embodiment,
R¹ is R⁷;
R⁷ is phenyl which is unsubstituted or substituted with R^{d}, R^{e}, or R^{f};
R^{d} and R^{e} are independently selected from alkyl, haloalkyl, haloalkoxy, alkoxy, alkylsulfonyl, halo, cyano, carboxy, alkoxycarbonyl, hydroxyalkyl, alkoxyalkyl, aminoalkyl, aminosulfonyl, alkylaminosulfonyl, dialkylaminosulfonyl, sulfonylamino, aminocarbonyl, alkylaminocarbonyl, dialkylaminocarbonyl, heterocyclylcarbonyl, and ureido; and
R^{f} is selected from alkyl, haloalkyl, haloalkoxy, alkoxy, hydroxy, halo, and cyano.

In another embodiment,
R¹ is R⁷;
R⁷ is phenyl which is unsubstituted or substituted with R^{d}, R^{e}, or R^{f};
R^{d} and R^{e} are independently selected from methyl, ethyl, fluoro, chloro, bromo, methoxy, ethoxy, cyclopropyl, cyano, methylsulfonyl, methoxymethyl, aminomethyl, 2-hydroxyethyl, or 3-hydroxypropyl; and
R^{f} is selected from hydroxy, fluoro, chloro, cyano. and methyl.

In yet another embodiment,
R¹ is R⁷;
R⁷ is phenyl which is unsubstituted or substituted with R^{f}; and
R^{f} is fluoro, chloro, bromo, or methyl, wherein R^{f} is attached to carbon atoms on the phenyl ring that is ortho to the carbon atom of the phenyl ring attached to quinazolone nitrogen.

In still another embodiment,
R¹ is R⁷;
R⁷ is heteroaryl which is unsubstituted or substituted with R^{d}, R^{e}, or R^{f};
R^{d} and R^{e} are independently selected from alkyl, haloalkyl, haloalkoxy, alkoxy, alkylsulfonyl, halo, cyano, carboxy, alkoxycarbonyl, hydroxyalkyl, alkoxyalkyl, aminoalkyl, aminosulfonyl, alkylaminosulfonyl, dialkylaminosulfonyl, sulfonylamino, aminocarbonyl, alkylaminocarbonyl, dialkylaminocarbonyl, heterocyclylcarbonyl, and ureido; and
R^{f} is selected from alkyl, haloalkyl, haloalkoxy, alkoxy, hydroxy, halo, and cyano.

In an embodiment,
R¹ is R⁷;
R⁷ is 5 or 6-membered heteroaryl ring which is unsubstituted or substituted with R^{d} or R^{e}.
R^{d} and R^{e} are each independently selected from methyl, ethyl, fluoro, chloro, bromo, methoxy, ethoxy, cyclopropyl, cyano, methylsulfonyl, methoxymethyl, aminomethyl, 2-hydroxyethyl, and 3-hydroxypropyl.
   In another embodiment,
R⁷ pyridinyl which is unsubstituted or substituted with R^{f};
   R^{f} is fluoro, chloro, bromo, or methyl and wherein R^{f} is attached to carbon atoms on the pyridinyl ring that is ortho to the carbon atom of the pyridinyl ring attached to quinazolone nitrogen.

In yet another embodiment, methionine adenosyltransferase II alpha (MAT2A) inhibitor is selected from the group consisting of a compound from Table 1, or a pharmaceutically acceptable salt thereof.

TABLE 1

| Cpd. No. | Structure | Name | Mass Spec. |
|---|---|---|---|
| 1 | 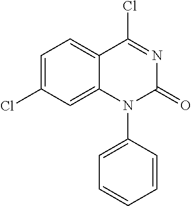 [SMILES: O=c1nc(Cl)c2ccc(Cl)cc2n1-c1ccccc1] | 4,7-dichloro-1-phenylquinazolin- 2(1H)-one | |
| 2 | 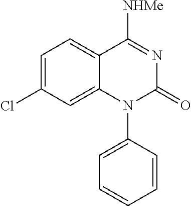 [SMILES: CNc1nc(=O)n(-c2ccccc2)c2cc(Cl)ccc12] | 7-chloro-4-(methylamino)-1- phenyl-quinazolin-2(1H)-one | |
| 3 | 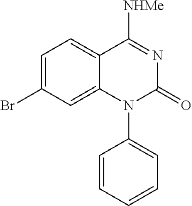 [SMILES: CNc1nc(=O)n(-c2ccccc2)c2cc(Br)ccc12] | 7-bromo-4-(methylamino)-1- phenyl-quinazolin-2(1H)-one | |
| 4 | 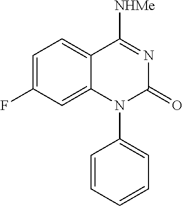 [SMILES: CNc1nc(=O)n(-c2ccccc2)c2cc(F)ccc12] | 7-fluoro-1-phenylquinazolin-2(1H)- one | |
| 5 | 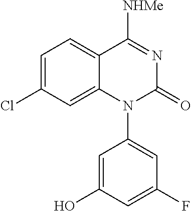 [SMILES: CNc1nc(=O)n(-c2cc(O)cc(F)c2)c2cc(Cl)ccc12] | 7-Chloro-1-(5-fluoro-3-hydroxy- phenyl)-4-(methylamino)- hydroquinazolin-2-one | |
| 6 | 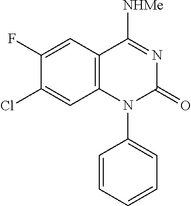 [SMILES: CNc1nc(=O)n(-c2ccccc2)c2cc(Cl)c(F)cc12] | 7-chloro-6-fluoro-4-(methylamino)- 1-phenyl-quinazolin-2(1H)-one | |
| 7 | 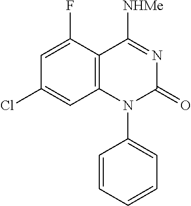 [SMILES: CNc1nc(=O)n(-c2ccccc2)c2cc(Cl)cc(F)c12] | 7-chloro-5-fluoro-4-(methylamino)- 1-phenyl-quinazolin-2(1H)-one | |
| 8 | 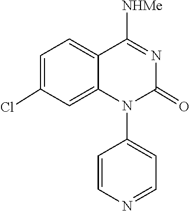 [SMILES: CNc1nc(=O)n(-c2ccncc2)c2cc(Cl)ccc12] | 7-chloro-4-(methylamino)-1- (pyridin-4-yl)-quinazolin-2(1H)-one | |
| 9 | 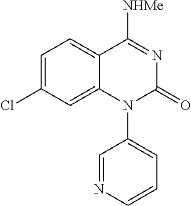 [SMILES: CNc1nc(=O)n(-c2cccnc2)c2cc(Cl)ccc12] | 7-chloro-4-(methylamino)-1- (pyridin-3-yl)-quinazolin-2(1H)-one | |
| 10 | 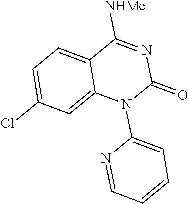 [SMILES: CNc1nc(=O)n(-c2ccccn2)c2cc(Cl)ccc12] | 7-chloro-4-(methylamino)-1- (pyridin-2-yl)-quinazolin-2(1H)-one | |
| 11 | 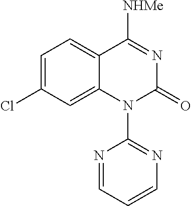 [SMILES: CNc1nc(=O)n(-c2ncccn2)c2cc(Cl)ccc12] | 7-chloro-4-(methylamino)-1- pyrimidin-2-yl-quinazolin-2(1H)-one | |
| 12 | 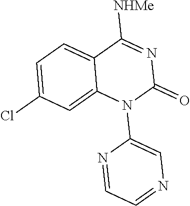 [SMILES: CNc1nc(=O)n(-c2cnccn2)c2cc(Cl)ccc12] | 7-chloro-4-(methylamino)-1- (pyrazin-2-yl)-quinazolin-2(1H)-one | |
| 13 | 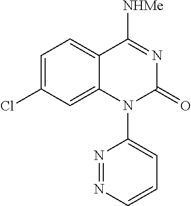 [SMILES: CNc1nc(=O)n(-c2cccnn2)c2cc(Cl)ccc12] | 7-chloro-4-(methylamino)-1- (pyridazin-3-yl)-quinazolin-2(1H)- one | |
| 14 | 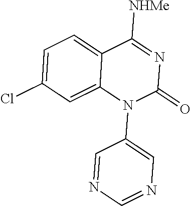 [SMILES: CNc1nc(=O)n(-c2cncnc2)c2cc(Cl)ccc12] | 7-chloro-4-(methylamino)-1- (pyrimidin-5-yl)-quinazolin-2(1H)- one | |
| 15 | 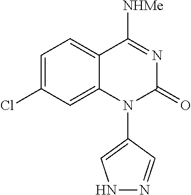 [SMILES: CNc1nc(=O)n(-c2cn[nH]c2)c2cc(Cl)ccc12] | 7-chloro-4-(methylamino)-1-(1H- pyrazol-4-yl)-quinazolin-2(1H)-one | |
| 16 | 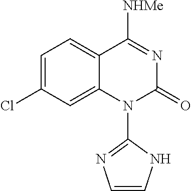 [SMILES: CNc1nc(=O)n(-c2ncc[nH]2)c2cc(Cl)ccc12] | 7-chloro-1-(1H-imidazol-2-yl)-4- (methylamino)-quinazolin-2(1H)- one | |
| 17 | 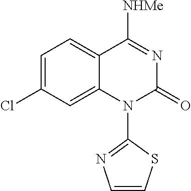 [SMILES: CNc1nc(=O)n(-c2nccs2)c2cc(Cl)ccc12] | 7-chloro-4-(methylamino)-1- (thiazol-2-yl)-quinazolin-2(1H)-one | |
| 18 | 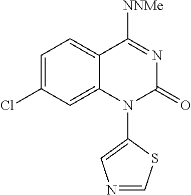 | 7-chloro-4-(methylamino)-1- (thiazol-5-yl)-quinazolin-2(1H)-one | |
| 19 | 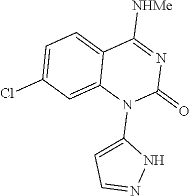 [SMILES: CNc1nc(=O)n(-c2ccn[nH]2)c2cc(Cl)ccc12] | 7-chloro-4-(methylamino)-1-(1H- pyrazol-5-yl)-quinazolin-2(1H)-one | |
| 20 | 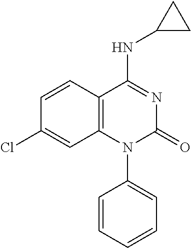 [SMILES: O=c1nc(NC2CC2)c2ccc(Cl)cc2n1-c1ccccc1] | 7-chloro-4-(cyclopropylamino)-1- phenylquinazolin-2(1H)-one | |
| 21 | 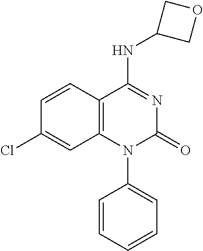 [SMILES: O=c1nc(NC2COC2)c2ccc(Cl)cc2n1-c1ccccc1] | 7-Chloro-4-(oxetan-3-ylamino)-1- phenylquinazolin-2(1H)-one | |
| 22 | 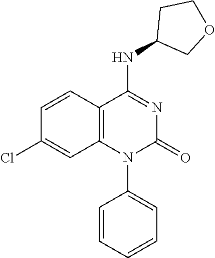 [SMILES: O=c1nc(N[C@H]2CCOC2)c2ccc(Cl)cc2n1-c1ccccc1] | (S)-7-chloro-1-phenyl-4- ((tetrahydro-furan-3-yl)amino)- quinazolin-2(1H)-one | |
| 23 | 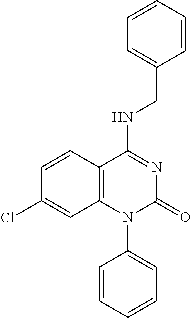 [SMILES: O=c1nc(NCc2ccccc2)c2ccc(Cl)cc2n1-c1ccccc1] | 4-(benzylamino)-7-chloro-1-phenyl- quinazolin-2(1H)-one | |
| 24 | 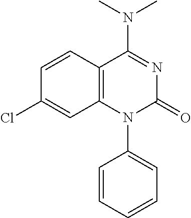 [SMILES: CN(C)c1nc(=O)n(-c2ccccc2)c2cc(Cl)ccc12] | 7-chloro-4-(dimethylamino)-1- phenyl-quinazolin-2(1H)-one | |
| 25 | 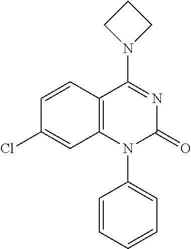 [SMILES: O=c1nc(N2CCC2)c2ccc(Cl)cc2n1-c1ccccc1] | 4-(azetidin-1-yl)-7-chloro-1-phenyl- quinazolin-2(1H)-one | |
| 26 | 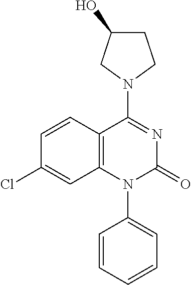 [SMILES: O=c1nc(N2CC[C@H](O)C2)c2ccc(Cl)cc2n1-c1ccccc1] | (S)-7-chloro-4-(3-hydroxypyrrolidin- 1-yl)-1-phenylquinazolin-2(1H)-one | |
| 27 | 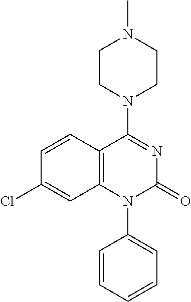 [SMILES: CN1CCN(c2nc(=O)n(-c3ccccc3)c3cc(Cl)ccc23)CC1] | 7-chloro-4-(4-methylpiperazin-1- yl)-1-phenyl-quinazolin-2(1H)-one | |
| 28 | 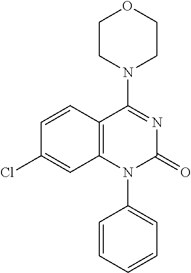 [SMILES: O=c1nc(N2CCOCC2)c2ccc(Cl)cc2n1-c1ccccc1] | 7-chloro-4-morpholino-1-phenyl- quinazolin-2(1H)-one | |
| 29 | 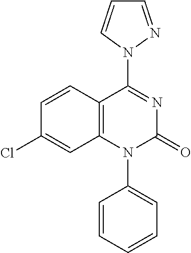 [SMILES: O=c1nc(-n2cccn2)c2ccc(Cl)cc2n1-c1ccccc1] | 7-chloro-1-phenyl-4-(1H-pyrazol-1- yl)quinazolin-2(1H)-one | |
| 30 | 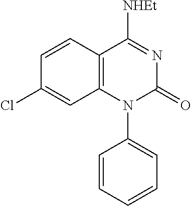 | 7-chloro-4-(ethylamino)-1-phenyl- quinazolin-2(1H)-one | |
| 31 | 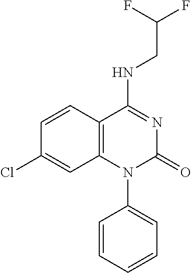 [SMILES: O=c1nc(NCC(F)F)c2ccc(Cl)cc2n1-c1ccccc1] | 7-chloro-4-((2,2- difluoroethyl)amino)-1-phenyl- quinazolin-2(1H)-one | |
| 32 | 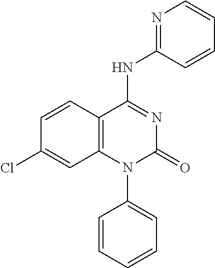 [SMILES: O=c1nc(Nc2ccccn2)c2ccc(Cl)cc2n1-c1ccccc1] | 7-chloro-1-phenyl-4-(pyridin-2-yl- amino)quinazolin-2(1H)-one | |
| 33 | 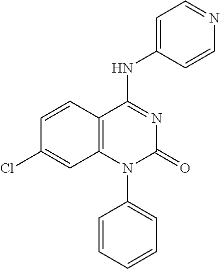 [SMILES: O=c1nc(Nc2ccncc2)c2ccc(Cl)cc2n1-c1ccccc1] | 7-chloro-1-phenyl-4-(pyridin-4-yl- amino)quinazolin-2(1H)-one | |
| 34 | 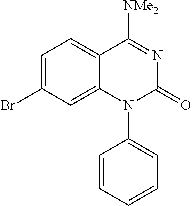 [SMILES: CN(C)c1nc(=O)n(-c2ccccc2)c2cc(Br)ccc12] | 7-bromo-4-(dimethylamino)-1- phenyl-quinazolin-2(1H)-one | |
| 35 | 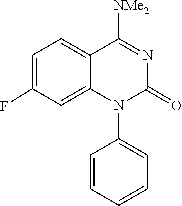 [SMILES: CN(C)c1nc(=O)n(-c2ccccc2)c2cc(F)ccc12] | 4-(dimethylamino)-7-fluoro-1- phenyl-quinazolin-2(1H)-one | |
| 36 | 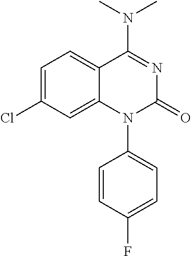 [SMILES: CN(C)c1nc(=O)n(-c2ccc(F)cc2)c2cc(Cl)ccc12] | 4-(dimethylamino)-7-chloro-1-(4- fluorophenyl)-hydroxyquinazolin-2- one | |
| 37 | 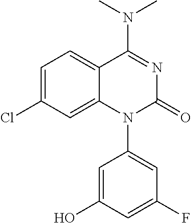 [SMILES: CN(C)c1nc(=O)n(-c2cc(O)cc(F)c2)c2cc(Cl)ccc12] | 4-(dimethylamino)-7-chloro-1-(5- fluoro-3-hydroxyphenyl)hydro- quinazolin-2-one | |
| 38 | 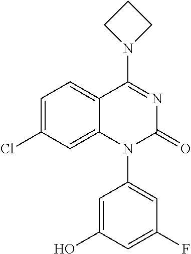 [SMILES: O=c1nc(N2CCC2)c2ccc(Cl)cc2n1-c1cc(O)cc(F)c1] | 4-azetidinyl-7-chloro-1-(5-fluoro-3- hydroxy-phenyl)hydroquinazolin-2- one | |
| 39 | 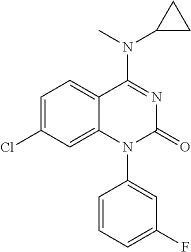 [SMILES: CN(c1nc(=O)n(-c2cccc(F)c2)c2cc(Cl)ccc12)C1CC1] | 7-chloro-4- (cyclopropylmethylamino)-1-(3- fluorophenyl)hydroquinazolin-2- one | |
| 40 | 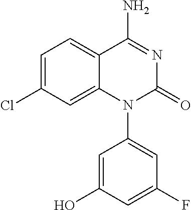 [SMILES: Nc1nc(=O)n(-c2cc(O)cc(F)c2)c2cc(Cl)ccc12] | 4-amino-7-chloro-1-(5-fluoro-3- hydroxyphenyl)-hydroquinazolin-2- one | |
| 41 | 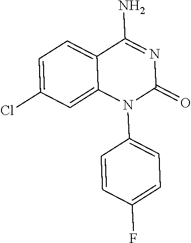 [SMILES: Nc1nc(=O)n(-c2ccc(F)cc2)c2cc(Cl)ccc12] | 4-amino-7-chloro-1-(4- fluorophenyl)-hydro-quinazolin-2- one | |
| 42 | 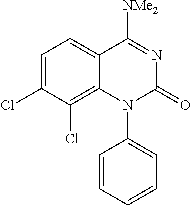 [SMILES: CN(C)c1nc(=O)n(-c2ccccc2)c2c(Cl)c(Cl)ccc12] | 7,8-dichloro-4-(dimethylamino)-1- phenylquinazolin-2(1H)-one | |
| 43 | 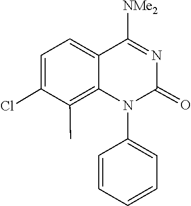 [SMILES: Cc1c(Cl)ccc2c(N(C)C)nc(=O)n(-c3ccccc3)c12] | 7-chloro-4-(dimethyalmino)-8- methyl-1-phenyl-quinazolin-2(1H)- one | |
| 44 | 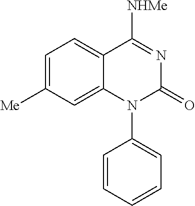 [SMILES: CNc1nc(=O)n(-c2ccccc2)c2cc(C)ccc12] | 7-methyl-4-(methylamino)-1- phenyl-quinazolin-2(1H)-one | |
| 45 | 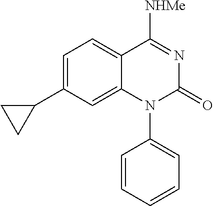 [SMILES: CNc1nc(=O)n(-c2ccccc2)c2cc(C3CC3)ccc12] | 7-cyclopropyl-4-(methylamino)-1- phenylquinazolin-2(1H)-one | |
| 46 | 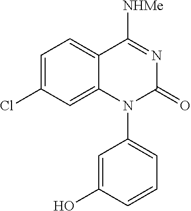 [SMILES: CNc1nc(=O)n(-c2cccc(O)c2)c2cc(Cl)ccc12] | 7-chloro-1-(3-hydroxyphenyl)-4- (methylamino)-hydroquinazolin-2- one | |
| 47 | 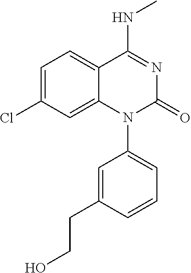 [SMILES: CNc1nc(=O)n(-c2cccc(CCO)c2)c2cc(Cl)ccc12] | 7-chloro-1-[3-(2- hydroxyethylphenyl]-4- (methylamino)-hydroquinazolin-2- one | |
| 48 | 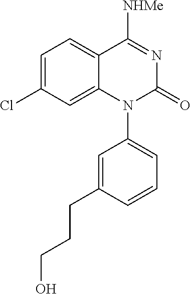 [SMILES: CNc1nc(=O)n(-c2cccc(CCCO)c2)c2cc(Cl)ccc12] | 7-chloro-1-[3-(3-hydroxypropyl)- phenyl]-4-(methylamino)hydro- quinazolin-2-one | |
| 49 | 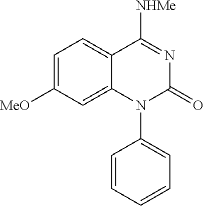 [SMILES: CNc1nc(=O)n(-c2ccccc2)c2cc(OC)ccc12] | 7-methoxy-4-(methylamino)-1- phenyl-quinazolin-2(1H)-one | |
| 50 | 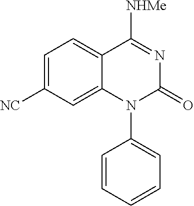 [SMILES: CNc1nc(=O)n(-c2ccccc2)c2cc(C#N)ccc12] | 4-(methylamino)-2-oxo-1-phenyl- 1,2-dihydro-quinazoline-7- carbonitrile | |
| 51 | 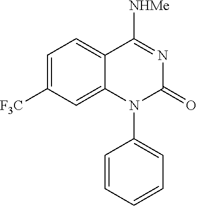 [SMILES: CNc1nc(=O)n(-c2ccccc2)c2cc(C(F)(F)F)ccc12] | 4-(methylamino)-1-phenyl-7- (trifluoro-methyl)-quinazolin-2(1H)- one | |
| 52 | 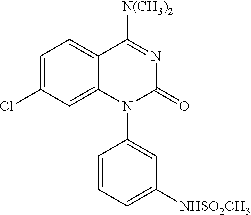 | N-(3-(7-chloro-4-(dimethylamino)- 2-oxoquinazolin-1(2H)-yl)phenyl)- methanesulfonamide | |
| 54 | 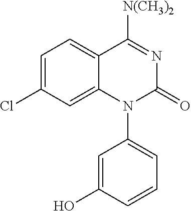 [SMILES: CN(C)c1nc(=O)n(-c2cccc(O)c2)c2cc(Cl)ccc12] | 4-(dimethylamino)-7-chloro-1-(3- hydroxyphenyl)hydroquinazolin-2- one | |
| 55 | 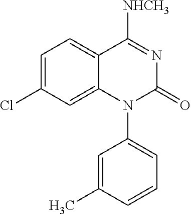 [SMILES: CNc1nc(=O)n(-c2cccc(C)c2)c2cc(Cl)ccc12] | 7-Chloro-4-(methylamino)-1-(3- methylphenyl)hydroquinazolin-2- one | |
| 56 | 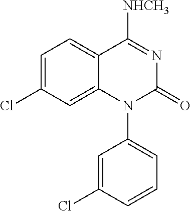 [SMILES: CNc1nc(=O)n(-c2cccc(Cl)c2)c2cc(Cl)ccc12] | 7-Chloro-1-(3-chlorophenyl)-4- (methylamino)hydroquinazolin-2- one | |
| 57 | 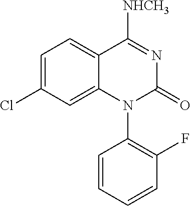 [SMILES: CNc1nc(=O)n(-c2ccccc2F)c2cc(Cl)ccc12] | 7-chloro-1-(2-fluorophenyl)-4- (methylamino)quinazolin-2(1H)- one | |
| 58 | 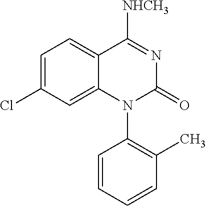 [SMILES: CNc1nc(=O)n(-c2ccccc2C)c2cc(Cl)ccc12] | 7-Chloro-4-(methylamino)-1-(2- methylphenyl)hydroquinazolin-2- one | |
| 59 | 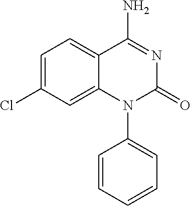 [SMILES: Nc1nc(=O)n(-c2ccccc2)c2cc(Cl)ccc12] | 4-amino-7-chloro-1- phenylquinazolin-2(1H)-one | |
| 60 | 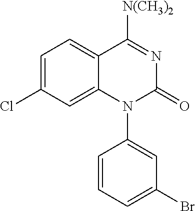 [SMILES: CN(C)c1nc(=O)n(-c2cccc(Br)c2)c2cc(Cl)ccc12] | 1-(3-bromophenyl)-7-chloro-4- (dimethylamino)quinazolin-2(1H)- one | |
| 61 | 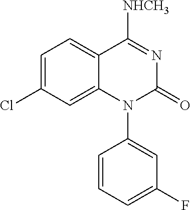 [SMILES: CNc1nc(=O)n(-c2cccc(F)c2)c2cc(Cl)ccc12] | 7-chloro-1-(3-fluorophenyl)-4- (methylamino)quinazolin-2(1H)- one | |
| 62 | 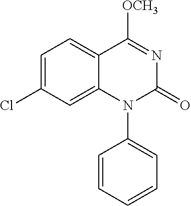 [SMILES: COc1nc(=O)n(-c2ccccc2)c2cc(Cl)ccc12] | 7-chloro-4-methoxy-1-phenyl- quinazolin-2(1H)-one | |
| 63 | 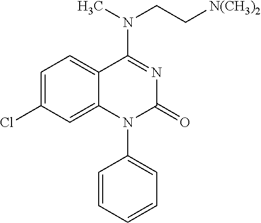 [SMILES: CN(C)CCN(C)c1nc(=O)n(-c2ccccc2)c2cc(Cl)ccc12] | 7-chloro-4-((2- (dimethylamino)ethyl)-(methyl)- amino)-1-phenylquinazolin-2(1H)- one | |
| 64 | 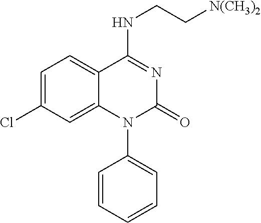 [SMILES: CN(C)CCNc1nc(=O)n(-c2ccccc2)c2cc(Cl)ccc12] | 7-chloro-4-((2- (dimethylamino)ethyl)-amino)-1- phenylquinazolin-2(1H)-one | |
| 65 | 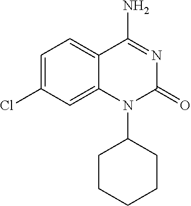 [SMILES: Nc1nc(=O)n(C2CCCCC2)c2cc(Cl)ccc12] | 4-amino-7-chloro-1-cyclohexyl- quinazolin-2(1H)-one | |
| 66 | 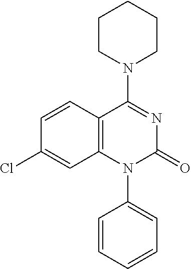 [SMILES: O=c1nc(N2CCCCC2)c2ccc(Cl)cc2n1-c1ccccc1] | 7-chloro-1-phenyl-4-(piperidin-1- yl)-quinazolin-2(1H)-one | |
| 67 | 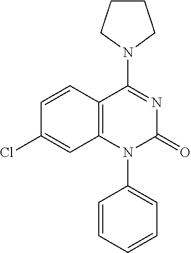 [SMILES: O=c1nc(N2CCCC2)c2ccc(Cl)cc2n1-c1ccccc1] | 7-chloro-1-phenyl-4-(pyrrolidin-1- yl)-quinazolin-2(1H)-one | |
| 68 | 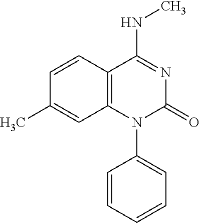 [SMILES: CNc1nc(=O)n(-c2ccccc2)c2cc(C)ccc12] | 7-methyl-4-(methylamino)-1- phenyl-pyrido[2,3-d]pyrimidin- 2(1H)-one | |
| 69 | 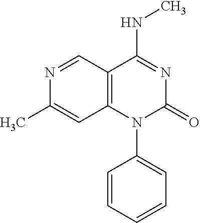 [SMILES: CNc1nc(=O)n(-c2ccccc2)c2cc(C)ncc12] | 7-methyl-4-(methylamino)-1- phenyl-pyrido[4,3-d]pyrimidin- 2(1H)-one | |
| 70 | 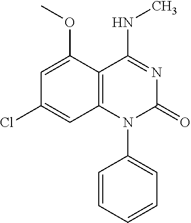 [SMILES: CNc1nc(=O)n(-c2ccccc2)c2cc(Cl)cc(OC)c12] | 7-chloro-5-methoxy-4- (methylamino)-1-phenylquinazolin- 2(1H)-one | |
| 71 | 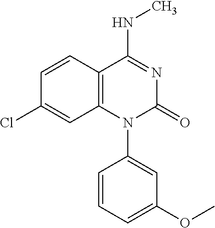 [SMILES: CNc1nc(=O)n(-c2cccc(OC)c2)c2cc(Cl)ccc12] | 7-chloro-1-(3-methoxyphenyl)-4- (methyalmino)quinazolin-2(1H)- one | |
| 73 | 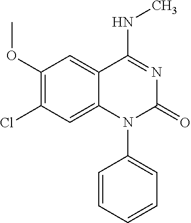 [SMILES: CNc1nc(=O)n(-c2ccccc2)c2cc(Cl)c(OC)cc12] | 7-chloro-6-methoxy-4- (methylamino)-1-phenylquinazolin- 2(1H)-one | |
| 74 | 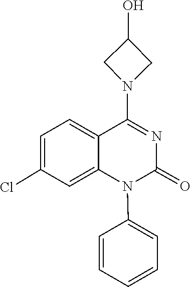 [SMILES: O=c1nc(N2CC(O)C2)c2ccc(Cl)cc2n1-c1ccccc1] | 7-chloro-4-(3-hydroxyazetidin-1-yl)- 1-phenylquinazolin-2(1H)-one | |
| 75 | 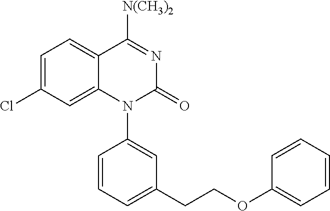 [SMILES: CN(C)c1nc(=O)n(-c2cccc(CCOc3ccccc3)c2)c2cc(Cl)ccc12] | 7-chloro-4-(dimethylamino)-1-(3-(2- phenoxyethyl)phenyl)quinazolin- 2(1H)-one | |
| 76 | 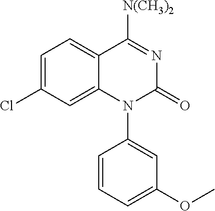 [SMILES: COc1cccc(-n2c(=O)nc(N(C)C)c3ccc(Cl)cc32)c1] | 4-(dimethylamino)-7-chloro-1-(3- methoxyphenyl)hydroquinazolin-2- one | |
| 77 | 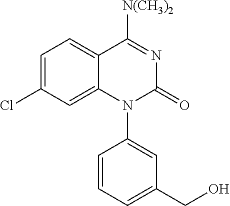 [SMILES: CN(C)c1nc(=O)n(-c2cccc(CO)c2)c2cc(Cl)ccc12] | 4-(dimethylamino)-7-chloro-1-[3- (hydroxymethyl)phenyl]hydroquina- zolin-2-one | |
| 78 | 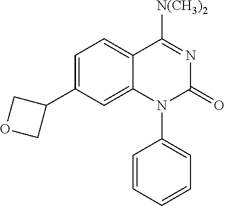 [SMILES: CN(C)c1nc(=O)n(-c2ccccc2)c2cc(C3COC3)ccc12] | 4-(dimethylamino)-7-(oxetan-3-yl)- 1-phenylquinazolin-2(1H0-one | |
| 79 | 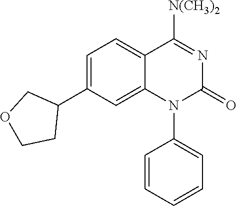 [SMILES: CN(C)c1nc(=O)n(-c2ccccc2)c2cc(C3CCOC3)ccc12] | 4-(dimethylamino)-1-phenyl-7- (tetrahydrofuran-3-yl)quinazolin- 2(1H)-one | |
| 80 | 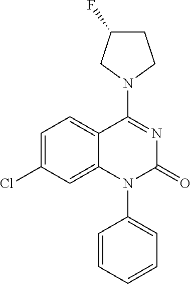 [SMILES: O=c1nc(N2CC[C@@H](F)C2)c2ccc(Cl)cc2n1-c1ccccc1] | (R)-7-chloro-4-(3-fluoropyrrolidin-1- yl)-1-phenylquinazolin-2(1H)-one | |
| 81 | 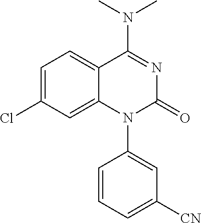 [SMILES: CN(C)c1nc(=O)n(-c2cccc(C#N)c2)c2cc(Cl)ccc12] | 3-[4-(dimethylamino)-7-chloro-2- oxohydroquinazolinyl]benzenecarbo- nitrile | |
| 82 | 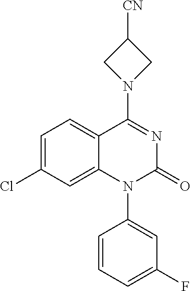 [SMILES: N#CC1CN(c2nc(=O)n(-c3cccc(F)c3)c3cc(Cl)ccc23)C1] | 1-(7-chloro-1-(3-fluorophenyl)-2- oxo-1,2-dihydroquinazolin-4- yl)azetidine-3-carbonitrile | |
| 83 | 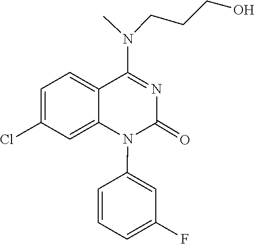 [SMILES: CN(CCCO)c1nc(=O)n(-c2cccc(F)c2)c2cc(Cl)ccc12] | 7-chloro-1-(3-fluorophenyl)-4-((3- hydroxypropyl)(methyl)amino)quin- azolin-2(1H)-one | |
| 85 | 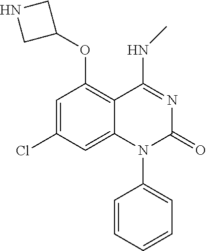 [SMILES: CNc1nc(=O)n(-c2ccccc2)c2cc(Cl)cc(OC3CNC3)c12] | 5-azetidin-3-yloxy-7-chloro-4- (methyl-amino)-1- phenylhydroquinazolin-2-one | m/z [M + H]+ 357.1 |
| 86 | 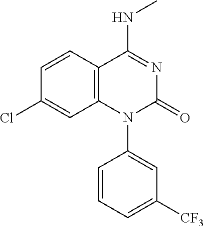 [SMILES: CNc1nc(=O)n(-c2cccc(C(F)(F)F)c2)c2cc(Cl)ccc12] | 7-chloro-4-(methylamino)-1-[3- (trifluoromethyl)phenyl]hydroquinazolin- 2-one | m/z [M + H]+ 354.04 |
| 87 | 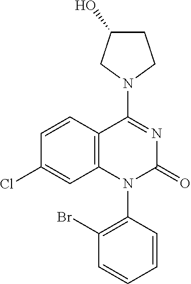 [SMILES: O=c1nc(N2CC[C@@H](O)C2)c2ccc(Cl)cc2n1-c1ccccc1Br] | 4-((3R)-3-hydroxypyrrolidin-1-yl)-1- (2-bromophenyl)-7- chlorohydroquinazolin-2-one | m/z [M + H]+ 420.04 |
| 88 | 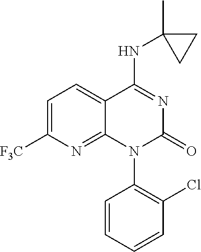 [SMILES: CC1(Nc2nc(=O)n(-c3ccccc3Cl)c3nc(C(F)(F)F)ccc23)CC1] | 1-(2-chlorophenyl)-4-((1-methylcyclo- propyl)amino)-7-(trifluoromethyl)- pyrido[2,3-d]pyrimidin-2(1H)-one | m/z [M + H]+ 395.05 |
| 89 | 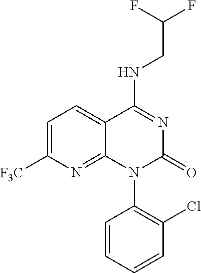 [SMILES: O=c1nc(NCC(F)F)c2ccc(C(F)(F)F)nc2n1-c1ccccc1Cl] | 1-(2-chlorophenyl)-4-[(2,2-difluoro- ethyl)amino]-7-(trifluoromethyl)- pyrido[2,3-d]-pyrimidin-2(1H)-one | m/z [M + H]+ 405.1 |
| 90 | 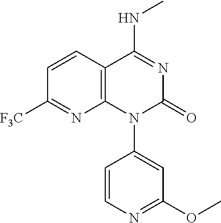 [SMILES: CNc1nc(=O)n(-c2ccnc(OC)c2)c2nc(C(F)(F)F)ccc12] | 1-(2-methoxypyridin-4-yl)-4-(methyl- amino)-7-(trifluoromethyl)pyrido[2,3- d]-pyrimidin-2(1H)-one | m/z [M + H]+ 352.0 |
| 91 | 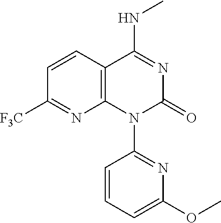 [SMILES: CNc1nc(=O)n(-c2cccc(OC)n2)c2nc(C(F)(F)F)ccc12] | 1-(6-methoxypyridin-2-yl)-4- (methylamino)-7-(trifluoromethyl)- pyrido[2,3-d]pyrimidin-2(1H)-one | m/z [M + H]+ 352.0 |
| 92 | 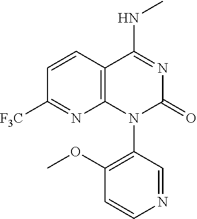 [SMILES: CNc1nc(=O)n(-c2cnccc2OC)c2nc(C(F)(F)F)ccc12] | 1-(4-methoxypyridin-3-yl)-4-(methyl- amino)-7-(trifluoromethyl)pyrido[2,3- d]pyrimidin-2(1H)-one | m/z [M + H]+ 352.0 |
| 93 | 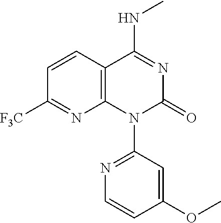 [SMILES: CNc1nc(=O)n(-c2cc(OC)ccn2)c2nc(C(F)(F)F)ccc12] | 1-(4-methoxypyridin-2-yl)-4-(methyl- amino)-7-(trifluoromethyl)pyrido[2,3- d]pyrimidin-2(1H)-one | m/z [M + H]+ 352.0 |
| 94 | 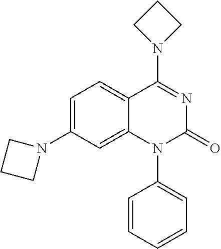 [SMILES: O=c1nc(N2CCC2)c2ccc(N3CCC3)cc2n1-c1ccccc1] | 4,7-di(azetidin-1-yl)-1-phenylhydro- quinazolin-2-one | m/z [M + H]+ 333.2 |
| 94 | 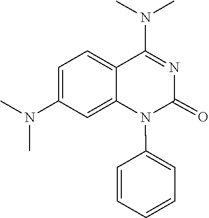 [SMILES: CN(C)c1ccc2c(N(C)C)nc(=O)n(-c3ccccc3)c2c1] | 4,7-bis(dimethylamino)-1- phenylhydro-quinazolin-2-one | m/z [M + H]+ 309.2 |
| 96 | 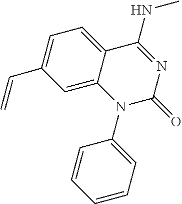 [SMILES: C=Cc1ccc2c(NC)nc(=O)n(-c3ccccc3)c2c1] | 4-(methylamino)-1-phenyl-7- vinylhydro-quinazolin-2-one | m/z [M + H]+ 278.15 |
| 97 | 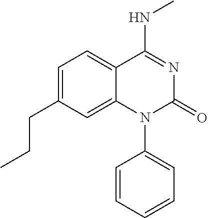 [SMILES: CCCc1ccc2c(NC)nc(=O)n(-c3ccccc3)c2c1] | 4-(methylamino)-1-phenyl-7- propylhydroquinazolin-2-one | m/z [M + H]+ 294.2 |
| 99 | 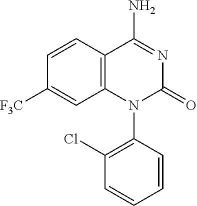 [SMILES: Nc1nc(=O)n(-c2ccccc2Cl)c2cc(C(F)(F)F)ccc12] | 4-amino-1-(2-chlorophenyl)-7- (trifluoromethyl)hydroquinazolin-2- one | m/z [M + H]+ 340.0 |
| 100 | 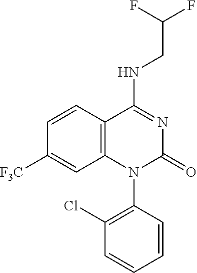 [SMILES: O=c1nc(NCC(F)F)c2ccc(C(F)(F)F)cc2n1-c1ccccc1Cl] | 1-(2-chlorophenyl)-4-[(2,2-difluoro- ethyl)amino]-7-(trifluoromethyl)hydro- quinazolin-2-one | m/z [M + H]+ 404.0 |
| 101 | 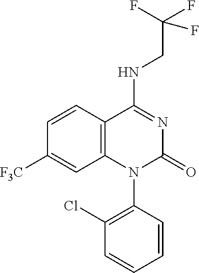 [SMILES: O=c1nc(NCC(F)(F)F)c2ccc(C(F)(F)F)cc2n1-c1ccccc1Cl] | 1-(2-chlorophenyl)-4-[(2,2,2-trifluoro- ethyl)amino]-7-(trifluoromethyl)hydro- quinazolin-2-one | m/z [M + H]+ 422.0 |
| 102 | 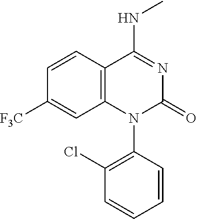 [SMILES: CNc1nc(=O)n(-c2ccccc2Cl)c2cc(C(F)(F)F)ccc12] | 1-(2-chlorophenyl)-4-(methylamino)- 7-(trifluoromethyl)hydroquinazolin-2- one | m/z [M + H]+ 354.1 |
| 103 | 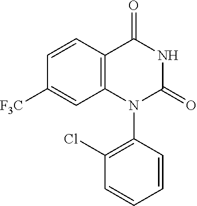 [SMILES: O=c1[nH]c(=O)n(-c2ccccc2Cl)c2cc(C(F)(F)F)ccc12] | 1-(2-chlorophenyl)-7- (trifluoromethyl)-1,3- dihydroquinazoline-2,4-dione | m/z [M + H]+ 341.0 |
| 104 | 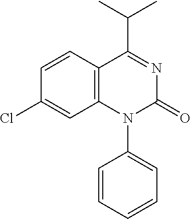 [SMILES: CC(C)c1nc(=O)n(-c2ccccc2)c2cc(Cl)ccc12] | 7-chloro-4-(methylethyl)-1- phenylhydro-quinazolin-2-one | m/z [M + H]+ 299.19 |
| 105 | 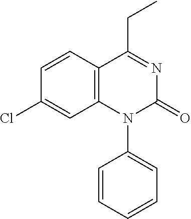 [SMILES: CCc1nc(=O)n(-c2ccccc2)c2cc(Cl)ccc12] | 7-chloro-4-ethyl-1-phenylhydro- quinazolin-1-one | m/z [M + H]+ 285.21 |
| 106 | 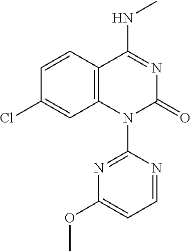 [SMILES: CNc1nc(=O)n(-c2nccc(OC)n2)c2cc(Cl)ccc12] | 7-chloro-1-(4-methoxypyrimidin-2-yl)- 4-(methylamino)hydroquinazolin-2- one | m/z [M + H]+ 318.12 |
| 107 | 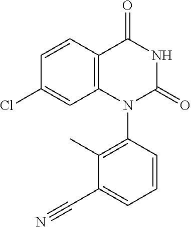 [SMILES: Cc1c(C#N)cccc1-n1c(=O)[nH]c(=O)c2ccc(Cl)cc21] | 3-(7-chloro-2,4-dioxo-2,3-dihydro- quinazolin-1(2H)-yl)-2-methyl- benzonitrile | m/z [M + H]+ 310.13 |
| 108 | 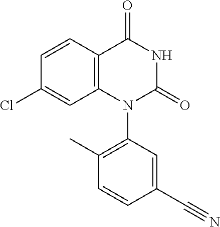 [SMILES: Cc1ccc(C#N)cc1-n1c(=O)[nH]c(=O)c2ccc(Cl)cc21] | 3-(7-chloro-2,4-dioxo-3,4-dihydro- quinazolin-1(2H)-yl)-4-methyl- benzonitrile | m/z [M + H]+ 310.06 |
| 109 | 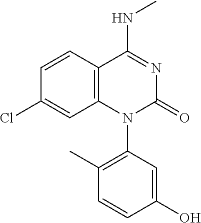 [SMILES: CNc1nc(=O)n(-c2cc(O)ccc2C)c2cc(Cl)ccc12] | 7-chloro-1-(3-hydroxy-6- methylphenyl)-4- (methylamino)hydroquinazolin-2-one | m/z [M + H]+ 316.18 |
| 110 | 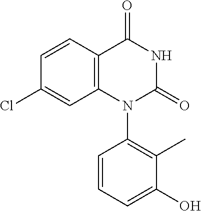 [SMILES: Cc1c(O)cccc1-n1c(=O)[nH]c(=O)c2ccc(Cl)cc21] | 7-chloro-1-(3-hydroxy-2- methylphenyl)-1,3- dihydroquinazolin-2,4-dione | m/z [M + H]+ 303.13 |
| 112 | 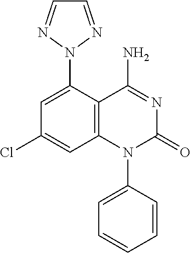 [SMILES: Nc1nc(=O)n(-c2ccccc2)c2cc(Cl)cc(-n3nccn3)c12] | 4-amino-7-chloro-1-phenyl-5-(1,2,3- triazol-2-yl)hydroquinazolin-2-one | m/z [M + H]+ 339.11 |
| 113 | 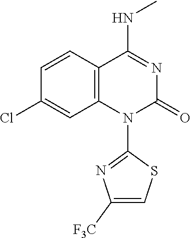 [SMILES: CNc1nc(=O)n(-c2nc(C(F)(F)F)cs2)c2cc(Cl)ccc12] | 7-chloro-4-(methylamino)-1-[4- (trifluoromethyl)(1,3-thiazol-2- yl)hydroquinazolin-2-one | m/z [M + H]+ 361.0 |
| 114 | 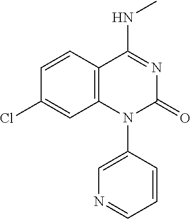 [SMILES: CNc1nc(=O)n(-c2cccnc2)c2cc(Cl)ccc12] | 7-chloro-4-(methylamino)-1-(3- pyridyl)-hydroquinazolin-2-one | m/z [M + H]+ 287.1 |
| 115 | 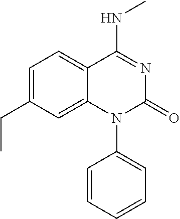 [SMILES: CCc1ccc2c(NC)nc(=O)n(-c3ccccc3)c2c1] | 7-ethyl-4-(methylamino)-1- phenylhydro-quinazolin-2-one | m/z [M + H]+ 280.1 |
| 116 | 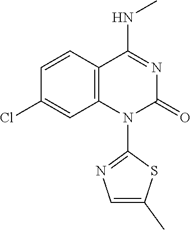 [SMILES: CNc1nc(=O)n(-c2ncc(C)s2)c2cc(Cl)ccc12] | 7-chloro-1-(5-methyl(1,3-thiazol-2- yl))-4-(methylamino)hydroquinazolin- 2-one | m/z [M + H]+ 307.0 |
| 117 | 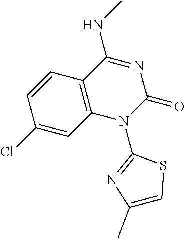 [SMILES: CNc1nc(=O)n(-c2nc(C)cs2)c2cc(Cl)ccc12] | 7-chloro-1-(4-methyl(1,3-thiazol-2- yl))-4-(methylamino)hydroquinazolin- 2-one | m/z [M + H]+ 307.0 |
| 118 | 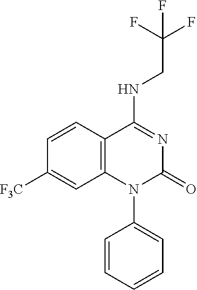 [SMILES: O=c1nc(NCC(F)(F)F)c2ccc(C(F)(F)F)cc2n1-c1ccccc1] | 1-phenyl-4-[(2,2,2- trifluoroethyl)amino]-7- (trifluoromethyl)hydroquinazolin-2- one | m/z [M + H]+ 388.0 |
| 119 | 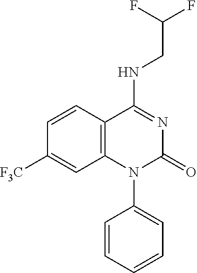 [SMILES: O=c1nc(NCC(F)F)c2ccc(C(F)(F)F)cc2n1-c1ccccc1] | 4-[(2,2-difluoroethyl)amino]-1-phenyl- 7-(trifluoromethyl)hydroquinazolin-2- one | m/z [M + H]+ 370.0 |
| 120 | 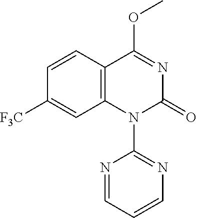 [SMILES: COc1nc(=O)n(-c2ncccn2)c2cc(C(F)(F)F)ccc12] | 4-methoxy-1-pyrimidin-2-yl-7- (trifluoro-methyl)hydroquinazolin-2- one | m/z [M + H]+ 323.0 |
| 121 | 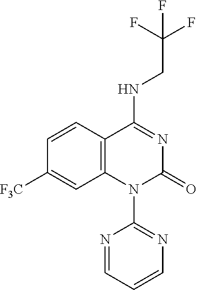 [SMILES: O=c1nc(NCC(F)(F)F)c2ccc(C(F)(F)F)cc2n1-c1ncccn1] | 1-pyrimidin-2-yl-4-[(2,2,2-trifluoro- ethyl)amino]-7-(trifluoromethyl)hydro- quinazolin-2-one | m/z [M + H]+ 390.0 |
| 122 | 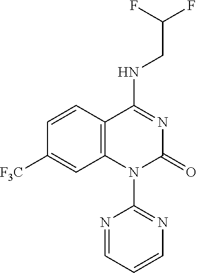 [SMILES: O=c1nc(NCC(F)F)c2ccc(C(F)(F)F)cc2n1-c1ncccn1] | 4-[(2,2-difluoroethyl)amino]-1- pyrimidin-2-yl-7- (trifluoromethyl)hydro-quinazolin-2- one | m/z [M + H]+ 372.0 |
| 123 | 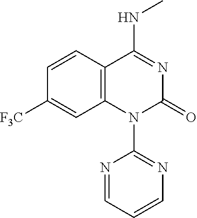 [SMILES: CNc1nc(=O)n(-c2ncccn2)c2cc(C(F)(F)F)ccc12] | 4-(methylamino)-1-pyrimidin-2-yl-7- (trifluoromethyl)hydroquinazolin-2- one | m/z [M + H]+ 322.0 |
| 124 | 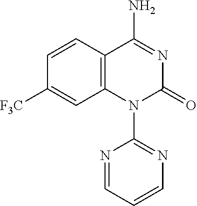 [SMILES: Nc1nc(=O)n(-c2ncccn2)c2cc(C(F)(F)F)ccc12] | 4-amino-1-pyrimidin-2-yl-7-(trifluoro- methyl)hydroquinazolin-2-one | m/z [M + H]+ 308.0 |
| 125 | 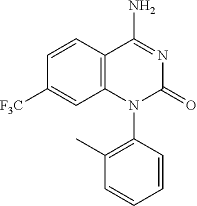 [SMILES: Cc1ccccc1-n1c(=O)nc(N)c2ccc(C(F)(F)F)cc21] | 4-amino-1-(2-methylphenyl)-7- (trifluoromethyl)hydroquinazolin-2- one | m/z [M + H]+ 32.0 |
| 126 | 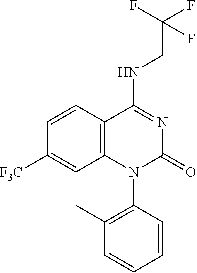 [SMILES: Cc1ccccc1-n1c(=O)nc(NCC(F)(F)F)c2ccc(C(F)(F)F)cc21] | 1-(2-methylphenyl)-4-[(2,2,2-trifluoro- ethyl)amino]-7-(trifluoromethyl)hydro- quinazolin-2-one | m/z [M + H]+ 402.0 |
| 127 | 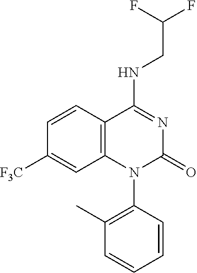 [SMILES: Cc1ccccc1-n1c(=O)nc(NCC(F)F)c2ccc(C(F)(F)F)cc21] | 4-[(2,2-difluoroethyl)amino]-1-(2- methylphenyl)-7- (trifluoromethyl)hydro-quinazolin-2- one | m/z [M + H]+ 384.0 |
| 128 | 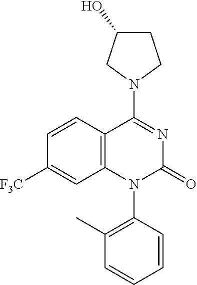 [SMILES: Cc1ccccc1-n1c(=O)nc(N2CC[C@@H](O)C2)c2ccc(C(F)(F)F)cc21] | 4-((3R)-3-hydroxypyrrolidin-1-yl)-1- (2-methylphenyl)-7- (trifluoromethyl)hydro-quinazolin-2- one | m/z [M + H]+ 390.0 |
| 129 | 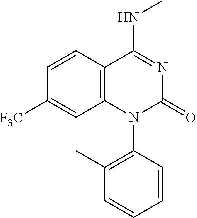 [SMILES: CNc1nc(=O)n(-c2ccccc2C)c2cc(C(F)(F)F)ccc12] | 4-(methylamino)-1-(2-methylphenyl)- 7-(trifluoromethyl)hydroquinazolin-2- one | m/z [M + H]+ 334.0 |
| 130 | 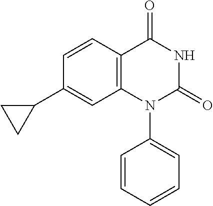 [SMILES: O=c1[nH]c(=O)n(-c2ccccc2)c2cc(C3CC3)ccc12] | 7-cyclopropyl-1-phenyl-1,3-dihydro- quinazoline-2,4-dione | m/z [M + H]+ 279.21 |
| 132 | 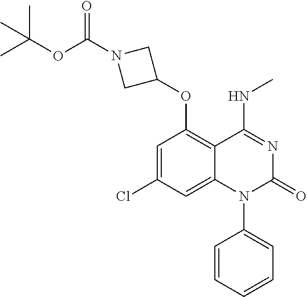 [SMILES: CNc1nc(=O)n(-c2ccccc2)c2cc(Cl)cc(OC3CN(C(=O)OC(C)(C)C)C3)c12] | tert-butyl 3-[7-chloro-4- (methylamino)-2-oxo-1- phenylhydroquinazolin-5-yl- oxy]azetidinecarboxylate | m/z [M + H]+ 457.13 |
| 133 | 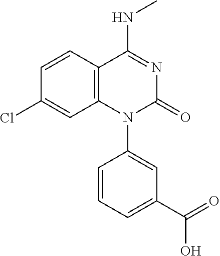 [SMILES: CNc1nc(=O)n(-c2cccc(C(=O)O)c2)c2cc(Cl)ccc12] | 3-[7-chloro-4-(methylamino)-2-oxo- hydroquinazolin-1-yl]benzoic acid | m/z [M + H]+ 330.11 |
| 134 | 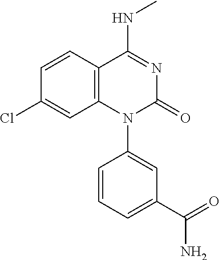 [SMILES: CNc1nc(=O)n(-c2cccc(C(N)=O)c2)c2cc(Cl)ccc12] | 3-[7-chloro-4-(methylamino)-2-oxo- hydroquinazolin-1-yl]benzamide | m/z [M + H]+ 329.11 |
| 135 | 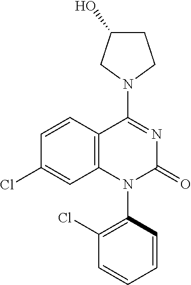 | 4-((3R)-3-hydroxypyrrolidin-1-yl)-7- chloro-1-(2-chlorophenyl)hydro- quinazolin-2-one | m/z [M + H]+ 376.10 |
| 136 | 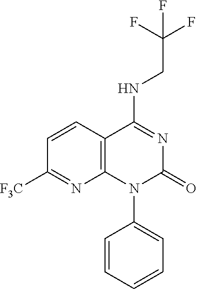 [SMILES: O=c1nc(NCC(F)(F)F)c2ccc(C(F)(F)F)nc2n1-c1ccccc1] | 1-phenyl-4-((2,2,2- trifluoroethyl)amino)-7- (trifluoromethyl)pyrido[2,3-d]- pyrimidin-2(1H)-one | m/z [M + H]+ 389.0 |
| 137 | 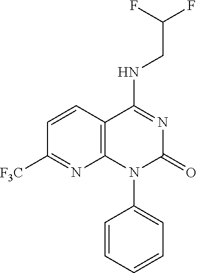 [SMILES: O=c1nc(NCC(F)F)c2ccc(C(F)(F)F)nc2n1-c1ccccc1] | 4-((2,2-difluoroethyl)amino)-1- phenyl-7-(trifluoromethyl)pyrido[2,3- d]pyrimidin-2(1H)-one | m/z [M + H]+ 371.0 |
| 138 | 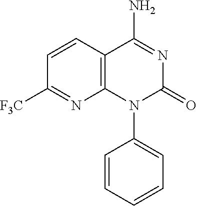 [SMILES: Nc1nc(=O)n(-c2ccccc2)c2nc(C(F)(F)F)ccc12] | 4-amino-1-phenyl-7-(trifluoromethyl)- hydroxypyrido[2,3-d]pyrimidin-2-one | m/z [M + H]+ 307.0 |
| 139 | 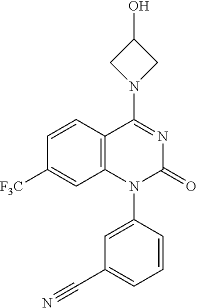 [SMILES: N#Cc1cccc(-n2c(=O)nc(N3CC(O)C3)c3ccc(C(F)(F)F)cc32)c1] | 3-[4-(3-hydroxyazetidinyl)-2-oxo-7- (trifluoromethyl)hydroquinazolinyl]- benzenecarbonitrile | m/z [M + H]+ 387.0 |
| 140 | 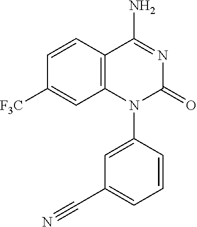 [SMILES: N#Cc1cccc(-n2c(=O)nc(N)c3ccc(C(F)(F)F)cc32)c1] | 3-[4-amino-2-oxo-7-(trifluoromethyl)- hydroquinazolinyl]benzenecarbonitrile | m/z [M + H]+ 331.0 |
| 141 | 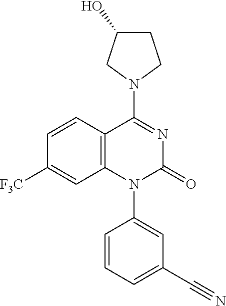 [SMILES: N#Cc1cccc(-n2c(=O)nc(N3CC[C@@H](O)C3)c3ccc(C(F)(F)F)cc32)c1] | 3-[4-((3R)-3-hydroxypyrrolidinyl)-2- oxo-7-(trifluoromethyl)hydro- quinazolinyl]-benzenecarbonitrile | m/z [M + H]+ 401.1 |
| 142 | 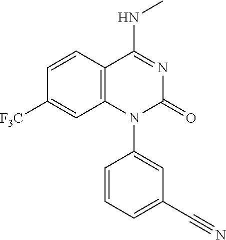 [SMILES: CNc1nc(=O)n(-c2cccc(C#N)c2)c2cc(C(F)(F)F)ccc12] | 3-[4-(methylamino)-2-oxo-7-(trifluoro- methyl)hydroquinazolinyl]benzene- carbonitrile | m/z [M + H]+ 345.1 |
| 143 | 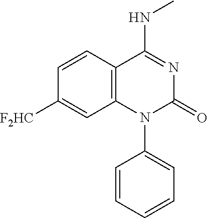 [SMILES: CNc1nc(=O)n(-c2ccccc2)c2cc(C(F)F)ccc12] | 7-(difluoromethyl)-4-(methylamino)- 1-phenylhydroquinazolin-2-one | m/z [M + H]+ 302.0 |
| 144 | 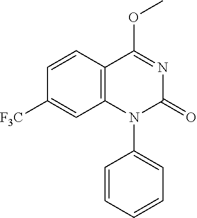 [SMILES: COc1nc(=O)n(-c2ccccc2)c2cc(C(F)(F)F)ccc12] | 4-methoxy-1-phenyl-7- (trifluoromethyl)-hydroquinazolin-2- one | m/z [M + H]⁺ 321.0 |
| 145 | 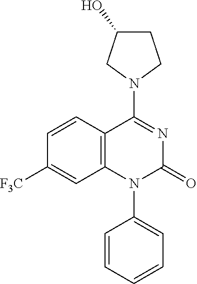 [SMILES: O=c1nc(N2CC[C@@H](O)C2)c2ccc(C(F)(F)F)cc2n1-c1ccccc1] | 4-((3R)-3-hydroxypyrrolidinyl)-1- phenyl-7-(trifluoromethyl)hydro- quinazolin-2-one | m/z [M + H]+ 376.0 |
| 146 | 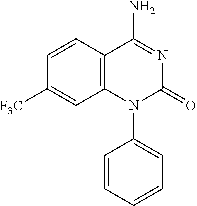 [SMILES: Nc1nc(=O)n(-c2ccccc2)c2cc(C(F)(F)F)ccc12] | 4-amino-1-phenyl-7-(trifluoromethyl)+- hydroquinazolin-2-one | m/z [M + H]+ 306.0 |
| 147 | 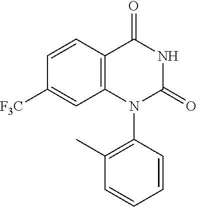 [SMILES: Cc1ccccc1-n1c(=O)[nH]c(=O)c2ccc(C(F)(F)F)cc21] | 1-(2-methylphenyl)-7- (trifluoromethyl)-1,3- dihydroquinazoline-2,4-dione | m/z [M + H]+ 321.0 |
| 148 | 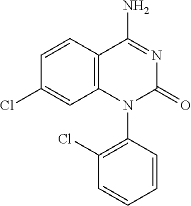 [SMILES: Nc1nc(=O)n(-c2ccccc2Cl)c2cc(Cl)ccc12] | 4-amino-7-chloro-1-(2-chlorophenyl)- hydroquinazolin-2-one | m/z [M + H]+ 307.06 |
| 149 | 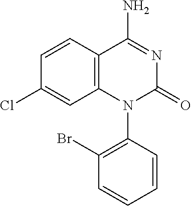 [SMILES: Nc1nc(=O)n(-c2ccccc2Br)c2cc(Cl)ccc12] | 4-amino-1-(2-bromophenyl)-7-chloro- hydroquinazolin-2-one | m/z [M + H]+ 349.98 |
| 150 | 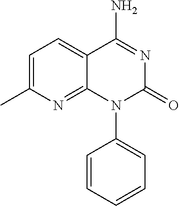 | 4-amino-7-methyl-1-phenylhydro- pyridino[2,3-d]pyrimidin-2-one | m/z [M + H]+ 353.24 |
| 151 | 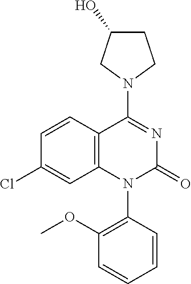 [SMILES: COc1ccccc1-n1c(=O)nc(N2CC[C@@H](O)C2)c2ccc(Cl)cc21] | 4-((3R)-3-hydroxypyrrolidin-1-yl)-7- chloro-1-(2-methoxyphenyl)hydro- quinazolin-2-one | m/z [M + H]+ 372.10 |
| 153 | 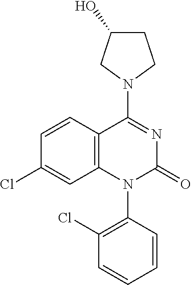 [SMILES: O=c1nc(N2CC[C@@H](O)C2)c2ccc(Cl)cc2n1-c1ccccc1Cl] | 4-((3R)-3-hydroxypyrrolidin-1-yl)-7- chloro-1-(2-chlorophenyl)hydro- quinazolin-2-one | m/z [M + H]+ 376.04 |
| 154 | 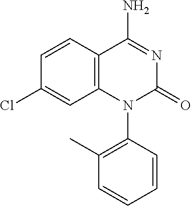 | 4-amino-7-chloro-1-(2- methylphenyl)-hydroquinazolin-2-one | m/z [M + H]+ 386.0 |
| 155 | 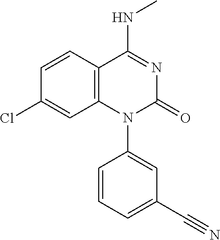 [SMILES: CNc1nc(=O)n(-c2cccc(C#N)c2)c2cc(Cl)ccc12] | 3-[7-chloro-4-(methylamino)-2-oxo- hydroquinazolinyl]benzenecarbonitrile | m/z [M + H]+ 311.12 |
| 156 | 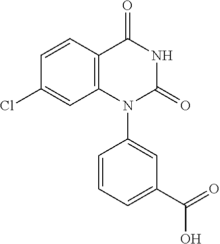 [SMILES: O=C(O)c1cccc(-n2c(=O)[nH]c(=O)c3ccc(Cl)cc32)c1] | 3-(7-chloro-4-hydroxy-2-oxohydro- quinazolinyl)benzoic acid | m/z [M + H]+ 317.06 |
| 157 | 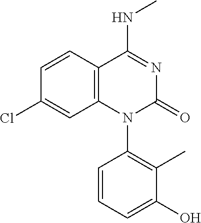 [SMILES: CNc1nc(=O)n(-c2cccc(O)c2C)c2cc(Cl)ccc12] | 7-chloro-1-(3-hydroxy-2- methylphenyl)-4- (methylamino)hydroquinazolin-2-one | m/z [M + H]+ 316.12 |
| 159 | 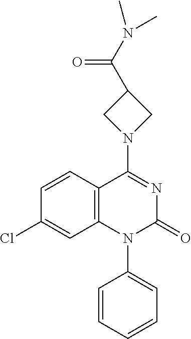 [SMILES: CN(C)C(=O)C1CN(c2nc(=O)n(-c3ccccc3)c3cc(Cl)ccc23)C1] | [1-(7-chloro-2-oxo-1-phenylhydro- quinazolin-4-yl)azetidin-3-yl]-N,N- dimethylcarboxamide | m/z [M + H]+ 383.1 |
| 160 | 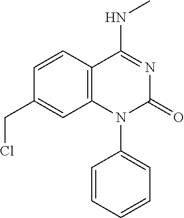 [SMILES: CNc1nc(=O)n(-c2ccccc2)c2cc(CCl)ccc12] | 7-(chloromethyl)-4-(methylamino)-1- phenylhydroquinazolin-2-one | m/z [M + H]+ 300.0 |
| 161 | 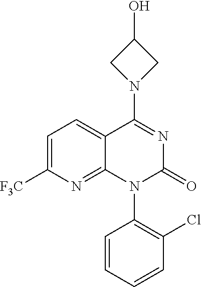 [SMILES: O=c1nc(N2CC(O)C2)c2ccc(C(F)(F)F)nc2n1-c1ccccc1Cl] | 1-(2-chlorophenyl)-4-(3-hydroxy- azetidinyl)-7-(trifluoromethyl)pyrido- [2,3-d]pyrimidin-2(1H)-one | m/z [M + H]+ 397.1 |
| 162 | 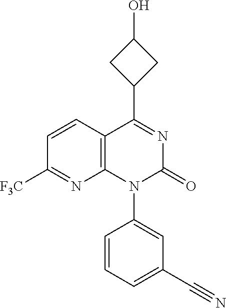 [SMILES: N#Cc1cccc(-n2c(=O)nc(C3CC(O)C3)c3ccc(C(F)(F)F)nc32)c1] | 3-(4-(3-hydroxyazetidin-1-yl)-2-oxo- 7-(trifluoromethyl)pyrido[2,3- d]pyrimidin-1(2H)-yl)benzonitrile | m/z [M + H]+ 388.1 |
| 163 | 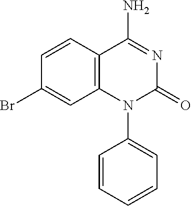 [SMILES: Nc1nc(=O)n(-c2ccccc2)c2cc(Br)ccc12] | 4-amino-7-bromo-1-phenylhydro- quinazolin-2-one | m/z [M + H]+ 316.0 |
| 164 | 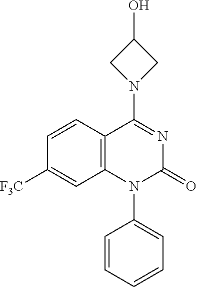 [SMILES: O=c1nc(N2CC(O)C2)c2ccc(C(F)(F)F)cc2n1-c1ccccc1] | 4-(3-hydroxyazetidin-1-yl)-1-phenyl- 7-(trifluoromethyl)pyrido[2,3- d]pyrimidin-2(1H)-one | m/z [M + H]+ 362.0 |
| 165 | 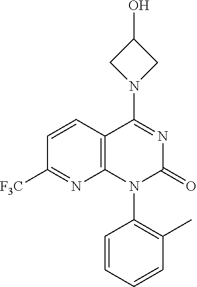 [SMILES: Cc1ccccc1-n1c(=O)nc(N2CC(O)C2)c2ccc(C(F)(F)F)nc21] | 4-(3-hydroxyazetidinyl)-1-(2-methyl- phenyl)-7-(trifluoromethyl)pyrido[2,3- d]pyrimidin-2(1H)-one | m/z [M + H]+ 377.24 |
| 166 | 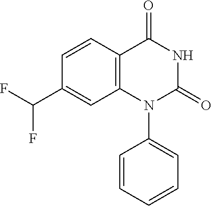 [SMILES: O=c1[nH]c(=O)n(-c2ccccc2)c2cc(C(F)F)ccc12] | 7-(difluoromethyl)-1-phenyl-1,3- dihydroquinazoline-2,4-dione | m/z [M + H]+ 289.0 |
| 167 | 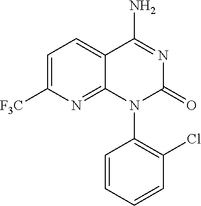 [SMILES: Nc1nc(=O)n(-c2ccccc2Cl)c2nc(C(F)(F)F)ccc12] | 4-amino-1-(2-chlorophenyl)-7- (trifluoro-methyl)pyrido[2,3- d]pyrimidin-2(1H)-one | m/z [M + H]+ 341.10 |
| 168 | 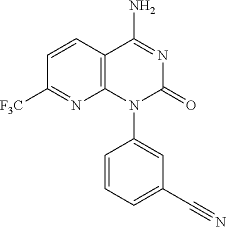 [SMILES: N#Cc1cccc(-n2c(=O)nc(N)c3ccc(C(F)(F)F)nc32)c1] | 3-(4-amino-2-oxo-7-(trifluoromethyl)- pyrido[2,3-d]pyrimidin-1(2H)-yl)- benzonitrile | m/z [M + H]+ 332.10 |
| 169 | 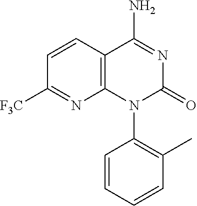 [SMILES: Cc1ccccc1-n1c(=O)nc(N)c2ccc(C(F)(F)F)nc21] | 4-amino-1-(2-methylphenyl)-7- (trifluoromethyl)pyrido[2,3- d]pyrimdiin-2(1H)-one | m/z [M + H]⁺ 321.1 |
| 170 | 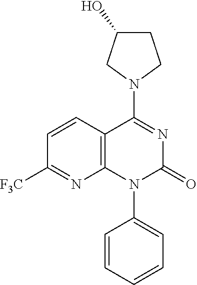 [SMILES: O=c1nc(N2CC[C@@H](O)C2)c2ccc(C(F)(F)F)nc2n1-c1ccccc1] | (R)-4-(3-hydroxypyrrolidin-1-yl)-1- phenyl-7-(trifluoromethyl)pyrido[2,3- d]-pyrimidin-2(1H)-one | m/z [M + H]+ 377.15 |
| 171 | 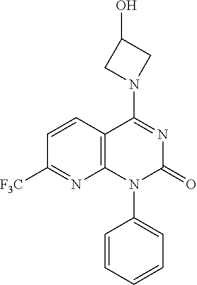 [SMILES: O=c1nc(N2CC(O)C2)c2ccc(C(F)(F)F)nc2n1-c1ccccc1] | 4-(3-hydroxyazetidin-1-yl)-1-phenyl- 7-(trifluoromethyl)pyrido[2,3- d]pyrimidin-2(1H)-one | m/z [M + H]+ 363.0 |
| 172 | 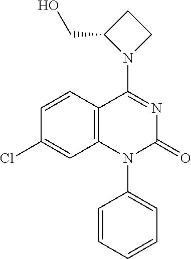 [SMILES: O=c1nc(N2CC[C@H]2CO)c2ccc(Cl)cc2n1-c1ccccc1] | (S)-7-chloro-4-(2-(hydroxymethyl)- azetidin-1-yl)-1-phenylquinazolin- 2(1H)-one | m/z [M + H]⁺ 342.1 |
| 173 | 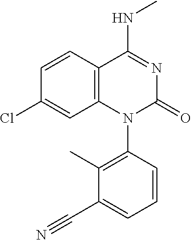 [SMILES: CNc1nc(=O)n(-c2cccc(C#N)c2C)c2cc(Cl)ccc12] | 3-[7-chloro-4-(methylamino)-2-oxo- hydroquinazolinyl]-2-methylbenzene- carbonitrile | m/z [M + H]+ 325.11 |
| 174 | 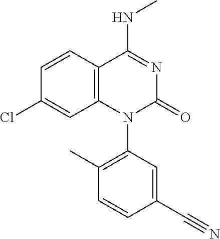 [SMILES: CNc1nc(=O)n(-c2cc(C#N)ccc2C)c2cc(Cl)ccc12] | 3-(7-chloro-4-(methylamino)-2-oxo- quinazolin-1(2H)-yl)-2-methyl- benzonitrile | m/z [M + H]+ 325.11 |
| 175 | 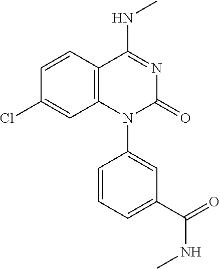 [SMILES: CNC(=O)c1cccc(-n2c(=O)nc(NC)c3ccc(Cl)cc32)c1] | 3-(7-chloro-4-(methylamino)-2-oxo- quinazolin-1(2H)-yl)benzamide | m/z [M + H]⁺ 343.1 |
| 176 | 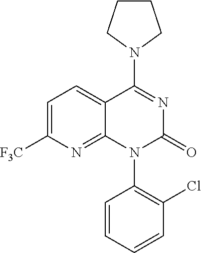 [SMILES: O=c1nc(N2CCCC2)c2ccc(C(F)(F)F)nc2n1-c1ccccc1Cl] | 1-(2-chlorophenyl)-4-(pyrrolidin-1-yl)- 7-(trifluoromethyl)pyrido[2,3- d]pyrimidin-2(1H)-one | m/z [M + H]+ 395.10 |
| 177 | 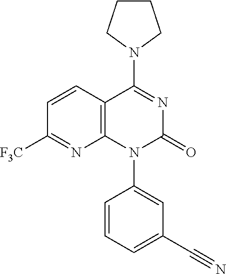 [SMILES: N#Cc1cccc(-n2c(=O)nc(N3CCCC3)c3ccc(C(F)(F)F)nc32)c1] | 3-(2-oxo-4-(pyrrolidin-1-yl)-7- (trifluoro-methylpyrido[2,3- d]pyrimidin-1(2H)-yl)benzonitrile | m/z [M + H]+ 385.15 |
| 178 | 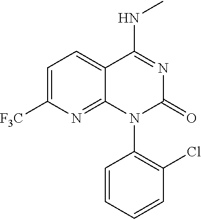 [SMILES: CNc1nc(=O)n(-c2ccccc2Cl)c2nc(C(F)(F)F)ccc12] | 1-(2-chlorophenyl)-4-(methylamino)- 7-(trifluoromethyl)pyrido[2,3- d]pyrimidin-2(1H)-one | m/z [M + H]+ 355.10 |
| 179 | 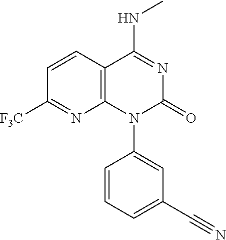 [SMILES: CNc1nc(=O)n(-c2cccc(C#N)c2)c2nc(C(F)(F)F)ccc12] | 3-[4-(methylamino)-2-oxo-7-(trifluoro- methyl)pyrido[2,3-d]pyrimidin-1(2H)- yl)benzonitrile | m/z [M + H]+ 346.1 |
| 180 | 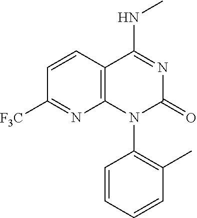 [SMILES: CNc1nc(=O)n(-c2ccccc2C)c2nc(C(F)(F)F)ccc12] | 4-(methylamino)-1-(2-methylphenyl)- 7-(trifluoromethyl)pyrido[2,3- d]pyrimidin-2(1H)-one | m/z [M + H]+ 335.10 |
| 181 | 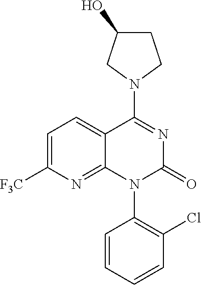 [SMILES: O=c1nc(N2CC[C@H](O)C2)c2ccc(C(F)(F)F)nc2n1-c1ccccc1Cl] | (S)-1-(2-chlorophenyl)-4-(3-hydroxy- pyrrolidin-1-yl)-7-(trifluoromethyl)- pyrido[2,3-d]pyrimidin-2(1H)-one | m/z [M + H]+ 411.10 |
| 182 | 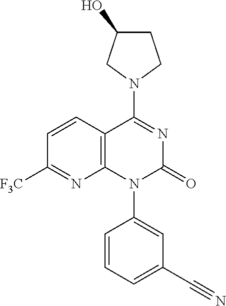 [SMILES: N#Cc1cccc(-n2c(=O)nc(N3CC[C@H](O)C3)c3ccc(C(F)(F)F)nc32)c1] | (S)-3-(4-(3-hydroxypyrrolidin-1-yl)-2- oxo-7-(trifluoromethyl)pyrido[2,3- d]pyrimidin-1(2H)-yl)benzonitrile | m/z [M + H]+ 402.1 |
| 183 | 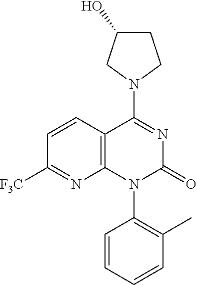 [SMILES: Cc1ccccc1-n1c(=O)nc(N2CC[C@@H](O)C2)c2ccc(C(F)(F)F)nc21] | (R)-4-(3-hydroxypyrrolidin-1-yl)-1-(o- tolyl)-7-(trifluoromethyl)pyrido[2,3-d]- pyrimidin-2(1H)-one | m/z [M + H]⁺ 391.2 |
| 184 | 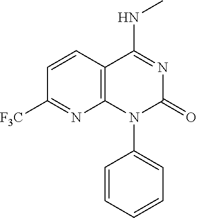 [SMILES: CNc1nc(=O)n(-c2ccccc2)c2nc(C(F)(F)F)ccc12] | 4-(methylamino)-1-phenyl-7- (trifluoromethyl)pyrido[2,3-d]- pyrimidin-2(1H)-one | m/z [M + H]+ 321.1 |
| 185 | 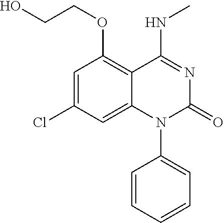 [SMILES: CNc1nc(=O)n(-c2ccccc2)c2cc(Cl)cc(OCCO)c12] | 7-chloro-5-(2-hydroxyethoxy)-4- (methylamino)-1-phenylhydro- quinazolin-2-one | m/z [M + H]+ 346.17 |
| 186 | 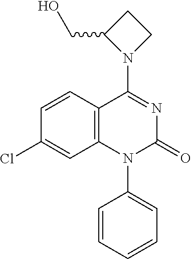 [SMILES: O=c1nc(N2CCC2CO)c2ccc(Cl)cc2n1-c1ccccc1] | 7-chloro-4-[2- (hydroxymethyl)azetidin-1-yl]-1- phenylhydroquinazolin-2-one | m/z [M + H]+ 342.17 |
| 187 | 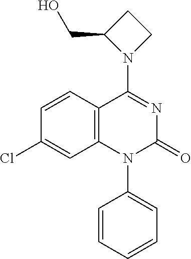 [SMILES: O=c1nc(N2CC[C@@H]2CO)c2ccc(Cl)cc2n1-c1ccccc1] | (R)-7-chloro-4-(2-(hydroxymethyl)- azetidin-1-yl)-1-phenylquinazolin- 2(1H)-one | m/z [M + H]+ 342.17 |
| 188 | 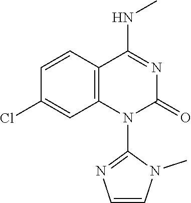 [SMILES: CNc1nc(=O)n(-c2nccn2C)c2cc(Cl)ccc12] | 7-chloro-4-(methylamino)-1-(1- methyl-imidazol-2- yl)hydroquinazolin-2-one | m/z [M + H]+ 290.0 |
| 189 | 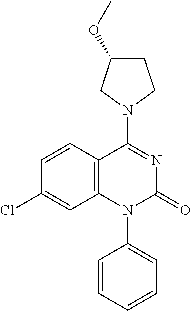 [SMILES: CO[C@@H]1CCN(c2nc(=O)n(-c3ccccc3)c3cc(Cl)ccc23)C1] | (R)-7-chloro-4-(3-methoxypyrrolidin- 1-yl)-1-phenylquinazolin-2(1H)-one | m/z [M + H]+ 356.24 |
| 190 | 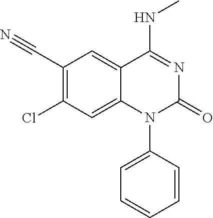 [SMILES: CNc1nc(=O)n(-c2ccccc2)c2cc(Cl)c(C#N)cc12] | 7-chloro-4-(methylamino)-2-oxo-1- phenylhydroquinazoline-6- carbonitrile | m/z [M + H]+ 311.18 |
| 191 | 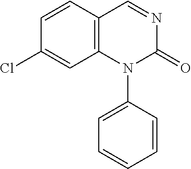 [SMILES: O=c1ncc2ccc(Cl)cc2n1-c1ccccc1] | 7-chloro-1-phenylhydroquinazolin-2- one | m/z [M + H]+ 257.26 |
| 192 | 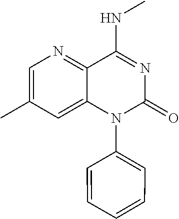 [SMILES: CNc1nc(=O)n(-c2ccccc2)c2cc(C)cnc12] | 7-methyl-4-(methylamino)-1-phenyl- pyrido[3,2-d]pyrimidin-2(1H)-one | m/z [M + H]+ 267.29 |
| 193 | 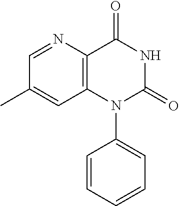 [SMILES: Cc1cnc2c(=O)[nH]c(=O)n(-c3ccccc3)c2c1] | 7-methyl-1-phenylpyrido[3,2-d]- pyrimidine-2,4(1H,3H)-dione | m/z [M + H]+ 254.24 |
| 194 | 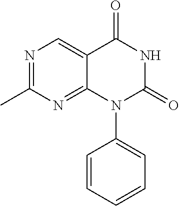 [SMILES: Cc1ncc2c(=O)[nH]c(=O)n(-c3ccccc3)c2n1] | 7-methyl-1-phenylpyrimido[4,5-d]- pyrimidine-2,4(1H,3H)-dione | m/z [M + H]⁺ 254.26 |
| 195 | 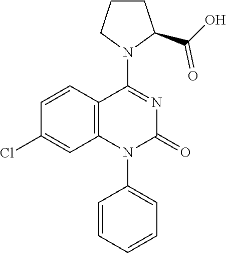 [SMILES: O=C(O)[C@@H]1CCCN1c1nc(=O)n(-c2ccccc2)c2cc(Cl)ccc12] | (2S)-1-(7-chloro-2-oxo-1- phenylhydro-quinazolin-4- yl)pyrrolidine-2-carobxylic acid | m/z [M + H]+ 370.12 |
| 196 | 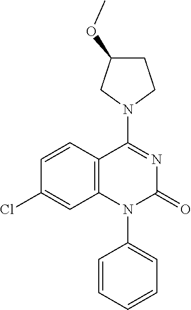 [SMILES: CO[C@H]1CCN(c2nc(=O)n(-c3ccccc3)c3cc(Cl)ccc23)C1] | (S)-7-chloro-4-(3-methoxypyrrolidin- 1-yl)-1-phenylquinazolin-2(1H)-one | m/z [M + H]+ 356.24 |
| 197 | 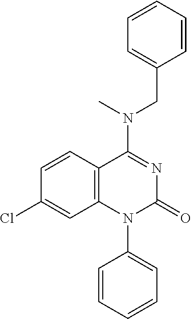 [SMILES: CN(Cc1ccccc1)c1nc(=O)n(-c2ccccc2)c2cc(Cl)ccc12] | 7-chloro-4-[methylbenzylamino]-1- phenylhydroquinazolin-2-one | m/z [M + H ]+ 376.24 |
| 188 | 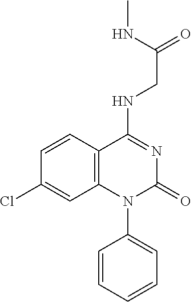 [SMILES: CNC(=O)CNc1nc(=O)n(-c2ccccc2)c2cc(Cl)ccc12] | 2-((7-chloro-2-oxo-1-phenyl-1,2- dihydroquinazolin-4- yl)(methyl)amino)-N- methylacetamide | m/z [M + H]+ 343.10 |
| 199 | 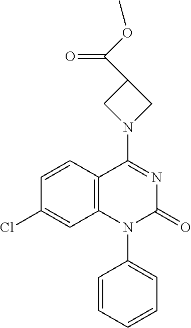 [SMILES: COC(=O)C1CN(c2nc(=O)n(-c3ccccc3)c3cc(Cl)ccc23)C1] | methyl 1-(7-chloro-2-oxo-1-phenyl- 1,2-dihydroquinazolin-4-yl)azetidine- 3-carboxylate | m/z [M + H]+ 370.10 |
| 200 | 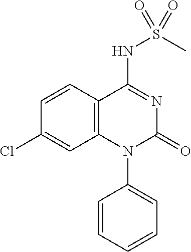 [SMILES: CS(=O)(=O)Nc1nc(=O)n(-c2ccccc2)c2cc(Cl)ccc12] | N-(7-chloro-2-oxo-1-phenyl-1,2- dihydroquinazolin-4-yl)methane- sulfonamide | m/z [M + H]+ 350.04 |
| 201 | 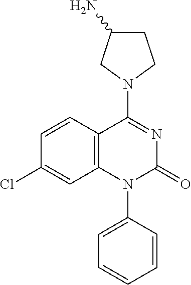 [SMILES: NC1CCN(c2nc(=O)n(-c3ccccc3)c3cc(Cl)ccc23)C1] | 4-(3-aminopyrrolidin-1-yl)-7-chloro-1- phenylquinazolin-2(1H)-one | m/z [M + H]+ 341.11 |
| 202 | 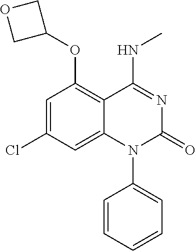 [SMILES: CNc1nc(=O)n(-c2ccccc2)c2cc(Cl)cc(OC3COC3)c12] | 7-chloro-4-(methylamino)-5-oxetan- 3-yloxy-1-phenylhydroquinazolin-2- one | m/z [M + H]+ 358.10 |
| 203 | 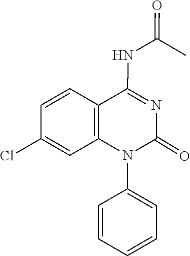 [SMILES: CC(=O)Nc1nc(=O)n(-c2ccccc2)c2cc(Cl)ccc12] | N-(7-chloro-2-oxo-1-phenyl-1,2- dihydroquinazolin-4-yl)acetamide | m/z [M + H]+ 314.12 |
| 204 | 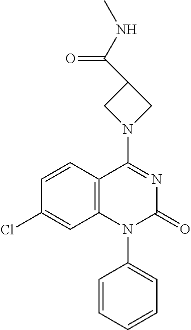 [SMILES: CNC(=O)C1CN(c2nc(=O)n(-c3ccccc3)c3cc(Cl)ccc23)C1] | 1-(7-chloro-2-oxo-1-phenyl-1,2- dihydroquinazolin-4-yl)-N-methyl- azetidine-3-carboxamide | m/z [M + H]+ 369.22 |
| 205 | 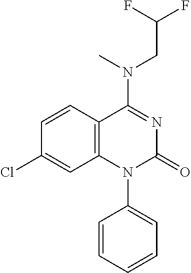 [SMILES: CN(CC(F)F)c1nc(=O)n(-c2ccccc2)c2cc(Cl)ccc12] | 4-[(2,2-difluoroethyl)methylamino]-7- chloro-1-phenylhydroquinazolin-2- one | m/z [M + H]+ 350.10 |
| 206 | 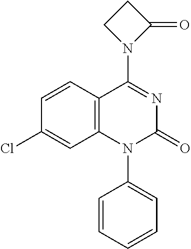 [SMILES: O=C1CCN1c1nc(=O)n(-c2ccccc2)c2cc(Cl)ccc12] | 7-chloro-4-(2-oxoazetidin-1-yl)-1- phenylhydroquinazolin-2-one | m/z [M + H]+ 326.17 |
| 207 | 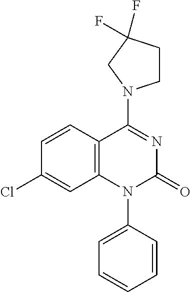 [SMILES: O=c1nc(N2CCC(F)(F)C2)c2ccc(Cl)cc2n1-c1ccccc1] | 4-(3,3-difluoropyrrolidin-1-yl)-7- chloro-1-phenylhydroquinazolin-2- one | m/z [M + H]+ 328.17 |
| 208 | 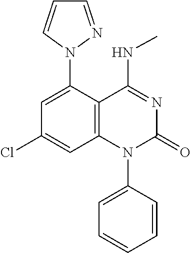 [SMILES: CNc1nc(=O)n(-c2ccccc2)c2cc(Cl)cc(-n3cccn3)c12] | 7-chloro-4-(methylamino)-1-phenyl- 5-(1H-pyrazol-1-yl)quinazolin-2(1H)- one | m/z [M + H]+ 352.18 |
| 209 | 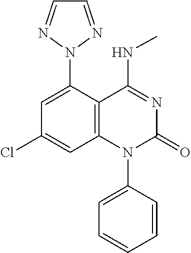 [SMILES: CNc1nc(=O)n(-c2ccccc2)c2cc(Cl)cc(-n3nccn3)c12] | 7-chloro-4-(methylamino)-1-phenyl- 5-(1,2,3-triazol-2-yl)quinazolin-2(1H)- one | m/z [M + H]+ 353.1 |
| 210 | 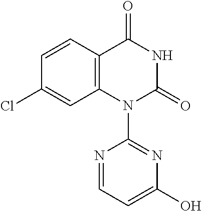 [SMILES: O=c1[nH]c(=O)n(-c2nccc(O)n2)c2cc(Cl)ccc12] | 7-chloro-1-(4-hydroxypyrimidin-2- yl)-1,3-dihydroquinazoline-2,4-dione | m/z [M + H]+ 291.0 |
| 211 | 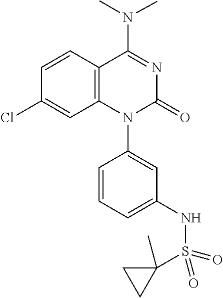 | 4-(dimethylamino)-7-chloro-1-(3- {[(methylcyclopropyl)sulfonyl]amino} phenyl)-hydroquinazolin-2-one | m/z [M + H]+ 3433.1 |
| 212 | 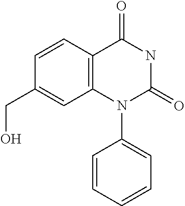 [SMILES: O=c1[nH]c(=O)n(-c2ccccc2)c2cc(CO)ccc12] | 7-(hydroxymethyl)-1-phenyl-1,3- dihydroquinazoline-2,4-dione | m/z [M + H]+ 269.0 |
| 213 | 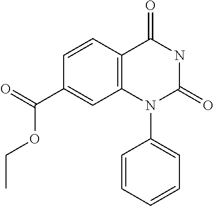 [SMILES: CCOC(=O)c1ccc2c(=O)[nH]c(=O)n(-c3ccccc3)c2c1] | ethyl 2,4-dioxo-1-phenyl-1,3- dihydro-quinazoline-7-carboxylate | m/z [M + H]+ 311.0 |
| 214 | 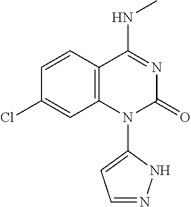 [SMILES: CNc1nc(=O)n(-c2ccn[nH]2)c2cc(Cl)ccc12] | 7-chloro-4-(methylamino)-1-pyrazol- 5-ylhydroquinazolin-2-one | m/z [M + H]+ 276.1 |
| 215 | 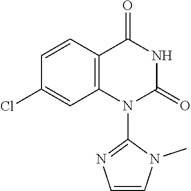 [SMILES: Cn1ccnc1-n1c(=O)[nH]c(=O)c2ccc(Cl)cc21] | 7-chloro-1-(1-methylimidazol-2-yl)- 1,3-dihydroquinazoline-2,4-dione | m/z [M + H]+ 277.1 |
| 216 | 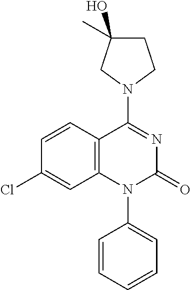 [SMILES: C[C@]1(O)CCN(c2nc(=O)n(-c3ccccc3)c3cc(Cl)ccc23)C1] | 4-((3S)-3-hydroxy-3- methylpyrrolidin-1-yl)-7-chloro-1- phenylhydroquinazolin-2-one | m/z [M + H]+ 256.12 |
| 217 | 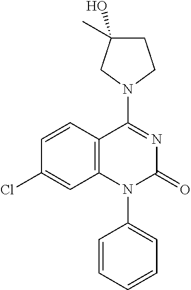 [SMILES: C[C@@]1(O)CCN(c2nc(=O)n(-c3ccccc3)c3cc(Cl)ccc23)C1] | 4-((3R)-3-hydroxy-3- methylpyrrolidin-1-yl)-7-chloro-1- phenylhydroquinazolin-2-one | m/z [M + H]+ 256.12 |
| 218 | 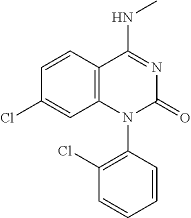 [SMILES: CNc1nc(=O)n(-c2ccccc2Cl)c2cc(Cl)ccc12] | 7-chloro-1-(2-chlorophenyl)-4- (methylamino)quinazolin-2(1H)-one | m/z [M + H]+ 320.0 |
| 219 | 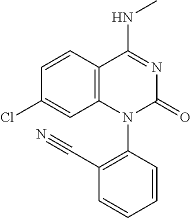 [SMILES: CNc1nc(=O)n(-c2ccccc2C#N)c2cc(Cl)ccc12] | 2-(7-chloro-4-(methylamino)-2-oxo- quinazolin-1(2H)-yl)benzonitrile | m/z [M + H]+ 311.0 |
| 220 | 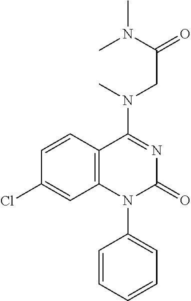 [SMILES: CN(C)C(=O)CN(C)c1nc(=O)n(-c2ccccc2)c2cc(Cl)ccc12] | 2-((7-chloro-2-oxo-1-phenyl-1,2- dihydroquinazolin-4- yl)(methyl)amino)-N,N- dimethylacetamide | m/z [M + H]+ 371.18 |
| 221 | 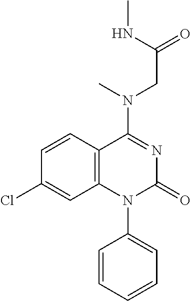 [SMILES: CNC(=O)CN(C)c1nc(=O)n(-c2ccccc2)c2cc(Cl)ccc12] | 2-((7-chloro-2-oxo-1-phenyl-1,2- dihydroquinazolin-4- yl)(methyl)amino)-,N- methylacetamide | m/z [M + H]+ 357.12 |
| 222 | 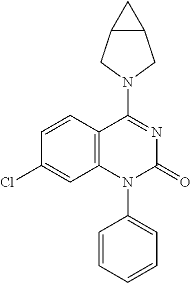 [SMILES: O=c1nc(N2CC3CC3C2)c2ccc(Cl)cc2n1-c1ccccc1] | 4-(3-azabicyclo[3.1.0]hex-3-yl)-7- chloro-1-phenylhydroquinazolin-2- one | m/z [M + H]+ 338.13 |
| 223 | 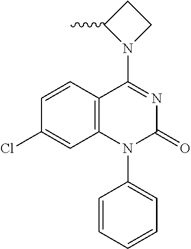 [SMILES: CC1CCN1c1nc(=O)n(-c2ccccc2)c2cc(Cl)ccc12] | 7-chloro-4-(2-methylazetidin-1-yl)-1- phenylhydroquinazolin-2-one | m/z [M + H]+ 326.13 |
| 224 | 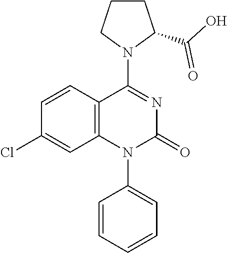 [SMILES: O=C(O)[C@H]1CCCN1c1nc(=O)n(-c2ccccc2)c2cc(Cl)ccc12] | (2R)-1-(7-chloro-2-oxo-1- phenylhydro-quinazolin-4- yl)pyrrolidine-2-carboxylic acid | m/z [M + H]+ 370.12 |
| 225 | 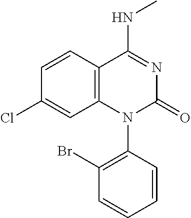 [SMILES: CNc1nc(=O)n(-c2ccccc2Br)c2cc(Cl)ccc12] | 1-(2-bromophenyl)-7-chloro-4- (methylamino)quinazolin-2(1H)-one | m/z [M + H]+ 364.0 |
| 226 | 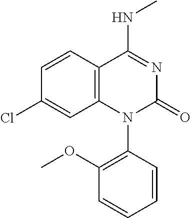 [SMILES: CNc1nc(=O)n(-c2ccccc2OC)c2cc(Cl)ccc12] | 7-chloro-1-(2-methoxyphenyl)-4- (methylamino)hydroquinazolin-2-one | m/z [M + H]+ 316.0 |
| 227 | 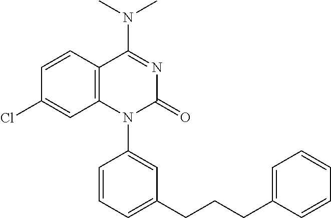 [SMILES: CN(C)c1nc(=O)n(-c2cccc(CCCc3ccccc3)c2)c2cc(Cl)ccc12] | 4-(dimethylamino)-7-chloro-1-[3-(3- phenylpropyl)phenyl]hydroquinazolin- 2-one | m/z [M + H]+ 418.2 |
| 228 | 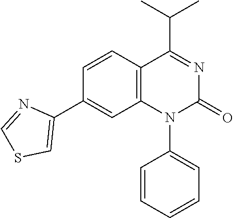 [SMILES: CC(C)c1nc(=O)n(-c2ccccc2)c2cc(-c3cscn3)ccc12] | 4-(dimethylamino)-1-phenyl-7-(1,3- thiazol-4-yl)hydroquinazolin-2-one | m/z [M + H]+ 349.0 |
| 229 | 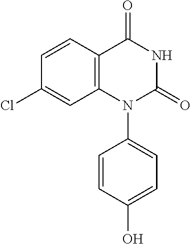 [SMILES: O=c1[nH]c(=O)n(-c2ccc(O)cc2)c2cc(Cl)ccc12] | 7-chloro-1-(4-hydroxyphenyl)-1,3- dihydroquinazoline-2,4-dione | m/z [M + H]+ 289.1 |
| 230 | 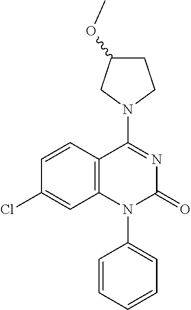 [SMILES: COC1CCN(c2nc(=O)n(-c3ccccc3)c3cc(Cl)ccc23)C1] | 7-chloro-4-(3-methoxypyrrolidin-1- yl)-1-phenylquinazolin-2(1H)-one | m/z [M + H]+ 356.12 |
| 231 | 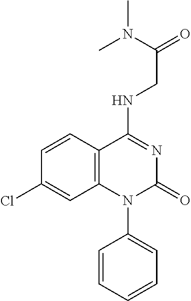 [SMILES: CN(C)C(=O)CNc1nc(=O)n(-c2ccccc2)c2cc(Cl)ccc12] | 2-[(7-chloro-2-oxo-1-phenyl-1,2- dihydroquinazolin-4-yl)amino]-N,N- dimethylacetamide | m/z [M + H]⁺ 357.12 |
| 232 | 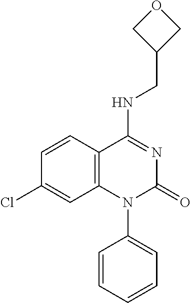 [SMILES: O=c1nc(NCC2COC2)c2ccc(Cl)cc2n1-c1ccccc1] | 7-chloro-4-[(oxetan-3- ylmethyl)amino]-1- phenylhydroquinazolin-2-one | m/z [M + H]+ 342.23 |
| 233 | 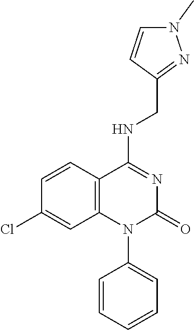 [SMILES: Cn1ccc(CNc2nc(=O)n(-c3ccccc3)c3cc(Cl)ccc23)n1] | 7-chloro-4-{[(1-methylpyrazol-3-yl)- methyl]amino}-1-phenylhydro- quinazolin-2-one | m/z [M + H]+ 366.24 |
| 234 | 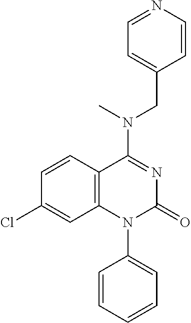 [SMILES: CN(Cc1ccncc1)c1nc(=O)n(-c2ccccc2)c2cc(Cl)ccc12] | 7-chloro-4-[methyl(pyridin-4- ylmethyl)-amino]-1- phenylhydroquinazolin-2-one | m/z [M + H]+ 377.1 |
| 235 | 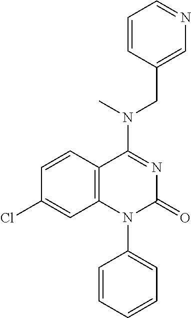 [SMILES: CN(Cc1cccnc1)c1nc(=O)n(-c2ccccc2)c2cc(Cl)ccc12] | 7-chloro-4-[methyl(pyridin-3- ylmethyl)-amino]-1- phenylhydroquinazolin-2-one | m/z [M + H]+ 377.24 |
| 236 | 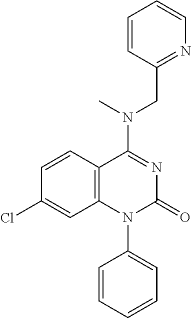 [SMILES: CN(Cc1ccccn1)c1nc(=O)n(-c2ccccc2)c2cc(Cl)ccc12] | 7-chloro-4-[methyl(pyridin-2- ylmethyl)-amino]-1- phenylhydroquinazolin-2-one | m/z [M + H]+ 377.12 |
| 237 | 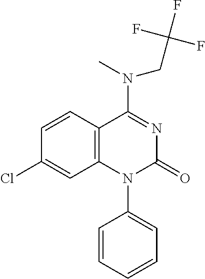 [SMILES: CN(CC(F)(F)F)c1nc(=O)n(-c2ccccc2)c2cc(Cl)ccc12] | 7-chloro-4-[methyl(2,2,2- trifluoroethyl)-amino]-1- phenylhydroquinazolin-2-one | m/z [M + H]+ 368.18 |
| 238 | 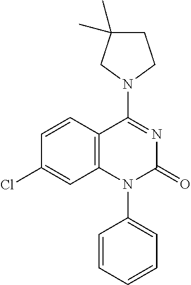 [SMILES: CC1(C)CCN(c2nc(=O)n(-c3ccccc3)c3cc(Cl)ccc23)C1] | 4-(3,3-dimethylpyrrolidin-1-yl)-7- chloro-1-phenylhydroquinazolin-2- one | m/z [M + H]+ 354.12 |
| 239 | 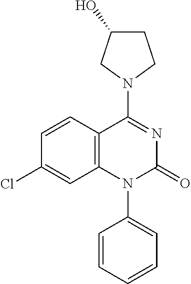 [SMILES: O=c1nc(N2CC[C@@H](O)C2)c2ccc(Cl)cc2n1-c1ccccc1] | 4-((3R)-3-hydroxypyrrolidinyl)-7- chloro-1-phenylhydroquinazolin-2- one | m/z [M + H]+ 342.19 |
| 240 | 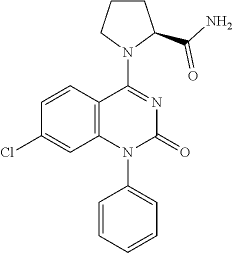 [SMILES: NC(=O)[C@@H]1CCCN1c1nc(=O)n(-c2ccccc2)c2cc(Cl)ccc12] | (2S)-1-(7-chloro-2-oxo-1- phenylhydro-quinazolin-4- yl)pyrrolidine-2-carboxamide | m/z [M + H]+ 369.18 |
| 241 | 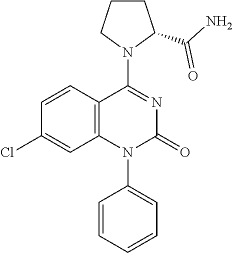 [SMILES: NC(=O)[C@H]1CCCN1c1nc(=O)n(-c2ccccc2)c2cc(Cl)ccc12] | (2R)-1-(7-chloro-2-oxo-1- phenylhydro-quinazolin-4- yl)pyrrolidine-2-carboxamide | m/z [M + H]+ 369.19 |
| 242 | 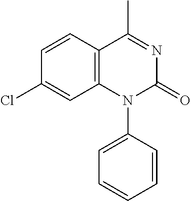 [SMILES: Cc1nc(=O)n(-c2ccccc2)c2cc(Cl)ccc12] | 7-chloro-4-methyl-1-phenylhydro- quinazolin-2-one | m/z [M + H]+ 271.1 |
| 243 | 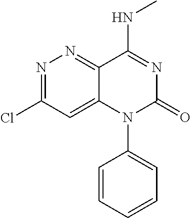 [SMILES: CNc1nc(=O)n(-c2ccccc2)c2cc(Cl)nnc12] | 3-chloro-8-(methylamino)-5-phenyl- 5-hydroxypyrimidino[5,4-c]pyridazin-6- one | m/z [M + H]+ 288.16 |
| 244 | 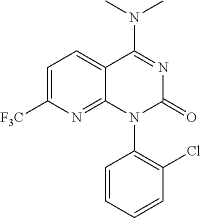 [SMILES: CN(C)c1nc(=O)n(-c2ccccc2Cl)c2nc(C(F)(F)F)ccc12] | 1-(2-chlorophenyl)-4- (dimethylamino)-7- (trifluoromethyl)pyrido[2,3-d]- pyrimidin-2(1H)-one | m/z [M + H]+ 369.05 |
| 245 | 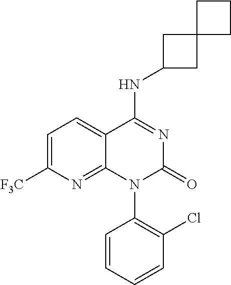 [SMILES: O=c1nc(NC2CC3(CCC3)C2)c2ccc(C(F)(F)F)nc2n1-c1ccccc1Cl] | 1-(2-chlorophenyl)-4- (spiro[3.3]heptan-2-ylamino)-7- (trifluoromethyl)pyrido-[2,3- d]pyrimidin-2(1H)-one | m/z [M + H]+ 435.10 |
| 246 | 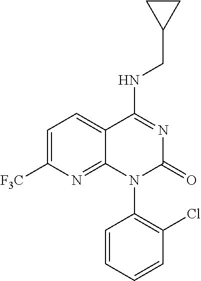 [SMILES: O=c1nc(NCC2CC2)c2ccc(C(F)(F)F)nc2n1-c1ccccc1Cl] | 1-(2-chlorophenyl)-4-((cyclopropyl- methyl)amino)-7-(trifluoromethyl)- pyrido[2,3-d]pyrimidin-2(1H)-one | m/z [M + H]+ 395.05 |
| 247 | 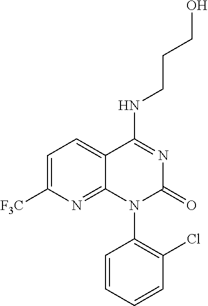 [SMILES: O=c1nc(NCCCO)c2ccc(C(F)(F)F)nc2n1-c1ccccc1Cl] | 1-(2-chlorophenyl)-4-[(3-hydroxy- propyl)amino}-7-(trifluoromethyl)- pyrido[2,3-d]pyrimidin-2(1H)-one | m/z [M + H]+ 399.05 |
| 248 | 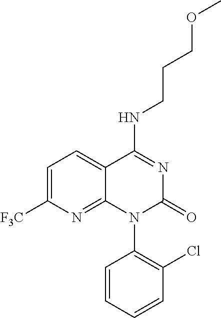 [SMILES: COCCCNc1nc(=O)n(-c2ccccc2Cl)c2nc(C(F)(F)F)ccc12] | 1-(2-chlorophenyl)-4-[(3-methoxy- propyl)amino]-7-(trifluoromethyl)- pyrido[2,3-d]pyrimidin-2(1H)-one | m/z [M + H]+ 413.10 |
| 249 | 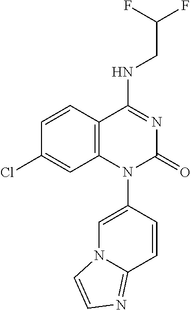 [SMILES: O=c1nc(NCC(F)F)c2ccc(Cl)cc2n1-c1ccc2nccn2c1] | 4-[(2,2-difluoroethyl)amino]-7- chloro-1-(4-hydroimidazo[1,2- a]pyridin-6-yl)-hydroquinazolin-2-one | m/z [M + H]+ 376.0 |
| 250 | 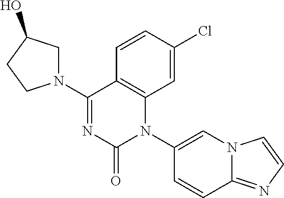 [SMILES: O=c1nc(N2CC[C@@H](O)C2)c2ccc(Cl)cc2n1-c1ccc2nccn2c1] | 4-((3R)-3-hydroxypyrrolidin-1-yl)-7- chloro-1-(4-hydroimidazo[1,2- a]pyridin-6-yl)-hydroquinazolin-2-one | m/z [M + H]+ 382.00 |
| 251 | 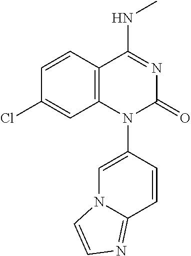 [SMILES: CNc1nc(=O)n(-c2ccc3nccn3c2)c2cc(Cl)ccc12] | 7-chloro-1-(4-hydroimidazo[1,2-a]- pyridin-6-yl)-4-(methylamino)hydro- quinazolin-2-one | m/z [M + H]+ 326.00 |
| 252 | 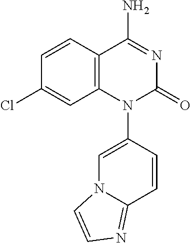 [SMILES: Nc1nc(=O)n(-c2ccc3nccn3c2)c2cc(Cl)ccc12] | 4-amino-7-chloro-1-(4- hydroimidazo-[1,2-a]pyridin-6- yl)hydroquinazolin-2-one | m/z [M + H]+ 312.00 |
| 253 | 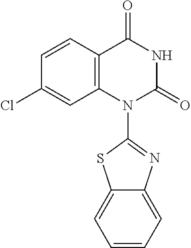 [SMILES: O=c1[nH]c(=O)n(-c2nc3ccccc3s2)c2cc(Cl)ccc12] | 1-benzothiazol-2-yl-7-chloro-1,3- dihydroquinazoline-2,4-dione | m/z [M + H]+ 330.0 |
| 254 | 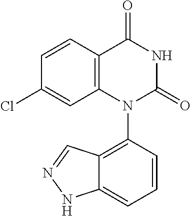 [SMILES: O=c1[nH]c(=O)n(-c2cccc3[nH]ncc23)c2cc(Cl)ccc12] | 1-(1H-indazol-4-yl)-7-chloro-1,3- dihydroquinazoline-2,4-dione | m/z [M + H]+ 313.0 |
| 255 | 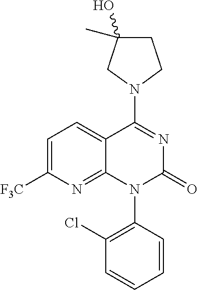 [SMILES: CC1(O)CCN(c2nc(=O)n(-c3ccccc3Cl)c3nc(C(F)(F)F)ccc23)C1] | 1-(2-chlorophenyl)-4-(3-hydroxy-3- methylpyrrolidin-1-yl)-7-(trifluoro- methyl)hydropyridino-[2,3- d]pyrimidin-2-one | m/z [M + H]+ 425.37 |
| 256 | 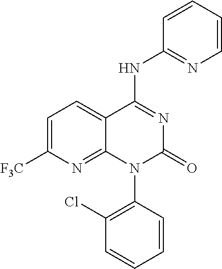 [SMILES: O=c1nc(Nc2ccccn2)c2ccc(C(F)(F)F)nc2n1-c1ccccc1Cl] | 1-(2-chlorophenyl)-4-(2- pyridylamino)-7- (trifluoromethyl)pyrido[2,3-d]- pyrimidin-2(1H)-one | m/z [M + H]+ 418.35 |
| 257 | 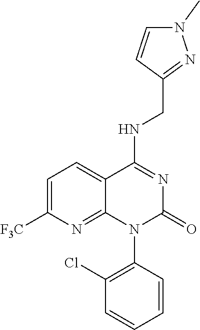 [SMILES: Cn1ccc(CNc2nc(=O)n(-c3ccccc3Cl)c3nc(C(F)(F)F)ccc23)n1] | 1-(2-chlorophenyl)-4-{[(1-methyl- pyrazol-3-yl)amino]amino}-7- (trifluoro-methyl)-pyrido[2,3- d]pyrimidin-2(1H)-one | m/z [M + H]+ 435.35 |
| 258 | 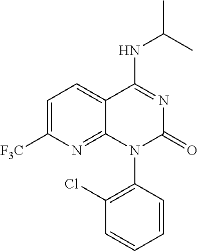 [SMILES: CC(C)Nc1nc(=O)n(-c2ccccc2Cl)c2nc(C(F)(F)F)ccc12] | 1-(2-chlorophenyl)-4-[(methylethyl)- amino]-7-(trifluoromethyl)pyrido[2,3- d]-pyrimidin-2(1H)-one | m/z [M + H]+ 383.34 |
| 259 | 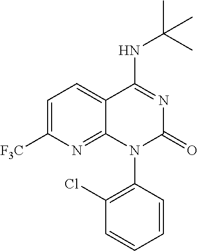 [SMILES: CC(C)(C)Nc1nc(=O)n(-c2ccccc2Cl)c2nc(C(F)(F)F)ccc12] | 4-[(tert-butyl)amino]-1-(2-chloro- phenyl)-7-(trifluoromethyl)pyrido- [2,3-d]pyrimidin-2(1H)-one | m/z [M + H]+ 397.39 |
| 260 | 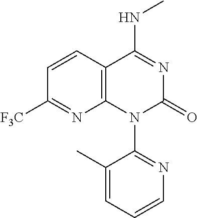 [SMILES: CNc1nc(=O)n(-c2ncccc2C)c2nc(C(F)(F)F)ccc12] | 1-(3-methyl(2-pyridyl))-4-(methyl- amino)-7-(trifluoromethyl)pyrido[2,3- d]-pyrimidin-2(1H)-one | m/z [M + H]+ 336.37 |
| 261 | 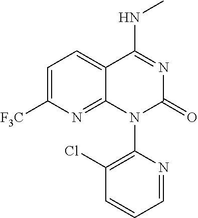 [SMILES: CNc1nc(=O)n(-c2ncccc2Cl)c2nc(C(F)(F)F)ccc12] | 1-(3-chloro(2-pyridyl))-4-(methyl- amino)-7-(trifluoromethyl)pyrido[2,3- d]-pyrimidin-2(1H)-one | m/z [M + H]+ 356.36 |
| 262 | 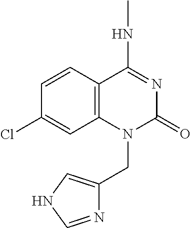 [SMILES: CNc1nc(=O)n(Cc2c[nH]cn2)c2cc(Cl)ccc12] | 7-chloro-1-(imidazol-4-ylmethyl)-4- (methylamino)hydroquinaozlin-2-one | m/z [M + H]+ 290.36 |
| 263 | 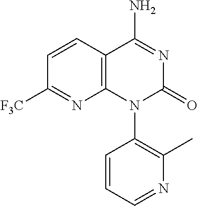 [SMILES: Cc1ncccc1-n1c(=O)nc(N)c2ccc(C(F)(F)F)nc21] | 4-amino-1-(2-methyl(3-pyridyl))-7- (trifluoromethyl pyrido[2,3- d]pyrimidin-2(1H)-one | m/z [M + H]+ 322.10 |
| 264 | 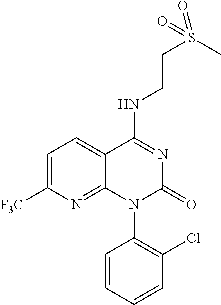 [SMILES: CS(=O)(=O)CCNc1nc(=O)n(-c2ccccc2Cl)c2nc(C(F)(F)F)ccc12] | 1-(2-chlorophenyl)-4-{[2-(methyl- sulfonyl)ethyl]amino}-7-(trifluoro- methyl)pyrido[2,3-d]pyrimidin-2(1H)- one | m/z [M + H]+ 447.00 |
| 265 | 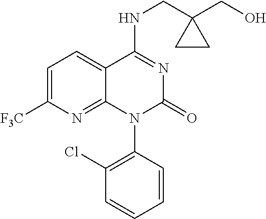 [SMILES: O=c1nc(NCC2(CO)CC2)c2ccc(C(F)(F)F)nc2n1-c1ccccc1Cl] | 1-(2-chlorophenyl)-4-(((1-(hydroxy- methyl)cyclopropyl)methyl)amino)-7- (trifluoromethyl)-pyrido[2,3-d]- pyrimidin-2(1H)-one | m/z [M + H]+ 425.00 |
| 266 | 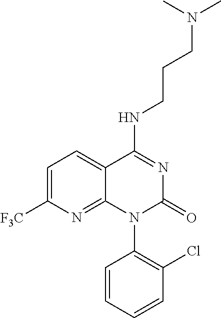 [SMILES: CN(C)CCCNc1nc(=O)n(-c2ccccc2Cl)c2nc(C(F)(F)F)ccc12] | 4-{[3-(dimethylamino)propyl]amino}- 1-(2-chlorophenyl)-7- (trifluoromethyl)-pyrido[2,3- d]pyrimidin-2(1H)-one | m/z [M + H]+ 426.00 |
| 267 | 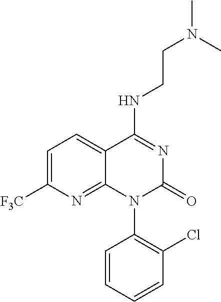 [SMILES: CN(C)CCNc1nc(=O)n(-c2ccccc2Cl)c2nc(C(F)(F)F)ccc12] | 4-{[2-(dimethylamino)ethyl]amino}- 1-(2-chlorophenyl)-7- (trifluoromethyl)-pyrido[2,3- d]pyrimidin-2(1H)-one | m/z [M + H]+ 412.00 |
| 268 | 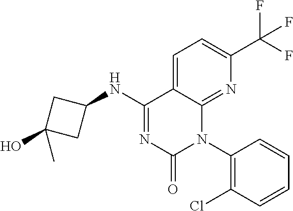 [SMILES: C[C@]1(O)C[C@@H](Nc2nc(=O)n(-c3ccccc3Cl)c3nc(C(F)(F)F)ccc23)C1] | 1-(2-chlorophenyl)-4-(((1s,3s)-3- hydroxy-3-methylcyclobutyl)amino)- 7-(trifluoromethyl)pyrido[2,3- d]pyrimidin-2(1H)-one | m/z [M + H]+ 425.00 |
| 269 | 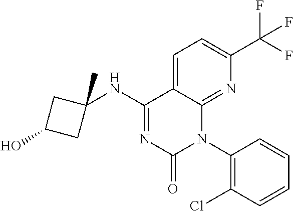 [SMILES: C[C@]1(Nc2nc(=O)n(-c3ccccc3Cl)c3nc(C(F)(F)F)ccc23)C[C@@H](O)C1] | 1-(2-chlorophenyl)-4-(((1s,3s)-3- hydroxy-1-methylcyclobutyl)amino)- 7-(trifluoromethyl)pyrido[2,3- d]pyrimidin-2(1H)-one | m/z [M + H]+ 425.00 |
| 270 | 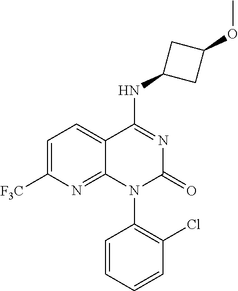 [SMILES: CO[C@H]1C[C@@H](Nc2nc(=O)n(-c3ccccc3Cl)c3nc(C(F)(F)F)ccc23)C1] | 1-(2-chlorophenyl)-4-(((1s,3s)-3- methoxycyclobutyl)amino)-7- (trifluoro-methyl)pyrido[2,3- d]pyrimidin-2(1H)-one | m/z [M + H]+ 425.00 |
| 271 | 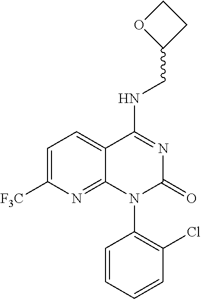 [SMILES: O=c1nc(NCC2CCO2)c2ccc(C(F)(F)F)nc2n1-c1ccccc1Cl] | 1-(2-chlorophenyl)-4-[(oxetan-2- ylmethyl)amino]-7-(trifluoromethyl)- pyrido[2,3-d]pyrimidin-2(1H)-one | m/z [M + H]+ 411.00 |
| 272 | 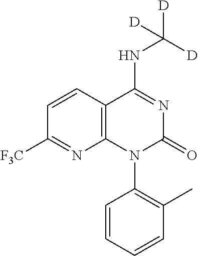 [SMILES: [2H]C([2H])([2H])Nc1nc(=O)n(-c2ccccc2C)c2nc(C(F)(F)F)ccc12] | 4-((methyl-d3)amino)-1-(2-methyl- phenyl)-7-(trifluoromethyl)pyrido[2,3- d]pyrimidin-2(1H)-one | m/z [M + H]+ 338.20 |
| 273 | 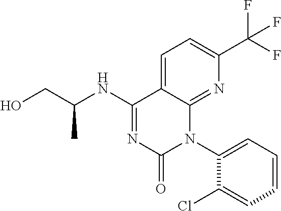 | 4-[((1S)-2-hydroxy- isopropyl)amino]-(1Ra)-(2- chlorophenyl)-7-(trifluoro-methyl)- pyrido[2,3-d]pyrimidin-2(1H)-one | m/z [M + H]+ 399.00 |
| 274 | 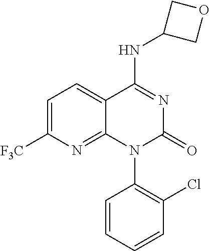 [SMILES: O=c1nc(NC2COC2)c2ccc(C(F)(F)F)nc2n1-c1ccccc1Cl] | 1-(2-chlorophenyl)-4-(oxetan-3-yl- amino)-7-(trifluoromethyl)pyrido[2,3- d]pyrimidin-2(1H)-one | m/z [M + H]+ 397.0 |
| 275 | 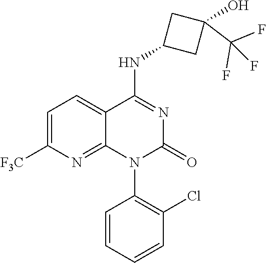 [SMILES: O=c1nc(N[C@H]2C[C@](O)(C(F)(F)F)C2)c2ccc(C(F)(F)F)nc2n1-c1ccccc1Cl] | 4-{[(cis)-3-hydroxy-3- (trifluoromethyl)-cyclobutyl]amino}-1- (2-chlorophenyl)-7- (trifluoromethyl)pyrido[2,3-d]- pyrimidin-2(1H)-one | m/z [M + H]+ 479.00 |
| 276 | 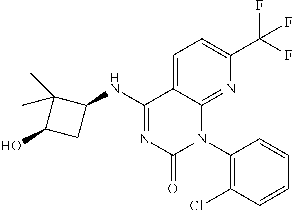 [SMILES: CC1(C)[C@H](O)C[C@@H]1Nc1nc(=O)n(-c2ccccc2Cl)c2nc(C(F)(F)F)ccc12] | 4-{((cis)-3-hydroxy-2,2- dimethylcyclo-butyl)amino]-1-(2- chlorophenyl)-7-(trifluoromethyl)- pyrido[2,3-d]-pyrimidin-2(1H)-one | m/z [M + H]+ 439.00 |
| 277 | 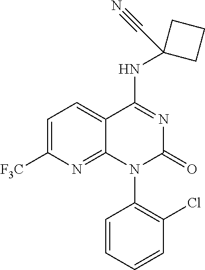 [SMILES: N#CC1(Nc2nc(=O)n(-c3ccccc3Cl)c3nc(C(F)(F)F)ccc23)CCC1] | 1-((1-(2-chlorophenyl)-2-oxo-7- (trifluoromethyl)-1,2- dihydropyrido[2,3-d]pyrimidin-4- yl)amino)cyclobutane-1-carbonitrile | m/z [M + H]+ 420.00 |
| 278 | 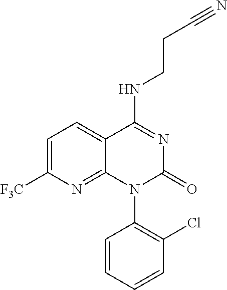 [SMILES: N#CCCNc1nc(=O)n(-c2ccccc2Cl)c2nc(C(F)(F)F)ccc12] | 3-((1-(2-chlorophenyl)-2-oxo-7- (trifluoro-methyl)-1,2-dihydropyrido- [2,3-d]pyrimidin-4-yl)amino)- propanenitrile | m/z [M + H]+ 394.00 |
| 279 | 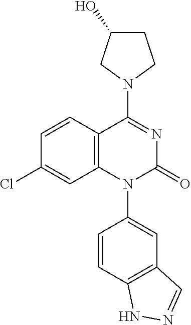 [SMILES: O=c1nc(N2CC[C@@H](O)C2)c2ccc(Cl)cc2n1-c1ccc2[nH]ncc2c1] | (R)-7-chloro-4-(3-hydroxypyrrolidin- 1-yl)-1-(1H-indazol-5-yl)quinazolin- 2(1H)-one | m/z [M + H]+ 382.0 |
| 280 | 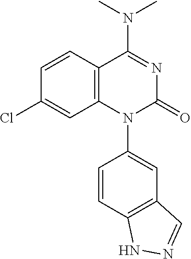 [SMILES: CN(C)c1nc(=O)n(-c2ccc3[nH]ncc3c2)c2cc(Cl)ccc12] | 7-chloro-4-(dimethylamino)-1-(1H- indazol-5-yl)quinazolin-2(1H)-one | m/z [M + H]+ 340.0 |
| 281 | 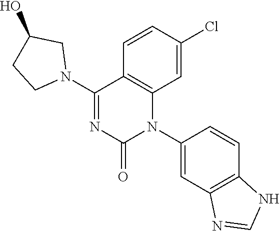 [SMILES: O=c1nc(N2CC[C@@H](O)C2)c2ccc(Cl)cc2n1-c1ccc2[nH]cnc2c1] | (R)-7-chloro-4-(3-hydroxypyrrolidin- 1-yl)-1-(1H-benzimidazol-5- yl)quinazolin-2(1H)-one | m/z [M + H]+ 382.0 |
| 282 | 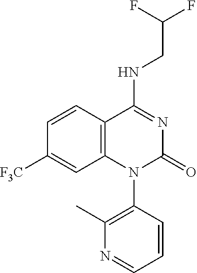 [SMILES: Cc1ncccc1-n1c(=O)nc(NCC(F)F)c2ccc(C(F)(F)F)cc21] | 4-((2,2-difluoroethyl)amino)-1-(2- methylpyridin-3-yl)-7- (trifluoromethyl)-quinazolin-2(1H)- one | m/z [M + H]+ 385.0 |
| 283 | 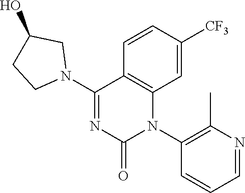 [SMILES: Cc1ncccc1-n1c(=O)nc(N2CC[C@@H](O)C2)c2ccc(C(F)(F)F)cc21] | 4-((3R)-3-hydroxypyrrolidinyl)-1-(2- methylpyridin-3-yl))-7- (trifluoromethyl)-hydroquinazolin-2- one | m/z [M + H]+ 391.0 |
| 284 | 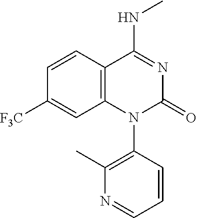 [SMILES: CNc1nc(=O)n(-c2cccnc2C)c2cc(C(F)(F)F)ccc12] | 1-(2-methylpyridin-3-yl))-4-(methyl- amino)-7-(trifluoromethyl)hydro- quinazolin-2-one | m/z [M + H]+ 335.0 |
| 285 | 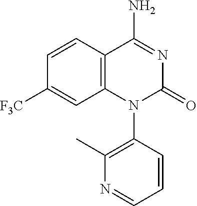 [SMILES: Cc1ncccc1-n1c(=O)nc(N)c2ccc(C(F)(F)F)cc21] | 4-amino-1-(2-methylpyridin-3-yl))-7- (trifluoromethyl)hydroquinazolin-2- one | m/z [M + H]+ 321.0 |
| 286 | 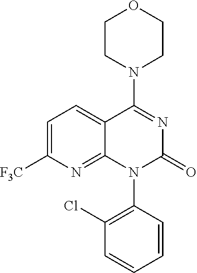 [SMILES: O=c1nc(N2CCOCC2)c2ccc(C(F)(F)F)nc2n1-c1ccccc1Cl] | 1-(2-chlorophenyl)-4-morpholin-4-yl- 7-(trifluoromethyl)pyrido[2,3- d]pyrimidin-2(1H)-one | m/z [M + H]+ 411.40 |
| 287 | 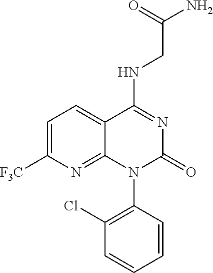 [SMILES: NC(=O)CNc1nc(=O)n(-c2ccccc2Cl)c2nc(C(F)(F)F)ccc12] | 2-((1-(2-chlorophenyl)-2-oxo-7- (trifluoromethyl)-1,2- dihydropyrido[2,3-d]pyrimidin-4- yl)amino)acetamide | m/z [M + H]+ 398.36 |
| 288 | 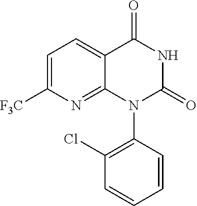 [SMILES: O=c1[nH]c(=O)n(-c2ccccc2Cl)c2nc(C(F)(F)F)ccc12] | 1-(2-chlorophenyl)-4-hydroxy-7- (trifluoromethyl)hydropyridino[2,3-d]- pyrimidin-2-one | m/z [M + H]+ 342.31 |
| 289 | 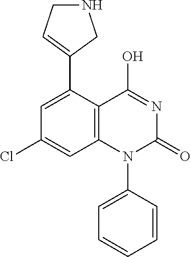 [SMILES: O=c1nc(O)c2c(C3=CCNC3)cc(Cl)cc2n1-c1ccccc1] | 7-chloro-5-(2,5-dihydro-1H-pyrrol-3- yl)-4-hydroxy-1-phenylquinazolin- 2(1H)-one | m/z [M + H]+ 340.37 |
| 290 | 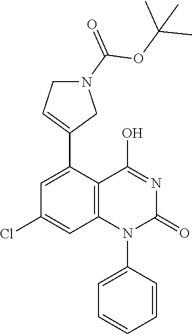 [SMILES: CC(C)(C)OC(=O)N1CC=C(c2cc(Cl)cc3c2c(O)nc(=O)n3-c2ccccc2)C1] | tert-butyl 3-(7-chloro-4-hydroxy-2- oxo-1-phenyl-1,2-dihydroquinazolin- 5-yl)-2,5-dihydro-1H-pyrrole-1- carboxylate | m/z [M + H]+ 440.12 |
| 291 | 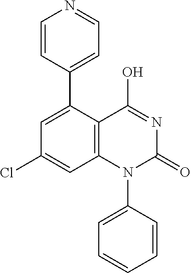 [SMILES: O=c1nc(O)c2c(-c3ccncc3)cc(Cl)cc2n1-c1ccccc1] | 7-chloro-4-hydroxy-1-phenyl-5- (pyridin-4-yl)quinazolin-2(1H)-one | m/z [M + H]+ 350.09 |
| 292 | 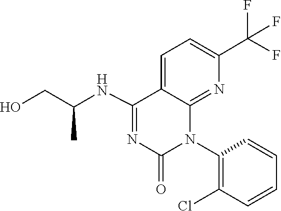 | 4-[((1S)-2-hydroxy- isopropyl)amino]-(1Sa)-(2- chlorophenyl)-7-(trifluoro-methyl)- pyrido[2,3-d]pyrimidin-2(1H)-one | m/z [M + H]+ 399.10 |
| 293 | 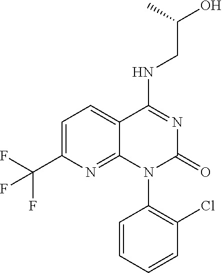 [SMILES: C[C@H](O)CNc1nc(=O)n(-c2ccccc2Cl)c2nc(C(F)(F)F)ccc12] | 4-[((2S)-2-hydroxypropyl)amino]-1- (2-chlorophenyl)-7-(trifluoromethyl)- pyrido[2,3-d]pyrimidin-2(1H)-one | m/z [M + H]+ 399.10 |
| 294 | 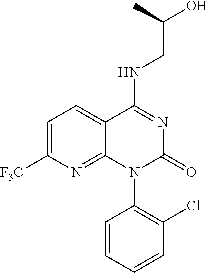 [SMILES: C[C@@H](O)CNc1nc(=O)n(-c2ccccc2Cl)c2nc(C(F)(F)F)ccc12] | 4-[((2R)-2-hydroxypropyl)amino]-1- (2-chlorophenyl)-7-(trifluoromethyl)- pyrido[2,3-d]pyrimidin-2(1H)-one | m/z [M + H]+ 399.10 |
| 295 | 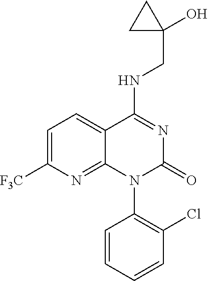 [SMILES: O=c1nc(NCC2(O)CC2)c2ccc(C(F)(F)F)nc2n1-c1ccccc1Cl] | 1-(2-chlorophenyl)-4- {[(hydroxycyclo- propyl)methyl]amino}-7-(trifluoro- methyl)-pyrido[2,3-d]pyrimidin- 2(1H)-one | m/z [M + H]+ 411.10 |
| 296 | 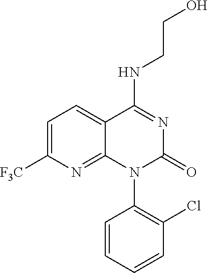 [SMILES: O=c1nc(NCCO)c2ccc(C(F)(F)F)nc2n1-c1ccccc1Cl] | 1-(2-chlorophenyl)-4-[(2-hydroxy- ethyl)amino]-7-(trifluoromethyl) pyrido[2,3-d]pyrimidin-2(1H)-one | m/z [M + H]+ 385.10 |
| 297 | 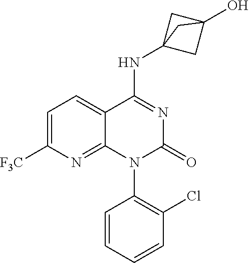 [SMILES: O=c1nc(NC23CC(O)(C2)C3)c2ccc(C(F)(F)F)nc2n1-c1ccccc1Cl] | 1-(2-chlorophenyl)-4-[(3-hydroxy- bicyclo[1.1.1]pentyl)amino]-7- (trifluoro-methyl)-pyrido[2,3- d]pyrimidin-2(1H)-one | m/z [M + H]+ 423.10 |
| 298 | 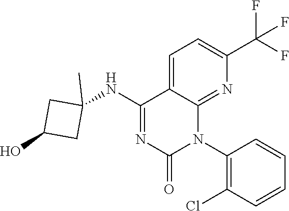 [SMILES: C[C@]1(Nc2nc(=O)n(-c3ccccc3Cl)c3nc(C(F)(F)F)ccc23)C[C@H](O)C1] | 4-[((trans)-3-hydroxy-1-methylcyclo- butyl)amino]-1-(2-chlorophenyl)-7- (trifluoromethyl)-pyrido[2,3- d]pyrimidin-2(1H)-one | m/z [M + H]+ 425.10 |
| 299 | 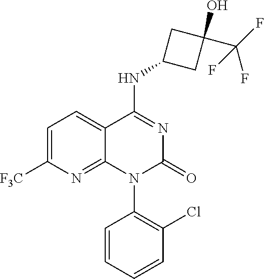 [SMILES: O=c1nc(N[C@H]2C[C@@](O)(C(F)(F)F)C2)c2ccc(C(F)(F)F)nc2n1-c1ccccc1Cl] | 4-{[(trans)-3-hydroxy-3-(trifluoro- methyl)cyclobutyl]amino]-1-(2- chlorophenyl)-7- (trifluoromethyl)pyrido-[2,3- d]pyrimidin-2(1H)-one | m/z [M + H]+ 479.10 |
| 300 | 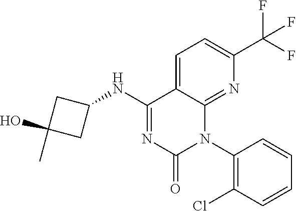 [SMILES: C[C@]1(O)C[C@H](Nc2nc(=O)n(-c3ccccc3Cl)c3nc(C(F)(F)F)ccc23)C1] | 4-[((trans)-3-hydroxy-3-methylcyclo- butyl)amino]-1-(2-chlorophenyl)-7- (trifluoromethyl)pyrido[2,3- d]pyrimidin-2(1H)-one | m/z [M + H]+ 425.10 |
| 301 | 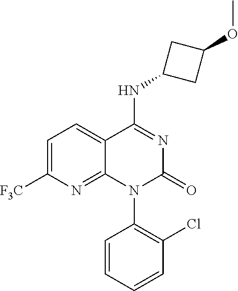 [SMILES: CO[C@H]1C[C@H](Nc2nc(=O)n(-c3ccccc3Cl)c3nc(C(F)(F)F)ccc23)C1] | 4-[(trans)-3- methoxycyclobutyl)amino]-1-(2- chlorophenyl)-7-(trifluoromethyl)- pyrido[2,3-d]pyrimidin-2(1H)-one | m/z [M + H]+ 425.10 |
| 302 | 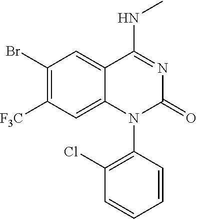 [SMILES: CNc1nc(=O)n(-c2ccccc2Cl)c2cc(C(F)(F)F)c(Br)cc12] | 6-bromo-1-(2-chlorophenyl)-4- (methylamino)-7-(trifluoromethyl)- quinazolin-2(1H)-one | m/z [M + H]+ 432.0 |
| 303 | 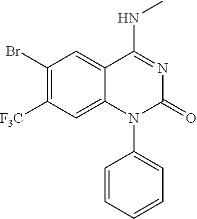 [SMILES: CNc1nc(=O)n(-c2ccccc2)c2cc(C(F)(F)F)c(Br)cc12] | 6-bromo-4-(methylamino)-1-phenyl- 7-(trifluoromethyl)quinazolin-2(1H)- one | m/z [M + H]+ 398.0 |
| 304 | 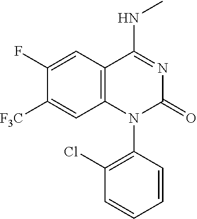 [SMILES: CNc1nc(=O)n(-c2ccccc2Cl)c2cc(C(F)(F)F)c(F)cc12] | 1-(2-chlorophenyl)-6-fluoro-4- (methyl-amino)-7- (trifluoromethyl)hydro-quinazolin-2- one | m/z [M + H]+ 372.0 |
| 305 | 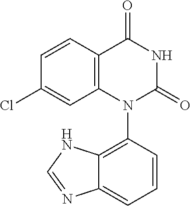 [SMILES: O=c1[nH]c(=O)n(-c2cccc3nc[nH]c23)c2cc(Cl)ccc12] | 1-benzimidazol-7-yl-7-chloro-1,3- dihydroquinazoline-2,4-dione | m/z [M + H]+ 313.0 |
| 306 | 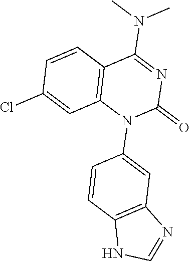 [SMILES: CN(C)c1nc(=O)n(-c2ccc3[nH]cnc3c2)c2cc(Cl)ccc12] | 1-benzimidazol-5-yl-4- (dimethylamino)-7- chlorohydroquinazolin-2-one | m/z [M + H]+ 340.0 |
| 307 | 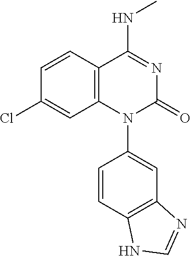 [SMILES: CNc1nc(=O)n(-c2ccc3[nH]cnc3c2)c2cc(Cl)ccc12] | 1-benzimidazol-5-yl-7-chloro-4- (methyl-amino)hydroquinazolin-2- one | m/z [M + H]+ 326.0 |
| 308 | 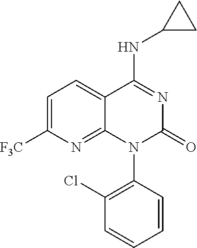 [SMILES: O=c1nc(NC2CC2)c2ccc(C(F)(F)F)nc2n1-c1ccccc1Cl] | 1-(2-chlorophenyl)-4-(cyclopropyl- amino)-7-(trifluoromethyl)pyrido[2,3- d]pyrimidin-2(1H)-one | m/z [M + H]+ 381.28 |
| 309 | 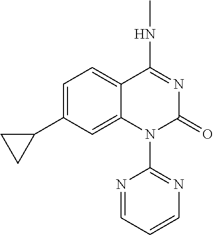 [SMILES: CNc1nc(=O)n(-c2ncccn2)c2cc(C3CC3)ccc12] | 7-cyclopropyl-4-(methylamino)-1- pyrimidin-2-ylhydroquinazolin-2-one | m/z [M + H]+ 294.30 |
| 310 | 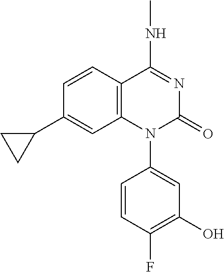 | 7-chloro-1-(4-fluoro-3- hydroxyphenyl)-4- (methylamino)hydroquinazolin-2-one | m/z [M + H]+ 320.34 |
| 311 | 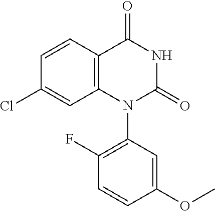 [SMILES: COc1ccc(F)c(-n2c(=O)[nH]c(=O)c3ccc(Cl)cc32)c1] | 7-chloro-1-(2-fluoro-5- methoxyphenyl)-quinazoline- 2,4(1H,3H)-dione | m/z [M + H]+ 321.31 |
| 312 | 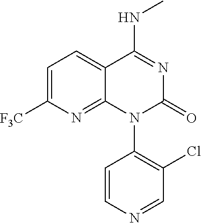 [SMILES: CNc1nc(=O)n(-c2ccncc2Cl)c2nc(C(F)(F)F)ccc12] | 1-(3-chloro-pyridin-4-yl))-4-(methyl- amino)-7-(trifluoromethyl)pyrido[2,3- d]-pyrimidin-2(1H)-one | m/z [M + H]+ 356.00 |
| 313 | 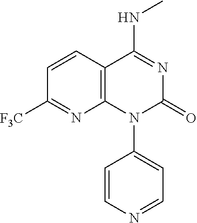 [SMILES: CNc1nc(=O)n(-c2ccncc2)c2nc(C(F)(F)F)ccc12] | 4-(methylamino)-1-(pyridin-4-yl))-7- (trifluoromethyl)pyrido[2,3- d]pyrimidin-2(1H)-one | m/z [M + H]+ 322.00 |
| 314 | 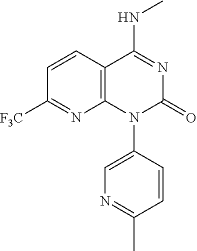 [SMILES: CNc1nc(=O)n(-c2ccc(C)nc2)c2nc(C(F)(F)F)ccc12] | 1-(6-methyl--pyridin-3-yl))-4- (methyl-amino)-7- (trifluoromethyl)pyrido[2,3-d]- pyrimidin-2(1H)-one | m/z [M + H]+ 336.0 |
| 315 | 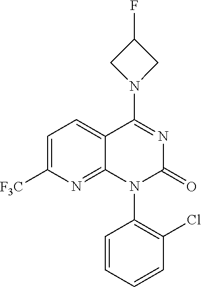 [SMILES: O=c1nc(N2CC(F)C2)c2ccc(C(F)(F)F)nc2n1-c1ccccc1Cl] | 1-(2-chlorophenyl)-4-(3- fluoroazetidin-1-yl)-7- (trifluoromethyl)pyrido[2,3- d]pyrimidin-2(1H)-one | m/z [M + H]+ 399.10 |
| 316 | 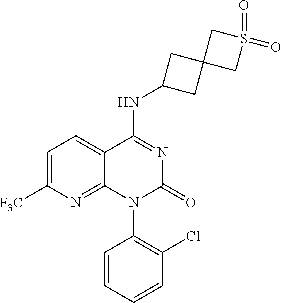 [SMILES: O=c1nc(NC2CC3(C2)CS(=O)(=O)C3)c2ccc(C(F)(F)F)nc2n1-c1ccccc1Cl] | 1-(2-chlorophenyl)-4-((2,2-dioxido- 2-thiaspiro[3.3]heptan-6-yl)amino)-7- (trifluoromethyl)pyrido[2,3- d]pyrimidin-2(1H)-one | m/z [M + H]+ 485.10 |
| 317 | 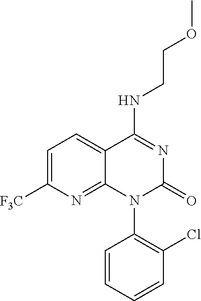 [SMILES: COCCNc1nc(=O)n(-c2ccccc2Cl)c2nc(C(F)(F)F)ccc12] | 1-(2-chlorophenyl)-4-[(2- methoxyethyl)-amino]-7- (trifluoromethyl)pyrido[2,3-d]- pyrimidin-2(1H)-one | m/z [M + H]+ 399.10 |
| 318 | 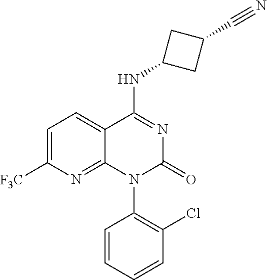 [SMILES: N#C[C@H]1C[C@@H](Nc2nc(=O)n(-c3ccccc3Cl)c3nc(C(F)(F)F)ccc23)C1] | (1s,3s)-3-((1-(2-chlorophenyl)-2- oxo-7-(trifluoromethyl)-1,2- dihydropyrido[2,3-d]pyrimidin-4- yl)amino)cyclobutane-1-carbonitrile | m/z [M + H]+ 420.10 |
| 319 | 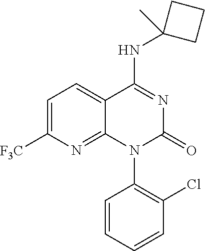 [SMILES: CC1(Nc2nc(=O)n(-c3ccccc3Cl)c3nc(C(F)(F)F)ccc23)CCC1] | 1-(2-chlorophenyl)-4-[(methylcyclo- butyl)amino]-7- (trifluoromethyl)pyrido-[2,3- d]pyrimidin-2(1H)-one | m/z [M + H]+ 409.20 |
| 320 | 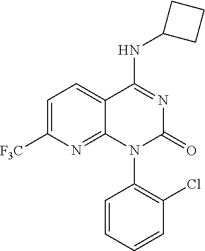 [SMILES: O=c1nc(NC2CCC2)c2ccc(C(F)(F)F)nc2n1-c1ccccc1Cl] | 1-(2-chlorophenyl)-4- (cyclobutylamino)-7- (trifluoromethyl)pyrido[2,3-d]- pyrimidin-2(1H)-one | m/z [M + H]+ 395.10 |
| 321 | 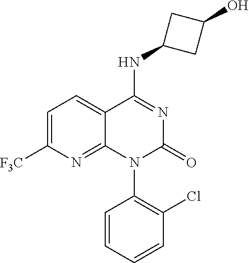 [SMILES: O=c1nc(N[C@H]2C[C@@H](O)C2)c2ccc(C(F)(F)F)nc2n1-c1ccccc1Cl] | (1s,3s)-3-((1-(2-chlorophenyl)-2- oxo-7-(trifluoromethyl)-1,2- dihydropyrido[2,3-d]pyrimidin-4- yl)amino)cyclobutane-1-carbonitrile | m/z [M + H]+ 411.10 |
| 322 | 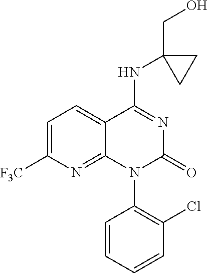 [SMILES: O=c1nc(NC2(CO)CC2)c2ccc(C(F)(F)F)nc2n1-c1ccccc1Cl] | 1-(2-chlorophenyl)-4-{[(hydroxy- methyl)cyclopropyl]amino]-7- (trifluoro-methyl)-pyrido[2,3- d]pyrimidin-2(1H)-one | m/z [M + H]+ 411.10 |
| 323 | 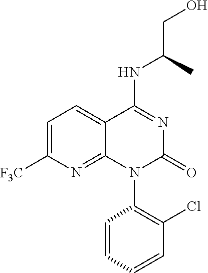 | (R)-(1Sₐ)-(2-chlorophenyl)-4-((1- hydroxypropan-2-yl)amino)-7- (trifluoro-methyl)pyrido-[2,3- d]pyrimidin-2(1H)-one | m/z [M + H]+ 399.10 |
| 324 | 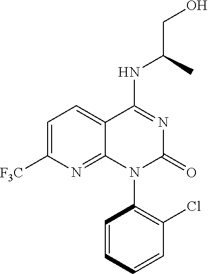 | (R)-(1Rₐ)-(2-chlorophenyl)-4-((1- hydroxypropan-2-yl)amino)-7- (trifluoro-methyl)pyrido-[2,3- d]pyrimidin-2(1H)-one | m/z [M + H]+ 399.10 |
| 325 | 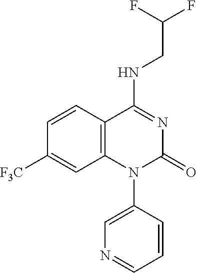 [SMILES: O=c1nc(NCC(F)F)c2ccc(C(F)(F)F)cc2n1-c1cccnc1] | 4-[(2,2-difluoroethyl)amino]-1- (pyridin-3-yl)-7- (trifluoromethyl)hydro-quinazolin-2- one | m/z [M + H]+ 371.0 |
| 326 | 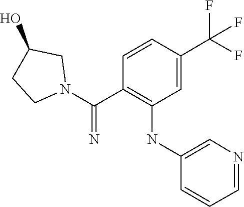 | 4-((3R)-3-hydroxypyrrolidinyl)-1- (pyridin-3-yl)-7-(trifluoromethyl)- hydroquinazolin-2-one | m/z [M + H]+ 372.0 |
| 327 | 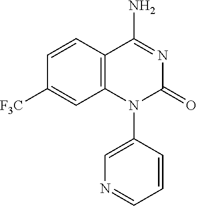 [SMILES: Nc1nc(=O)n(-c2cccnc2)c2cc(C(F)(F)F)ccc12] | 4-amino-1-(pyridin-3-yl)-7-(trifluoro- methyl)hydroquinazolin-2-one | m/z [M + H]+ 307.0 |
| 328 | 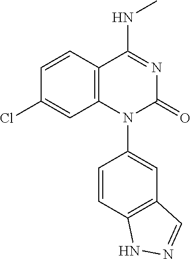 [SMILES: CNc1nc(=O)n(-c2ccc3[nH]ncc3c2)c2cc(Cl)ccc12] | 1-(1H-indazol-5-yl)-7-chloro-4- (methyl-amino)hydroquinazolin-2- one | m/z [M + H]+ 326.0 |
| 329 | 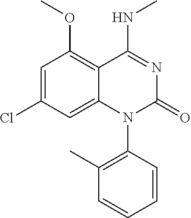 [SMILES: CNc1nc(=O)n(-c2ccccc2C)c2cc(Cl)cc(OC)c12] | 7-chloro-4-methoxy-4- (methylamino)-1-(2- methylphenyl)hydroquinazolin-2-one | m/z [M + H]+ 330.1 |
| 330 | 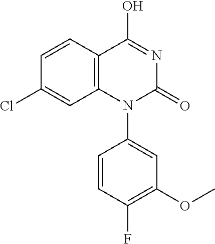 [SMILES: COc1cc(-n2c(=O)nc(O)c3ccc(Cl)cc32)ccc1F] | 7-chloro-1-(4-fluoro-3- methoxyphenyl)-4- hydroxyhydroquinazolin-2-on | m/z [M + H]+ 319.05 |
| 331 | 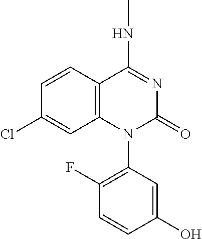 [SMILES: CNc1nc(=O)n(-c2cc(O)ccc2F)c2cc(Cl)ccc12] | 7-chloro-1-(6-fluoro-3- hydroxyphenyl)-4- (methylamino)hydroquinazolin-2-one | m/z [M + H]+ 319.06 |
| 332 | 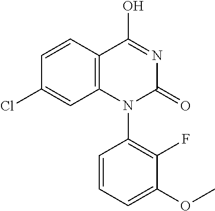 [SMILES: COc1cccc(-n2c(=O)nc(O)c3ccc(Cl)cc32)c1F] | 7-chloro-1-(2-fluoro-3- methoxyphenyl)-4- hydroxyhydroquinazolin-2-one | m/z [M + H]+ 320.06 |
| 333 | 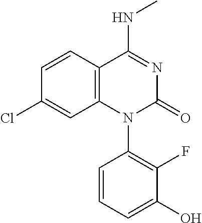 [SMILES: CNc1nc(=O)n(-c2cccc(O)c2F)c2cc(Cl)ccc12] | 7-chloro-1-(2-fluoro-3- hydroxyphenyl)-4- (methylamino)quinazolin-2(1H)-one | m/z [M + H]+ 320.27 |
| 334 | 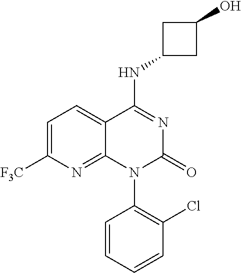 [SMILES: O=c1nc(N[C@H]2C[C@H](O)C2)c2ccc(C(F)(F)F)nc2n1-c1ccccc1Cl] | 1-(2-chlorophenyl)-4-(((1r,3r)-3- hydroxycyclobutyl)amino)-7- (trifluoro-methyl)pyrido[2,3- d]pyrimidin-2(1H)-one | m/z [M + H]+ 411.00 |
| 335 | 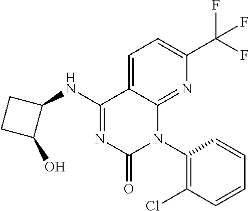 | 4-[((2S,1R)-2-hydroxycyclobutyl)- amino]-(1Sₐ)-(2-chlorophenyl)-7- (trifluoromethyl)hydro-pyrido[2,3-d]- pyrimidin-2(1H)-one | m/z [M + H]+ 411.00 |
| 336 | 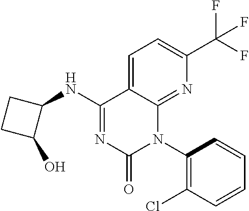 | 4-[((2S,1R)-2-hydroxycyclo- butyl)amino]-(1Rₐ)-(2-chlorophenyl)- 7-(trifluoromethyl)hydro-pyridino[2,3- d]-pyrimidin-2-one | m/z [M + H]+ 411.00 |
| 337 | 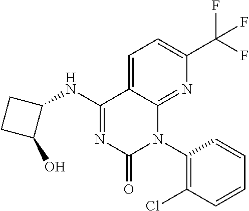 | 4-[((1S,2S)-2-hydroxycyclobutyl)- amino]-(1Sa)-(2-chlorophenyl)-7- (trifluoromethyl)hydro-pyridino[2,3-d]- pyrimidin-2-one | m/z [M + H]+ 411.00 |
| 338 | 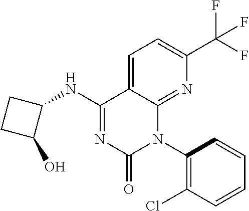 | 4-[((1S,2S)-2-hydroxycyclobutyl)- amino]-(1Ra)-(2-chlorophenyl)-7- (trifluoromethyl)hydro-pyridino[2,3-d]- pyrimidin-2-one | m/z [M + H]+ 411.00 |
| 339 | 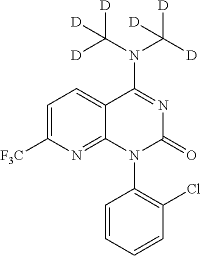 [SMILES: [2H]C([2H])([2H])N(c1nc(=O)n(-c2ccccc2Cl)c2nc(C(F)(F)F)ccc12)C([2H])([2H])[2H]] | 4-(bis(methyl-d3)amino)-1-(2- chloro-phenyl)-7-(trifluoromethyl)- pyrido[2,3-d]pyrimidin-2(1H)-one | m/z [M + H]+ 375.00 |
| 340 | 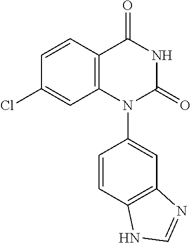 [SMILES: O=c1[nH]c(=O)n(-c2ccc3[nH]cnc3c2)c2cc(Cl)ccc12] | 1-(1H-benzo[d]imidazol-5-yl)-7- chloro-quinazoline-2,4-(1H,3H)-dione | m/z [M + H]+ 313.0 |
| 341 | 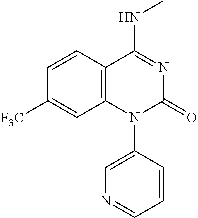 [SMILES: CNc1nc(=O)n(-c2cccnc2)c2cc(C(F)(F)F)ccc12] | 4-(methylamino)-1-(pyridin-3-yl)-7- (trifluoromethyl)hydroquinazolin-2- one | m/z [M + H]+ 321.0 |
| 342 | 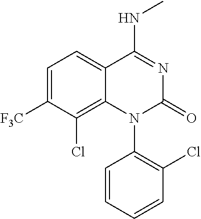 [SMILES: CNc1nc(=O)n(-c2ccccc2Cl)c2c(Cl)c(C(F)(F)F)ccc12] | 8-chloro-1-(2-chlorophenyl)-4- (methylamino)-7-(trifluoromethyl)- hydroquinazolin-2-one | m/z [M + H]+ 388.0 |
| 343 | 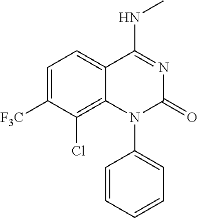 [SMILES: CNc1nc(=O)n(-c2ccccc2)c2c(Cl)c(C(F)(F)F)ccc12] | 8-chloro-4-(methylamino)-1-phenyl- 7-(trifluoromethyl)hydroquinazolin-2- one | m/z [M + H]+ 354.04 |
| 344 | 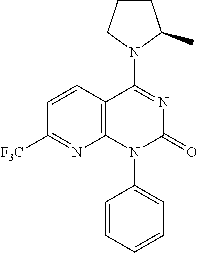 [SMILES: C[C@@H]1CCCN1c1nc(=O)n(-c2ccccc2)c2nc(C(F)(F)F)ccc12] | 4-((2R)-2-methylpyrrolidin-1-yl)-1- phenyl-7-(trifluoromethyl)pyrido[2,3- d]-pyrimidin-2(1H)-one | m/z [M + H]+ 375.1 |
| 345 | 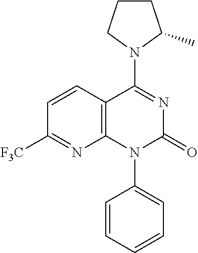 [SMILES: C[C@H]1CCCN1c1nc(=O)n(-c2ccccc2)c2nc(C(F)(F)F)ccc12] | 4-((2S)-2-methylpyrrolidin-1-yl)-1- phenyl-7-(trifluoromethyl)pyrido[2,3- d]-pyrimidin-2(1H)-one | m/z [M + H]+ 375.1 |
| 346 | 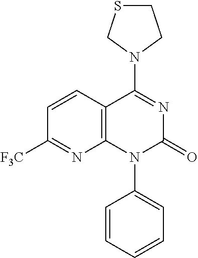 [SMILES: O=c1nc(N2CCSC2)c2ccc(C(F)(F)F)nc2n1-c1ccccc1] | 1-phenyl-4-(1,3-thiazolidin-3-yl)-7- (trifluoromethyl)pyrido[2,3-d]- pyrimidin-2(1H)-one | m/z [M + H]+ 379.1 |
| 347 | 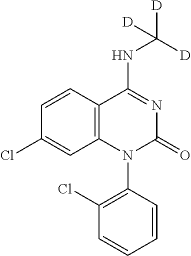 [SMILES: [2H]C([2H])([2H])Nc1nc(=O)n(-c2ccccc2Cl)c2cc(Cl)ccc12] | 7-chloro-1-(2-chlorophenyl)-4- ((methyl-d3)amino)quinazolin-2(1H)- one | m/z [M + H]+ 324.1 |
| 348 | 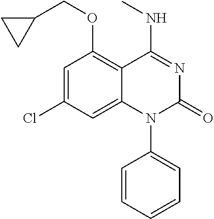 [SMILES: CNc1nc(=O)n(-c2ccccc2)c2cc(Cl)cc(OCC3CC3)c12] | 7-chloro-5-(cyclopropylmethoxy)-4- (methylamino)-1-phenylhydro- quinazolin-2-one | m/z [M + H]+ 356.32 |
| 349 | 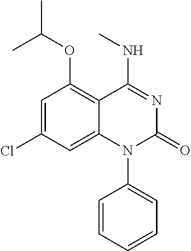 [SMILES: CNc1nc(=O)n(-c2ccccc2)c2cc(Cl)cc(OC(C)C)c12] | 7-chloro-4-(methylamino)-5-(methyl- ethoxy)-1-phenylhydroquinazolin-2- one | m/z [M + H]+ 344.33 |
| 350 | 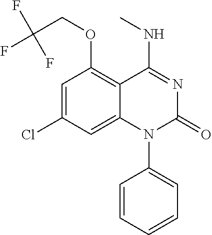 [SMILES: CNc1nc(=O)n(-c2ccccc2)c2cc(Cl)cc(OCC(F)(F)F)c12] | 7-chloro-4-(methylamino)-1-phenyl- 5-(2,2,2- trifluoroethoxy)hydroquinazolin-2- one | m/z [M + H]+ 384.31 |
| 351 | 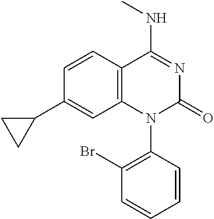 [SMILES: CNc1nc(=O)n(-c2ccccc2Br)c2cc(C3CC3)ccc12] | 1-(2-bromophenyl)-7-cyclopropyl-4- (methylamino)hydroquinazolin-2-one | m/z [M + H]+ 370.26 |
| 352 | 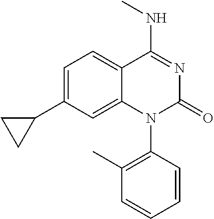 [SMILES: CNc1nc(=O)n(-c2ccccc2C)c2cc(C3CC3)ccc12] | 7-cyclopropyl-4-(methylamino)-1-(2- methylphenyl)hydroquinazolin-2-one | m/z [M + H]+ 306.33 |
| 353 | 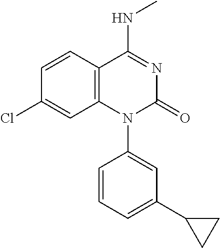 [SMILES: CNc1nc(=O)n(-c2cccc(C3CC3)c2)c2cc(Cl)ccc12] | 7-chloro-1-(3-cyclopropylphenyl)-4- (methylamino)hydroquinazolin-2-one | m/z [M + H]+ 326.32 |
| 354 | 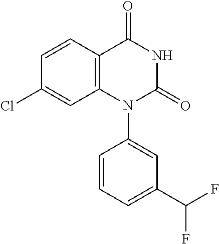 [SMILES: O=c1[nH]c(=O)n(-c2cccc(C(F)F)c2)c2cc(Cl)ccc12] | 7-chloro-1-(3-difluoromethylphenyl)- quinazoline-2,4(1H,3H)-dione | m/z [M + H]+ 323.26 |
| 355 | 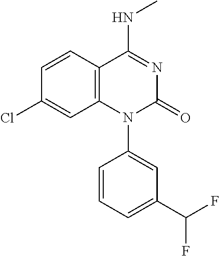 [SMILES: CNc1nc(=O)n(-c2cccc(C(F)F)c2)c2cc(Cl)ccc12] | 7-chloro-1-(3- (difluoromethyl)phenyl)-4- (methylamino)-3,4- dihydroquinazolin-2(1H)-one | m/z [M + H]+ 336.30 |
| 356 | 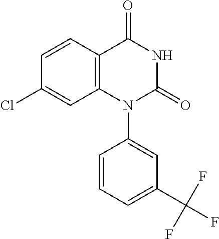 [SMILES: O=c1[nH]c(=O)n(-c2cccc(C(F)(F)F)c2)c2cc(Cl)ccc12] | 7-chloro-1-(3-trifluoromethylphenyl)- quinazoline-2,4(1H,3H)-dione | m/z [M + H]+ 339.29 |
| 357 | 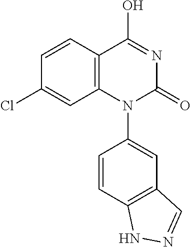 [SMILES: O=c1nc(O)c2ccc(Cl)cc2n1-c1ccc2[nH]ncc2c1] | 1-(1H-indazol-5-yl)-7-chloro-4- hydroxy-hydroquinazolin-2-one | m/z [M + H]+ 313.0 |
| 358 | 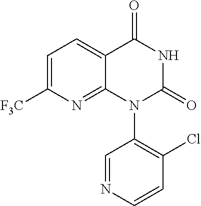 [SMILES: O=c1[nH]c(=O)n(-c2cnccc2Cl)c2nc(C(F)(F)F)ccc12] | 1-(4-chloropyridin-3-yl)-7-(trifluoro- methyl)pyrido[2,3-d]pyrimidine- 2,4(1H,3H)-dione | m/z [M + H]+ 343.0 |
| 359 | 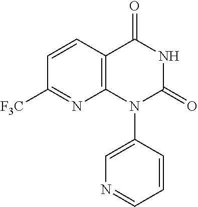 [SMILES: O=c1[nH]c(=O)n(-c2cccnc2)c2nc(C(F)(F)F)ccc12] | 1-(pyridin-3-yl)-7-(trifluoromethyl)- pyrido[2,3-d]pyrimidine-2,4(1H,3H)- dione | m/z [M + H]+ 309.00 |
| 360 | 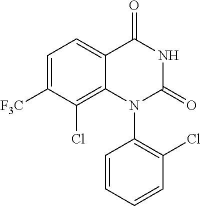 [SMILES: O=c1[nH]c(=O)n(-c2ccccc2Cl)c2c(Cl)c(C(F)(F)F)ccc12] | 8-chloro-1-(2-chlorophenyl)-7- (trifluoro-methyl)pyrido[2,3- d]pyrimidine-2,4(1H,3H)-dione-1 | m/z [M − H]− 373.0 |
| 361 | 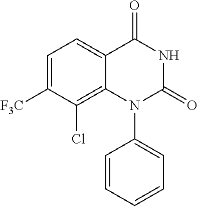 [SMILES: O=c1[nH]c(=O)n(-c2ccccc2)c2c(Cl)c(C(F)(F)F)ccc12] | 8-chloro-1-phenyl-7-(trifluoromethyl)- pyrido[2,3-d]pyrimidine-2,4(1H,3H)- dione | m/z [M − H]− 339.0 |
| 362 | 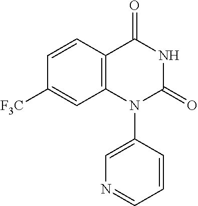 [SMILES: O=c1[nH]c(=O)n(-c2cccnc2)c2cc(C(F)(F)F)ccc12] | 1-(pyridin-3-yl)-7-(trifluoromethyl)- 1,3-dihydroquinazoline-2,4-dione | m/z [M + H]+ 308.0 |
| 363 | 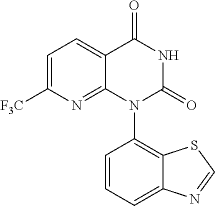 [SMILES: O=c1[nH]c(=O)n(-c2cccc3ncsc23)c2nc(C(F)(F)F)ccc12] | 1-benzothiazol-7-yl-7- (trifluoromethyl)-pyrido[2,3- d]pyrimidine-2,4(1H,3H)-dione | m/z [M + H]+ 365.0 |
| 364 | 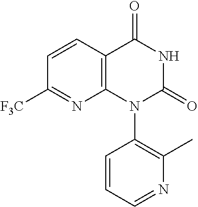 [SMILES: Cc1ncccc1-n1c(=O)[nH]c(=O)c2ccc(C(F)(F)F)nc21] | 1-(2-methylpyridin-3-yl)-7-(trifluroo- methyl)pyrido[2,3-d]pyrimidine- 2,4(1H,3H)-dione | m/z [M + H]+ 323.1 |
| 365 | 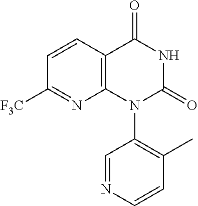 [SMILES: Cc1ccncc1-n1c(=O)[nH]c(=O)c2ccc(C(F)(F)F)nc21] | 1-(4-methylpyridin-3-yl)-7-(trifluoro- methyl)pyrido[2,3-d]pyrimidine- 2,4(1H,3H)-dione | m/z [M + H]+ 323.1 |
| 366 | 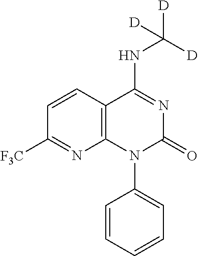 [SMILES: [2H]C([2H])([2H])Nc1nc(=O)n(-c2ccccc2)c2nc(C(F)(F)F)ccc12] | 4-((methyl-d3)amino)-1-phenyl-7- (trifluoromethyl)pyrido[2,3- d]pyrimidin-2(1H)-one | m/z [M + H]+ 324.1 |
| 367 | 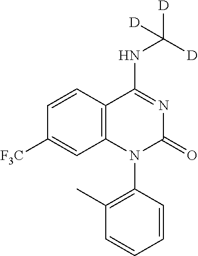 [SMILES: [2H]C([2H])([2H])Nc1nc(=O)n(-c2ccccc2C)c2cc(C(F)(F)F)ccc12] | 4-((methyl-d3)amino)-1-(o-tolyl)-7- (trfluoromethyl)quinazolin-2(1H)-one | m/z [M + H]+ 337.2 |
| 368 | 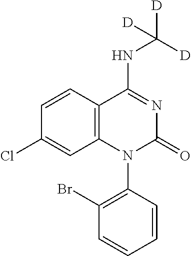 [SMILES: [2H]C([2H])([2H])Nc1nc(=O)n(-c2ccccc2Br)c2cc(Cl)ccc12] | 1-(2-bromophenyl)-7-chloro-4- ((methyl-d3)amino)quinazolin-2(1H)- one | m/z [M + H]+ 367.0 |
| 369 | 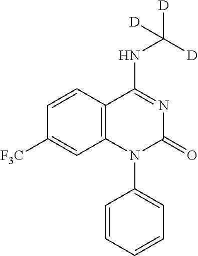 [SMILES: [2H]C([2H])([2H])Nc1nc(=O)n(-c2ccccc2)c2cc(C(F)(F)F)ccc12] | 4-((methyl-d3)amino)-1-phenyl-7- (trifluoromethyl)quinazolin-2(1H)-one | m/z [M + H]+ 323.2 |
| 370 | 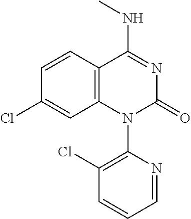 [SMILES: CNc1nc(=O)n(-c2ncccc2Cl)c2cc(Cl)ccc12] | 7-chloro-1-(3-chloro(2-pyridyl))-4- (methylamino)hydroquinazolin-2-one | m/z [M + H]+ 321.26 |
| 371 | 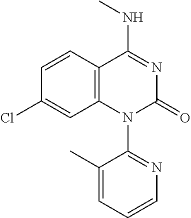 [SMILES: CNc1nc(=O)n(-c2ncccc2C)c2cc(Cl)ccc12] | 7-chloro-1-(3-methylpyridin-2-yl))-4- (methylamino)hydroquinazolin-2-one | m/z [M + H]+ 301.3 |
| 372 | 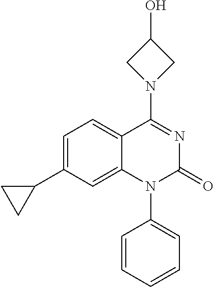 [SMILES: O=c1nc(N2CC(O)C2)c2ccc(C3CC3)cc2n1-c1ccccc1] | 7-cyclopropyl-4-(3-hydroxyazetidin-1- yl)-1-phenylhydroquinazolin-2-one | m/z [M + H]+ 334.36 |
| 373 | 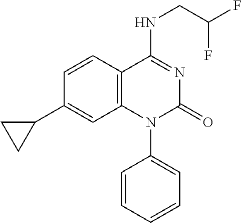 [SMILES: O=c1nc(NCC(F)F)c2ccc(C3CC3)cc2n1-c1ccccc1] | 7-cyclopropyl-4-((2-fluoropropyl)- amino)-1-phenylquinazolin-2(1H)- one | m/z [M + H]+ 342.35 |
| 374 | 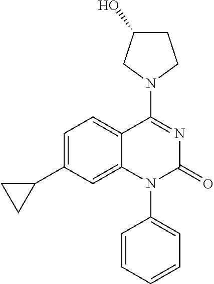 [SMILES: O=c1nc(N2CC[C@@H](O)C2)c2ccc(C3CC3)cc2n1-c1ccccc1] | 4-((3R)-3-hydroxypyrrolidinyl)-7- cyclopropyl-1- phenylhydroquinazolin-2-one | m/z [M + H]+ 348.37 |
| 375 | 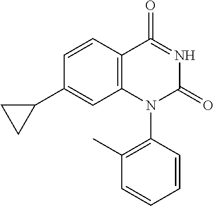 | 7-cyclopropyl-1-(o-tolyl)quinazoline- 2,4(1H,3H)-dione | m/z [M + H]+ 293.25 |
| 376 | 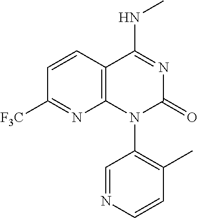 [SMILES: CNc1nc(=O)n(-c2cnccc2C)c2nc(C(F)(F)F)ccc12] | 4-(methylamino)-1-(4-methylpyridin- 3-yl)-7-(trifluoromethyl)pyrido[2,3-d]- pyrimidin-2(1H)-one | m/z [M + H]+ 336.1 |
| 377 | 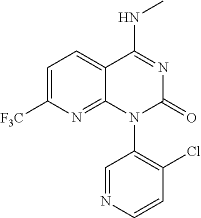 [SMILES: CNc1nc(=O)n(-c2cnccc2Cl)c2nc(C(F)(F)F)ccc12] | 1-(4-chloropyridin-3-yl)-4-(methyl- amino)-7-(trifluoromethyl)pyrido[2,3- d]pyrimidin-2(1H)-one | m/z [M + H]+ 356.00 |
| 378 | 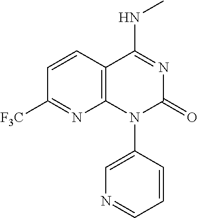 [SMILES: CNc1nc(=O)n(-c2cccnc2)c2nc(C(F)(F)F)ccc12] | 4-(methylamino)-1-(pyridin-3-yl)-7- (trifluoromethyl)pyrido[2,3- d]pyrimidin-2(1H)-one | m/z [M + H]+ 322.10 |
| 379 | 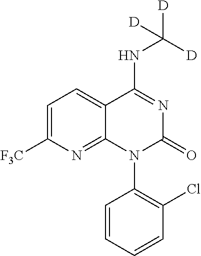 [SMILES: [2H]C([2H])([2H])Nc1nc(=O)n(-c2ccccc2Cl)c2nc(C(F)(F)F)ccc12] | 1-(2-chlorophenyl)-4-((methyl-d3)- amino)-7-(trifluoromethyl)pyrido[2,3- d]pyrimidin-2(1H)-one | m/z [M + H]+ 358.00 |
| 380 | 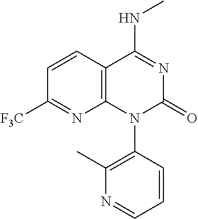 [SMILES: CNc1nc(=O)n(-c2cccnc2C)c2nc(C(F)(F)F)ccc12] | 4-(methylamino)-1-(2-methylpyridin- 3-yl)-7-(trifluoromethyl)pyrido[2,3-d]- pyrimidin-2(1H)-one | m/z [M + H]+ 336.1 |
| 381 | 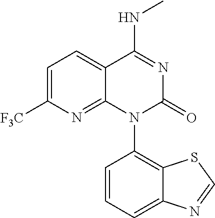 [SMILES: CNc1nc(=O)n(-c2cccc3ncsc23)c2nc(C(F)(F)F)ccc12] | 1-benzothiazol-7-yl-4-(methylamino)- 7-(trifluoromethyl)pyrido[2,3- d]pyrimidin-2(1H)-one | m/z [M + H]+ 378.0 |
| 382 | 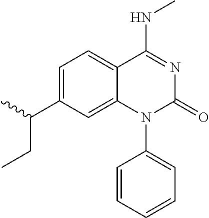 [SMILES: CCC(C)c1ccc2c(NC)nc(=O)n(-c3ccccc3)c2c1] | 4-(methylamino)-7-(methylpropyl)-1- phenylhydroquinazolin-2-one | m/z [M + H]+ 308.2 |
| 383 | 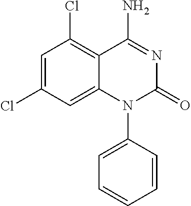 [SMILES: Nc1nc(=O)n(-c2ccccc2)c2cc(Cl)cc(Cl)c12] | 4-amino-5,7-dichloro-1-phenyl- hydroquinazolin-2-one | m/z [M + H]+ 306.23 |
| 384 | 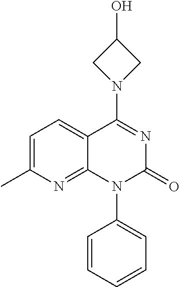 [SMILES: Cc1ccc2c(N3CC(O)C3)nc(=O)n(-c3ccccc3)c2n1] | 4-(3-hydroxyazetidin-1-yl)-7-methyl- 1-phenylpyrido[2,3-d]pyrimidin- 2(1H)-one | m/z [M + H]+ 309.32 |
| 385 | 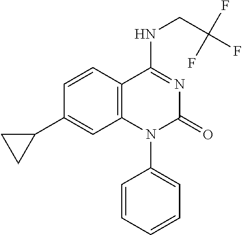 [SMILES: O=c1nc(NCC(F)(F)F)c2ccc(C3CC3)cc2n1-c1ccccc1] | 7-cyclopropyl-1-phenyl-4-[(2,2,2- trifluoroethyl)amino]hydroquinazolin- 2-one | m/z [M + H]+ 360.36 |
| 386 | 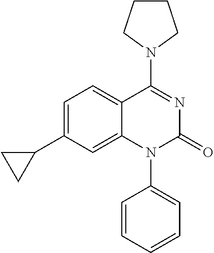 [SMILES: O=c1nc(N2CCCC2)c2ccc(C3CC3)cc2n1-c1ccccc1] | 7-cyclopropyl-1-phenyl-4-pyrrolidin- 1-ylhydroquinazolin-2-one | m/z [M + H]+ 332.38 |
| 387 | 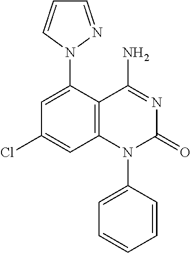 [SMILES: Nc1nc(=O)n(-c2ccccc2)c2cc(Cl)cc(-n3cccn3)c12] | 4-amino-7-chloro-1-phenyl-5-pyrazol- 1-ylhydroquinazolin-2-one | m/z [M + H]+ 338.31 |
| 388 | 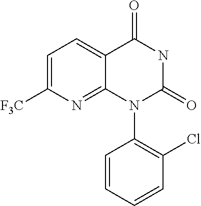 [SMILES: O=c1[nH]c(=O)n(-c2ccccc2Cl)c2nc(C(F)(F)F)ccc12] | 1-(2-chlorophenyl)-7- (trifluoromethyl)-1,3- dihydropyridino[2,3-d]pyrimidine-2,4- dione | m/z [M + H]+ 342.0 |
| 389 | 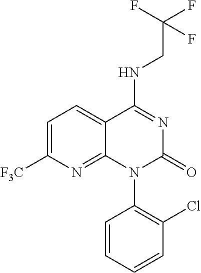 [SMILES: O=c1nc(NCC(F)(F)F)c2ccc(C(F)(F)F)nc2n1-c1ccccc1Cl] | 1-(2-chlorophenyl)-4-[(2,2,2-trifluoro- ethyl)amino]-7-(trifluoro-methyl)- pyrido[2,3-d]pyrimidin-2(1H)-one | m/z [M + H]+ 423.10 |
| 390 | 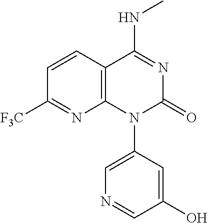 [SMILES: CNc1nc(=O)n(-c2cncc(O)c2)c2nc(C(F)(F)F)ccc12] | 1-(5-hydroxypyridin-3-yl)-4-(methyl- amino)-7-(trifluoromethyl)pyrido[2,3- d]-pyrimidin-2(1H)-one | m/z [M + H]+ 338.10 |
| 391 | 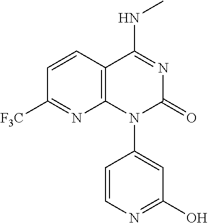 [SMILES: CNc1nc(=O)n(-c2ccnc(O)c2)c2nc(C(F)(F)F)ccc12] | 1-(2-hydroxypyridin-4-yl)-4-(methyl- amino)-7-(trifluoromethyl)-pyrido[2,3- d]pyrimidin-2(1H)-one | m/z [M + H]+ 338.10 |
| 392 | 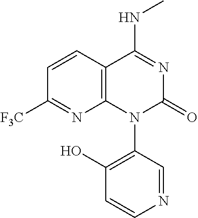 [SMILES: CNc1nc(=O)n(-c2cnccc2O)c2nc(C(F)(F)F)ccc12] | 1-(4-hydroxypyridin-3-yl)-4-(methyl- amino)-7-(trifluoromethyl)pyrido[2,3- d]-pyrimidin-2(1H)-one | m/z [M + H]+ 338.10 |
| 393 | 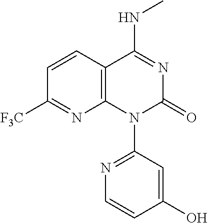 [SMILES: CNc1nc(=O)n(-c2cc(O)ccn2)c2nc(C(F)(F)F)ccc12] | 1-(5-hydroxypyridin-2-yl)-4-(methyl- amino)-7-(trifluoromethyl)pyrido[2,3- d]-pyrimidin-2(1H)-one | m/z [M + H]+ 338.10 |
| 394 | 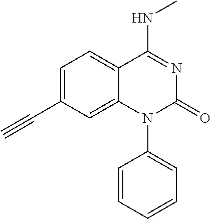 [SMILES: C#Cc1ccc2c(NC)nc(=O)n(-c3ccccc3)c2c1] | 7-ethynyl-4-(methylamino)-1-phenyl- hydroquinazolin-2-one | m/z [M + H]+ 276.0 |
| 395 | 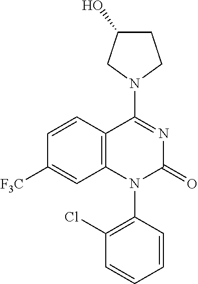 [SMILES: O=c1nc(N2CC[C@@H](O)C2)c2ccc(C(F)(F)F)cc2n1-c1ccccc1Cl] | 4-((3R)-3-hydroxypyrrolidin-1-yl)-1- (2-chlorophenyl)-7- (trifluoromethyl)hydro-quinazolin-2- one | m/z [M + H]+ 410.0 |
| 396 | 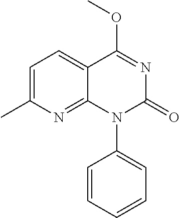 [SMILES: COc1nc(=O)n(-c2ccccc2)c2nc(C)ccc12] | 4-methoxy-7-methyl-1-phenylpyrido- [2,3-d]pyrimidin-2(1H)-one | m/z [M + H]+ 268.12 |
| 397 | 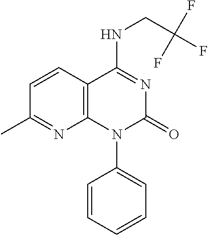 [SMILES: Cc1ccc2c(NCC(F)(F)F)nc(=O)n(-c3ccccc3)c2n1] | 7-methyl-1-phenyl-4-[(1,1,1-trifluoro- ethyl)amino]pyrido[2,3-d]pyrimidin- 2(1H)-one | m/z [M + H]+ 335.16 |
| 398 | 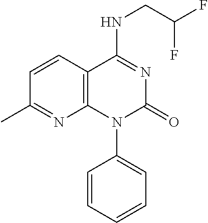 [SMILES: Cc1ccc2c(NCC(F)F)nc(=O)n(-c3ccccc3)c2n1] | 4-[(2,2-difluoroethyl)amino]-7-methyl- 1-phenylpyrido[2,3-d]pyrimidin- 2(1H)-one | m/z [M + H]+ 317.17 |
| 399 | 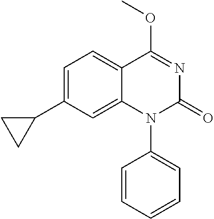 [SMILES: COc1nc(=O)n(-c2ccccc2)c2cc(C3CC3)ccc12] | 7-cyclopropyl-4-methoxy-1-phenyl- hydroquinazolin-2-one | m/z [M + H]+ 293.1 |
| 400 | 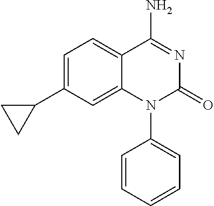 [SMILES: Nc1nc(=O)n(-c2ccccc2)c2cc(C3CC3)ccc12] | 4-amino-7-cyclopropyl-1- phenylhydro-quinazolin-2-one | m/z [M + H]+ 278.21 |
| 401 | 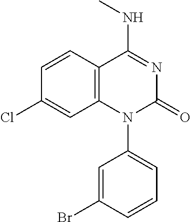 [SMILES: CNc1nc(=O)n(-c2cccc(Br)c2)c2cc(Cl)ccc12] | 1-(3-bromophenyl)-7-chloro-4- (methylamino)hydroquinazolin-2-one | m/z [M + H]+ 363.97 |
| 402 | 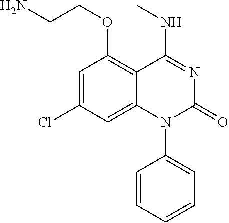 [SMILES: CNc1nc(=O)n(-c2ccccc2)c2cc(Cl)cc(OCCN)c12] | 5-(2-aminoethoxy)-7-chloro-4- (methyl-amino)-1- phenylhydroquinazolin-2-one | m/z [M + H]+ 345.1 |
| 403 | 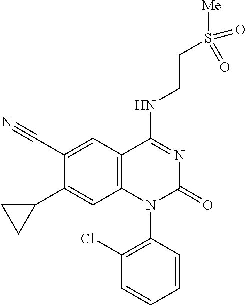 [SMILES: CS(=O)(=O)CCNc1nc(=O)n(-c2ccccc2Cl)c2cc(C3CC3)c(C#N)cc12] | 1-(2-chlorophenyl)-7-cyclopropyl-4- ((2-(methylsulfonyl)ethyl)amino)-2- oxo-1,2-dihydroquinazoline-6- carbonitrile | m/z [M + H]+ 443.1 |
| 404 | 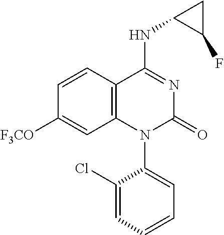 | 4-[((1R,2R)-2-fluorocyclopropyl)- amino]-1-(2-chlorophenyl)-7- (trifluoromethoxy)hydroquinazolin-2- one | m/z [M + H]+ 414.0. |
| 405 | 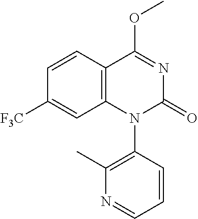 [SMILES: COc1nc(=O)n(-c2cccnc2C)c2cc(C(F)(F)F)ccc12] | 4-methoxy-7-methyl-1-(2-methyl- pyridin-3-yl)quinazolin-2(1H)-one | m/z [M + H]+ 336.40 |
| 406 | 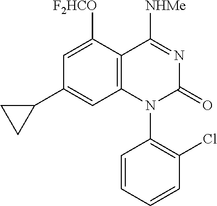 [SMILES: CNc1nc(=O)n(-c2ccccc2Cl)c2cc(C3CC3)cc(OC(F)F)c12] | 5-(difluoromethoxy)-1-(2- chlorophenyl)-7-cyclopropyl-4- (methylamino)hydro-quinazolin-2- one | m/z [M + H]+ 392.3. |
| 407 | 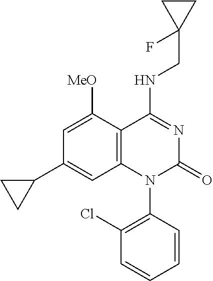 [SMILES: COc1cc(C2CC2)cc2c1c(NCC1(F)CC1)nc(=O)n2-c1ccccc1Cl] | 1-(2-chlorophenyl)-7-cyclopropyl-4- {[(fluorocyclopropyl)methyl]amino}-5- methoxyhydroquinazolin-2-one | m/z [M + H]+ 414.39 |
| 408 | 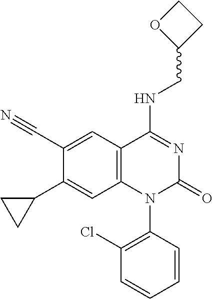 [SMILES: N#Cc1cc2c(NCC3CCO3)nc(=O)n(-c3ccccc3Cl)c2cc1C1CC1] | 1-(2-chlorophenyl)-7-cyclopropyl-4- ((oxetan-2-ylmethyl)amino)-2-oxo- 1,2-dihydroquinazoline-6-carbonitrile | m/z [M + H]+ 407.0 |
| 409 | 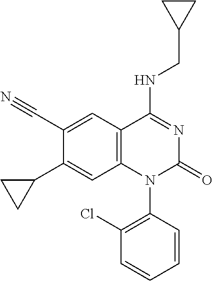 [SMILES: N#Cc1cc2c(NCC3CC3)nc(=O)n(-c3ccccc3Cl)c2cc1C1CC1] | 1-(2-chlorophenyl)-7-cyclopropyl-4- ((cyclopropylmethyl)amino)-2-oxo- 1,2-dihydroquinazoline-6-carbonitrile | m/z [M + H]+ 391.0 |
| 410 | 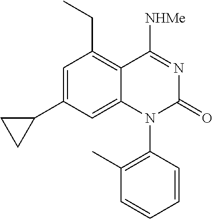 [SMILES: CCc1cc(C2CC2)cc2c1c(NC)nc(=O)n2-c1ccccc1C] | 7-cyclopropyl-5-ethyl-4- (methylamino)-1-(2- methylphenyl)hydroquinazolin-2-one | m/z [M + H]+ 334.37. |
| 411 | 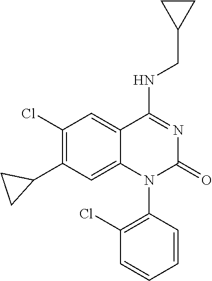 [SMILES: O=c1nc(NCC2CC2)c2cc(Cl)c(C3CC3)cc2n1-c1ccccc1Cl] | 6-chloro-1-(2-chlorophenyl)-7-cyclo- propyl-4-[(cyclopropylmethyl)amino]- hydroquinazolin-2-one | m/z [M + H]+ 400.1. |
| 412 | 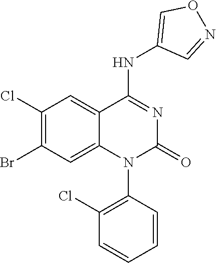 [SMILES: O=c1nc(Nc2cnoc2)c2cc(Cl)c(Br)cc2n1-c1ccccc1Cl] | 7-bromo-6-chloro-1-(2-chlorophenyl)- 4-(isoxazol-4- ylamino)hydroquinazolin-2-one | m/z [M + H ]+ 452.9 (major) |
| 413 | 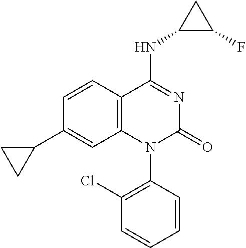 [SMILES: O=c1nc(N[C@@H]2C[C@@H]2F)c2ccc(C3CC3)cc2n1-c1ccccc1Cl] | 4-[((2S,1R)-2- fluorocyclopropyl)amino]-1-(2- chlorophenyl)-7-cyclopropyl- hydroquinazolin-2-one | m/z [M + H]+ 370.0 |
| 414 | 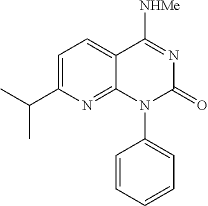 [SMILES: CNc1nc(=O)n(-c2ccccc2)c2nc(C(C)C)ccc12] | 4-(methylamino)-7-(methylethyl)-1- phenylpyrido[2,3-d]pyrimidin-2(1H)- one | m/z [M + H]+ 295.1 |
| 415 | 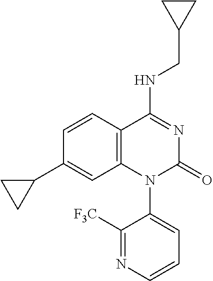 [SMILES: O=c1nc(NCC2CC2)c2ccc(C3CC3)cc2n1-c1cccnc1C(F)(F)F] | 7-cyclopropyl-4-[(cyclopropylmethyl)- amino]-1-[2-(trifluoromethyl)(3- pyridyl))hydroquinazolin-2-one | m/z [M + H]+ 399.1 |
| 416 | 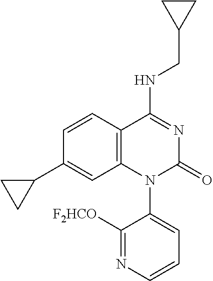 [SMILES: O=c1nc(NCC2CC2)c2ccc(C3CC3)cc2n1-c1cccnc1OC(F)F] | 1-[2-(difluoromethoxy)-(3-pyridyl)]-7- cyclopropyl-4-[(cyclopropylmethyl)- amino]hydroquinazolin-2-one | m/z [M + H]+ 401.1. |
| 417 | 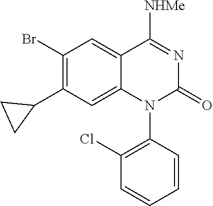 [SMILES: CNc1nc(=O)n(-c2ccccc2Cl)c2cc(C3CC3)c(Br)cc12] | 6-bromo-1-(2-chlorophenyl)-7-cyclo- propyl-4-(methylamino)hydro- quinazolin-2-one | m/z [M + H]+ 404.0 |
| 418 | 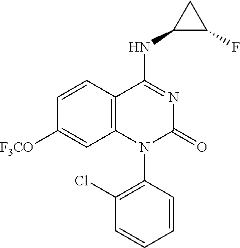 [SMILES: O=c1nc(N[C@H]2C[C@@H]2F)c2ccc(OC(F)(F)F)cc2n1-c1ccccc1Cl] | 1-(2-chlorophenyl)-4-(((1S,2S)-2- fluorocyclopropyl)amino)-7-(trifluoro- methoxy)quinazolin-2(1H)-one | m/z [M + H]+ 414.0. |
| 419 | 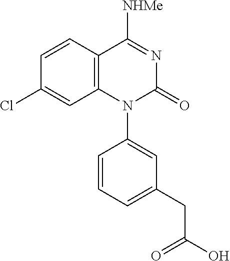 [SMILES: CNc1nc(=O)n(-c2cccc(CC(=O)O)c2)c2cc(Cl)ccc12] | 2-(3-(7-chloro-4-(methylamino)-2- oxoquinazolin-1(2H)-yl)phenyl)acetic acid | m/z [M + H]+ 344.30. |
| 420 | 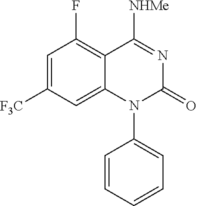 [SMILES: CNc1nc(=O)n(-c2ccccc2)c2cc(C(F)(F)F)cc(F)c12] | 5-fluoro-4-(methylamino)-1-phenyl-7- (trifluoromethyl)hydroquinazolin-2- one | m/z [M + H]+ 338.1. |
| 421 | 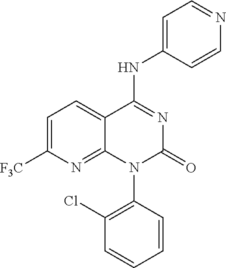 [SMILES: O=c1nc(Nc2ccncc2)c2ccc(C(F)(F)F)nc2n1-c1ccccc1Cl] | 1-(2-chlorophenyl)-4-(pyridin-4-yl- amino)-7-(trifluoromethyl)pyrido[2,3- d]-pyrimidin-2(1H)-one | m/z [M + H]+ 418.00. |
| 422 | 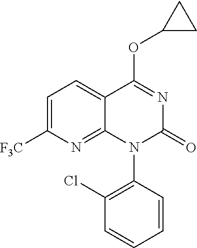 [SMILES: O=c1nc(OC2CC2)c2ccc(C(F)(F)F)nc2n1-c1ccccc1Cl] | 1-(2-chlorophenyl)-4-cyclopropoxy-7- (trifluoromethyl)pyrido[2,3- d]pyrimidin-2(1H)-one | m/z [M + H]+ 382.33 |
| 423 | 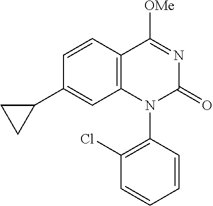 [SMILES: COc1nc(=O)n(-c2ccccc2Cl)c2cc(C3CC3)ccc12] | 1-(2-chlorophenyl)-7-cyclopropyl-4- methoxyhydroquinazolin-2-one | m/z [M + H]+ 327.16. |
| 424 | 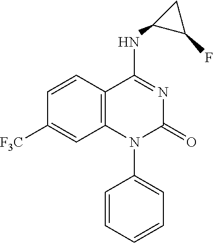 [SMILES: O=c1nc(N[C@H]2C[C@H]2F)c2ccc(C(F)(F)F)cc2n1-c1ccccc1] | 4-(((1S,2R)-2- fluorocyclopropyl)amino)-1-phenyl-7- (trifluoromethyl)quinazolin-2(1H)-one | m/z [M + H]+ 364.1 |
| 425 | 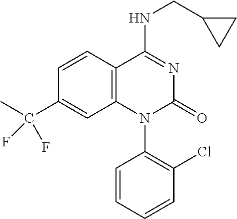 [SMILES: CC(F)(F)c1ccc2c(NCC3CC3)nc(=O)n(-c3ccccc3Cl)c2c1] | 1-(2-chlorophenyl)-4-((cyclopropyl- methyl)amino)-7-(1,1-difluoroethyl)- quinazolin-2(1H)-one | m/z [M + H]+ 390.1 |
| 426 | 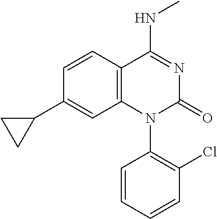 [SMILES: CNc1nc(=O)n(-c2ccccc2Cl)c2cc(C3CC3)ccc12] | 1-(2-chlorophenyl)-7-cyclopropyl-4- (methylamino)quinazolin-2(1H)-one | m/z [M + H]+ 326.0 |
| 427 | 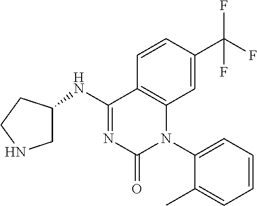 [SMILES: Cc1ccccc1-n1c(=O)nc(N[C@H]2CCNC2)c2ccc(C(F)(F)F)cc21] | (S)-4-(pyrrolidin-3-ylamino)-1-(o- tolyl)-7-(trifluoromethyl)quinazolin- 2(1H)-one | m/z [M + H]⁺ 389.15 |
| 428 | 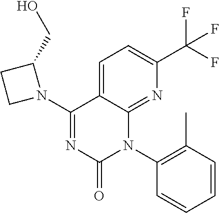 [SMILES: Cc1ccccc1-n1c(=O)nc(N2CC[C@@H]2CO)c2ccc(C(F)(F)F)nc21] | (R)-4-(2-(hydroxymethyl)azetidin-1- yl)-1-(o-tolyl)-7- (trifluoromethyl)pyrido-[2,3- d]pyrimidin-2(1H)-one | m/z [M + H]⁺ 391.1 |
| 429 | 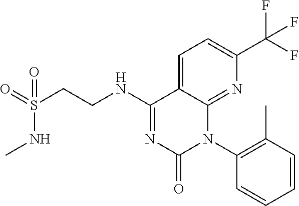 [SMILES: CNS(=O)(=O)CCNc1nc(=O)n(-c2ccccc2C)c2nc(C(F)(F)F)ccc12] | N-methyl-2-((2-oxo-1-(o-tolyl)-7- (trifluoromethyl)-1,2- dihydropyrido[2,3-d]pyrimidin-4- yl)amino)ethane-1-sulfonamide | m/z [M + H]⁺ 442.1 |
| 430 | 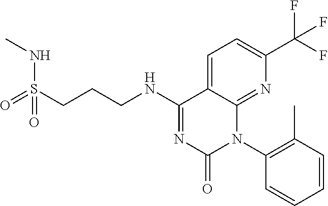 [SMILES: CNS(=O)(=O)CCCNc1nc(=O)n(-c2ccccc2C)c2nc(C(F)(F)F)ccc12] | N-methyl-3-((2-oxo-1-(o-tolyl)-7- (trifluoromethyl)-1,2- dihydropyrido[2,3-d]pyrimidin-4- yl)amino)propane-1-sulfonamide | m/z [M + H]⁺ 456.1 |
| 431 | 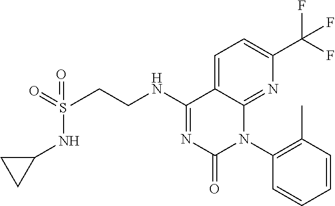 | N-cyclopropyl-2-((2-oxo-1-(o-tolyl)-7- (trifluoromethyl)-1,2- dihydropyrido[2,3-d]pyrimidin-4- yl)amino)ethane-1-sulfonamide | m/z [M + H]⁺ 468.1 |
| 432 | 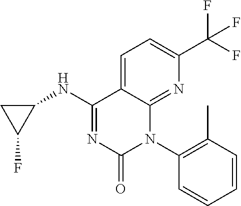 [SMILES: Cc1ccccc1-n1c(=O)nc(N[C@H]2C[C@H]2F)c2ccc(C(F)(F)F)nc21] | 4-(((1S,2R)-2- fluorocyclopropyl)amino)-1-(o-tolyl)- 7-(trifluoromethyl)pyrido-[2,3- d]pyrimidin-2(1H)-one | m/z [M + H]⁺ 379.1 |
| 433 | 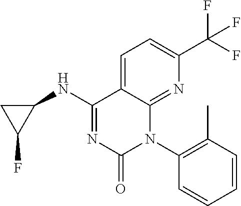 [SMILES: Cc1ccccc1-n1c(=O)nc(N[C@@H]2C[C@@H]2F)c2ccc(C(F)(F)F)nc21] | 4-(((1R,2S)-2- fluorocyclopropyl)amino)-1-(o-tolyl)- 7-(trifluoromethyl)pyrido-[2,3- d]pyrimidin-2(1H)-one | m/z [M + H]⁺ 379.1 |
| 434 | 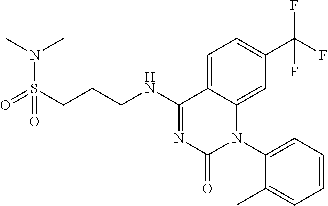 [SMILES: Cc1ccccc1-n1c(=O)nc(NCCCS(=O)(=O)N(C)C)c2ccc(C(F)(F)F)cc21] | N,N-dimethyl-3-((2-oxo-1-(o-tolyl)-7- (trifluoromethyl)-1,2- dihydroquinazolin-4- yl)amino)propane-1-sulfonamide | m/z [M + H]⁺ 469.1 |
| 435 | 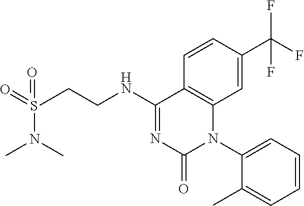 | N,N-dimethyl-2-((2-oxo-1-(o-tolyl)-7- (trifluoromethyl)-1,2- dihydroquinazolin-4- yl)amino)ethane-1-sulfonamide | m/z [M + H]⁺ 455.1 |
| 436 | 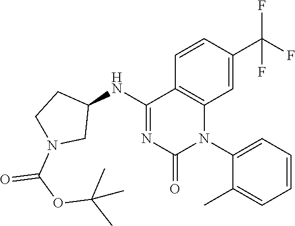 [SMILES: Cc1ccccc1-n1c(=O)nc(N[C@@H]2CCN(C(=O)OC(C)(C)C)C2)c2ccc(C(F)(F)F)cc21] | tert-butyl (R)-3-(2-oxo-1-(o-tolyl)-7- (trifluoromethyl)-1,2- dihydroquinazolin-4- yl)amino)pyrrolidine-1-carboxylate | m/z [M + H]⁺ 489.2 |
| 437 | 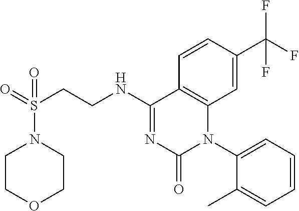 [SMILES: Cc1ccccc1-n1c(=O)nc(NCCS(=O)(=O)N2CCOCC2)c2ccc(C(F)(F)F)cc21] | 4-((2- (morpholinosulfonyl)ethyl)amino)-1- (o-tolyl)-7-(trifluoromethyl)- quinazolin-2(1H)-one | m/z [M + H]⁺ 497.1 |
| 438 | 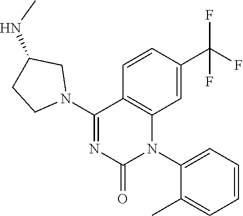 [SMILES: CN[C@H]1CCN(c2nc(=O)n(-c3ccccc3C)c3cc(C(F)(F)F)ccc23)C1] | (S)-4-(3-(methylamino)pyrrolidin-1- yl)-1-(o-tolyl)-7-(trifluoromethyl)- quinazolin-2(1H)-one | m/z [M + H]⁺ 403.1 |
| 439 | 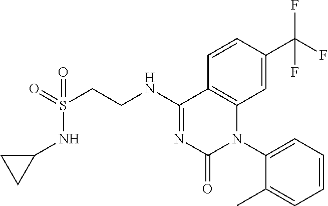 [SMILES: Cc1ccccc1-n1c(=O)nc(NCCS(=O)(=O)NC2CC2)c2ccc(C(F)(F)F)cc21] | N-cyclopropyl-2-((2-oxo-1-(o-tolyl)-7- (trifluoromethyl)-1,2- dihydroquinazolin-4- yl)amino)ethane-1-sulfonamide | m/z [M + H]⁺ 467.1 |
| 440 | 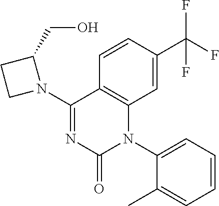 [SMILES: Cc1ccccc1-n1c(=O)nc(N2CC[C@@H]2CO)c2ccc(C(F)(F)F)cc21] | (R)-4-(2-(hydroxymethyl)azetidin-1- yl)-1-(o-tolyl)-7-(trifluoromethyl)- quinazolin-2(1H)-one | m/z [M + H]⁺ 390.1 |
| 441 | 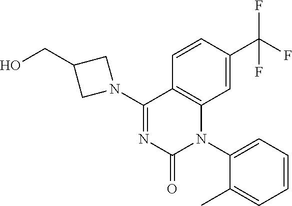 [SMILES: Cc1ccccc1-n1c(=O)nc(N2CC(CO)C2)c2ccc(C(F)(F)F)cc21] | 4-(3-(hydroxymethyl)azetidin-1-yl)-1- (o-tolyl)-7-(trifluoromethyl)quinazolin- 2(1H)-one | m/z [M + H]⁺ 390.1 |
| 442 | 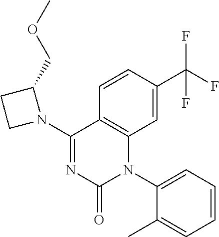 [SMILES: COC[C@H]1CCN1c1nc(=O)n(-c2ccccc2C)c2cc(C(F)(F)F)ccc12] | (R)-4-(2-(methoxymethyl)azetidin-1- yl)-1-(o-tolyl)-7-(trifluoromethyl)- quinazolin-2(1H)-one | m/z [M + H]⁺ 404 |
| 443 | 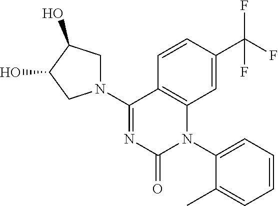 [SMILES: Cc1ccccc1-n1c(=O)nc(N2C[C@H](O)[C@@H](O)C2)c2ccc(C(F)(F)F)cc21] | 4-((3S.4S)-3,4-dihydroxypyrrolidin-1- yl)-1-(o-tolyl)-7-(trifluoromethyl)- quinazolin-2(1H)-one | m/z [M + H]⁺ 406.1 |
| 444 | 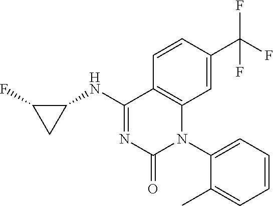 [SMILES: Cc1ccccc1-n1c(=O)nc(N[C@@H]2C[C@@H]2F)c2ccc(C(F)(F)F)cc21] | 4-(((1R,2S)-2- fluorocyclopropyl)amino)-1-(o-tolyl)- 7-(trifluoromethyl)-quinazolin-2(1H)- one | m/z [M + H]⁺ 378.1 |
| 445 | 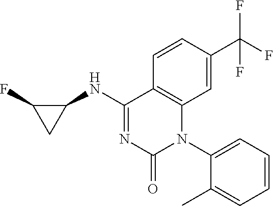 [SMILES: Cc1ccccc1-n1c(=O)nc(N[C@H]2C[C@H]2F)c2ccc(C(F)(F)F)cc21] | 4-(((1S,2R)-2- fluorocyclopropyl)amino)-1-(o-tolyl)- 7-(trifluoromethyl)-quinazolin-2(1H)- one | m/z [M + H]⁺ 378.1 |
| 446 | 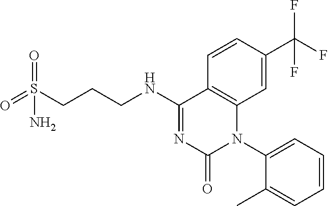 [SMILES: Cc1ccccc1-n1c(=O)nc(NCCCS(N)(=O)=O)c2ccc(C(F)(F)F)cc21] | 3-((2-oxo-1-(o-tolyl)-7-(trifluoro- methyl)-1,2-dihydroquinazolin-4-yl)- amino)propane-1-sulfonamide | m/z [M + H]⁺ 441.1 |
| 447 | 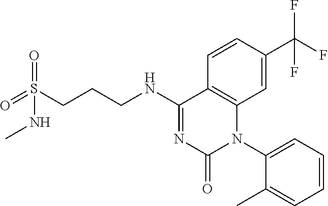 [SMILES: CNS(=O)(=O)CCCNc1nc(=O)n(-c2ccccc2C)c2cc(C(F)(F)F)ccc12] | N-methyl-3-((2-oxo-1-(o-tolyl)-7- (trifluoromethyl)-1,2- dihydroquinazolin-4- yl)amino)propane-1-sulfonamide | m/z [M + H]⁺ 455.1 |
| 448 | 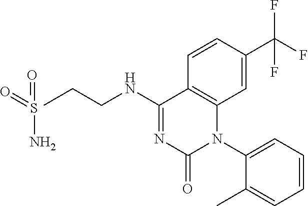 | 2-((2-oxo-1-(o-tolyl)-7-(trifluoro- methyl)-1,2-dihydroquinazolin-4-yl)- amino)ethane-1-sulfonamide | m/z [M + H]⁺ 427.05 |
| 449 | 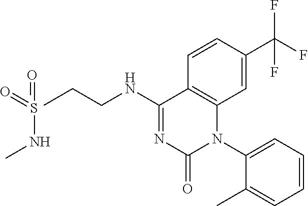 | N-methyl-2-((2-oxo-1-(o-tolyl)-7- (trifluoromethyl)-1,2- dihydroquinazolin-4- yl)amino)ethane-1-sulfonamide | m/z [M + H]⁺ 441.1 |
| 450 | 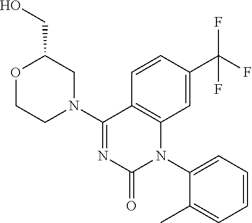 [SMILES: Cc1ccccc1-n1c(=O)nc(N2CCO[C@H](CO)C2)c2ccc(C(F)(F)F)cc21] | (S)-4-(2- (hydroxymethyl)morpholino)-1-(o- tolyl)-7-(trifluoromethyl)quinazolin- 2(1H)-one | m/z [M + H]⁺ 420.1 |
| 451 | 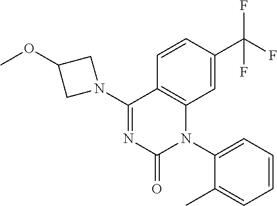 [SMILES: COC1CN(c2nc(=O)n(-c3ccccc3C)c3cc(C(F)(F)F)ccc23)C1] | 4-(3-methoxyazetidin-1-yl)-1-(o- tolyl)-7-(trifluoromethyl)quinazolin- 2(1H)-one | m/z [M + H]⁺ 390.1 |
| 452 | 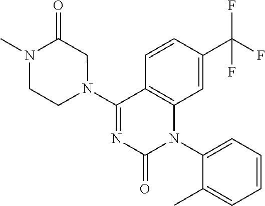 [SMILES: Cc1ccccc1-n1c(=O)nc(N2CCN(C)C(=O)C2)c2ccc(C(F)(F)F)cc21] | (4-(4-methyl-3-oxopiperazin-1-yl)-1- (o-tolyl)-7-(trifluoromethyl)quinazolin- 2(1H)-one | m/z [M + H]⁺ 417.1 |
| 453 | 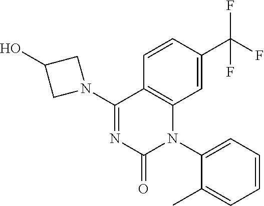 [SMILES: Cc1ccccc1-n1c(=O)nc(N2CC(O)C2)c2ccc(C(F)(F)F)cc21] | 4-(3-hydroxyazetidin-1-yl)-1-(o-tolyl)- 7-(trifluoromethyl)quinazolin-2(1H)- one | m/z [M + H]⁺ 376.1 |
| 454 | 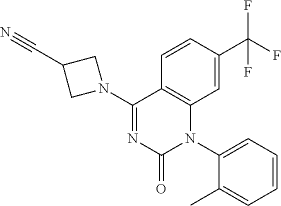 [SMILES: Cc1ccccc1-n1c(=O)nc(N2CC(C#N)C2)c2ccc(C(F)(F)F)cc21] | 1-(2-oxo-1-(o-tolyl)-7- (trifluoromethyl)-1,2- dihydroquinazolin-4-yl)azetidine-3- carbonitrile | m/z [M + H]⁺ 385.1 |
| 455 | 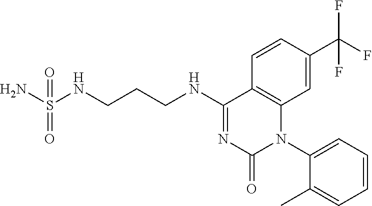 [SMILES: Cc1ccccc1-n1c(=O)nc(NCCCNS(N)(=O)=O)c2ccc(C(F)(F)F)cc21] | amino(3-{[1-(2-methylphenyl)-2-oxo- 7-(trifluoromethyl)hydroquinazolin-4- yl]-amino}propyl)sulfonamide | m/z [M + H]⁺ 456.1 |
| 456 | 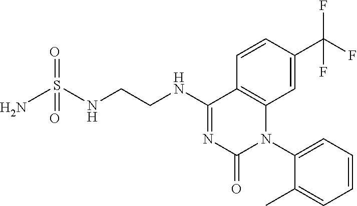 | amino(2-{[1-(2-methylphenyl)-2-oxo- 7-(trifluoromethyl)hydroquinazolin-4- yl]-amino}ethyl)sulfonamide | m/z [M + H]⁺ 442.1 |
| 457 | 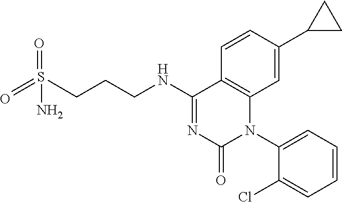 | (3-((1-(2-chlorophenyl)-7- cyclopropyl-2-oxo-1,2- dihydroquinazolin-4-yl)- amino)propane-1-sulfonamide | m/z [M + H]⁺ 433.1 |
| 458 | 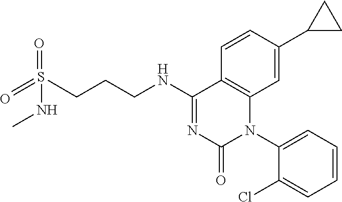 | 3-((1-(2-chlorophenyl)-7-cyclopropyl- 2-oxo-1,2-dihydroquinazolin-4- yl)amino)-N-methylpropane-1- sulfonamide | m/z [M + H]⁺ 447.0 |
| 459 | 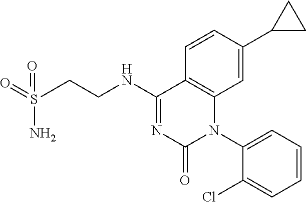 | 2-((1-(2-chlorophenyl)-7-cyclopropyl- 2-oxo-1,2-dihydroquinazolin-4-yl)- amino)ethane-1-sulfonamide | m/z [M + H]⁺ 419.1 |
| 460 | 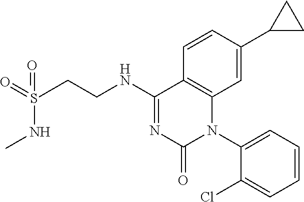 | 2-((1-(2-chlorophenyl)-7-cyclopropyl- 2-oxo-1,2-dihydroquinazolin-4- yl)amino)-N-methylethane-1- sulfonamide | m/z [M + H]⁺ 433.1 |
| 461 | 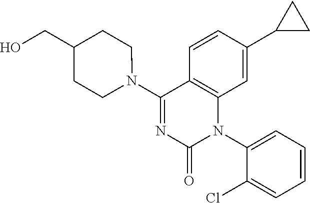 [SMILES: O=c1nc(N2CCC(CO)CC2)c2ccc(C3CC3)cc2n1-c1ccccc1Cl] | 1-(2-chlorophenyl)-7-cyclopropyl-4- (4-(hydroxymethyl)piperidin-1-yl)- quinazolin-2(1H)-one | m/z [M + H]⁺ 410.1 |
| 462 | 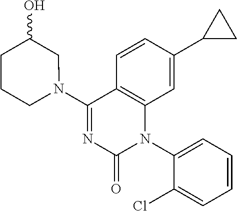 [SMILES: O=c1nc(N2CCCC(O)C2)c2ccc(C3CC3)cc2n1-c1ccccc1Cl] | 1-(2-chlorpohenyl)-7-cyclopropyl-4- (3-hydroxypiperidin-1-yl)quinazolin- 2(1H)-one | m/z [M + H]⁺ 396.1 |
| 463 | 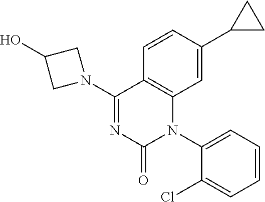 [SMILES: O=c1nc(N2CC(O)C2)c2ccc(C3CC3)cc2n1-c1ccccc1Cl] | 1-(2-chlorophenyl)-7-cyclopropyl-4- (3-hydroxyazetidin-1-yl)quinazolin- 2(1H)-one | m/z [M + H]⁺ 368.1 |
| 464 | 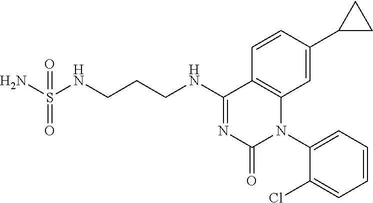 [SMILES: NS(=O)(=O)NCCCNc1nc(=O)n(-c2ccccc2Cl)c2cc(C3CC3)ccc12] | (3-{[1-(2-chlorophenyl)-7- cyclopropyl-2-oxohydroquinazolin-4- yl]amino}-propyl)sulfonamide | m/z [M + H]⁺ 448.1 |
| 465 | 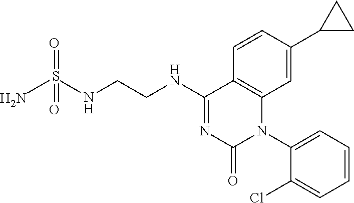 | (2-{[1-(2-chlorophenyl)-7- cyclopropyl-2-oxohydroquinazolin-4- yl]amino}-ethyl)sulfonamide | m/z [M + H]⁺ 434.0 |
| 466 | 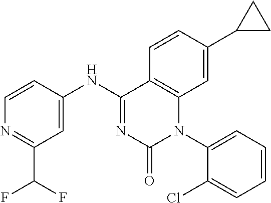 [SMILES: O=c1nc(Nc2ccnc(C(F)F)c2)c2ccc(C3CC3)cc2n1-c1ccccc1Cl] | 1-(2-chlorophenyl)-7-cyclopropyl-4- ((2-(difluoromethyl)pyridin-4- yl)amino)-quinazolin-2(1H)-on | m/z [M + H]⁺ 439.1 |
| 467 | 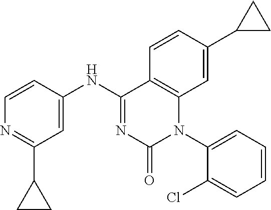 [SMILES: O=c1nc(Nc2ccnc(C3CC3)c2)c2ccc(C3CC3)cc2n1-c1ccccc1Cl] | 1-(2-chlorophenyl)-7-cyclopropyl-4- ((2-cyclopropylpyridin-4-yl)amino)- quinazolin-2(1H)-one | m/z [M + H]⁺ 429.1 |
| 468 | 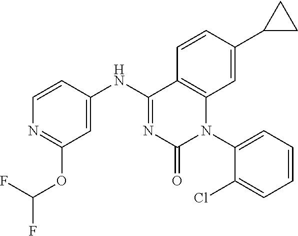 [SMILES: O=c1nc(Nc2ccnc(OC(F)F)c2)c2ccc(C3CC3)cc2n1-c1ccccc1Cl] | 1-(2-chlorophenyl)-7-cyclopropyl-4- ((2-(difluoromethoxy)pyridin-4- yl)amino)-quinazolin-2(1H)-one | m/z [M + H]⁺ 455.1 |
| 469 | 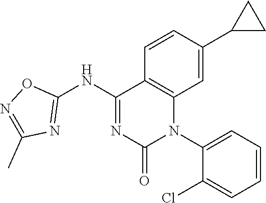 [SMILES: Cc1noc(Nc2nc(=O)n(-c3ccccc3Cl)c3cc(C4CC4)ccc23)n1] | 1-(2-chlorophenyl)-7-cyclopropyl-4- ((3-methyl-1,2,4-oxadiazol-5- yl)amino)-quinazolin-2(1H)-one | m/z [M + H]⁺ 394.1 |
| 470 | 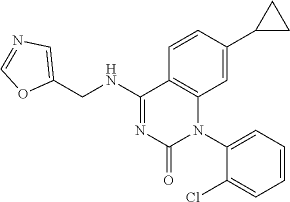 [SMILES: O=c1nc(NCc2cnco2)c2ccc(C3CC3)cc2n1-c1ccccc1Cl] | 1-(2-chlorophenyl)-7-cyclopropyl-4- ((oxazol-5- ylmethyl)amino)quinazolin-2(1H)-one | m/z [M + H]⁺ 393.1 |
| 471 | 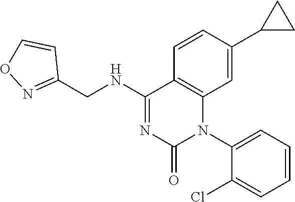 [SMILES: O=c1nc(NCc2ccon2)c2ccc(C3CC3)cc2n1-c1ccccc1Cl] | 1-(2-chlorophenyl)-7-cyclopropyl-4- ((isoxazol-3- ylmethyl)amino)quinazolin-2(1H)-one | m/z [M + H]⁺ 393.1 |
| 472 | 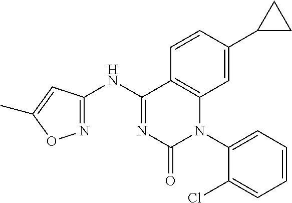 [SMILES: Cc1cc(Nc2nc(=O)n(-c3ccccc3Cl)c3cc(C4CC4)ccc23)no1] | 1-(2-chlorophenyl)-7-cyclopropyl-4- ((-methylisoxazol-3- yl)amino)quinazolin-2(1H)-one | m/z [M + H]⁺ 393.1 |
| 473 | 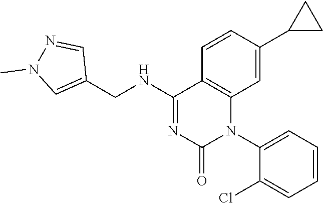 [SMILES: Cn1cc(CNc2nc(=O)n(-c3ccccc3Cl)c3cc(C4CC4)ccc23)cn1] | 1-(2-chlorophenyl)-7-cyclopropyl-4- (((1-methyl-1H-pyrazol-4- yl)methyl)amino)-quinazolin-2(1H)- one | m/z [M + H]⁺ 406.1 |
| 474 | 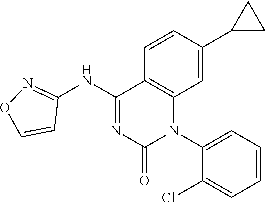 [SMILES: O=c1nc(Nc2ccon2)c2ccc(C3CC3)cc2n1-c1ccccc1Cl] | 1-(2-chlorophenyl)-7-cyclopropyl-4- (isoxazol-3-ylamino)quinazolin- 2(1H)-one | m/z [M + H]⁺ 379.05 |
| 475 | 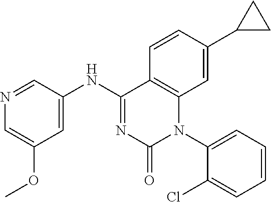 [SMILES: COc1cncc(Nc2nc(=O)n(-c3ccccc3Cl)c3cc(C4CC4)ccc23)c1] | 1-(2-chlorophenyl)-7-cyclopropyl-4- ((5-methoxypyridin-3- yl)amino)quinazolin-2(1H)-one | m/z [M + H]⁺ 419.01 |
| 476 | 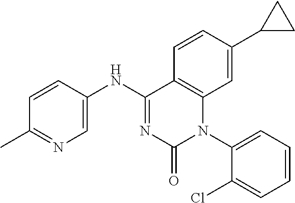 [SMILES: Cc1ccc(Nc2nc(=O)n(-c3ccccc3Cl)c3cc(C4CC4)ccc23)cn1] | 1-(2-chlorophenyl)-7-cyclopropyl-4- ((6-methylpyridin-3- yl)amino)quinazolin-2(1H)-one | m/z [M + H]⁺ 403.1 |
| 477 | 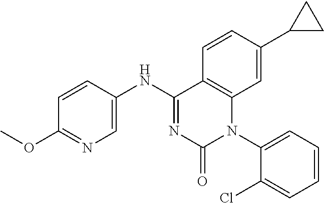 [SMILES: COc1ccc(Nc2nc(=O)n(-c3ccccc3Cl)c3cc(C4CC4)ccc23)cn1] | 1-(2-chlorophenyl)-7-cyclopropyl-4- ((6-methoxypyridin-3- yl)amino)quinazolin-2(1H)-one | m/z [M + H]⁺ 419.1 |
| 478 | 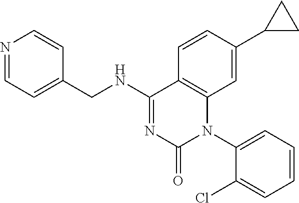 [SMILES: O=c1nc(NCc2ccncc2)c2ccc(C3CC3)cc2n1-c1ccccc1Cl] | 1-(2-chlorophenyl)-7-cyclopropyl-4- ((pyridin-4- ylmethyl)amino)quinazolin-2(1H)-one | m/z [M + H]⁺ 403.1 |
| 479 | 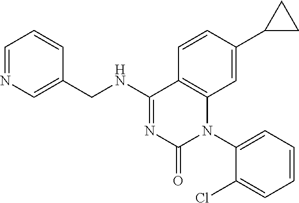 [SMILES: O=c1nc(NCc2cccnc2)c2ccc(C3CC3)cc2n1-c1ccccc1Cl] | 1-(2-chlorophenyl)-7-cyclopropyl-4- ((pyridin-3- ylmethyl)amino)quinazolin-2(1H)-one | m/z [M + H]⁺ 403.1 |
| 480 | 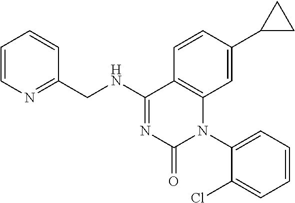 [SMILES: O=c1nc(NCc2ccccn2)c2ccc(C3CC3)cc2n1-c1ccccc1Cl] | 1-(2-chlorophenyl)-7-cyclopropyl-4- ((pyridin-2- ylmethyl)amino)quinazolin-2(1H)-one | m/z [M + H]⁺ 403.1 |
| 481 | 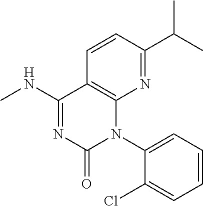 [SMILES: CNc1nc(=O)n(-c2ccccc2Cl)c2nc(C(C)C)ccc12] | 1-(2-chlorophenyl)-7-isopropyl-4- (methylamino)pyrido[2,3-d]pyrimidin- 2(1H)-one | m/z [M + H]⁺ 329.1 |
| 482 | 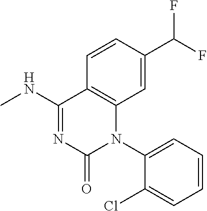 [SMILES: CNc1nc(=O)n(-c2ccccc2Cl)c2cc(C(F)F)ccc12] | 1-(2-chlorophenyl)-7- (difluoromethyl)-4- (methylamino)quinazolin-2(1H)-one | m/z [M + H]⁺ 336.0 |
| 483 | 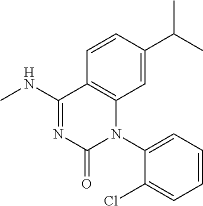 [SMILES: CNc1nc(=O)n(-c2ccccc2Cl)c2cc(C(C)C)ccc12] | 1-(2-chlorophenyl)-7-isopropyl-4- (methylamino)quinazolin-2(1H)-one | m/z [M + H]⁺ 328.1 |
| 484 | 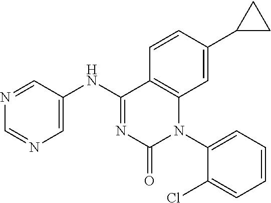 [SMILES: O=c1nc(Nc2cncnc2)c2ccc(C3CC3)cc2n1-c1ccccc1Cl] | 1-(2-chlorophenyl)-7-cyclopropyl-4- (pyrimidin-5-ylamino)quinazolin- 2(1H)-one | m/z [M + H]⁺ 390.05 |
| 485 | 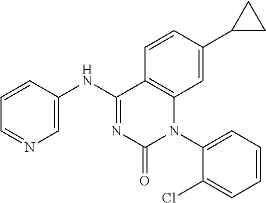 [SMILES: O=c1nc(Nc2cccnc2)c2ccc(C3CC3)cc2n1-c1ccccc1Cl] | 1-(2-chlorophenyl)-7-cyclopropyl-4- (pyridin-3-ylamino)quinazolin-2(1H)- one | m/z [M + H]⁺ 389.05 |
| 486 | 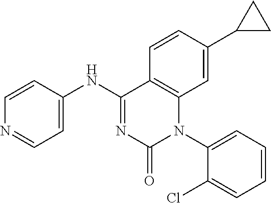 [SMILES: O=c1nc(Nc2ccncc2)c2ccc(C3CC3)cc2n1-c1ccccc1Cl] | 1-(2-chlorophenyl)-7-cyclopropyl-4- (pyridin-4-ylamino)quinazolin-2(1H)- one | m/z [M + H]⁺ 389.05 |
| 487 | 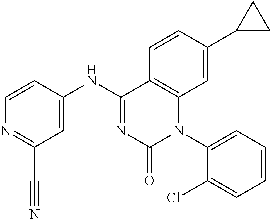 [SMILES: N#Cc1cc(Nc2nc(=O)n(-c3ccccc3Cl)c3cc(C4CC4)ccc23)ccn1] | 4-((1-(2-chlorophenyl)-7-cyclopropyl- 2-oxo-1,2-dihydroquinazolin-4- yl)amino)picolinonotrile | m/z [M + H]⁺ 414.1 |
| 488 | 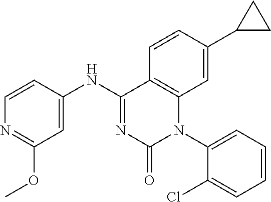 [SMILES: COc1cc(Nc2nc(=O)n(-c3ccccc3Cl)c3cc(C4CC4)ccc23)ccn1] | 1-(2-chlorophenyl)-7-cyclopropyl-4- ((2-methoxypyridin-4- yl)amino)quinazolin-2(1H)-one | m/z [M + H]⁺ 419.1 |
| 489 | 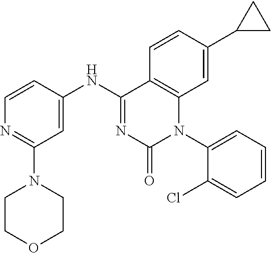 [SMILES: O=c1nc(Nc2ccnc(N3CCOCC3)c2)c2ccc(C3CC3)cc2n1-c1ccccc1Cl] | 1-(2-chlorophenyl)-7-cyclopropyl-4- ((2-morpholinopyridin-4-yl)amino)- quinazolin-2(1H)-one | m/z [M + H]⁺ 474.1 |
| 490 | 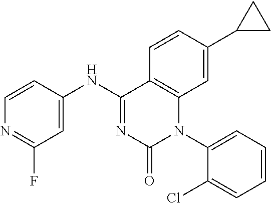 [SMILES: O=c1nc(Nc2ccnc(F)c2)c2ccc(C3CC3)cc2n1-c1ccccc1Cl] | 1-(2-chlorophenyl)-7-cyclopropyl-4- ((2-fluoropyridin-4- yl)amino)quinazolin-2(1H)-one | m/z [M + H]⁺ 407.0 |
| 491 | 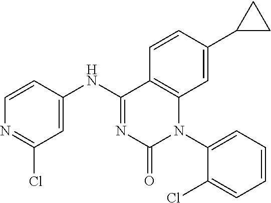 [SMILES: O=c1nc(Nc2ccnc(Cl)c2)c2ccc(C3CC3)cc2n1-c1ccccc1Cl] | 1-(2-chlorophenyl)-4-((2- chloropyridin-4-yl)amino)-7- cyclopropylquinazolin-2(1H)-one | m/z [M + H]⁺ 423.0 |
| 492 | 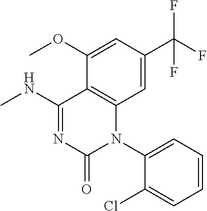 [SMILES: CNc1nc(=O)n(-c2ccccc2Cl)c2cc(C(F)(F)F)cc(OC)c12] | 1-(2-chlorophenyl)-5-methoxy-4- (methylamino)-7-(trifluoromethyl)- quinazolin-2(1H)-one | m/z [M + H]⁺ 384.1 |
| 493 | 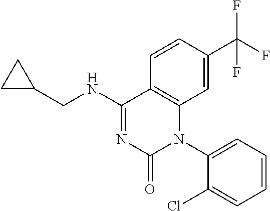 [SMILES: O=c1nc(NCC2CC2)c2ccc(C(F)(F)F)cc2n1-c1ccccc1Cl] | 1-(2-chlorophenyl)-4-((cyclopropyl- methyl)amino)-7-(trifluoromethyl)- quinazolin-2(1H)-one | m/z [M + H]⁺ 394.1 |
| 494 | 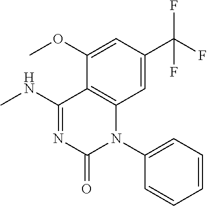 [SMILES: CNc1nc(=O)n(-c2ccccc2)c2cc(C(F)(F)F)cc(OC)c12] | 5-methoxy-4-(methylamino)-1- phenyl-7-(trifluoromethyl)quinazolin- 2(1H)-one | m/z [M + H]⁺ 350.1 |
| 495 | 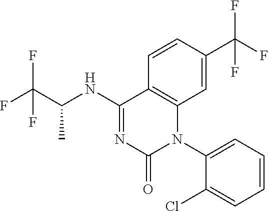 [SMILES: C[C@@H](Nc1nc(=O)n(-c2ccccc2Cl)c2cc(C(F)(F)F)ccc12)C(F)(F)F] | (R)-1-(2-chlorophenyl)-7-(trifluoro- methyl-4-((1,1,1-trifluoropropan-2- yl)-amino)quinazolin-2(1H)-one | m/z [M + H]⁺ 436.0 |
| 496 | 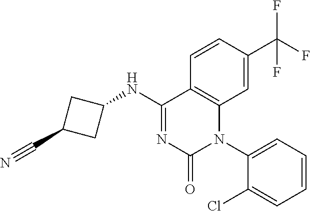 [SMILES: N#C[C@H]1C[C@H](Nc2nc(=O)n(-c3ccccc3Cl)c3cc(C(F)(F)F)ccc23)C1] | (1,3-trans)-3-((1-(2-chlorophenyl)-2- oxo-7-(trifluoromethyl)-1,2-dihydro- quinazolin-4-yl)amino)-cyclobutane-3- 1-carbonitrile | m/z [M + H]⁺ 419.05 |
| 497 | 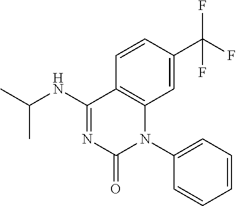 [SMILES: CC(C)Nc1nc(=O)n(-c2ccccc2)c2cc(C(F)(F)F)ccc12] | 4-(isopropylamino)-1-phenyl-7- (trifluoromethyl)quinazolin-2(1H)-one | m/z [M + H]⁺ 348.1 |
| 498 | 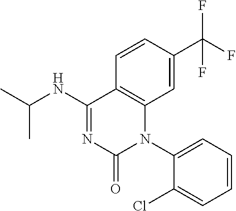 [SMILES: CC(C)Nc1nc(=O)n(-c2ccccc2Cl)c2cc(C(F)(F)F)ccc12] | 1-(2-chlorophenyl)-4- (isopropylamino)-7- (trifluoromethyl)quinazolin-2(1H)-one | m/z [M + H]⁺ 382.0 |
| 499 | 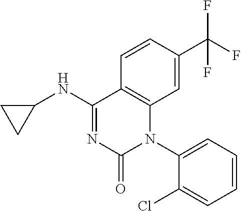 [SMILES: O=c1nc(NC2CC2)c2ccc(C(F)(F)F)cc2n1-c1ccccc1Cl] | 1-(2-chlorophenyl)-4-(cyclopropyl- amino)-7-(tirfluoromethyl)quinazolin- 2(1H)-one | m/z [M + H]⁺ 380.0 |
| 500 | 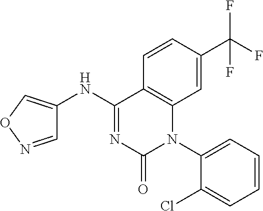 [SMILES: O=c1nc(Nc2cnoc2)c2ccc(C(F)(F)F)cc2n1-c1ccccc1Cl] | 1-(2-chlorophenyl)-4-(isoxazol-4-yl- amino)-7-(trifluoromethyl)quinazolin- 2(1H)-one | m/z [M + H]⁺ 406.75 |
| 501 | 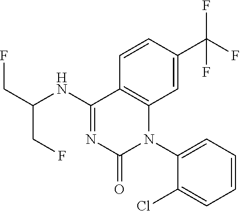 [SMILES: O=c1nc(NC(CF)CF)c2ccc(C(F)(F)F)cc2n1-c1ccccc1Cl] | 1-(2-chlorophenyl)-4-((1,3-difluoro- propan-2-yl)amino)-7- (trifluoromethyl)quinazolin-2(1H)- one | m/z [M + H]⁺ 418.1 |
| 502 | 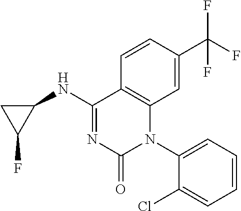 [SMILES: O=c1nc(N[C@@H]2C[C@@H]2F)c2ccc(C(F)(F)F)cc2n1-c1ccccc1Cl] | 1-(2-chlorophenyl)-4-(((1R,2S)-2- fluorocyclopropyl)amimno)-7-(trifluoro- methyl)quinazolin-2(1H)-one | m/z [M + H]⁺ 398.05 |
| 503 | 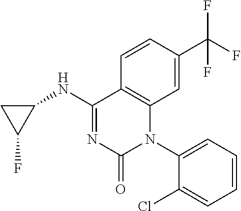 [SMILES: O=c1nc(N[C@H]2C[C@H]2F)c2ccc(C(F)(F)F)cc2n1-c1ccccc1Cl] | 1-(2-chlorophenyl)-4-(((1S,2R)-2- fluorocyclopropyl)amino)-7-(trifluoro- methyl)quinazolin-2(1H)-one | m/z [M + H]⁺ 398.1 |
| 504 | 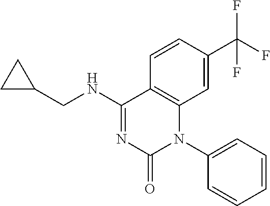 [SMILES: O=c1nc(NCC2CC2)c2ccc(C(F)(F)F)cc2n1-c1ccccc1] | 4-((cyclopropylmethyl)amino)-1- phenyl-7-(trifluoromethyl)quinazolin- 2(1H)-one | m/z [M + H]⁺ 360.1 |
| 505 | 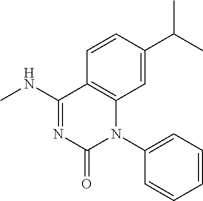 [SMILES: CNc1nc(=O)n(-c2ccccc2)c2cc(C(C)C)ccc12] | 7-isopropyl-4-(methylamino)-1- phenyl-quinazolin-2(1H)-one | m/z [M + H]⁺ 294.2 |
| 506 | 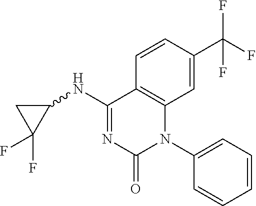 [SMILES: O=c1nc(NC2CC2(F)F)c2ccc(C(F)(F)F)cc2n1-c1ccccc1] | 4-((2,2-difluorocyclopropyl)amino)-1- phenyl-7-(trifluoromethyl)quinazolin- 2(1H)-one | m/z [M + H]⁺ 382.05 |
| 507 | 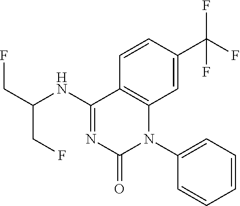 [SMILES: O=c1nc(NC(CF)CF)c2ccc(C(F)(F)F)cc2n1-c1ccccc1] | 4-((1,3-difluoropropan-2-yl)amino)-1- phenyl-7-(trifluoromethyl)quinazolin- 2(1H)-one | m/z [M + H]⁺ 384.1 |
| 508 | 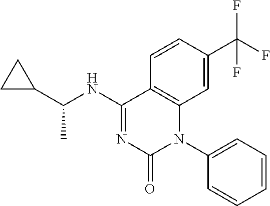 [SMILES: C[C@@H](Nc1nc(=O)n(-c2ccccc2)c2cc(C(F)(F)F)ccc12)C1CC1] | (R)-4-((1-cyclopropylethyl)amino)-1- phenyl-7-(trifluoromethyl)quinazolin- 2(1H)-one | m/z [M + H]⁺ 374.1 |
| 509 | 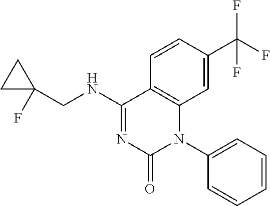 [SMILES: O=c1nc(NCC2(F)CC2)c2ccc(C(F)(F)F)cc2n1-c1ccccc1] | 4-(((1- fluorocyclopropyl)methyl)amino)-1- phenyl-7-(trifluoromethyl)quinazolin- 2(1H)-one | m/z [M + H]⁺ 378.1 |
| 510 | 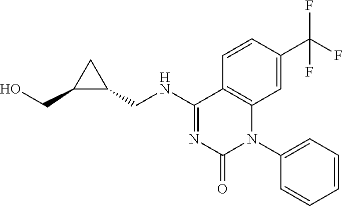 [SMILES: O=c1nc(NC[C@H]2C[C@@H]2CO)c2ccc(C(F)(F)F)cc2n1-c1ccccc1] | 4-((((trans)-2-(hydroxymethyl)cyclo- propyl)methyl)amino)-1-phenyl-7- (trifluoromethyl)quinazolin-2(1H)-one | m/z [M + H]⁺ 390.1 |
| 511 | 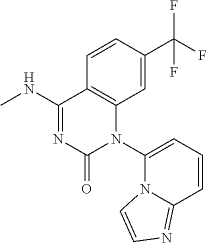 [SMILES: CNc1nc(=O)n(-c2cccc3nccn23)c2cc(C(F)(F)F)ccc12] | 1-(imidazo[1,2-a]pyridin-5-yl)-4- (methylamino)-7-(trifluoromethyl)- quinazolin-2(1H)-one | m/z [M + H]⁺ 360.1 |
| 512 | 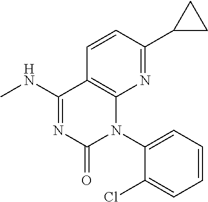 [SMILES: CNc1nc(=O)n(-c2ccccc2Cl)c2nc(C3CC3)ccc12] | 1-(2-chlorophenyl)-7-cyclopropyl-4- (methylamino)pyrido[2,3-d]pyrimidin- 2(1H)-one | m/z [M + H]⁺ 327.1 |
| 513 | 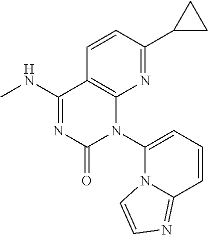 [SMILES: CNc1nc(=O)n(-c2cccc3nccn23)c2nc(C3CC3)ccc12] | 7-cyclopropyl-1-(imidazo[1,2- a]pyridin-5-yl)-4- (methylamino)pyrido[2,3-d]- pyrimidin-2(1H)-one | m/z [M + H]⁺ 333.1 |
| 514 | 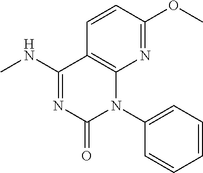 | 7-methoxy-4-(methylamino)-1- phenyl-pyrido[2,3-d]pyrimidin-2(1H)- one | m/z [M + H]⁺ 283.1 |
| 515 | 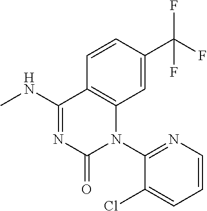 [SMILES: CNc1nc(=O)n(-c2ncccc2Cl)c2cc(C(F)(F)F)ccc12] | 1-(3-chloropyridin-2-yl)-4-(methyl- amino)-7-(trifluoromethyl)quinazolin- 2(1H)-one | m/z [M + H]⁺ 355.0 |
| 516 | 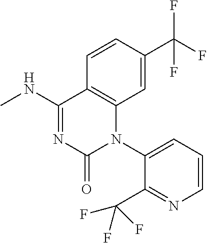 [SMILES: CNc1nc(=O)n(-c2cccnc2C(F)(F)F)c2cc(C(F)(F)F)ccc12] | 4-(methylamino)-7-(trifluoromethyl)- 1-(2-(trifluoromethyl)pyridin-3-yl)- quinazolin-2(1H)-one | m/z [M + H]⁺ 389.1 |
| 517 | 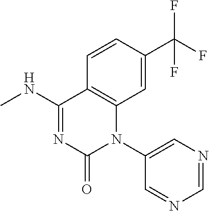 [SMILES: CNc1nc(=O)n(-c2cncnc2)c2cc(C(F)(F)F)ccc12] | 4-(methylamino)-1-(pyrimidin-5-yl)- 7-(trifluoromethyl)quinazolin-2(1H)- one | m/z [M + H]⁺ 322.1 |
| 518 | 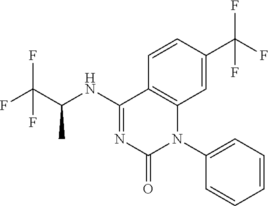 [SMILES: C[C@H](Nc1nc(=O)n(-c2ccccc2)c2cc(C(F)(F)F)ccc12)C(F)(F)F] | (S)-1-phenyl-7-(trifluoromethyl)-4- ((1,1,1-trifluoropropan-2-yl)amino)- quinazolin-2(1H)-one | m/z [M + H]⁺ 402.1 |
| 519 | 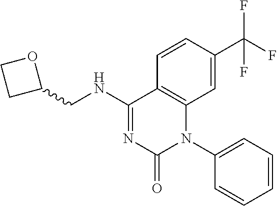 [SMILES: O=c1nc(NCC2CCO2)c2ccc(C(F)(F)F)cc2n1-c1ccccc1] | 4-((oxetan-2-ylmethyl)amino)-1- phenyl-7-(trifluoromethyl)quinazolin- 2(1H)-one | m/z [M + H]⁺ 376.1 |
| 520 | 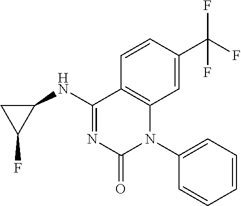 [SMILES: O=c1nc(N[C@@H]2C[C@@H]2F)c2ccc(C(F)(F)F)cc2n1-c1ccccc1] | 4-(((1R,2S)-2- fluorocyclopropyl)amino)-1-phenyl-7- (trifluoromethyl)quinazolin-2(1H)-one | m/z [M + H]⁺ 364.1 |
| 521 | 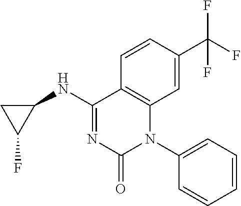 [SMILES: O=c1nc(N[C@@H]2C[C@H]2F)c2ccc(C(F)(F)F)cc2n1-c1ccccc1] | 4-(((1,2-trans)-2-fluorocyclopropyl)- amino)-1-phenyl-7- (trifluoromethyl)quinazolin-2(1H)-one | m/z [M + H]⁺ 364.1 |
| 522 | 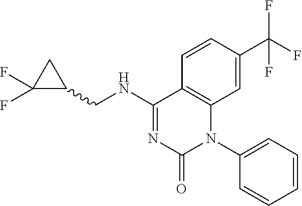 [SMILES: O=c1nc(NCC2CC2(F)F)c2ccc(C(F)(F)F)cc2n1-c1ccccc1] | 4-(((2,2-difluorocyclopropyl)methyl)- amino)-1-phenyl-7-(trifluoromethyl)- quinazolin-2(1H)-one | m/z [M + H]⁺ 396.1 |
| 523 | 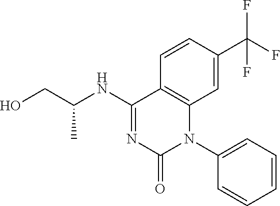 [SMILES: C[C@H](CO)Nc1nc(=O)n(-c2ccccc2)c2cc(C(F)(F)F)ccc12] | (R)-4-((1-hydroxypropan-2- yl)amino)-1-phenyl-7- (trifluoromethyl)quinazolin-2(1H)-one | m/z [M + H]⁺ 364.1 |
| 524 | 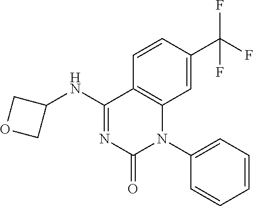 [SMILES: O=c1nc(NC2COC2)c2ccc(C(F)(F)F)cc2n1-c1ccccc1] | 4-(oxetan-3-ylamino)-1-phenyl-7- (trifluoromethyl)quinazolin-2(1H)-one | m/z [M + H]⁺ 362.1 |
| 525 | 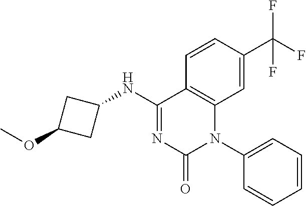 [SMILES: CO[C@H]1C[C@H](Nc2nc(=O)n(-c3ccccc3)c3cc(C(F)(F)F)ccc23)C1] | 4-(((1,3-trans)-3- methoxycyclobutyl)-amino)-1-phenyl- 7-(trifluoromethyl)-quinazolin-2(1H)- one | m/z [M + H]⁺ 390.1 |
| 526 | 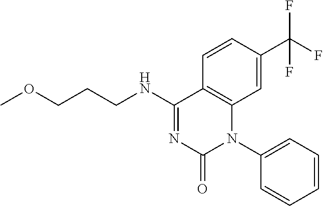 [SMILES: COCCCNc1nc(=O)n(-c2ccccc2)c2cc(C(F)(F)F)ccc12] | 4-((3-methoxypropyl)amino)-1- phenyl-7-(trifluoromethyl)quinazolin- 2(1H)-one | m/z [M + H]⁺ 378.1 |
| 527 | 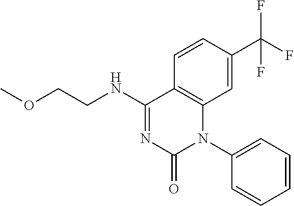 [SMILES: COCCNc1nc(=O)n(-c2ccccc2)c2cc(C(F)(F)F)ccc12] | 4-((2-methoxyethyl)amino)-1- phenyl-7-(trifluoromethyl)quinazolin- 2(1H)-one | m/z [M + H]⁺ 364.1 |
| 528 | 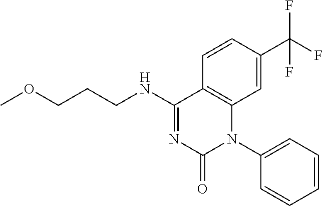 [SMILES: COCCCNc1nc(=O)n(-c2ccccc2)c2cc(C(F)(F)F)ccc12] | 4-((3-methoxypropyl)amino)-1- phenyl-7-(trifluoromethyl)quinazolin- 2(1H)-one | m/z [M + H]⁺ 378.1 |
| 530 | 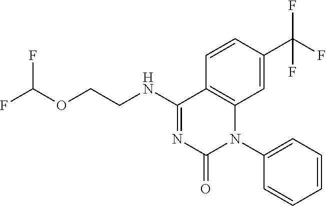 [SMILES: O=c1nc(NCCOC(F)F)c2ccc(C(F)(F)F)cc2n1-c1ccccc1] | 4-((2-(difluoromethoxy)ethyl)amino)- 1-phenyl-7- (trifluoromethyl)quinazolin-2(1H)-one | m/z [M + H]⁺ 400.1 |
| 531 | 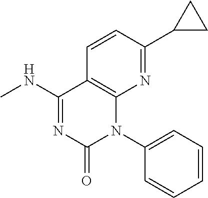 [SMILES: CNc1nc(=O)n(-c2ccccc2)c2nc(C3CC3)ccc12] | 7-cyclopropyl-4-(methylamino)-1- phenylpyrido[2,3-d]pyrimidin-2(1H)- one | m/z [M + H]⁺ 293.1 |
| 532 | 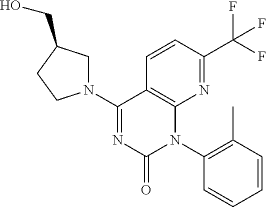 [SMILES: Cc1ccccc1-n1c(=O)nc(N2CC[C@@H](CO)C2)c2ccc(C(F)(F)F)nc21] | (R)-4-(3-(hydroxymethyl)pyrrolidin- 1-yl)-1-(o-tolyl)-7-(trifluoromethyl)- pyrido[2,3-d]pyrimidin-2(1H)-one | m/z [M + H]⁺ 405.00 |
| 533 | 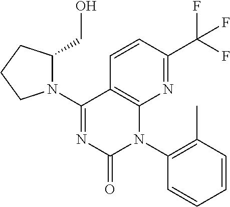 [SMILES: Cc1ccccc1-n1c(=O)nc(N2CCC[C@@H]2CO)c2ccc(C(F)(F)F)nc21] | (R)-4-(2-(hydroxymethyl)pyrrolidin- 1-yl)-1-(o-tolyl)-7- (trifluoromethyl)pyrido-[2,3- d]pyrimidin-2(1H)-one | m/z [M + H]⁺ 405.00 |
| 534 | 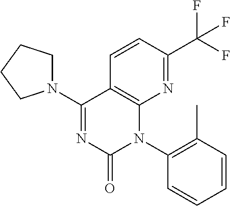 [SMILES: Cc1ccccc1-n1c(=O)nc(N2CCCC2)c2ccc(C(F)(F)F)nc21] | 4-(pyrrolidin-1-yl)-1-(o-tolyl)-7- (trifluoromethyl)pyrido[2,3- d]pyrimidin-2(1H)-one | m/z [M + H]⁺ 375.2 |
| 535 | 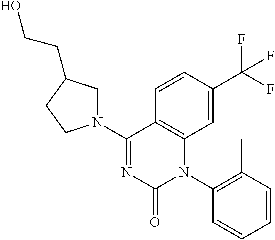 [SMILES: Cc1ccccc1-n1c(=O)nc(N2CCC(CCO)C2)c2ccc(C(F)(F)F)cc21] | 4-(3-(2-hydroxyethyl)pyrrolidin-1-yl)- 1-(o-tolyl)-7- (trifluoromethyl)quinazolin-2(1H)-one | m/z [M + H]⁺ 418.1 |
| 536 | 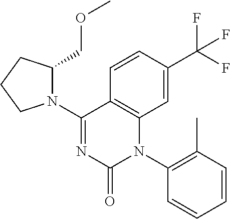 [SMILES: COC[C@H]1CCCN1c1nc(=O)n(-c2ccccc2C)c2cc(C(F)(F)F)ccc12] | (R)-4-(2-(methoxymethyl)pyrrolidin- 1-yl)-1-(o-tolyl)-7-(trifluoromethyl)- quinazolin-2(1H)-one | m/z [M + H]⁺ 418.1 |
| 537 | 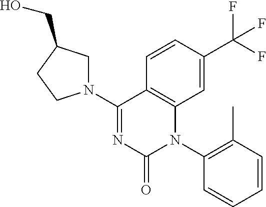 [SMILES: Cc1ccccc1-n1c(=O)nc(N2CC[C@@H](CO)C2)c2ccc(C(F)(F)F)cc21] | (R)-4-(3-(hydroxymethyl)pyrrolidin- 1-yl)-1-(o-tolyl)-7-(trifluoromethyl)- quinazolin-2(1H)-one | m/z [M + H]⁺ 404.1 |
| 538 | 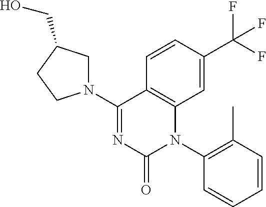 [SMILES: Cc1ccccc1-n1c(=O)nc(N2CC[C@H](CO)C2)c2ccc(C(F)(F)F)cc21] | (S)-4-(3-(hydroxymethyl)pyrrolidin- 1-yl)-1-(o-tolyl)-7-(trifluoromethyl)- quinazolin-2(1H)-one | m/z [M + H]⁺ 404.1 |
| 539 | 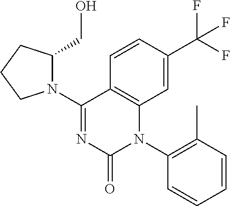 [SMILES: Cc1ccccc1-n1c(=O)nc(N2CCC[C@@H]2CO)c2ccc(C(F)(F)F)cc21] | (R)-4-(2-(hydroxymethyl)pyrrolidin- 1-yl)-1-(o-tolyl)-7-(trifluoromethyl)- quinazolin-2(1H)-one | m/z [M + H]⁺ 404.1 |
| 540 | 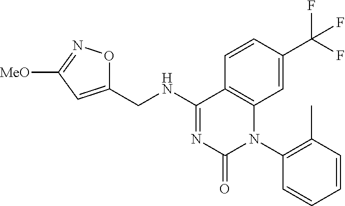 [SMILES: COc1cc(CNc2nc(=O)n(-c3ccccc3C)c3cc(C(F)(F)F)ccc23)on1] | 4-(((3-methoxyisoxazol-5-yl)methyl)- amino)-1-(o-tolyl)-7-(trifluoromethyl)- quinazolin-2(1H)-one | m/z [M + H]⁺ 431.1 |
| 541 | 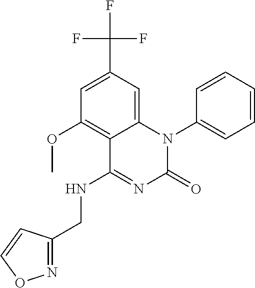 [SMILES: COc1cc(C(F)(F)F)cc2c1c(NCc1ccon1)nc(=O)n2-c1ccccc1] | 4-((isoxazol-3-ylmethyl)amino)-5- methoxy-1-phenyl-7-(trifluoromethyl)- quinazolin-2(1H)-one | m/z [M + H]]⁺ 417.1 |
| 542 | 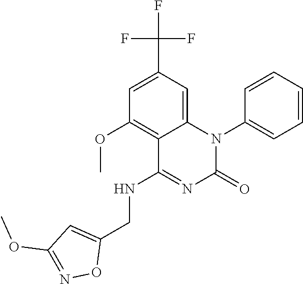 [SMILES: COc1cc(CNc2nc(=O)n(-c3ccccc3)c3cc(C(F)(F)F)cc(OC)c23)on1] | 5-methoxy-4-(((3-methoxyisoxazol- 5-yl)methyl)amino)-1-phenyl-7- (trifluoromethyl)quinazolin-2(1H)-one | m/z [M + H]⁺ 447.1 |
| 543 | 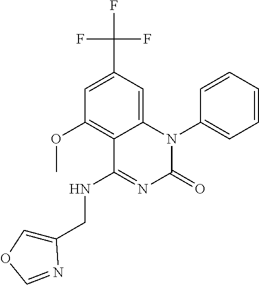 [SMILES: COc1cc(C(F)(F)F)cc2c1c(NCc1cocn1)nc(=O)n2-c1ccccc1] | 5-methoxy-4-((oxazol-4-ylmethyl)- amino)-1-phenyl-7-(trifluoromethyl)- quinazolin-2(1H)-one | m/z [M + H]⁺ 417.1 |
| 544 | 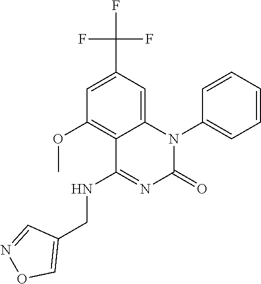 [SMILES: COc1cc(C(F)(F)F)cc2c1c(NCc1cnoc1)nc(=O)n2-c1ccccc1] | 4-((isoxazol-4-ylmethyl)amino)-5- methoxy-1-phenyl-7-(trifluoromethyl)- quinazolin-2(1H)-one | m/z [M + H]⁺ 417.1 |
| 545 | 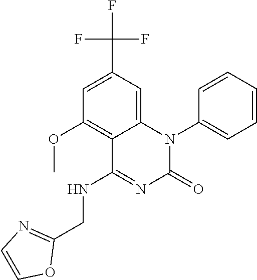 [SMILES: COc1cc(C(F)(F)F)cc2c1c(NCc1ncco1)nc(=O)n2-c1ccccc1] | 5-methoxy-4-((oxazol-2-ylmethyl)- amino)-1-phenyl-7- (trifluoromethyl)quinazolin-2(1H)-one | m/z [M + H]⁺ 417.1 |
| 546 | 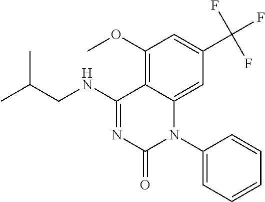 [SMILES: COc1cc(C(F)(F)F)cc2c1c(NCC(C)C)nc(=O)n2-c1ccccc1] | 4-(isobutylamino)-5-methoxy-1- phenyl-7-(trifluoromethyl)quinazolin- 2(1H)-one | m/z [M + H]⁺ 392.14 |
| 547 | 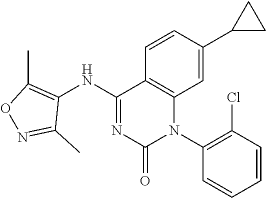 [SMILES: Cc1noc(C)c1Nc1nc(=O)n(-c2ccccc2Cl)c2cc(C3CC3)ccc12] | 1-(2-chlorophenyl)-7-cyclopropyl-4- ((3,5-dimethylisoxazol-4-yl)amino)- quinazolin-2(1H)-one | m/z [M + H]⁺ 407.1 |
| 548 | 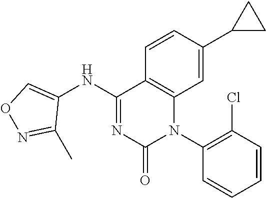 [SMILES: Cc1nocc1Nc1nc(=O)n(-c2ccccc2Cl)c2cc(C3CC3)ccc12] | 1-(2-chlorophenyl)-7-cyclopropyl-4- ((3-methylisoxazol-4- yl)amino)quinazolin-2(1H)-one | m/z [M + H]⁺ 393.1 |
| 549 | 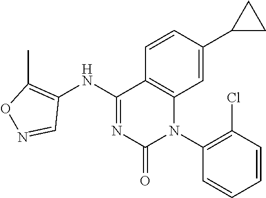 [SMILES: Cc1oncc1Nc1nc(=O)n(-c2ccccc2Cl)c2cc(C3CC3)ccc12] | 1-(2-chlorophenyl)-7-cyclopropyl-4- ((5-methylisoxazol-4- yl)amino)quinazolin-2(1H)-one | m/z [M + H]⁺ 393.1 |
| 550 | 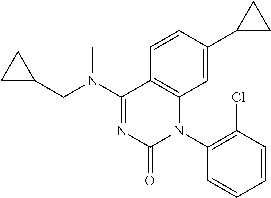 [SMILES: CN(CC1CC1)c1nc(=O)n(-c2ccccc2Cl)c2cc(C3CC3)ccc12] | 1-(2-chlorophenyl)-7-cyclopropyl-4- ((cyclopropylmethyl)(methyl)amino)quin- azolin-2(1H)-one | m/z [M + H]⁺ 380.15 |
| 551 | 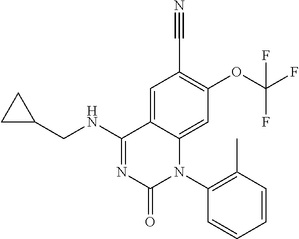 [SMILES: Cc1ccccc1-n1c(=O)nc(NCC2CC2)c2cc(C#N)c(OC(F)(F)F)cc21] | 4-((cyclopropylmethyl)amino)-2-oxo- 1-(o-tolyl)-7-(trifluoromethoxy)-1,2- dihydroquinazoline-6-carbonitrile | m/z [M + H]⁺ 415.1 |
| 552 | 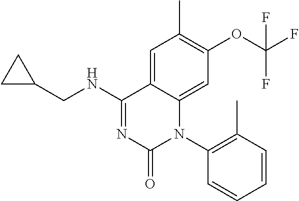 [SMILES: Cc1cc2c(NCC3CC3)nc(=O)n(-c3ccccc3C)c2cc1OC(F)(F)F] | 4-((cyclopropylmethyl)amino)-6- methyl-1-(o-tolyl)-7- (trifluoromethoxy)-quinazolin-2(1H)- one | m/z [M + H]⁺ 404.1 |
| 553 | 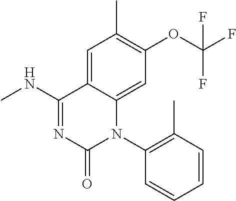 [SMILES: CNc1nc(=O)n(-c2ccccc2C)c2cc(OC(F)(F)F)c(C)cc12] | 6-methyl-4-(methylamino)-1-(o- tolyl)-7-(trifluoromethoxy)quinazolin- 2(1H)-one | m/z [M + H]⁺ 364.1 |
| 554 | 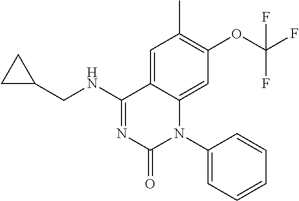 [SMILES: Cc1cc2c(NCC3CC3)nc(=O)n(-c3ccccc3)c2cc1OC(F)(F)F] | 4-((cyclopropylmethyl)amino)-6- methyl-1-phenyl-7-(trifluoromethoxy)- quinazolin-2(1H)-one | m/z [M + H]⁺ 390.1 |
| 555 | 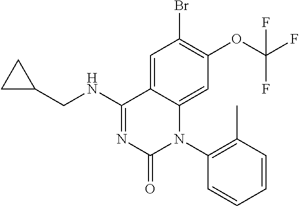 [SMILES: Cc1ccccc1-n1c(=O)nc(NCC2CC2)c2cc(Br)c(OC(F)(F)F)cc21] | 6-bromo-4- ((cyclopropylmethyl)amino)-1-(o- tolyl)-7-(trifluoromethoxy)-quinazolin- 2(1H)-one | m/z [M + H]⁺ 468.0, 470.0 |
| 556 | 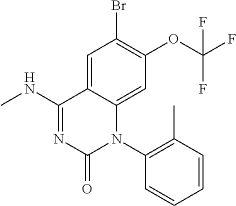 [SMILES: CNc1nc(=O)n(-c2ccccc2C)c2cc(OC(F)(F)F)c(Br)cc12] | 6-bromo-4-(methylamino)-1-(o- tolyl)-7-(trifluoromethoxy)quinazolin- 2(1H)-one | m/z [M + H]⁺ 430.0, 428.0 |
| 557 | 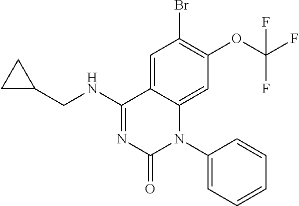 [SMILES: O=c1nc(NCC2CC2)c2cc(Br)c(OC(F)(F)F)cc2n1-c1ccccc1] | 6-bromo-4- ((cyclopropylmethyl)amino)-1- phenyl-7-(trifluoromethoxy)- quinazolin-2(1H)-one | m/z [M + H]⁺ 456.1, 454.1 |
| 558 | 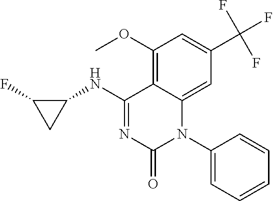 [SMILES: COc1cc(C(F)(F)F)cc2c1c(N[C@@H]1C[C@@H]1F)nc(=O)n2-c1ccccc1] | 4-(((1R,2S)-2- fluorocyclopropyl)amino)-5-methoxy- 1-phenyl-7-(trifluoromethyl)- quinazolin-2(1H)-one | m/z [M + H]⁺ 394.05 |
| 559 | 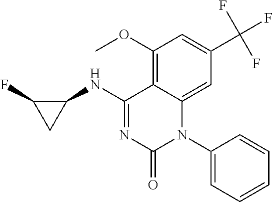 [SMILES: COc1cc(C(F)(F)F)cc2c1c(N[C@H]1C[C@H]1F)nc(=O)n2-c1ccccc1] | 4-(((1S,2R)-2- fluorocyclopropyl)amino)-5-methoxy- 1-phenyl-7-(trifluoromethyl)- quinazolin-2(1H)-one | m/z [M + H]⁺ 394.05 |
| 560 | 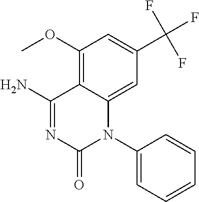 [SMILES: COc1cc(C(F)(F)F)cc2c1c(N)nc(=O)n2-c1ccccc1] | 4-amino-5-methoxy-1-phenyl-7- (trifluoromethyl)quinazolin-2(1H)-one | m/z [M + H]⁺ 336.0 |
| 561 | 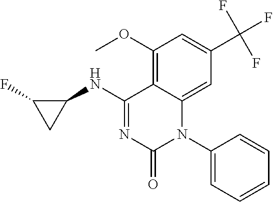 [SMILES: COc1cc(C(F)(F)F)cc2c1c(N[C@H]1C[C@@H]1F)nc(=O)n2-c1ccccc1] | 4-(((trans)-2- fluorocyclopropyl)amino)-5-methoxy- 1-phenyl-7-(trifluoromethyl)- quinazolin-2(1H)-one | m/z [M + H]⁺ 394.05 |
| 562 | 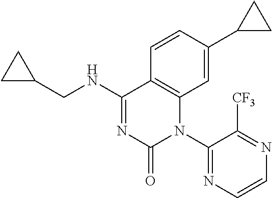 [SMILES: O=c1nc(NCC2CC2)c2ccc(C3CC3)cc2n1-c1nccnc1C(F)(F)F] | 7-cyclopropyl-4- (cyclopropylmethylamino)-1-(3- (trifluoromethyl)pyrazin-2-yl)- quinazolin-2(1H)-one | m/z [M + H]⁺ 402.1 |
| 563 | 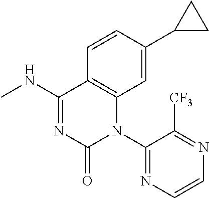 [SMILES: CNc1nc(=O)n(-c2nccnc2C(F)(F)F)c2cc(C3CC3)ccc12] | 7-cyclopropyl-4-(methylamino)-1-(3- (trifluoromethyl)pyrazin-2- yl)quinazolin-2(1H)-one | m/z [M + H]⁺ 362.1 |
| 564 | 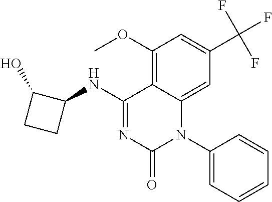 [SMILES: COc1cc(C(F)(F)F)cc2c1c(N[C@H]1CC[C@@H]1O)nc(=O)n2-c1ccccc1] | 4-(((trans)-2- hydroxycyclobutyl)amino)-5- methoxy-1-phenyl-7-(trifluoromethyl)- quinazolin-2(1H)-one | m/z [M + H]⁺ 406.1 |
| 565 | 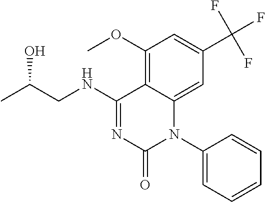 [SMILES: COc1cc(C(F)(F)F)cc2c1c(NC[C@H](C)O)nc(=O)n2-c1ccccc1] | (S)-4-((2-hydroxypropyl)amino)-5- methoxy-1-phenyl-7-(trifluoromethyl)- quinaozlin-2(1H)-one | m/z [M + H]⁺ 394.1 |
| 566 | 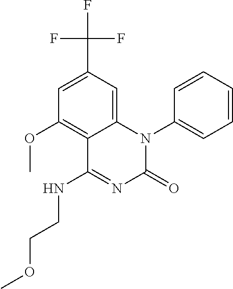 [SMILES: COCCNc1nc(=O)n(-c2ccccc2)c2cc(C(F)(F)F)cc(OC)c12] | 5-methoxy-4-((2- methoxyethyl)amino)-1-phenyl-7- (trifluoromethyl)quinazolin-2(1H)-one | m/z [M + H]⁺ 394.1 |
| 567 | 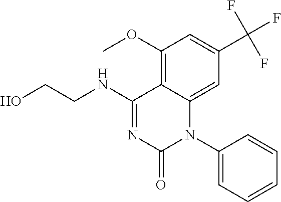 [SMILES: COc1cc(C(F)(F)F)cc2c1c(NCCO)nc(=O)n2-c1ccccc1] | 4-((2-hydroxyethyl)amino)-5- methoxy-1-phenyl-7- (trifluoromethyl)quinazolin-2(1H)-one | m/z [M + H]⁺ 380.1 |
| 568 | 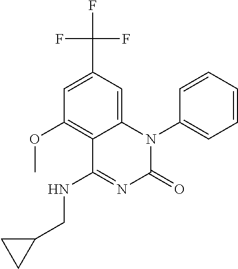 [SMILES: COc1cc(C(F)(F)F)cc2c1c(NCC1CC1)nc(=O)n2-c1ccccc1] | 4-((cyclopropylmethyl)amino)-5- methoxy-1-phenyl-7-(trifluoromethyl)- quinazolin-2(1H)-one | m/z [M + H]⁺ 390.1 |
| 569 | 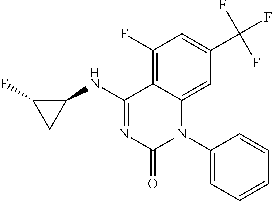 [SMILES: O=c1nc(N[C@H]2C[C@@H]2F)c2c(F)cc(C(F)(F)F)cc2n1-c1ccccc1] | 5-fluoro-4-((trans-2- fluorocyclopropyl)-amino)-1-phenyl- 7-(trifluoromethyl)-quinazolin-2(1H)- one | m/z [M + H]⁺ 382.1 |
| 570 | 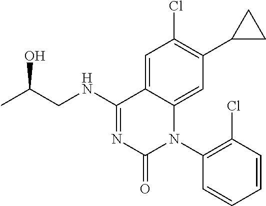 [SMILES: C[C@@H](O)CNc1nc(=O)n(-c2ccccc2Cl)c2cc(C3CC3)c(Cl)cc12] | (R)-6-chloro-1-(2-chlorophenyl)-7- cyclopropyl-4-((2-hydroxypropyl)- amino)-quinazolin-2(1H)-one | m/z [M + H]⁺ 405.05 |
| 571 | 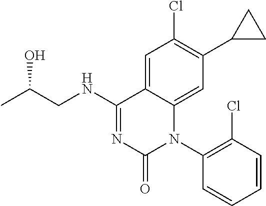 [SMILES: C[C@H](O)CNc1nc(=O)n(-c2ccccc2Cl)c2cc(C3CC3)c(Cl)cc12] | (S)-6-chloro-1-(2-chlorophenyl)-7- cyclopropyl-4-((2-hydroxypropyl)- amino)-quinazolin-2(1H)-one | m/z [M + H]⁺ 405.1 |
| 572 | 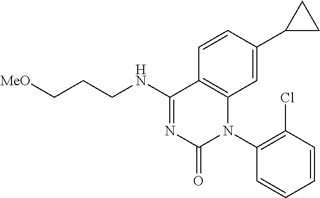 [SMILES: COCCCNc1nc(=O)n(-c2ccccc2Cl)c2cc(C3CC3)ccc12] | 1-(2-chlorophenyl)-7-cyclopropyl-4- ((3-methoxypropyl)amino)quinazolin- 2(1H)-one | m/z [M + H]⁺ 384.1 |
| 573 | 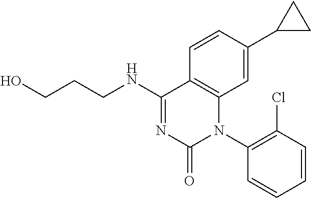 [SMILES: O=c1nc(NCCCO)c2ccc(C3CC3)cc2n1-c1ccccc1Cl] | 1-(2-chlorophenyl)-7-cyclopropyl-4- ((3-hydroxypropyl)amino)quinazolin- 2(1H)-one | m/z [M + H]⁺ 370.1 |
| 574 | 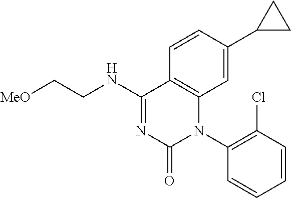 [SMILES: COCCNc1nc(=O)n(-c2ccccc2Cl)c2cc(C3CC3)ccc12] | 1-(2-chlorophenyl)-7-cyclopropyl-4- ((2-methoxyethyl)amino)quinazolin- 2(1H)-one | m/z [M + H]⁺ 370.1 |
| 575 | 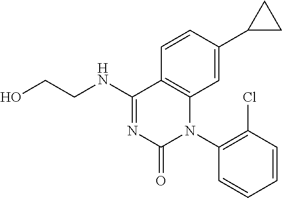 [SMILES: O=c1nc(NCCO)c2ccc(C3CC3)cc2n1-c1ccccc1Cl] | 1-(2-chlorophenyl)-7-cyclopropyl-4- ((2-hydroxyethyl)amino)quinazolin- 2(1H)-one | m/z [M + H]⁺ 356.1 |
| 576 | 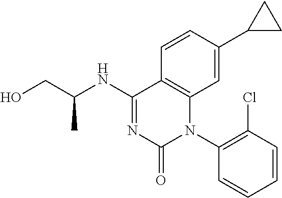 [SMILES: C[C@@H](CO)Nc1nc(=O)n(-c2ccccc2Cl)c2cc(C3CC3)ccc12] | 1-(2-chlorophenyl)-7-cyclopropyl-4- (((S)-1-hydroxypropan-2-yl)amino)- quinazolin-2(1H)-one | m/z [M + H]⁺ 370.1 |
| 577 | 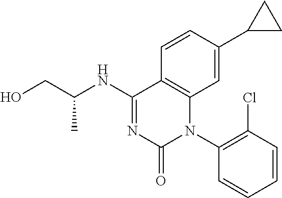 [SMILES: C[C@H](CO)Nc1nc(=O)n(-c2ccccc2Cl)c2cc(C3CC3)ccc12] | 1-(2-chlorophenyl)-7-cyclopropyl-4- (((R)-1-hydroxypropan-2-yl)amino)- quinazolin-2(1H)-one | m/z [M + H]⁺ 370.1 |
| 578 | 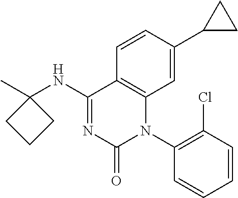 [SMILES: CC1(Nc2nc(=O)n(-c3ccccc3Cl)c3cc(C4CC4)ccc23)CCC1] | 1-(2-chlorophenyl)-7-cyclopropyl-4- ((1- methylcyclobutyl)amino)quinazolin- 2(1H)-one | m/z [M + H ]⁺ 380.15 |
| 579 | 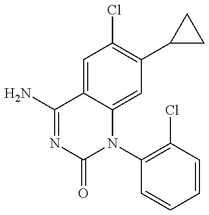 [SMILES: Nc1nc(=O)n(-c2ccccc2Cl)c2cc(C3CC3)c(Cl)cc12] | 4-amino-6-chloro-1-(2- chlorophenyl)-7- cyclopropylquinazolin-2(1H)-one | m/z [M + H]⁺ 347.0 |
| 580 | 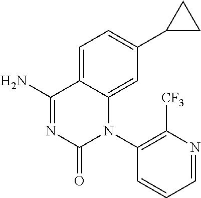 [SMILES: Nc1nc(=O)n(-c2cccnc2C(F)(F)F)c2cc(C3CC3)ccc12] | 4-amino-7-cyclopropyl-1-(2- (trifluoro-methyl)pyridin-3- yl)quinazolin-2(1H)-one | m/z [M + H]⁺ 347.0 |
| 581 | 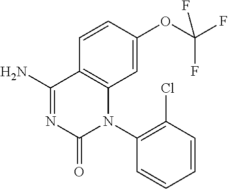 [SMILES: Nc1nc(=O)n(-c2ccccc2Cl)c2cc(OC(F)(F)F)ccc12] | 4-amino-1-(2-chlorophenyl)-7- (trifluoromethoxy)quinazolin-2(1H)- one | m/z [M + H]⁺ 356.0 |
| 582 | 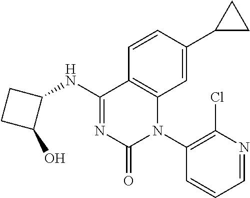 [SMILES: O=c1nc(N[C@H]2CC[C@@H]2O)c2ccc(C3CC3)cc2n1-c1cccnc1Cl] | 1-(2-chlorophenyl)-7-cyclopropyl-4- (((trans)-2-hydroxycyclobutyl)amino)- quinazolin-2(1H)-one | m/z [M + H]⁺ 382.1 |
| 583 | 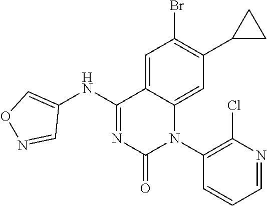 [SMILES: O=c1nc(Nc2cnoc2)c2cc(Br)c(C3CC3)cc2n1-c1cccnc1Cl] | 6-bromo-1-(2-chlorophenyl)-7- cyclopropyl-4-(isoxazol-4-ylamino)- quinazolin-2(1H)-one | m/z [M + H]⁺ 458.0 |
| 584 | 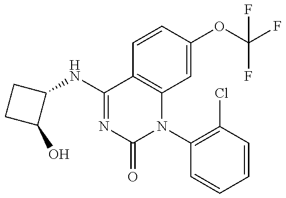 [SMILES: O=c1nc(N[C@H]2CC[C@@H]2O)c2ccc(OC(F)(F)F)cc2n1-c1ccccc1Cl] | 1-(2-chlorophenyl)-4-(((trans)-2- hydroxycyclobutyl)amino)-7- (trifluoromethoxy)-quinazolin-2(1H)- one | m/z [M + H]⁺ 426.1 |
| 585 | 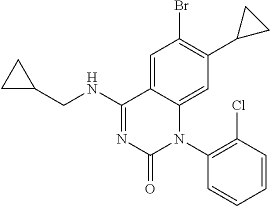 [SMILES: O=c1nc(NCC2CC2)c2cc(Br)c(C3CC3)cc2n1-c1ccccc1Cl] | 6-bromo-1-(2-chlorophenyl)-7- cyclopropyl-4-((cyclopropylmethyl)- amino)-quinazolin-2(1H)-one | m/z [M + H]⁺ 445.0 |
| 586 | 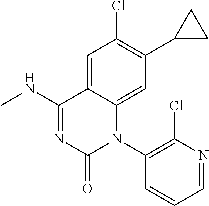 [SMILES: CNc1nc(=O)n(-c2cccnc2Cl)c2cc(C3CC3)c(Cl)cc12] | 6-chloro-1-(2-chlorophenyl)-7- cyclopropyl-4- (methylamino)quinazolin-2(1H)-one | m/z [M + H]⁺ 360.0 |
| 587 | 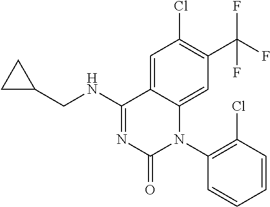 [SMILES: O=c1nc(NCC2CC2)c2cc(Cl)c(C(F)(F)F)cc2n1-c1ccccc1Cl] | 6-chloro-1-(2-chlorophenyl)-4- ((cyclopropylmethyl)amino)-7- (trifluoromethyl)-quinazolin-2(1H)- one | m/z [M + H]⁺ 428.0 |
| 588 | 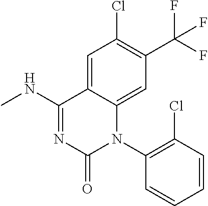 [SMILES: CNc1nc(=O)n(-c2ccccc2Cl)c2cc(C(F)(F)F)c(Cl)cc12] | 6-chloro-1-(2-chlorophenyl)-4- (methylamino)-7-(trifluoromethyl) quinazolin-2(1H)-one | m/z [M + H]⁺ 388.0 |
| 589 | 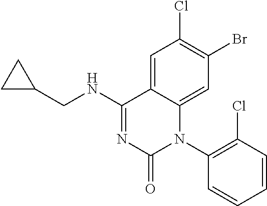 [SMILES: O=c1nc(NCC2CC2)c2cc(Cl)c(Br)cc2n1-c1ccccc1Cl] | 7-bromo-6-chloro-1-(2- chlorophenyl)-4- ((cyclopropylmethyl)amino)quinaozlin- 2(1H)-one | m/z [M + H]⁺ 439.9 |
| 590 | 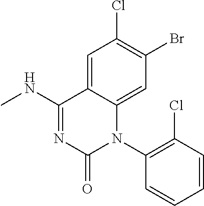 [SMILES: CNc1nc(=O)n(-c2ccccc2Cl)c2cc(Br)c(Cl)cc12] | 7-bromo-6-chloro-1-(2- chlorophenyl)-4- (methylamino)quinazolin-2(1H)-one | m/z [M + H]⁺ 399.9 |
| 591 | 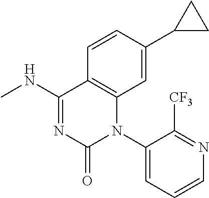 [SMILES: CNc1nc(=O)n(-c2cccnc2C(F)(F)F)c2cc(C3CC3)ccc12] | 7-cyclopropyl-4-(methylamino)-1-(2- (trifluoromethyl)pyridin-3- yl)quinazolin-2(1H)-one | m/z [M + H]⁺ 361.1 |
| 592 | 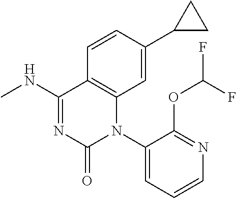 [SMILES: CNc1nc(=O)n(-c2cccnc2OC(F)F)c2cc(C3CC3)ccc12] | 7-cyclopropyl-1-(2-(difluoro- methoxy)pyridin-3-yl)-4- (methylamino)-quinaozlin-2(1H)-one | m/z [M + H]⁺ 359.1 |
| 593 | 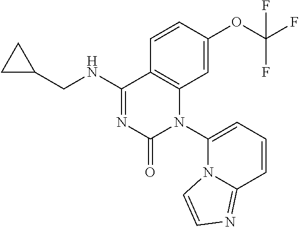 [SMILES: O=c1nc(NCC2CC2)c2ccc(OC(F)(F)F)cc2n1-c1cccc2nccn12] | 4-((cyclopropylmethyl)amino)-1- (imidazo[1,2-a]pyridin-5-yl)-7- (trifluoromethoxy)-quinazolin-2(1H)- one | m/z [M + H]⁺ 416.1 |
| 594 | 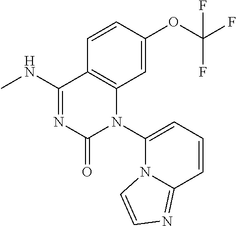 [SMILES: CNc1nc(=O)n(-c2cccc3nccn23)c2cc(OC(F)(F)F)ccc12] | 1-(imidazo[1,2-a]pyridin-5-yl)-4- (methylamino)-7-(trifluoromethxoy)- quinaozlin-2(1H)-one | m/z [M + H]⁺ 376.1 |
| 595 | 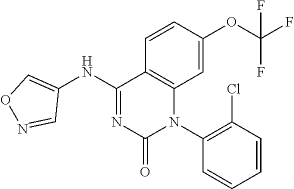 [SMILES: O=c1nc(Nc2cnoc2)c2ccc(OC(F)(F)F)cc2n1-c1ccccc1Cl] | 1-(2-chlorophenyl)-4-(isoxaozl-4- ylamino)-7-(trifluoromethoxy)- quinazolin-2(1H)-one | m/z [M + H]⁺ 423.0 |
| 596 | 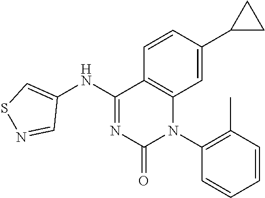 [SMILES: Cc1ccccc1-n1c(=O)nc(Nc2cnsc2)c2ccc(C3CC3)cc21] | 7-cyclopropyl-4-(isothiazol-4- ylamino)-1-(o-tolyl)quinazolin-2(1H0- one | m/z [M + H]⁺ 375.1 |
| 597 | 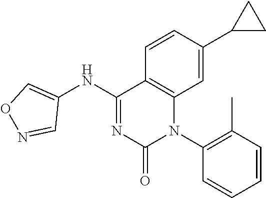 [SMILES: Cc1ccccc1-n1c(=O)nc(Nc2cnoc2)c2ccc(C3CC3)cc21] | 7-cyclopropyl-4-(isoxazol-4- ylamino)-1-(o-tolyl)quinazolin-2(1H)- one | m/z [M + H]⁺ 359.1 |
| 598 | 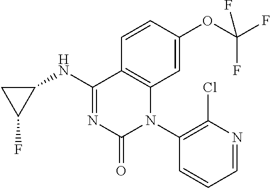 [SMILES: O=c1nc(N[C@H]2C[C@H]2F)c2ccc(OC(F)(F)F)cc2n1-c1cccnc1Cl] | 1-(2-chlorophenyl)-4-(((1S,2R)-2- fluorocyclopropyl)amino)-7- (trifluoromethoxy)-quinazolin-2(1H)- one | m/z [M + H]⁺ 414.05 |
| 599 | 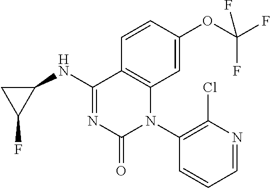 [SMILES: O=c1nc(N[C@@H]2C[C@@H]2F)c2ccc(OC(F)(F)F)cc2n1-c1cccnc1Cl] | 1-(2-chlorophenyl)-4-(((1R,2S)-2- fluorocyclopropyl)amino)-7- (trifluoromethoxy)-quinazolin-2(1H)- one | m/z [M + H]⁺ 414.05 |
| 600 | 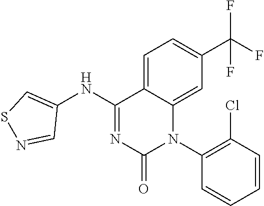 [SMILES: O=c1nc(Nc2cnsc2)c2ccc(C(F)(F)F)cc2n1-c1ccccc1Cl] | 1-(2-chlorophenyl)-4-(isothiazol-4- ylamino)-7- (trifluoromethyl)pyrido[2,3- d]pyrimidin-2(1H)-one | m/z [M + H]⁺ 424.0 |
| 601 | 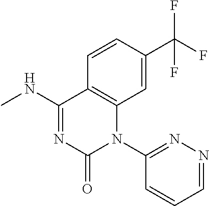 [SMILES: CNc1nc(=O)n(-c2cccnn2)c2cc(C(F)(F)F)ccc12] | 4-(methylamino)-1-(pyridazin-3-yl)- 7-(trifluoromethyl)quinazolin-2(1H)- one | m/z [M + H]⁺ 322.1 |
| 602 | 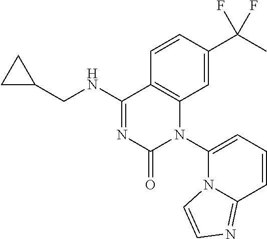 [SMILES: CC(F)(F)c1ccc2c(NCC3CC3)nc(=O)n(-c3cccc4nccn34)c2c1] | 4-((cyclopropylmethyl)amino)-7- (1,1-difluoroethyl)-1-(imidazo[1,2- a]pyridin-5-yl)quinazolin-2(1H)-one | m/z [M + H]⁺ 396.2 |
| 603 | 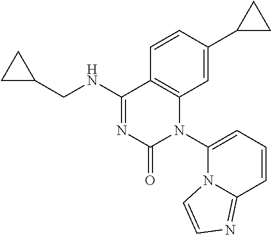 [SMILES: O=c1nc(NCC2CC2)c2ccc(C3CC3)cc2n1-c1cccc2nccn12] | 7-cyclopropyl-4- ((cyclopropylmethyl)-amino)-1- (imidazo[1,2-a]pyridin-5-yl)- quinazolin-2(1H)-one | m/z [M + H]⁺ 372.15 |
| 604 | 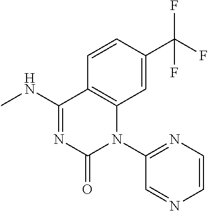 [SMILES: CNc1nc(=O)n(-c2cnccn2)c2cc(C(F)(F)F)ccc12] | 4-(methylamino)-1-(pyrazin-2-yl)-7- (trifluoromethyl)quinazolin-2(1H)-one | m/z [M + H]⁺ 322.1 |
| 605 | 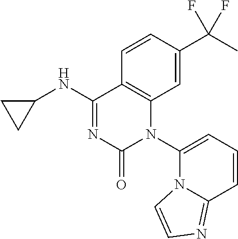 [SMILES: CC(F)(F)c1ccc2c(NC3CC3)nc(=O)n(-c3cccc4nccn34)c2c1] | 4-(cyclopropylamino)-7-(1,1- difluoroethyl)-1-(imidazo[1,2- a]pyridin-5-yl)-quinazolin-2(1H)-one | m/z [M + H]⁺ 382.2 |
| 606 | 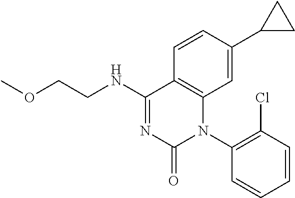 [SMILES: COCCNc1nc(=O)n(-c2ccccc2Cl)c2cc(C3CC3)ccc12] | 1-(2-chlorophenyl)-7-cyclopropyl-4- ((2-methoxyethyl)amino)quinazolin- 2(1H)-one | m/z [M + H]⁺ 370.1 |
| 607 | 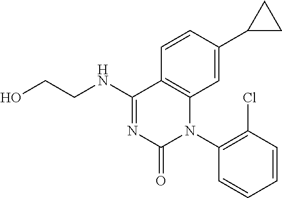 [SMILES: O=c1nc(NCCO)c2ccc(C3CC3)cc2n1-c1ccccc1Cl] | 1-(2-chlorophenyl)-7-cyclopropyl-4- ((2-hydroxyethyl)amino)quinazolin- 2(1H)-one | m/z [M + H]⁺ 356.1 |
| 608 | 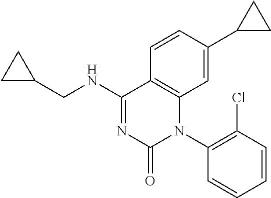 [SMILES: O=c1nc(NCC2CC2)c2ccc(C3CC3)cc2n1-c1ccccc1Cl] | 1-(2-chlorophenyl)-7-cyclopropyl-4- ((cyclopropylmethyl)amino)quinazolin- 2(1H)-one | m/z [M + H]⁺ 366.1 |
| 609 | 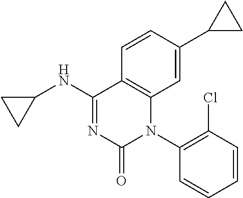 [SMILES: O=c1nc(NC2CC2)c2ccc(C3CC3)cc2n1-c1ccccc1Cl] | 1-(2-chlorophenyl)-7-cyclopropyl-4- (cyclopropylamino)quinazolin-2(1H)- one | m/z [M + H]⁺ 352.1 |
| 610 | 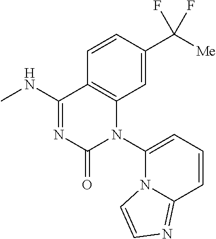 [SMILES: CNc1nc(=O)n(-c2cccc3nccn23)c2cc(C(C)(F)F)ccc12] | 7-(1,1-difluoroethyl)-1-(imidazo[1,2- a]pyridin-5-yl)-4-(methylamino)- quinazolin-2(1H)-one | m/z [M + H]⁺ 356.1 |
| 611 | 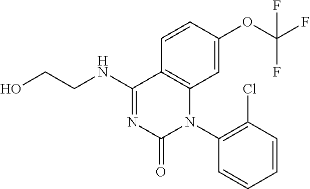 [SMILES: O=c1nc(NCCO)c2ccc(OC(F)(F)F)cc2n1-c1ccccc1Cl] | 1-(2-chlorophenyl)-4-((2- hydroxyethyl)-amino)-7- (trifluoromethoxy)quinazolin-2(1H)- one | m/z [M + H]⁺ 400.1 |
| 612 | 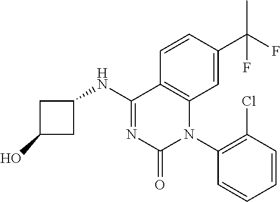 [SMILES: CC(F)(F)c1ccc2c(N[C@H]3C[C@H](O)C3)nc(=O)n(-c3ccccc3Cl)c2c1] | 1-(2-chlorophenyl)-7-(1,1- difluoroethyl)-4-(((trans)-3- hydroxycyclobutyl)amino)-quinazolin- 2(1H)-one | m/z [M + H]⁺ 406.1 |
| 613 | 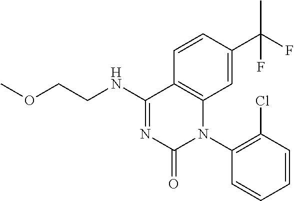 [SMILES: COCCNc1nc(=O)n(-c2ccccc2Cl)c2cc(C(C)(F)F)ccc12] | 1-(2-chlorophenyl)-7-(1,1- difluoroethyl)-4-((2- methoxyethyl)amino)quinazolin- 2(1H)-one | m/z [M + H]⁺ 394.05 |
| 614 | 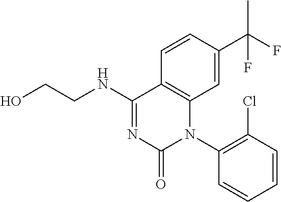 [SMILES: CC(F)(F)c1ccc2c(NCCO)nc(=O)n(-c3ccccc3Cl)c2c1] | 1-(2-chlorophenyl)-7-(1,1- difluoroethyl)-4-((2- hydroxyethyl)amino)quinazolin- 2(1H)-one | m/z [M + H]⁺ 380.05 |
| 615 | 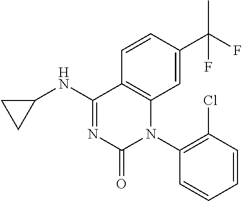 [SMILES: CC(F)(F)c1ccc2c(NC3CC3)nc(=O)n(-c3ccccc3Cl)c2c1] | 1-(2-chlorophenyl)-4-(cyclopropyl- amino)-7-(1,1- difluoroethyl)quinazolin-2(1H)-one | m/z [M + H]⁺ 376.1 |
| 616 | 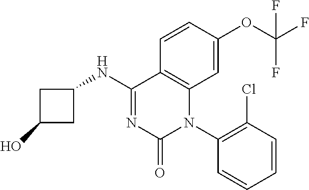 [SMILES: O=c1nc(N[C@H]2C[C@H](O)C2)c2ccc(OC(F)(F)F)cc2n1-c1ccccc1Cl] | 1-(2-chlorophenyl)-4-(((trans)-3- hydroxycyclobutyl)amino)-7- (trifluoromethoxy)-quinazolin-2(1H)- one | m/z [M + H]⁺ 426.1 |
| 617 | 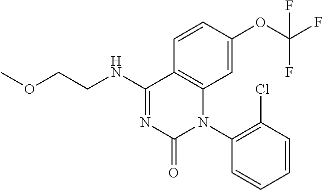 [SMILES: COCCNc1nc(=O)n(-c2ccccc2Cl)c2cc(OC(F)(F)F)ccc12] | 1-(2-chlorophenyl)-4-((2- methoxyethyl)-amino)-7- (trifluoromethoxy)quinazolin-2(1H0- one | m/z [M + H]⁺ 414.0 |
| 618 | 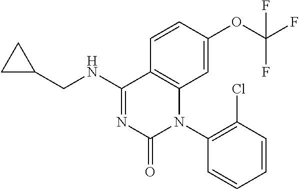 [SMILES: O=c1nc(NCC2CC2)c2ccc(OC(F)(F)F)cc2n1-c1ccccc1Cl] | 1-(2-chlorophenyl)-4-((cyclopropyl- methyl)amino)-7-(trifluoromethoxy)- quinazolin-2(1H)-one | m/z [M + H]⁺ 410.0 |
| 619 | 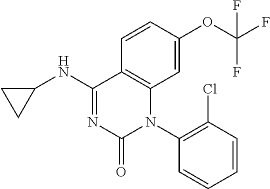 [SMILES: O=c1nc(NC2CC2)c2ccc(OC(F)(F)F)cc2n1-c1ccccc1Cl] | 1-(2-chlorophenyl)-4-(cyclopropyl- amino)-7- (trifluoromethoxy)quinazolin-2(1H)- one | m/z [M + H]⁺ 396.0 |
| 620 | 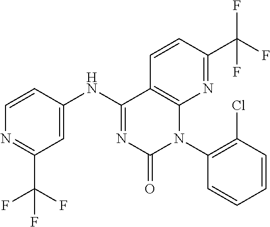 [SMILES: O=c1nc(Nc2ccnc(C(F)(F)F)c2)c2ccc(C(F)(F)F)nc2n1-c1ccccc1Cl] | 1-(2-chlorophenyl)-7- (trifluoromethyl)-4-((2- (trifluoromethyl)pyridin-4-yl)-amino)- pyrido[2,3-d]pyrimidin-2(1H)-one | m/z [M + H]⁺ 486.05 |
| 621 | 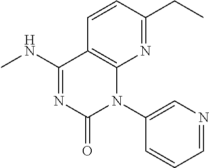 [SMILES: CCc1ccc2c(NC)nc(=O)n(-c3cccnc3)c2n1] | 7-ethyl-4-(methylamino)-1-(pyridin- 3-yl)pyrido[2,3-d]pyrimidin-2(1H)-one | m/z [M + H]⁺ 282.15 |
| 622 | 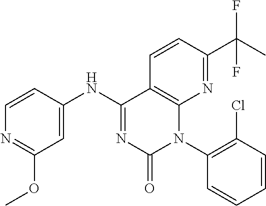 | 1-(2-chlorophenyl)-4-((2-methoxy- pyridin-4-yl)amino)-7- (trifluoromethyl)-pyrido-[2,3- d]pyrimidin-2(1H)-one | m/z [M + H]⁺ 448.1 |
| 623 | 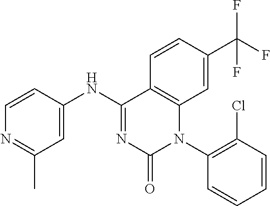 [SMILES: Cc1cc(Nc2nc(=O)n(-c3ccccc3Cl)c3cc(C(F)(F)F)ccc23)ccn1] | 1-(2-chlorophenyl)-4-((2- methylpyridin-4-yl)amino)-7- (trifluoromethyl)-pyrido[2,3- d]pyrimidin-2(1H)-one | |
| 624 | 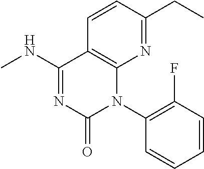 [SMILES: CCc1ccc2c(NC)nc(=O)n(-c3ccccc3F)c2n1] | 7-ethyl-1-(2-fluorophenyl)-4-(methyl- amino)pyrido[2,3-d]pyrimidin-2(1H)- one | m/z [M + H]⁺ 299.1 |
| 625 | 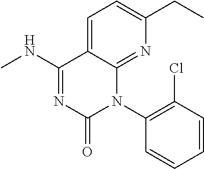 [SMILES: CCc1ccc2c(NC)nc(=O)n(-c3ccccc3Cl)c2n1] | 1-(2-chlorophenyl)-7-ethyl-4- (methyl-amino)pyrido[2,3- d]pyrimidin-2(1H)-one | m/z [M + H]⁺ 315.1 |
| 626 | 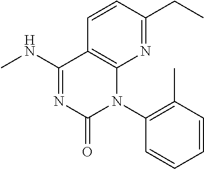 [SMILES: CCc1ccc2c(NC)nc(=O)n(-c3ccccc3C)c2n1] | 7-ethyl-4-(methylamino)-1-(o-tolyl)- pyrido[2,3-d]pyrimidin-2(1H)-one | m/z [M + H]⁺ 295.15 |
| 627 | 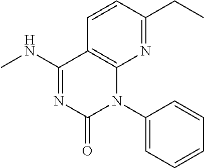 [SMILES: CCc1ccc2c(NC)nc(=O)n(-c3ccccc3)c2n1] | 7-ethyl-4-(methylamino)-1-phenyl- pyrido[2,3-d]pyrimidin-2(1H)-one | m/z [M + H]⁺ 281.2 |
| 628 | 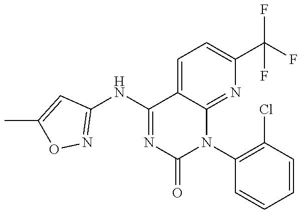 [SMILES: Cc1cc(Nc2nc(=O)n(-c3ccccc3Cl)c3nc(C(F)(F)F)ccc23)no1] | 1-(2-chlorophenyl)-4-((5-methyl- isoxazol-3-yl)amino)-7-(trifluoro- methyl)pyrido[2,3-d]pyrimidin-2(1H)- one | m/z [M + H]⁺ 422.05 |
| 629 | 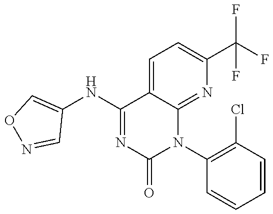 [SMILES: O=c1nc(Nc2cnoc2)c2ccc(C(F)(F)F)nc2n1-c1ccccc1Cl] | 1-(2-chlorophenyl)-4-(isoxazol-4-yl- amino)-7-(trifluoromethyl)pyrido[2,3- d]-pyrimidin-2(1H)-one | m/z [M + H]⁺ 408.0 |
| 630 | 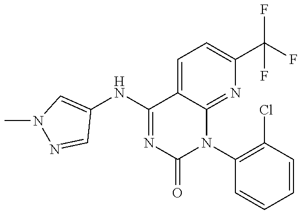 [SMILES: Cn1cc(Nc2nc(=O)n(-c3ccccc3Cl)c3nc(C(F)(F)F)ccc23)cn1] | 1-(2-chlorophenyl)-4-((1-methyl-1H- pyrazol-4-yl)amino)-7- (trifluoromethyl)-pyrido[2,3- d]pyrimidin-2(1H)-one | m/z [M + H]⁺ 421.0 |
| 631 | 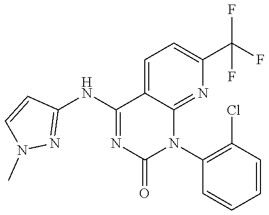 [SMILES: Cn1ccc(Nc2nc(=O)n(-c3ccccc3Cl)c3nc(C(F)(F)F)ccc23)n1] | 1-(2-chlorophenyl)-4-((1-methyl-1H- pyrazol-3-yl)amino)-7-(trifluoro- methyl)pyrido-[2,3-d]pyrimidin-2(1H)- one | m/z [M + H]⁺ 421.0 |
| 632 | 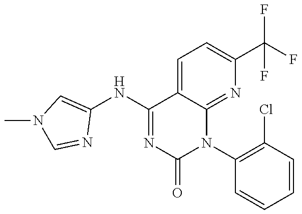 [SMILES: Cn1cnc(Nc2nc(=O)n(-c3ccccc3Cl)c3nc(C(F)(F)F)ccc23)c1] | 1-(2-chlorophenyl)-4-((1-methyl-1H- imidazol-4-yl)amino)-7-(trifluoro- methyl)-pyrido[2,3-d]pyrimidin-2(1H)- one | m/z [M + H]⁺ 421.0 |
| 633 | 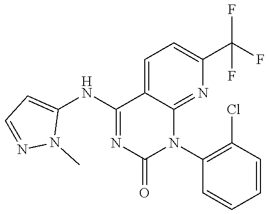 [SMILES: Cn1nccc1Nc1nc(=O)n(-c2ccccc2Cl)c2nc(C(F)(F)F)ccc12] | 1-(2-chlorophenyl)-4-((1-methyl-1H- pyrazol-4-yl)amino)-7- (trifluoromethyl)-pyrido[2,3- d]pyrimidin-2(1H)-one | m/z [M + H]⁺ 421.0 |
| 634 | 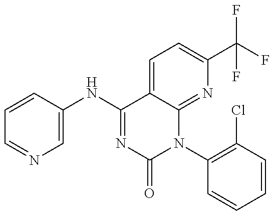 [SMILES: O=c1nc(Nc2cccnc2)c2ccc(C(F)(F)F)nc2n1-c1ccccc1Cl] | 1-(2-chlorophenyl)-4-(pyridin-3-yl- amino)-7-(trifluoromethyl)pyrido[2,3- d]-pyrimidin-2(1H)-one | m/z [M + H]⁺ 418.0 |
| 635 | 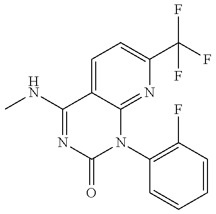 [SMILES: CNc1nc(=O)n(-c2ccccc2F)c2nc(C(F)(F)F)ccc12] | 1-(2-fluorophenyl)-4-(methylamino)- 7-(trifluoromethyl)pyrido[2,3- d]pyrimidin-2(1H)-one | m/z [M + H]⁺ 339.1 |
| 636 | 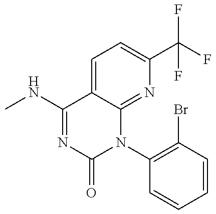 [SMILES: CNc1nc(=O)n(-c2ccccc2Br)c2nc(C(F)(F)F)ccc12] | 1-(2-bromophenyl)-4- (methylamino)-7- (trifluoromethyl)pyrido[2,3- d]pyrimidin-2(1H)-one | m/z [M + H]⁺ 401.0, 399.0 |
| 637 | 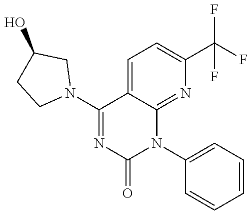 [SMILES: O=c1nc(N2CC[C@@H](O)C2)c2ccc(C(F)(F)F)nc2n1-c1ccccc1] | (R)-4-(3-hydroxypyrrolidin-1-yl)-1- phenyl-7-(trifluoromethyl)pyrido[2,3- d]-pyrimidin-2(1H)-one | m/z [M + H]⁺ 377.1 |
| 638 | 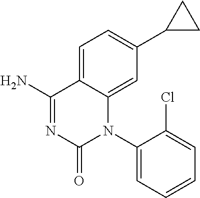 [SMILES: Nc1nc(=O)n(-c2ccccc2Cl)c2cc(C3CC3)ccc12] | 4-amino-1-(2-chlorophenyl)-7- cyclopropylquinazolin-2(1H)-one | m/z [M + H]⁺ 312.00 |
| 639 | 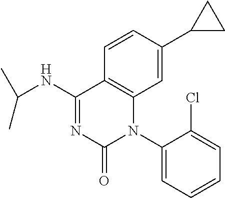 [SMILES: CC(C)Nc1nc(=O)n(-c2ccccc2Cl)c2cc(C3CC3)ccc12] | 1-(2-chlorophenyl)-7-cyclopropyl-4- (isopropylamino)quinazolin-2(1H)- one | m/z [M + H]⁺ 354.20 |
| 640 | 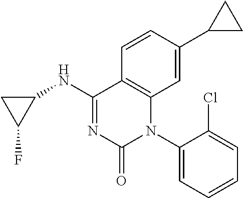 | 1-(2-chlorophenyl)-7-cyclopropyl-4- (((1S,2R)-2- fluorocyclopropyl)amino)-quinazolin- 2(1H)-one | m/z [M + H]⁺ 370.00 |
| 641 | 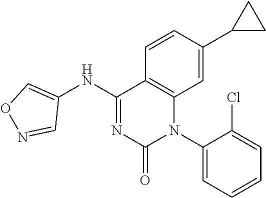 [SMILES: O=c1nc(Nc2cnoc2)c2ccc(C3CC3)cc2n1-c1ccccc1Cl] | 1-(2-chlorophenyl)-7-cyclopropyl-4- (isoxazol-4-ylamino)quinazolin- 2(1H)-one | m/z [M + H]⁺ 379.00 |
| 642 | 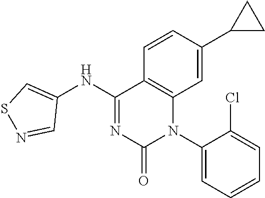 [SMILES: O=c1nc(Nc2cnsc2)c2ccc(C3CC3)cc2n1-c1ccccc1Cl] | 1-(2-chlorophenyl)-7-cyclopropyl-4- (isothiazol-4-ylamino)quinazolin- 2(1H)-one | m/z [M + H]⁺ 395.00 |
| 643 | 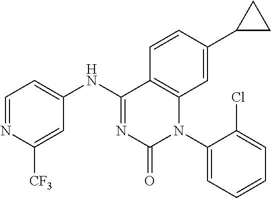 [SMILES: O=c1nc(Nc2ccnc(C(F)(F)F)c2)c2ccc(C3CC3)cc2n1-c1ccccc1Cl] | 1-(2-chlorophenyl)-7-cyclopropyl-4- ((2-(trifluoromethyl)pyridin-4- yl(amino)-quinazolin-2(1H)-one | m/z [M + H]⁺ 457.00 |
| 644 | 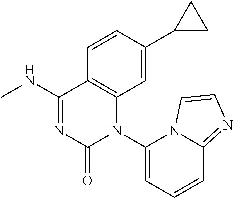 [SMILES: CNc1nc(=O)n(-c2cccc3nccn23)c2cc(C3CC3)ccc12] | 7-cyclopropyl-1-(imidazo[1,2- a]pyridin-5-yl)-4- (methylamino)quinazolin-2(1H)-one | m/z [M + H]⁺ 332.00 |
| 645 | 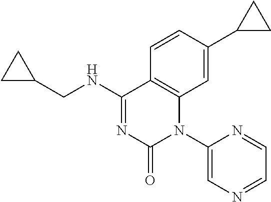 [SMILES: O=c1nc(NCC2CC2)c2ccc(C3CC3)cc2n1-c1cnccn1] | 7-cyclopropyl-4- ((cyclopropylmethyl)-amino)-1- (pyrazin-2-yl)quinazolin-2(1H)-one | m/z [M + H]⁺ 334.20 |
| 646 | 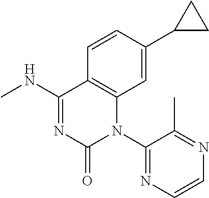 [SMILES: CNc1nc(=O)n(-c2nccnc2C)c2cc(C3CC3)ccc12] | 7-cyclopropyl-4-(methylamino)-1-(3- methylpyrazin-2-yl)quinazolin-2(1H)- one | m/z [M + H]⁺ 308.20 |
| 647 | 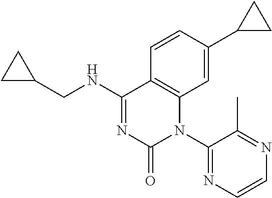 [SMILES: Cc1nccnc1-n1c(=O)nc(NCC2CC2)c2ccc(C3CC3)cc21] | 7-cyclopropyl-4- ((cyclopropylmethyl)-amino)-1-(3- methylpyrazin-2-yl)-quinazolin-2(1H)- one | m/z [M + H]⁺ 348.20 |
| 648 | 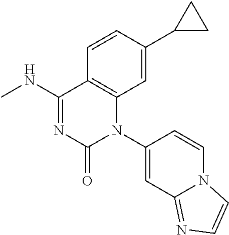 [SMILES: CNc1nc(=O)n(-c2ccn3ccnc3c2)c2cc(C3CC3)ccc12] | 7-cyclopropyl-1-(imidazo[1,2- a]pyridin-7-yl)-4- (methylamino)quinazolin-2(1H)-one | m/z [M + H]⁺ 332.00 |
| 649 | 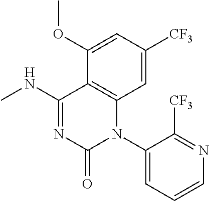 [SMILES: CNc1nc(=O)n(-c2cccnc2C(F)(F)F)c2cc(C(F)(F)F)cc(OC)c12] | 5-methoxy-4-(methylamino)-7- (trifluoromethyl)-1-(2- (trifluoromethyl)-pyridin-3- yl)quinazolin-2(1H)-one | m/z [M + H]⁺ 419.00 |
| 650 | 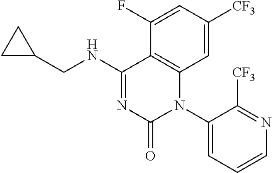 [SMILES: O=c1nc(NCC2CC2)c2c(F)cc(C(F)(F)F)cc2n1-c1cccnc1C(F)(F)F] | 4-((cyclopropylmethyl)amino)-5- fluoro-7-(trifluoromethyl)-1-(2- (trifluoro-methyl)-pyridin-3- yl)quinazolin-2(1H)-one | m/z [M + H]⁺ 447.00 |
| 651 | 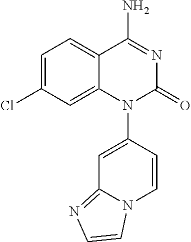 [SMILES: Nc1nc(=O)n(-c2ccn3ccnc3c2)c2cc(Cl)ccc12] | 4-amino-7-chloro-1-(imidazo[1,2-a]- pyridin-7-yl)quinazolin-2(1H)-one | m/z [M + H]⁺ 312.0 |
| 652 | 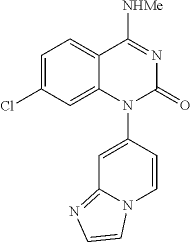 [SMILES: CNc1nc(=O)n(-c2ccn3ccnc3c2)c2cc(Cl)ccc12] | 7-chloro-1-(imidazo[1,2-a]pyridin-7- yl)-4-(methylamino)quinazolin-2(1H)- one | m/z [M + H]⁺ 326.0 |
| 653 | 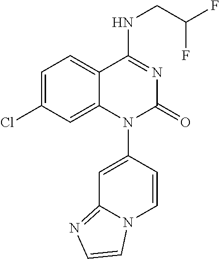 [SMILES: O=c1nc(NCC(F)F)c2ccc(Cl)cc2n1-c1ccn2ccnc2c1] | 7-chloro-4-((2,2- difluoroethyl)amino)-1-(imidazo[1,2- a]pyridin-7-yl)quinazolin-2(1H)-one | m/z [M + H]⁺ 376.0 |
| 654 | 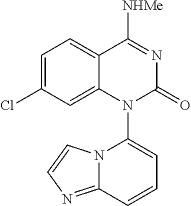 [SMILES: CNc1nc(=O)n(-c2cccc3nccn23)c2cc(Cl)ccc12] | 7-chloro-1-(imidazo[1,2-a]pyridin-5- yl)-4-(methylamino)quinazolin-2(1H)- one | m/z [M + H]⁺ 326.0 |
| 655 | 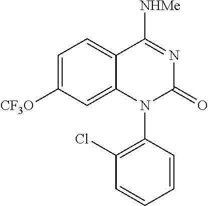 | 1-(2-chlorophenyl)-4-(methylamino)- 7-(trifluoromethoxy)quinazolin-2(1H)- one | m/z [M + H]⁺ 370.1 |
| 656 | 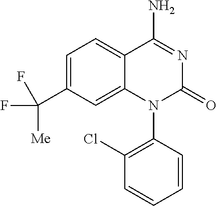 [SMILES: CC(F)(F)c1ccc2c(N)nc(=O)n(-c3ccccc3Cl)c2c1] | 4-amino-1-(2-chlorophenyl)-7-(1,1- difluoroethyl)quinazolin-2(1H)-one | m/z [M + H]⁺ 336.1 |
| 657 | 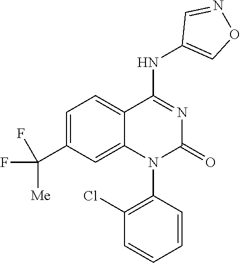 [SMILES: CC(F)(F)c1ccc2c(Nc3cnoc3)nc(=O)n(-c3ccccc3Cl)c2c1] | 1-(2-chlorophenyl)-7-(1,1- difluoroethyl)-4-(isoxazol-4- yl)amino)quinazolin-2(1H)-one | m/z [M + H]⁺ 403.0 |
| 658 | 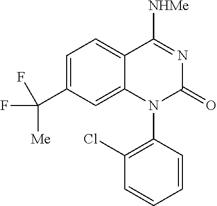 [SMILES: CNc1nc(=O)n(-c2ccccc2Cl)c2cc(C(C)(F)F)ccc12] | 1-(2-chlorophenyl)-7-(1,1- difluoroethyl)-4- (methylamino)quinazolin-2(1H)-one | m/z [M + H]⁺ 350.1 |
| 659 | 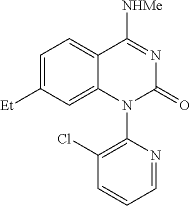 [SMILES: CCc1ccc2c(NC)nc(=O)n(-c3ncccc3Cl)c2c1] | 1-(3-chloropyridin-2-yl)-7-ethyl-4- (methylamino)quinazolin-2(1H)-one | m/z [M + H]⁺ 315.0 |
| 660 | 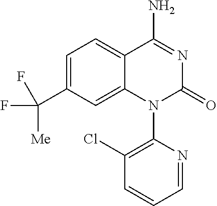 [SMILES: CC(F)(F)c1ccc2c(N)nc(=O)n(-c3ncccc3Cl)c2c1] | 4-amino-1-(3-chloropyridin-2-yl)-7- (1,1-difluoroethyl)quinazolin-2(1H)- one | m/z [M + H]⁺ 337.0. |
| 661 | 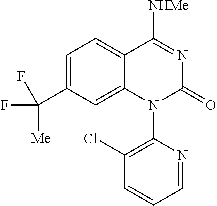 [SMILES: CNc1nc(=O)n(-c2ncccc2Cl)c2cc(C(C)(F)F)ccc12] | 1-(3-chloropyridin-2-yl)-7-(1,1- difluoroethyl)-4-(methylamino)- quinazolin-2(1H)-one | m/z [M + H]⁺ 351.0 |
| 662 | 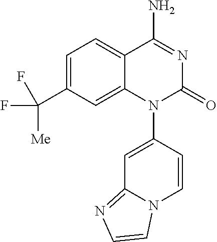 [SMILES: CC(F)(F)c1ccc2c(N)nc(=O)n(-c3ccn4ccnc4c3)c2c1] | 4-amino-7-(1,1-difluoroethyl)-1- (imidazo[1,2-a]pyridin-7- yl)quinazolin-2(1H)-one | m/z [M + H]⁺ 342.1 |
| 663 | 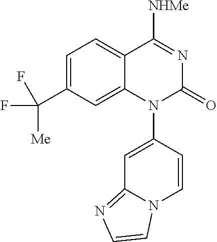 [SMILES: CNc1nc(=O)n(-c2ccn3ccnc3c2)c2cc(C(C)(F)F)ccc12] | 7-(1,1-difluoroethyl)-1-(imidazo[1,2- a]pyridin-7-yl)-4-(methylamino)- quinazolin-2(1H)-one | m/z [M + H]⁺ 356.0 |
| 664 | 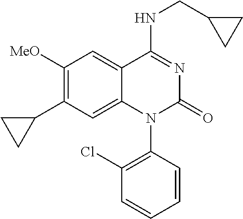 [SMILES: COc1cc2c(NCC3CC3)nc(=O)n(-c3ccccc3Cl)c2cc1C1CC1] | 1-(2-chlorophenyl)-7-cyclopropyl-4- ((cyclopropylmethyl)amino)-6- methoxyquinazolin-2(1H)-one | m/z [M + H]⁺ 396.2 |
| 665 | 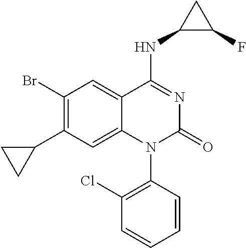 [SMILES: O=c1nc(N[C@H]2C[C@H]2F)c2cc(Br)c(C3CC3)cc2n1-c1ccccc1Cl] | 6-bromo-1-(2-chlorophenyl)-7- cyclopropyl-4-(((1S,2R)-2- fluorocyclo-propyl)amino)quinazolin- 2(1H)-one | m/z [M + H]⁺ 448.0/450.0 |
| 666 | 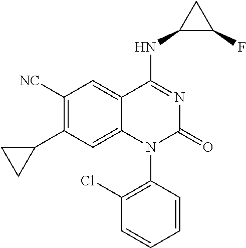 [SMILES: N#Cc1cc2c(N[C@H]3C[C@H]3F)nc(=O)n(-c3ccccc3Cl)c2cc1C1CC1] | 1-(2-chlorophenyl)-7-cyclopropyl-4- (((1S,2R)-2- fluorocyclopropyl)amino)-2-oxo-1,2- dihydroquinazoline-6-carbonitrile | m/z [M + H]⁺ 395.0 |
| 667 | 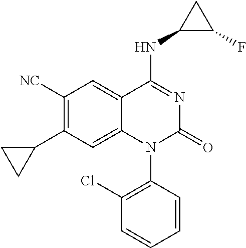 [SMILES: N#Cc1cc2c(N[C@H]3C[C@@H]3F)nc(=O)n(-c3ccccc3Cl)c2cc1C1CC1] | 1-(2-chlorophenyl)-7-cyclopropyl-4- (((trans)-2-fluorocyclopropyl)amino)- 2-oxo-1,2-dihydroquinazoline-6- carbonitrile | m/z [M + H]⁺ 395.0 |
| 668 | 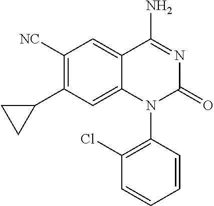 [SMILES: N#Cc1cc2c(N)nc(=O)n(-c3ccccc3Cl)c2cc1C1CC1] | 4-amino-1-(2-chlorophenyl)-7- cyclopropyl-2-oxo-1,2-dihydro- quinazoline-6-carbonitrile | m/z [M + H]⁺ 337.0 |
| 669 | 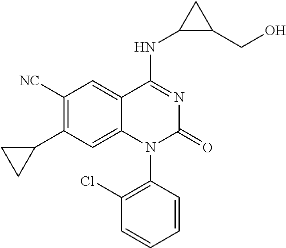 [SMILES: N#Cc1cc2c(NC3CC3CO)nc(=O)n(-c3ccccc3Cl)c2cc1C1CC1] | 1-(2-chlorophenyl)-7-cyclopropyl-4- ((1- (hydroxymethyl)cyclopropyl)amino)- 2-oxo-1,2-dihydroquinazoline-6- carbonitrile | m/z [M + H]⁺ 421.0 |
| 670 | 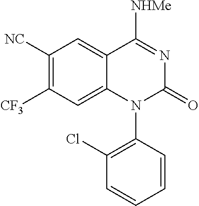 [SMILES: CNc1nc(=O)n(-c2ccccc2Cl)c2cc(C(F)(F)F)c(C#N)cc12] | 1-(2-chlorophenyl)-4-(methylamino)- 2-oxo-7-(trifluoromethyl)-1,2-dihydro- quinazoline-6-carbonitrile | m/z [M + H]⁺ 379.0 |
| 671 | 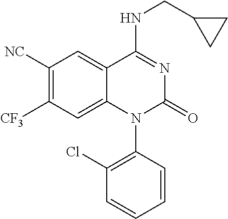 [SMILES: N#Cc1cc2c(NCC3CC3)nc(=O)n(-c3ccccc3Cl)c2cc1C(F)(F)F] | 1-(2-chlorophenyl)-4-((cyclopropyl- methyl)amino)-2-oxo-7-(trifluoro- methyl)-1,2-dihydroquinazoline-6- carbonitrile | m/z [M + H]⁺ 419.0 |
| 672 | 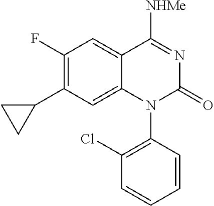 [SMILES: CNc1nc(=O)n(-c2ccccc2Cl)c2cc(C3CC3)c(F)cc12] | 1-(2-chlorophenyl)-7-cyclopropyl-6- fluoro-4-(methylamino)quinazolin- 2(1H)-one | m/z [M + H]⁺ 344.0 |
| 673 | 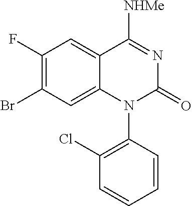 [SMILES: CNc1nc(=O)n(-c2ccccc2Cl)c2cc(Br)c(F)cc12] | 7-bromo-1-(2-chlorophenyl)-6- fluoro-4-(methylamino)quinazolin- 2(1H)-one | m/z [M + H]⁺ 381.9/384.0. |
| 674 | 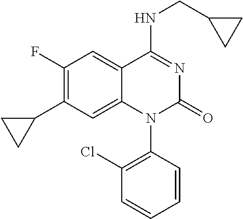 [SMILES: O=c1nc(NCC2CC2)c2cc(F)c(C3CC3)cc2n1-c1ccccc1Cl] | 1-(2-chlorophenyl)-7-cyclopropyl-4- ((cyclopropylmethyl)amino)-6-fluoro- quinazolin-2(1H)-one | m/z [M + H]⁺ 384.0 |
| 675 | 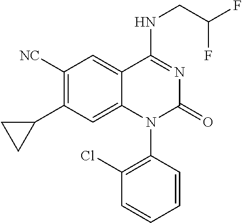 [SMILES: N#Cc1cc2c(NCC(F)F)nc(=O)n(-c3ccccc3Cl)c2cc1C1CC1] | 1-(2-chlorophenyl)-7-cyclopropyl-4- ((2,2-difluoroethyl)amino)-2-oxo-1,2- dihydro-quinazoline-6-carbonitrile | m/z [M + H]⁺ 410.1 |
| 676 | 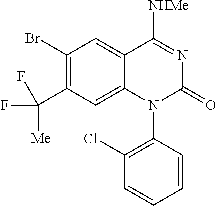 [SMILES: CNc1nc(=O)n(-c2ccccc2Cl)c2cc(C(C)(F)F)c(Br)cc12] | 6-bromo-1-(2-chlorophenyl)-7-(1,1- difluoroethyl)-4-(methylamino)- quinazolin-2(1H)-one | m/z [M + H]⁺ 428.0/430.0 |
| 677 | 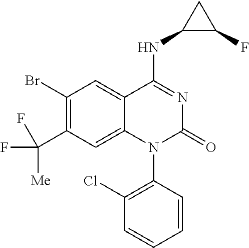 [SMILES: CC(F)(F)c1cc2c(cc1Br)c(N[C@H]1C[C@H]1F)nc(=O)n2-c1ccccc1Cl] | 6-bormo-1-(2-chlorophenyl)-7-(1,1- difluoroethyl)-4-(((1S,2R)-2- fluorocyclopropyl)amino)quinazolin- 2(1H)-one | m/z [M + H]⁺ 472.0/474.0 |
| 678 | 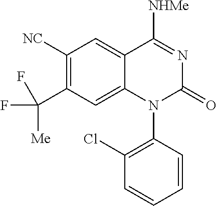 [SMILES: CNc1nc(=O)n(-c2ccccc2Cl)c2cc(C(C)(F)F)c(C#N)cc12] | 1-(2-chlorophenyl)-7-(1,1- difluoroethyl)-4-(methylamino)-2-oxo- 1,2-dihydro-quinazoline-6- carbonitrile | m/z [M + H]⁺ 375.0 |
| 679 | 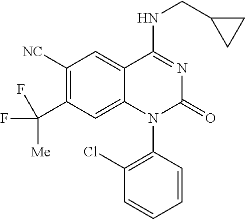 [SMILES: CC(F)(F)c1cc2c(cc1C#N)c(NCC1CC1)nc(=O)n2-c1ccccc1Cl] | 1-(2-chlorophenyl)-4-((cyclopropyl- methyl)amino)-7-(1,1-difluoroethyl)- 2-oxo-1,2-dihydroquinazoline-6- carbonitrile | m/z [M + H]⁺ 415.0 |
| 680 | 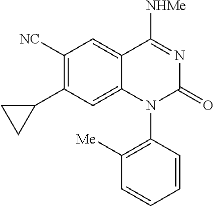 [SMILES: CNc1nc(=O)n(-c2ccccc2C)c2cc(C3CC3)c(C#N)cc12] | 7-cyclopropyl-4-(methylamino)-2- oxo-1-(o-tolyl)-1,2- dihydroquinazoline-6-carbonitrile | m/z [M + H]⁺ 331.2 |
| 681 | 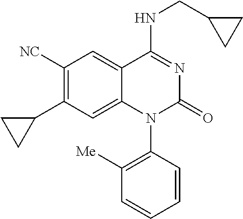 [SMILES: Cc1ccccc1-n1c(=O)nc(NCC2CC2)c2cc(C#N)c(C3CC3)cc21] | 7-cyclopropyl-4- ((cyclopropylmethyl)-amino)-2-oxo-1- (o-tolyl)-1,2-dihydro-quinazoline-6- carbonitrile | m/z [M + H]⁺ 371.2 |
| 682 | 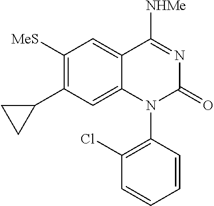 [SMILES: CNc1nc(=O)n(-c2ccccc2Cl)c2cc(C3CC3)c(SC)cc12] | 1-(2-chlorophenyl)-7-cyclopropyl-4- (methylamino)-6-(methylthio)- quinazolin-2(1H)-one | m/z [M + H]⁺ 372.0 |
| 683 | 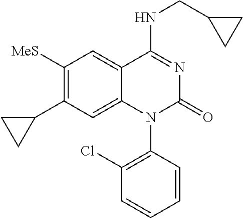 [SMILES: CSc1cc2c(NCC3CC3)nc(=O)n(-c3ccccc3Cl)c2cc1C1CC1] | 1-(2-chlorophenyl)-7-cyclopropyl-4- ((cyclopropylmethyl)amino)-6- (methylthio)quinazolin-2(1H)-one | m/z [M + H]⁺ 412.0 |
| 684 | 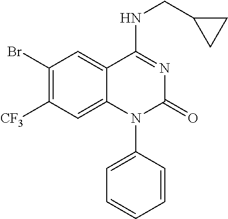 [SMILES: O=c1nc(NCC2CC2)c2cc(Br)c(C(F)(F)F)cc2n1-c1ccccc1] | 6-bromo-4- ((cyclopropylmethyl)amino)-1- phenyl-7-(trifluoromethyl)quinazolin- 2(1H)-one | m/z [M + H]⁺ 438.0/440.0 |
| 685 | 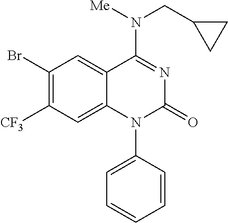 [SMILES: CN(CC1CC1)c1nc(=O)n(-c2ccccc2)c2cc(C(F)(F)F)c(Br)cc12] | 6-bromo-4-((cyclopropylmethyl)- (methylamino)-1-phenyl-7- (trifluoromethyl)-quinazolin-2(1H)- one | m/z [M + H]⁺ 452.0/454.0 |
| 686 | 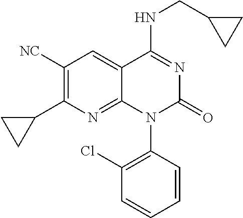 [SMILES: N#Cc1cc2c(NCC3CC3)nc(=O)n(-c3ccccc3Cl)c2nc1C1CC1] | 1-(2-chlorophenyl)-7-cyclopropyl-4- ((cyclopropylmethyl)amino)-2-oxo- 1,2-dihydropyrido[2,3-d]pyrimidine-6- carbonitrile | m/z [M + H]⁺ 392.1 |
| 687 | 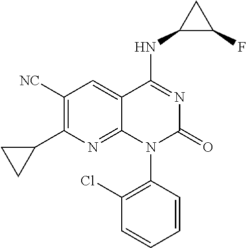 [SMILES: N#Cc1cc2c(N[C@H]3C[C@H]3F)nc(=O)n(-c3ccccc3Cl)c2nc1C1CC1] | 1-(2-chlorophenyl)-7-cyclopropyl-4- (((1S,2R)-2- fluorocyclopropyl)amino)-2-oxo-1,2- dihydropyrido[2,3-d]-pyrimidine-6- carbonitriel | m/z [M + H]⁺ 396.0 |
| 688 | 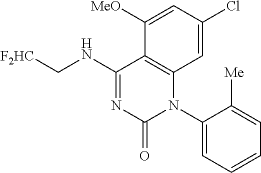 [SMILES: COc1cc(Cl)cc2c1c(NCC(F)F)nc(=O)n2-c1ccccc1C] | 7-chloro-4-((2,2- difluoroethyl)amino)-5-methoxy-1-(o- tolyl)quinazolin-2(1H)-one | m/z [M + H]⁺ 380.35 |
| 689 | 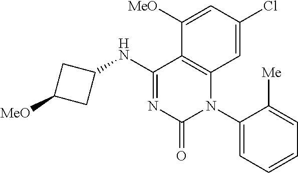 | 7-chloro-5-fluoro-4-(((1r,3r)-3- methoxy-cyclobutyl)amino)-1-(o- tolyl)quinazolin-2(1H)-one | m/z [M + H]⁺ 400.33 |
| 690 | 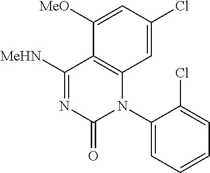 [SMILES: CNc1nc(=O)n(-c2ccccc2Cl)c2cc(Cl)cc(OC)c12] | 7-chloro-1-(2-chlorophenyl)-5- methoxy-4-(methylamino)quinazolin- 2(1H)-one | m/z [M + H]⁺ 350.2 |
| 691 | 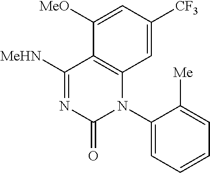 [SMILES: CNc1nc(=O)n(-c2ccccc2C)c2cc(C(F)(F)F)cc(OC)c12] | 5-methoxy-4-(methylamino)-1-(o- tolyl)-7-(trifluoromethyl)quinazolin- 2(1H)-one | m/z [M + H]⁺ 364.19 |
| 692 | 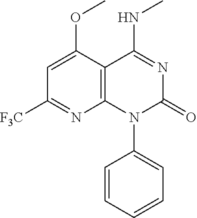 [SMILES: CNc1nc(=O)n(-c2ccccc2)c2nc(C(F)(F)F)cc(OC)c12] | 5-methoxy-4-(methylamino)-1- phenyl-7-(trifluoromethyl)pyrido[2,3- d]pyrimidin-2(1H)-one | m/z [M + H]⁺ 351.3 |
| 693 | 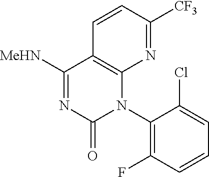 [SMILES: CNc1nc(=O)n(-c2c(F)cccc2Cl)c2nc(C(F)(F)F)ccc12] | 1-(2-chloro-6-fluorophenyl)-4- (methylamino)-7- (trifluoromethyl)pyrido[2,3-d]- pyrimidin-2(1H)-one | m/z [M + H]⁺ 373.2 |
| 694 | 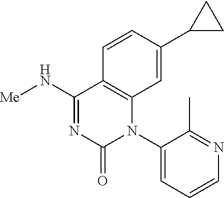 [SMILES: CNc1nc(=O)n(-c2cccnc2C)c2cc(C3CC3)ccc12] | 7-cyclopropyl-4-(methylamino)-1-(2- methylpyridin-3-yl)quinazolin-2(1H) one | m/z [M + H]⁺ 307.22 |
| 695 | 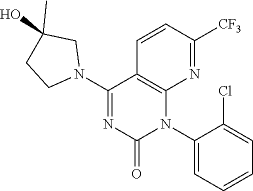 [SMILES: C[C@@]1(O)CCN(c2nc(=O)n(-c3ccccc3Cl)c3nc(C(F)(F)F)ccc23)C1] | 1-(2-chlorophenyl)-4-(3-hydroxy-3- methylpyrrolidin-1-yl)-7-(trifluoro- methyl)pyrido[2,3-d]pyrimidin-2(1H)- one, single unknown enantiomer | m/z [M + H]⁺ 425.1 |
| 696 | 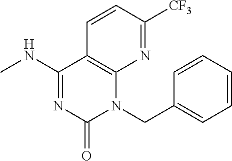 [SMILES: CNc1nc(=O)n(Cc2ccccc2)c2nc(C(F)(F)F)ccc12] | 1-benzyl-4-(methylamino)-7- (trifluoro-methyl)pyrido[2,3- d]pyrimidin-2(1H)-one | m/z [M + H]⁺ 335.3 |
| 697 | 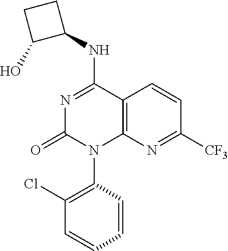 | 1-(2-chlorophenyl)-4-(((trans)-2- hydroxycyclobutyl)amino)-7- (trifluro-methyl)-pyrido[2,3- d]pyrimidin-2(1H)-one, single unknown enantiomer/atropisomer | m/z [M + H]⁺ 411.3 |
| 698 | 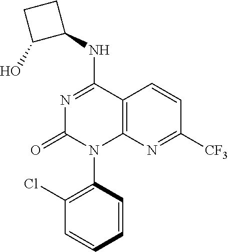 | 1-(2-chlorophenyl)-4-(((trans)-2- hydroxycyclobutyl)amino)-7- (trifluoro-methyl)-pyrido[2,3- d]pyrimidin-2(1H)-one, single unknown enantiomer/atropisomer | m/z [M + H]⁺ 411.3 |
| 699 | 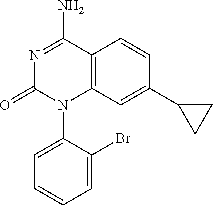 [SMILES: Nc1nc(=O)n(-c2ccccc2Br)c2cc(C3CC3)ccc12] | 4-amino-1-(2-bromophenyl)-7- cyclopropylquinazolin-2(1H)-one | m/z [M + H]⁺ 356.1 |
| 700 | 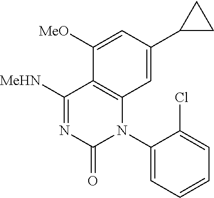 [SMILES: CNc1nc(=O)n(-c2ccccc2Cl)c2cc(C3CC3)cc(OC)c12] | 1-(2-chlorophenyl)-7-cylcopropyl-5- methoxy-(methylamino)quinazolin- 2(1H)-one | m/z [M + H]⁺ 356.35 |
| 701 | 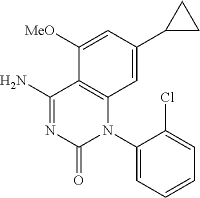 [SMILES: COc1cc(C2CC2)cc2c1c(N)nc(=O)n2-c1ccccc1Cl] | 4-amino-1-(2-chlorophenyl)-7- cyclopropyl-5-methoxyquinazolin- 2(1H)-one | m/z [M + H]⁺ 342.31 |
| 702 | 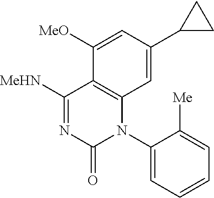 [SMILES: CNc1nc(=O)n(-c2ccccc2C)c2cc(C3CC3)cc(OC)c12] | 7-cyclopropyl-5-methoxy-4- (methylamino)-1-(o-tolyl)quinazolin- 2(1H)-one | m/z [M + H]⁺ 366.44 |
| 703 | 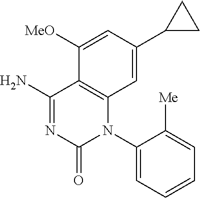 [SMILES: COc1cc(C2CC2)cc2c1c(N)nc(=O)n2-c1ccccc1C] | 4-amino-7-cyclopropyl-5-methoxy-1- (o-tolyl)quinazolin-2(1H)-one | m/z [M + H]⁺ 322.36 |
| 704 | 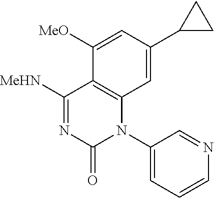 [SMILES: CNc1nc(=O)n(-c2cccnc2)c2cc(C3CC3)cc(OC)c12] | 7-cyclopropyl-5-methoxy-4-(methyl- amino)-1-(pyridin-3-yl)quinazolin- 2(1H)-one | m/z [M + H]⁺ 323.36 |
| 705 | 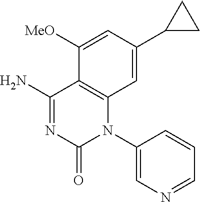 [SMILES: COc1cc(C2CC2)cc2c1c(N)nc(=O)n2-c1cccnc1] | 4-amino-7-cyclopropyl-5-methoxy-1- (pyridin-3-yl)quinazolin-2(1H)-one | m/z [M + H]⁺ 309.36 |
| 706 | 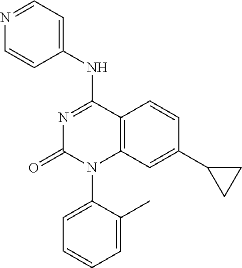 [SMILES: Cc1ccccc1-n1c(=O)nc(Nc2ccncc2)c2ccc(C3CC3)cc21] | 7-cyclopropyl-4-(pyridin-4-ylamino)- 1-(o-tolyl)quinazolin-2(1H)-one | m/z [M + H]⁺ 369.39 |
| 707 | 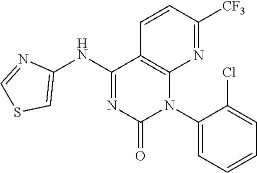 [SMILES: O=c1nc(Nc2cscn2)c2ccc(C(F)(F)F)nc2n1-c1ccccc1Cl] | 1-(2-chlorophenyl)-4-(thiazol-4- ylamino)-7-(trifluoromethyl) pyrido[2,3-d]-pyrimidin-2(1H)-one | m/z [M + H]⁺ 424.2 |
| 708 | 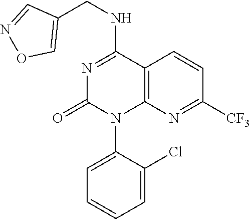 [SMILES: O=c1nc(NCc2cnoc2)c2ccc(C(F)(F)F)nc2n1-c1ccccc1Cl] | 1-(2-chlorophenyl)-4-((isoxazol-4-yl- methyl)amino)-7-(trifluoromethyl) pyrido[2,3-d]pyrimidin-2(1H)-one | m/z [M + H]⁺ 422.3 |
| 709 | 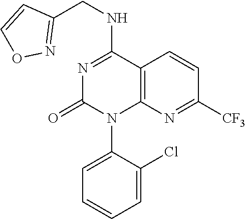 [SMILES: O=c1nc(NCc2ccon2)c2ccc(C(F)(F)F)nc2n1-c1ccccc1Cl] | 1-(2-chlorophenyl)-4-((isoxazol-3-yl- methyl)amino)-7-(trifluoromethyl) pyrido[2,3-d]pyrimidin-2(1H)-one | m/z [M + H]⁺ 422.3 |
| 710 | 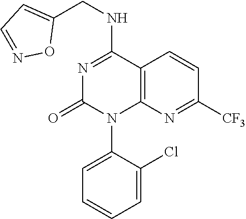 [SMILES: O=c1nc(NCc2ccno2)c2ccc(C(F)(F)F)nc2n1-c1ccccc1Cl] | 1-(2-chlorophenyl)-4-((isoxazol-5-yl- methyl)amino)-7-(trifluoromethyl) pyrido[2,3-d]pyrimidin-2(1H)-one | m/z [M + H]⁺ 422.2 |
| 711 | 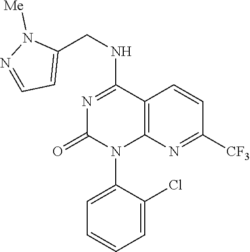 [SMILES: Cn1nccc1CNc1nc(=O)n(-c2ccccc2Cl)c2nc(C(F)(F)F)ccc12] | 1-(2-chlorophenyl)-4-(((1-methyl- 1H-pyrazol-5-yl)methyl)amino)-7- (trifluoro-methyl)pyrido[2,3- d]pyrimidin-2(1H)-one | m/z [M + H]⁺: 435.27 |
| 712 | 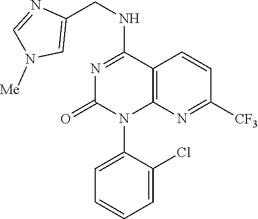 [SMILES: Cn1cnc(CNc2nc(=O)n(-c3ccccc3Cl)c3nc(C(F)(F)F)ccc23)c1] | 2-chlorophenyl)-4-(((1-methyl-1H- imidazol-4-yl)methyl)amino)-7- (trifluoro-methyl)-pyrido[2,3-d]- pyrimidin-2(1H)-one | m/z [M + H]⁺ 435.27 |
| 713 | 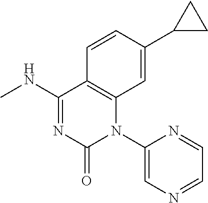 [SMILES: CNc1nc(=O)n(-c2cnccn2)c2cc(C3CC3)ccc12] | cyclopropyl-4-(methylamino)-1- (pyrazin-2-yl)quinazolin-2(1H)-one | m/z [M + H]⁺ 294.4 |
| 714 | 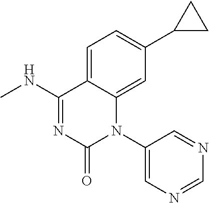 [SMILES: CNc1nc(=O)n(-c2cncnc2)c2cc(C3CC3)ccc12] | 7-cyclopropyl-4-(methylamino)-1- (pyrimidin-5-yl)quinazolin-2(1H)-one | m/z [M + H]⁺ 294.33 |
| 715 | 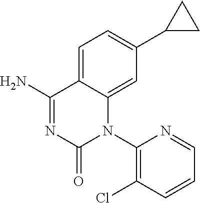 [SMILES: Nc1nc(=O)n(-c2ncccc2Cl)c2cc(C3CC3)ccc12] | 4-amino-1-(3-chloropyridin-2-yl)-7- cyclopropylquinazolin-2(1H)-one | m/z [M + H]⁺ 313.3 |
| 716 | 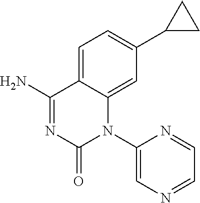 [SMILES: Nc1nc(=O)n(-c2cnccn2)c2cc(C3CC3)ccc12] | 4-amino-7-cyclopropyl-1-(pyrazin-2- yl)-quinazolin-2(1H)-one | m/z [M + H]⁺ 280.4 |
| 717 | 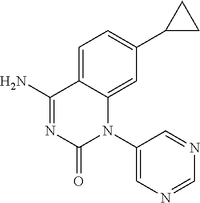 [SMILES: Nc1nc(=O)n(-c2cncnc2)c2cc(C3CC3)ccc12] | 4-amino-7-cyclopropyl-1-(pyrimidin- 5-yl)quinazolin-2(1H)-one | m/z [M + H]⁺ 280.26 |
| 718 | 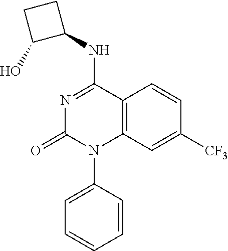 [SMILES: O=c1nc(N[C@@H]2CC[C@H]2O)c2ccc(C(F)(F)F)cc2n1-c1ccccc1] | 4-((trans-2-hydroxycyclobutyl) amino)-1-phenyl-7-(trifluoromethyl) quinazolin-2(1H)-one | m/z [M + H]⁺ 376.36 |
| 719 | 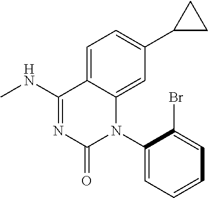 | 1-(2-bromophenyl)-7-cyclopropyl-4- (methylamino)quinazolin-2(1H)-one, a single atropisomer | m/z [M + H] 370.3 |
| 720 | 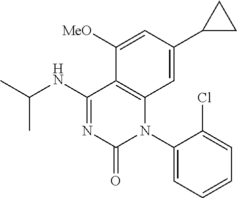 [SMILES: COc1cc(C2CC2)cc2c1c(NC(C)C)nc(=O)n2-c1ccccc1Cl] | 1-(2-chlorophenyl)-7-cyclopropyl-4- (isopropylamino)-5-methoxy- quinazolin-2(1H)-one | m/z [M + H]⁺ 384.4 |
| 721 | 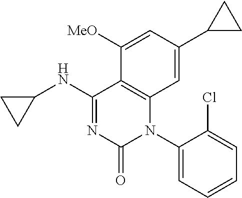 [SMILES: COc1cc(C2CC2)cc2c1c(NC1CC1)nc(=O)n2-c1ccccc1Cl] | 1-(2-chlorophenyl)-7-cyclopropyl-4- (cyclopropylamino)-5-methoxy- quinazolin-2(1H)-one | m/z [M + H]⁺ 382.35 |
| 722 | 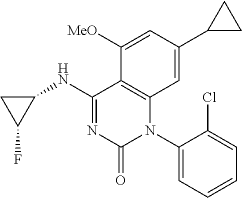 [SMILES: COc1cc(C2CC2)cc2c1c(N[C@H]1C[C@H]1F)nc(=O)n2-c1ccccc1Cl] | 1-(2-chlorophenyl)-7-cyclopropyl-4- (((1S,2R)-2-fluorocyclopropyl) amino)-5-methoxyquinazolin-2(1H)- one | m/z [M + H]⁺ 400.33 |
| 723 | 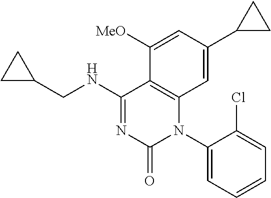 [SMILES: COc1cc(C2CC2)cc2c1c(NCC1CC1)nc(=O)n2-c1ccccc1Cl] | 1-(2-chlorophenyl)-7-cyclopropyl-4- ((cyclopropylmethyl)amino)-5- methoxy-quinazolin-2(1H)-one | m/z [M + H]⁺ 396.4 |
| 724 | 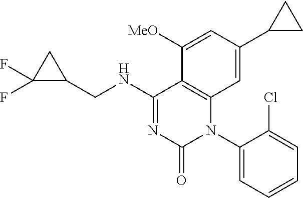 [SMILES: COc1cc(C2CC2)cc2c1c(NCC1CC1(F)F)nc(=O)n2-c1ccccc1Cl] | 1-(2-chlorophenyl)-7-cyclopropyl-4- (((2,2-difluorocyclopropyl)methyl)- amino)-5-methoxyquinazolin-2(1H)- one | m/z [M + H]⁺ 432.4 |
| 725 | 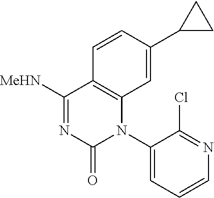 [SMILES: CNc1nc(=O)n(-c2cccnc2Cl)c2cc(C3CC3)ccc12] | 1-(2-chloropyridin-3-yl)-7- cyclopropyl-4- (methylamino)quinazolin-2(1H)-one | m/z [M + H]⁺ 327.3 |
| 726 | 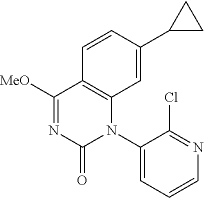 [SMILES: COc1nc(=O)n(-c2cccnc2Cl)c2cc(C3CC3)ccc12] | 1-(2-chloropyridin-3-yl)-7- cyclopropyl-4-methoxyquinazolin- 2(1H)-one | m/z [M + H]⁺ 328.3 |
| 727 | 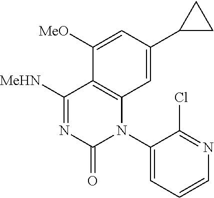 [SMILES: CNc1nc(=O)n(-c2cccnc2Cl)c2cc(C3CC3)cc(OC)c12] | 1-(2-chloropyridin-3-yl)-7- cyclopropyl-5-methoxy-4- (methylamino)quinazolin-2(1H)-one | m/z [M + H]⁺ 357.37 |
| 728 | 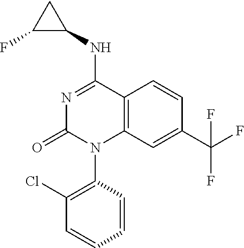 | 1-(2-chlorophenyl)-4-(((1S,2S)-2- fluorocyclopropyl)amino)-7- (trifluoromethyl)-quinazolin-2(1H)- one, single unknown enantiomer/atropisomer | m/z [M + H]⁺ 398.3 |
| 729 | 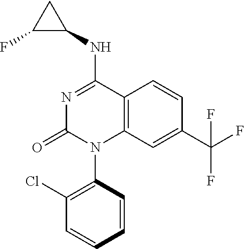 | 1-(2-chlorophenyl)-4-(((1S,2S)-2- fluorocyclopropyl)amino)-7- (trifluoromethyl)-quinazolin-2(1H)- one, single unknown enantiomer/atropisomer | m/z [M + H]⁺ 398.3 |
| 730 | 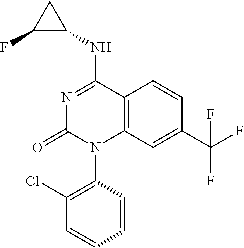 | 1-(2-chlorophenyl)-4-(((1R,2R)-2- fluorocyclopropyl)amino)-7- (trifluoromethyl)-quinazolin-2(1H)- one, single unknown enantiomer/atropisomer | m/z [M + H]⁺ 398.3 |
| 731 | 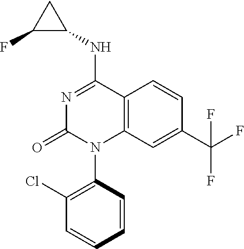 | 1-(2-chlorophenyl)-4-(((1R,2R)-2- fluorocyclopropyl)amino)-7- (trifluoromethyl)-quinazolin-2(1H)- one, single unknwon enantiomer/atropisomer | m/z [M + H]⁺ 398.3 |
| 732 | 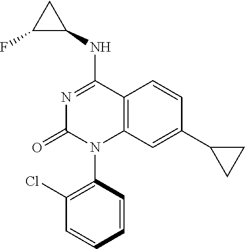 | 1-(2-chlorophenyl)-7-cyclopropyl-4- (((1S,2S)-2- fluorocyclopropyl)amino)-quinazolin- 2(1H)-one, single unknown enantiomer/atropisomer | m/z [M + H]⁺ 370.34 |
| 733 | 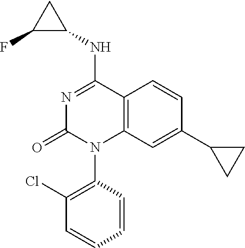 | 1-(2-chlorophenyl)-7-cyclopropyl-4- (((1S,2S)-2- fluorocyclopropyl)amino)-quinazolin- 2(1H)-one, single unknown enantiomer/atropisomer | m/z [M + H]⁺ 370.34 |
| 734 | 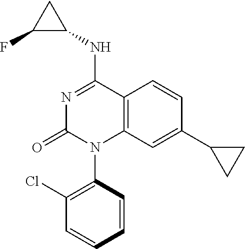 | 1-(2-chlorophenyl)-7-cyclopropyl-4- (((1S,2S)-2- fluorocylcopropyl)amino)-quinazolin- 2(1H)-one, single unknown enantiomer/atropisomer | m/z [M + H]⁺ 370.34 |
| 735 | 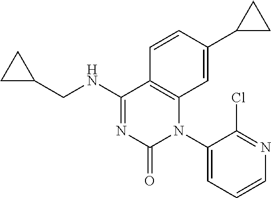 [SMILES: O=c1nc(NCC2CC2)c2ccc(C3CC3)cc2n1-c1cccnc1Cl] | 1-(2-chloropyridin-3-yl)-7- cyclopropyl-4- ((cyclopropylmethyl)amino)- quinazolin-2(1H)-one | m/z [M + H]⁺ 367.4 |
| 736 | 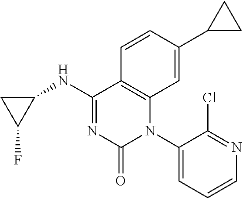 [SMILES: O=c1nc(N[C@H]2C[C@H]2F)c2ccc(C3CC3)cc2n1-c1cccnc1Cl] | 1-(2-chloropyridin-3-yl)-7- cyclopropyl-4-(((1S,2R)-2- fluorocyclopropyl)amino)-quinazolin- 2(1H)-one | m/z [M + H]⁺ 371.3 |
| 737 | 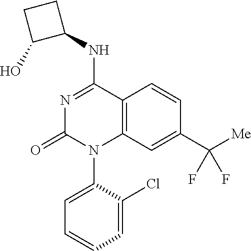 | 1-(2-chlorophenyl)-7-(1,1- difluoroethyl)-4-(((1R,2R)-2- hydroxycyclobutyl)-amino)quinazolin- 2(1H)-one, single unknown enantiomer/atropisomer | m/z [M + H]⁺ 406.37 |
| 738 | 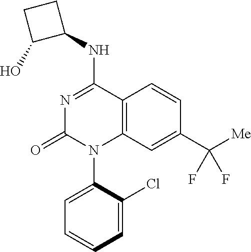 | 1-(2-chlorophenyl)-7-(1,1- difluoroethyl)-4-(((1R,2R)-2- hydroxycyclobutyl)-amino)quinazolin- 2(1H)-one, single unknwon enantiomer/atropisomer | m/z [M + H]⁺ 406.37 |
| 739 | 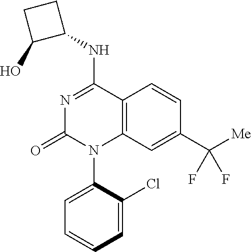 | 1-(2-chlorophenyl)-7-(1,1- difluoroethyl)-4-(((1S,2S)-2- hydroxycyclobutyl)-amino)quinazolin- 2(1H)-one, single unknown enantiomer/atropisomer | m/z [M + H]⁺ 406.37 |
| 740 | 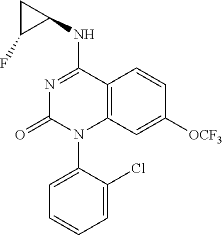 [SMILES: O=c1nc(N[C@@H]2C[C@H]2F)c2ccc(OC(F)(F)F)cc2n1-c1ccccc1Cl] | 1-(2-chlorophenyl)-4-(((1R,2R)-2- fluorocyclopropyl)amino)-7- (trifluoromethoxy)quinazolin-2(1H)- one, single unknown enantiomer | m/z [M + H]⁺ 414.3 |
| 741 | 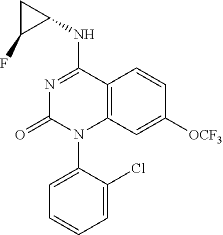 [SMILES: O=c1nc(N[C@H]2C[C@@H]2F)c2ccc(OC(F)(F)F)cc2n1-c1ccccc1Cl] | 1-(2-chlorophenyl)-4-(((1S,2S)-2- fluorocyclopropyl)amino)-7- (trifluoromethoxy)quinazolin-2(1H)- one, single unknown enantioemr | m/z [M + H]⁺ 414.3 |
| 742 | 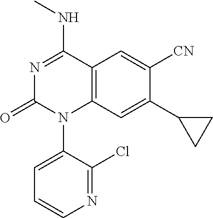 [SMILES: CNc1nc(=O)n(-c2cccnc2Cl)c2cc(C3CC3)c(C#N)cc12] | 1-(2-chloropyridin-3-yl)-7- cyclopropyl-4-(methylamino)-2-oxo- 1,2-dihydro-quinazoline-6- carbonitrile | m/z [M + H]⁺ 352.4 |
| 743 | 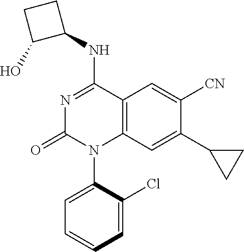 | 1-(2-chlorophenyl)-7-cyclopropyl-4- (((1S,2S)-2- hydroxycyclobutyl)amino)-2-oxo-1,2- dihydroquinazoline-6-carbonitrile, single unknown enantiomer/atropisomer | m/z [M + H]⁺ 407.47 |
| 744 | 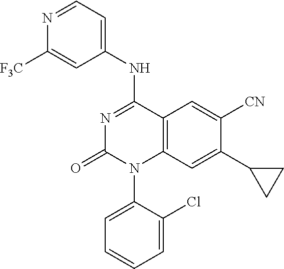 [SMILES: N#Cc1cc2c(Nc3ccnc(C(F)(F)F)c3)nc(=O)n(-c3ccccc3Cl)c2cc1C1CC1] | 1-(2-chlorophenyl)-7-cyclopropyl-2- oxo-4-((2-(trifluoromethyl)pyridin-4- yl)-amino)-1,2-dihydroquinazoline-6- carbonitrile | m/z [M + H]⁺ 482.5 |
| 745 | 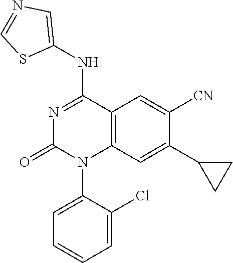 [SMILES: N#Cc1cc2c(Nc3cncs3)nc(=O)n(-c3ccccc3Cl)c2cc1C1CC1] | 1-(2-chlorophenyl)-7-cyclopropyl-2- oxo-4-(thiazol-5-ylamino)-1,2- dihydro-quinazoline-6-carbonitrile | m/z [M + H]⁺ 420.1 |
| 746 | 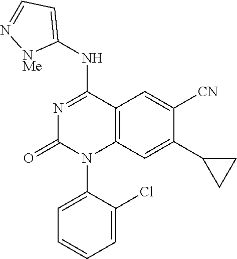 [SMILES: Cn1nccc1Nc1nc(=O)n(-c2ccccc2Cl)c2cc(C3CC3)c(C#N)cc12] | 1-(2-chlorophenyl)-7-cyclopropyl-4- ((1-methyl-1H-pyrazol-5-yl)amino)-2- oxo-1,2-dihydroquinazoline-6- carbonitrile | m/z [M + H]⁺ 417.36 |
| 747 | 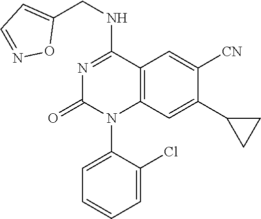 [SMILES: N#Cc1cc2c(NCc3ccno3)nc(=O)n(-c3ccccc3Cl)c2cc1C1CC1] | 1-(2-chlorophenyl)-7-cyclopropyl-4- ((isoxazol-5-ylmethyl)amino)-2-oxo- 1,2-dihydroquinazoline-6-carbonitrile | m/z [M + H]⁺ 418.35 |
| 748 | 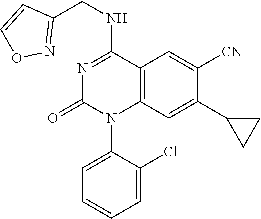 [SMILES: N#Cc1cc2c(NCc3ccon3)nc(=O)n(-c3ccccc3Cl)c2cc1C1CC1] | 1-(2-chlorophenyl)-7-cyclopropyl-4- ((isoxazol-3-ylmethyl)amino)-2-oxo- 1,2-dihydroquinazoline-6-carbonitrile | m/z [M + H]⁺ 418.3 |
| 749 | 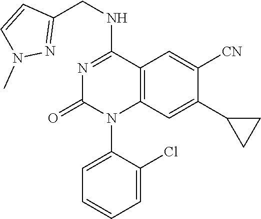 [SMILES: Cn1ccc(CNc2nc(=O)n(-c3ccccc3Cl)c3cc(C4CC4)c(C#N)cc23)n1] | 1-(2-chlorophenyl)-7-cyclopropyl-4- (((1-methyl-1H-pyrazol-3- yl)methyl)amino)-2-oxo-1,2- dihydroquinazoline-6-carbonitrile | m/z [M + H]⁺ 431.38 |
| 750 | 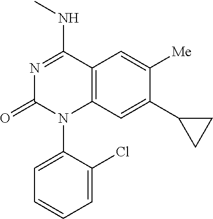 [SMILES: CNc1nc(=O)n(-c2ccccc2Cl)c2cc(C3CC3)c(C)cc12] | 1-(2-chlorophenyl)-7-cyclopropyl-6- methyl-4-(methylamino)quinazolin- 2(1H)-one | m/z [M + H]⁺ 340.34 |
| 751 | 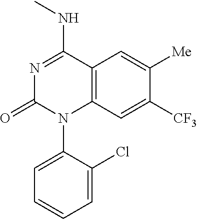 [SMILES: CNc1nc(=O)n(-c2ccccc2Cl)c2cc(C(F)(F)F)c(C)cc12] | 1-(2-chlorophenyl)-6-methyl-4- (methylamino)-7-(trifluoromethyl)- quinazolin-2(1H)-one | m/z [M + H]⁺ 368.4 |
| 752 | 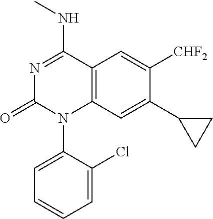 [SMILES: CNc1nc(=O)n(-c2ccccc2Cl)c2cc(C3CC3)c(C(F)F)cc12] | 1-(2-chlorophenyl)-7-cyclopropyl-6- (difluoromethyl)-4-(methylamino)- quinazolin-2(1H)-one | m/z [M + H]⁺ 376.4 |
| 753 | 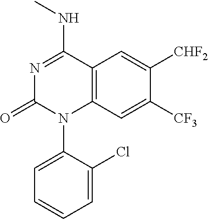 [SMILES: CNc1nc(=O)n(-c2ccccc2Cl)c2cc(C(F)(F)F)c(C(F)F)cc12] | 1-(2-chlorophenyl)-6- (difluoromethyl)-4-(methylamino)-7- (trifluoromethyl)-quinazolin-2(1H)- one | m/z [M + H]⁺ 404.4 |
| 754 | 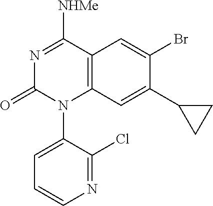 [SMILES: CNc1nc(=O)n(-c2cccnc2Cl)c2cc(C3CC3)c(Br)cc12] | 6-bromo-1-(2-chloropyridin-3-yl)-7- cyclopropyl-4- (methylamino)quinazolin-2(1H)-one | m/z [M + H]⁺ 405.2 |
| 755 | 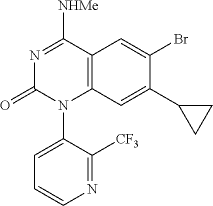 [SMILES: CNc1nc(=O)n(-c2cccnc2C(F)(F)F)c2cc(C3CC3)c(Br)cc12] | 6-bromo-7-cyclopropyl-4-(methyl- amino)-1-(2-(trifluoromethyl)pyridin- 3-yl)-quinazolin-2(1H)-one | m/z [M + H]⁺ 439.4 |
| 756 | 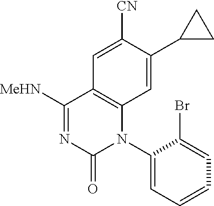 | 1-(2-bromophenyl)-7-cyclopropyl-4- (methylamino)-2-oxo-1,2-dihydro- quinazoline-6-carbonitrile, single unknown atropisomer | m/z [M + H]⁺ 395.41 |
| 757 | 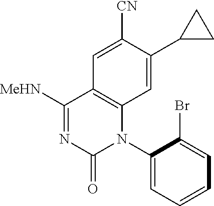 | 1-(2-bromophenyl)-7-cyclopropyl-4- (methylamino)-2-oxo-1,2-dihydro- quinazoline-6-carbonitrile, single unknown atropisomer | m/z [M + H]⁺ 395.41 |
| 758 | 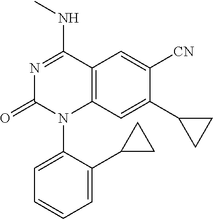 [SMILES: CNc1nc(=O)n(-c2ccccc2C2CC2)c2cc(C3CC3)c(C#N)cc12] | 7-cyclopropyl-1-(2- cyclopropylphenyl)-4-(methylamino)- 2-oxo-1,2-dihydro-quinazoline-6- carbonitrile | m/z [M + H]⁺ 357.40 |
| 759 | 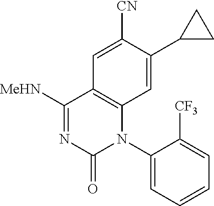 [SMILES: CNc1nc(=O)n(-c2ccccc2C(F)(F)F)c2cc(C3CC3)c(C#N)cc12] | 7-cyclopropyl-4-(methylamino)-2- oxo-1-(2-(trifluoromethyl)phenyl)-1,2- dihydro-quinazoline-6-carbonitrile | m/z [M + H]⁺ 385.44 |
| 760 | 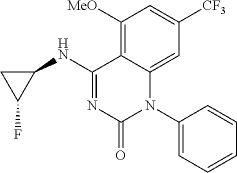 [SMILES: COc1cc(C(F)(F)F)cc2c1c(N[C@@H]1C[C@H]1F)nc(=O)n2-c1ccccc1] | 4-(((1R,2R)-2-fluorocyclopropyl)- amino)-5-methoxy-1-phenyl-7- (trifluoromethyl)quinazolin-2(1H)- one, single unknown enantiomer | m/z [M + H]⁺ 394.4 |
| 761 | 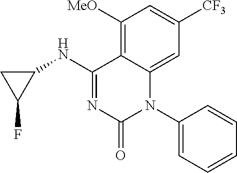 | 4-(((1S,2S)-2- fluorocyclopropyl)amino)-5-methoxy- 1-phenyl-7- (trifluoromethyl)quinazolin-2(1H)- one, single unknown enantiomer | m/z [M + H]⁺ 394.4 |
| 762 | 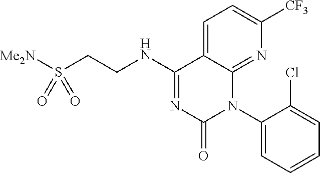 [SMILES: CN(C)S(=O)(=O)CCNc1nc(=O)n(-c2ccccc2Cl)c2nc(C(F)(F)F)ccc12] | 2-((1-(2-chlorophenyl)-2-oxo-7- (trifluoromethyl)-1,2- dihydropyrido[2,3-d]-pyrimidin-4- yl)amino)-N,N- dimethylethanesulfonamide | m/z [M + H]⁺ 454.19 |
| 763 | 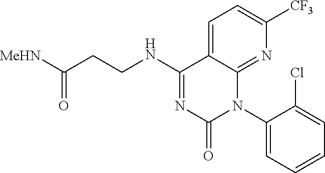 [SMILES: CNC(=O)CCNc1nc(=O)n(-c2ccccc2Cl)c2nc(C(F)(F)F)ccc12] | 3-((1-(2-chlorophenyl)-2-oxo-7- (trifluoromethyl)-1,2- dihydropyrido[2,3-d]-pyrimidin-4- yl)amino)-N-methylpropanamide | m/z [M + H]⁺ 426.4 |
| 764 | 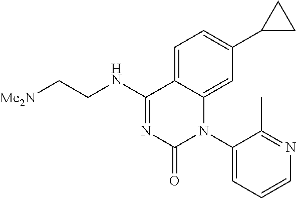 [SMILES: Cc1ncccc1-n1c(=O)nc(NCCN(C)C)c2ccc(C3CC3)cc21] | 7-cyclopropyl-4-((2-(dimethylamino)- ethyl)amino)-1-(2-methylpyridin-3-yl)- quinazolin-2(1H)-one | m/z [M − H]⁺ 362.52 |
| 765 | 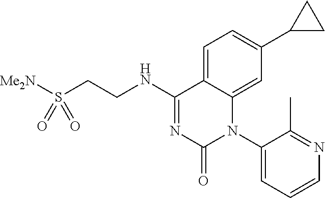 [SMILES: Cc1ncccc1-n1c(=O)nc(NCCS(=O)(=O)N(C)C)c2ccc(C3CC3)cc21] | 2-((7-cyclopropyl-1-(2-methylpyridin- 3-yl)-2-oxo-1,2-dihydroquinazolin-4- yl)-amino)-N,N-dimethylethane-1- sulfonamide | m/z [M + H]⁺ 428.49 |
| 766 | 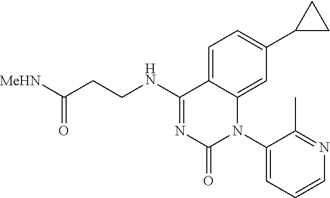 [SMILES: CNC(=O)CCNc1nc(=O)n(-c2cccnc2C)c2cc(C3CC3)ccc12] | 3-((7-cyclopropyl-1-(2-methylpyridin- 3-yl)-2-oxo-1,2-dihydroquinazolin-4- yl)-amino)-N-methylpropanamide | m/z [M + H]⁺ 378.47 |
| 767 | 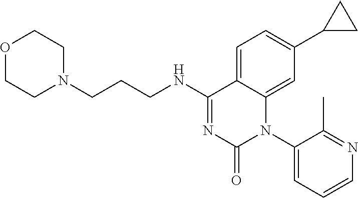 [SMILES: Cc1ncccc1-n1c(=O)nc(NCCCN2CCOCC2)c2ccc(C3CC3)cc21] | 7-cyclopropyl-1-(2-methylpyridin-3- yl)-4-((3-morpholinopropyl)amino)- quinazolin-2(1H)-one | m/z [M + H]⁺ 420.31 |
| 768 | 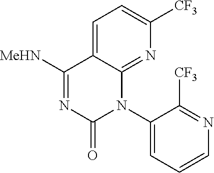 [SMILES: CNc1nc(=O)n(-c2cccnc2C(F)(F)F)c2nc(C(F)(F)F)ccc12] | 4-(methylamino)-7-(trifluoromethyl)- 1-(2-(trifluoromethyl)pyridin-3- yl)pyrido[2,3-d]-pyrimidin-2(1H)-one | m/z [M + H]⁺ 390.4 |
| 769 | 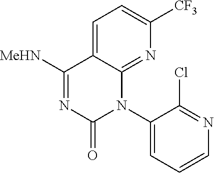 [SMILES: CNc1nc(=O)n(-c2cccnc2Cl)c2nc(C(F)(F)F)ccc12] | 1-(2-chloropyridin-3-yl)-4- (methylamino)-7-(trifluoromethyl)- pyrido[2,3-d]-pyrimidin-2(1H)-one | m/z [M + H]⁺ 356.3 |
| 770 | 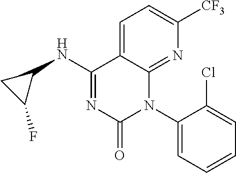 | 1-(2-chlorophenyl)-4-(((1S,2R)-2- fluorocyclopropyl)amino)-7- (trifluoromethyl)pyrido[2,3- d]pyrimidin-2(1H)-one, single unknown enantiomer | m/z [M + H]⁺ 399.4. |
| 771 | 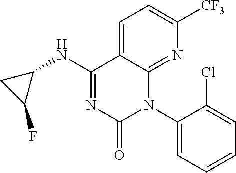 [SMILES: O=c1nc(N[C@H]2C[C@@H]2F)c2ccc(C(F)(F)F)nc2n1-c1ccccc1Cl] | 1-(2-chlorophenyl)-4-(((1R,2S)-2- fluorocyclopropyl)amino)-7- (trifluoromethyl)pyrido[2,3- d]pyrimidin-2(1H)-one, single unknown enantiomer | m/z [M + H]⁺ 399.4 |
| 772 | 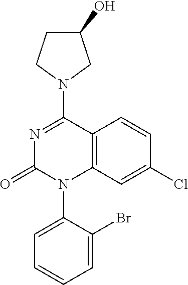 [SMILES: O=c1nc(N2CC[C@@H](O)C2)c2ccc(Cl)cc2n1-c1ccccc1Br] | (R)-1-(2-bromophenyl)-7-chloro-4-(3- hydroxypyrrolidin-1-yl)quinazolin- 2(1H)-one | m/z [M + H]⁺ 420.0, 422.0 |
| 773 | 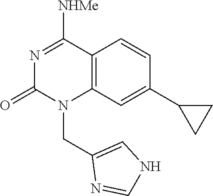 [SMILES: CNc1nc(=O)n(Cc2c[nH]cn2)c2cc(C3CC3)ccc12] | 1-((1H-imidazol-4-yl)methyl)-7- cyclopropyl-4-(methylamino) quinazolin-2(1H)-one | m/z [M + H]⁺ 294.4 |
| 774 | 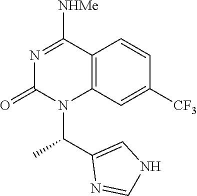 [SMILES: CNc1nc(=O)n([C@@H](C)c2c[nH]cn2)c2cc(C(F)(F)F)ccc12] | (S)-1-(1-(1H-imidazol-4-yl)ethyl)-4- (methylamino)-7-(trifluoromethyl)- quinazolin-2(1H)-one, Single unknown enantiomer | m/z [M + H]⁺ 338.3. |
| 775 | 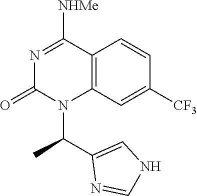 [SMILES: CNc1nc(=O)n([C@H](C)c2c[nH]cn2)c2cc(C(F)(F)F)ccc12] | (R)-1-(1-(1H-imidazol-4-yl)ethyl)-4- (methylamino)-7- (trifluoromethyl)quinazolin-2(1H)- one, single unknown enantiomer | m/z [M + H] 338.3 |
| 776 | 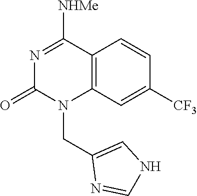 [SMILES: CNc1nc(=O)n(Cc2c[nH]cn2)c2cc(C(F)(F)F)ccc12] | 1-(2-chlorophenyl)-7-cyclopropyl-4- (methylamino)-6-(methylthio)- quinazolin-2(1H)-one | m/z [M + H]⁺ 324.35 |

In another embodiment, methionine adenosyltransferase II alpha (MAT2A) inhibitor is Compound A:

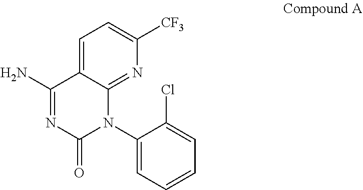 [SMILES: Nc1nc(=O)n(-c2ccccc2Cl)c2nc(C(F)(F)F)ccc12]

or a pharmaceutically acceptable salt thereof. Compound A is also referred to as compound 167 in Table 1.

The methionine adenosyltransferase II alpha (MAT2A) inhibitors described herein, their syntheses, and biological activity against MAT2A can be found in PCT/US2019/065260 (WO2020123395), which is incorporated by reference in its entirety.

In another embodiment, the Type I PRMT inhibitor is a protein arginine methyltransferase 1 (PRMT1) inhibitor, a protein arginine methyltransferase 3 (PRMT3) inhibitor, a protein arginine methyltransferase 4 (PRMT4) inhibitor, a protein arginine methyltransferase 6 (PRMT6) inhibitor, or a protein arginine methyltransferase 8 (PRMT8) inhibitor.

In still another embodiment, the Type I PRMT inhibitor is a protein arginine methyltransferase 1 (PRMT1) inhibitor.

In an embodiment, the Type I PRMT inhibitor is a compound of Formula II:

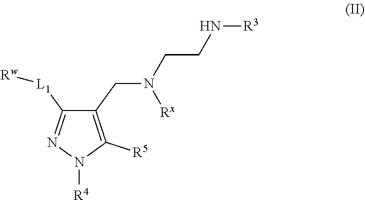

or a pharmaceutically acceptable salt thereof;

wherein
R^{x} is optionally substituted C₁₋₄ alkyl or optionally substituted C₃₋₄ cycloalkyl;
L₁ is a bond, —O—, —N(R^{B})—, —S—, —C(O)—, —C(O)O—, —C(O)S—, —C(O)N(R^{B})—, —C(O)N(R^{B})N(R^{B})—, —OC(O)—, —OC(O)N(R^{B})—, —NR^{B}C(O)—, —NR^{B}C(O)N(R^{B})—, —NR^{B}C(O)N(R^{B})N(R^{B})—, —NR^{B}C(O)O—, —SC(O)—, —C(═NR^{B})—, —C(═NNB^{b})—, —C(═NOR^{A})—, —C(═NR^{B})N(R^{B})—NR^{B}C(═NR^{B})—, —CCS)—, —C(S)N(R^{B})—, —NR^{B}C(S)—, —SCO)—, —OS(O)₂—, —S(O)₂O—, —SO₂—, —N(R^{B})SO₂—, —SO₂N(R^{B})—, or an optionally substituted C₁₋₆ saturated or unsaturated hydrocarbon chain, wherein one or more methylene units of the hydrocarbon chain is optionally and independently replaced with —O—, —N(R^{B})—, —S—, —C(O)—, —C(O)O—, —C—(O)S—, —C(O)N(R^{B})—C(O)N(R^{B})N(R^{B})—, —OC(O)—, —OC(O)N(R^{B})—, —NR^{B}C(O)—, —NR^{B}C(O)N(R^{B})—, —NR^{B}C(O)N(R^{B})N(R^{B})—, —NR^{B}C(O)O—, —SC(O)—, —C(═NR^{B})—, —C(═NNR^{B})—, —C(═NOR^{A})—, —C(═NR^{B})N(R^{B})—, —NR^{B}C(═NR^{B})—, —CCS)—, —C(S)N(R^{B})—, —NR^{B}C(S)—, —S(O)—, —OS(O)₂—, —S(O)₂O—, —SO2- —N(R^{B})SO₂—, or —SO₂N(R^{B})—;
each R^{A} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, and a sulfur protecting group when attached to a sulfur atom;
each R^{B} is independently selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, and a nitrogen protecting group, or an R^{B} and R^{w} on the same nitrogen atom may be taken together with the intervening nitrogen to form an optionally substituted heterocyclic ring;
R^{w} is hydrogen, optionally substituted alkyl, optionally substituted alkenyl, optionally substituted alkynyl, optionally substituted carbocyclyl, optionally substituted heterocyclyl, optionally substituted aryl, or optionally substituted heteroaryl; provided that when Li is a bond, R^{w} is not hydrogen, optionally substituted aryl, or optionally substituted heteroaryl;
R³ is hydrogen, C₁₋₄ alkyl, or C₃₋₄ cycloalkyl;
R⁴ is hydrogen, optionally substituted C₁₋₆ alkyl, optionally substituted C₂₋₆ alkenyl, optionally substituted C₂₋₆ alkynyl, optionally substituted C₃₋₇ cycloalkyl, optionally substituted 4- to 7-membered heterocyclyl; or optionally substituted C₁₋₄ alkyl-Cy;
Cy is optionally substituted C₃₋₇ cycloalkyl, optionally substituted 4- to 7-membered heterocyclyl, optionally substituted aryl, or optionally substituted heteroaryl; and
R⁵ is hydrogen, halo, —CN, optionally substituted C₁₋₄ alkyl, or optionally substituted C3-4 cycloalkyl.

In another embodiment, the Type I PRMT inhibitor is selected from the group consisting of a compound from Table 2. The compounds of Table 2 can be found in Table 1A of WO 2014/153226, which is incorporated by reference in its entirety.

TABLE 2

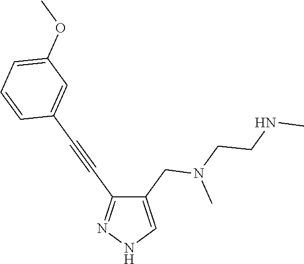 [SMILES: CNCCN(C)Cc1c[nH]nc1C#Cc1cccc(OC)c1]

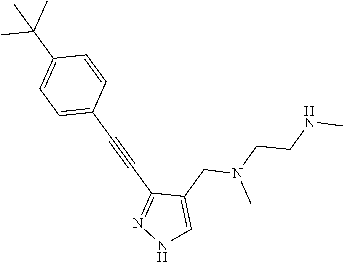 [SMILES: CNCCN(C)Cc1c[nH]nc1C#Cc1ccc(C(C)(C)C)cc1]

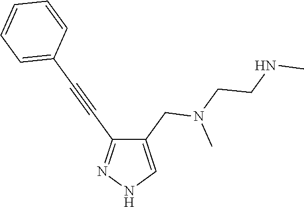 [SMILES: CNCCN(C)Cc1c[nH]nc1C#Cc1ccccc1]

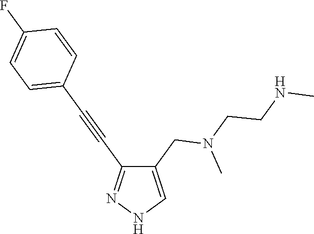 [SMILES: CNCCN(C)Cc1c[nH]nc1C#Cc1ccc(F)cc1]

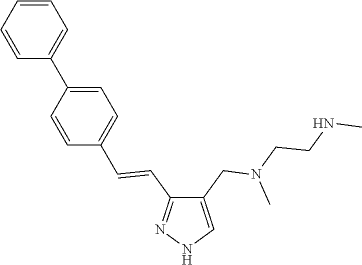 [SMILES: CNCCN(C)Cc1c[nH]nc1/C=C/c1ccc(-c2ccccc2)cc1]

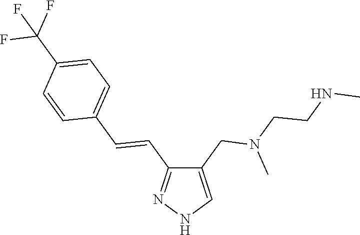 [SMILES: CNCCN(C)Cc1c[nH]nc1/C=C/c1ccc(C(F)(F)F)cc1]

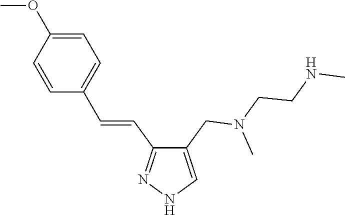

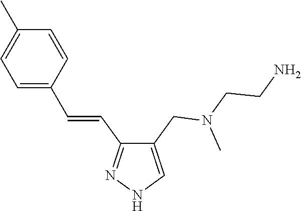 [SMILES: Cc1ccc(/C=C/c2n[nH]cc2CN(C)CCN)cc1]

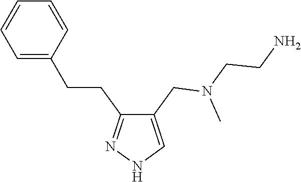 [SMILES: CN(CCN)Cc1c[nH]nc1CCc1ccccc1]

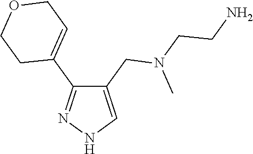 [SMILES: CN(CCN)Cc1c[nH]nc1C1=CCOCC1]

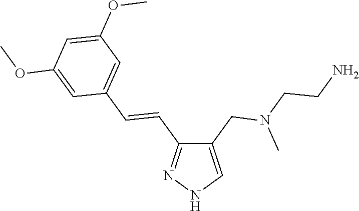 [SMILES: COc1cc(/C=C/c2n[nH]cc2CN(C)CCN)cc(OC)c1]

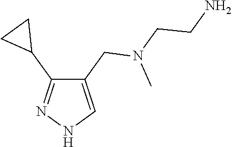

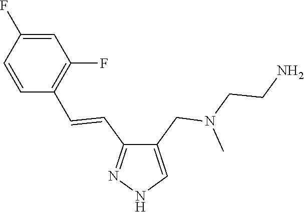 [SMILES: CN(CCN)Cc1c[nH]nc1/C=C/c1ccc(F)cc1F]

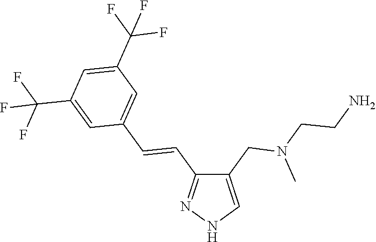 [SMILES: CN(CCN)Cc1c[nH]nc1/C=C/c1cc(C(F)(F)F)cc(C(F)(F)F)c1]

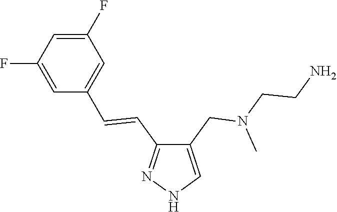 [SMILES: CN(CCN)Cc1c[nH]nc1/C=C/c1cc(F)cc(F)c1]

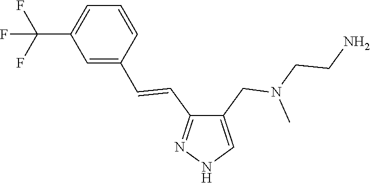 [SMILES: CN(CCN)Cc1c[nH]nc1/C=C/c1cccc(C(F)(F)F)c1]

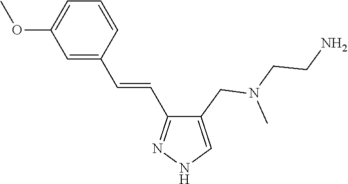 [SMILES: COc1cccc(/C=C/c2n[nH]cc2CN(C)CCN)c1]

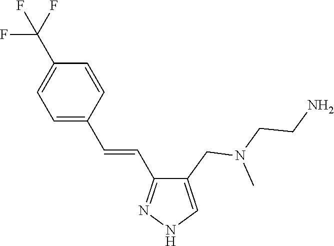 [SMILES: CN(CCN)Cc1c[nH]nc1/C=C/c1ccc(C(F)(F)F)cc1]

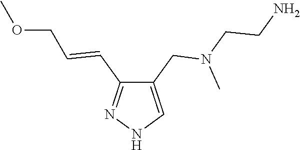 [SMILES: COC/C=C/c1n[nH]cc1CN(C)CCN]

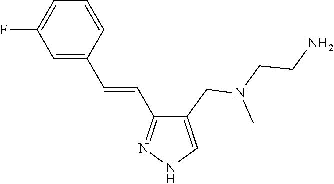 [SMILES: CN(CCN)Cc1c[nH]nc1/C=C/c1cccc(F)c1]

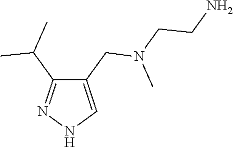

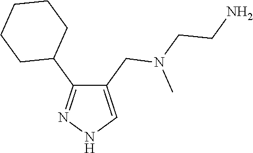 [SMILES: CN(CCN)Cc1c[nH]nc1C1CCCCC1]

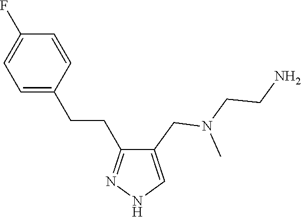 [SMILES: CN(CCN)Cc1c[nH]nc1CCc1ccc(F)cc1]

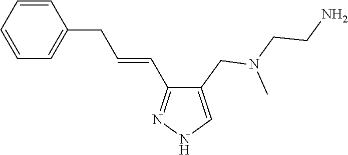 [SMILES: CN(CCN)Cc1c[nH]nc1/C=C/Cc1ccccc1]

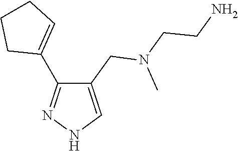 [SMILES: CN(CCN)Cc1c[nH]nc1C1=CCCC1]

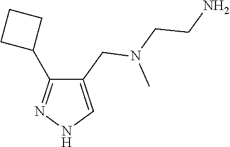

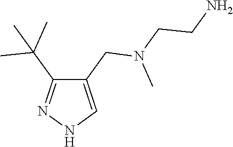

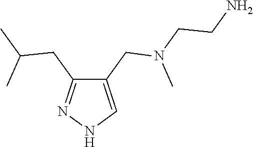

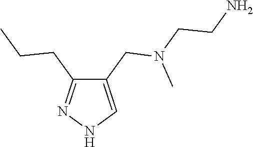

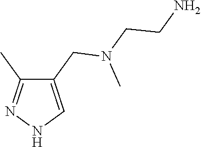

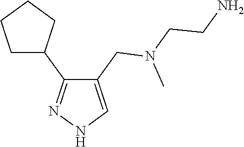 [SMILES: CN(CCN)Cc1c[nH]nc1C1CCCC1]

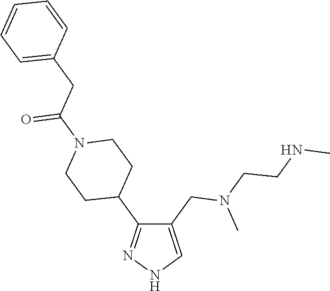 [SMILES: CNCCN(C)Cc1c[nH]nc1C1CCN(C(=O)Cc2ccccc2)CC1]

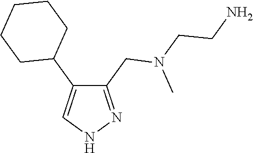 [SMILES: CN(CCN)Cc1n[nH]cc1C1CCCCC1]

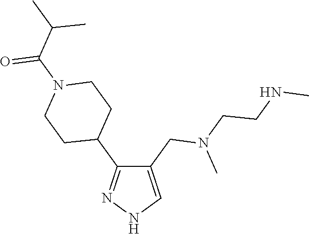 [SMILES: CNCCN(C)Cc1c[nH]nc1C1CCN(C(=O)C(C)C)CC1]

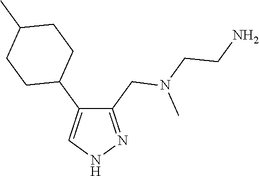 [SMILES: CC1CCC(c2c[nH]nc2CN(C)CCN)CC1]

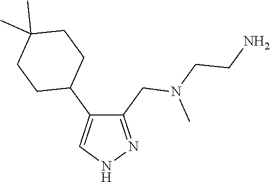 [SMILES: CN(CCN)Cc1n[nH]cc1C1CCC(C)(C)CC1]

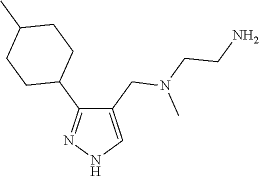 [SMILES: CC1CCC(c2n[nH]cc2CN(C)CCN)CC1]

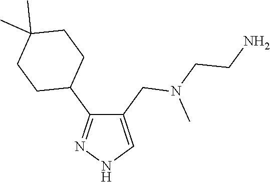 [SMILES: CN(CCN)Cc1c[nH]nc1C1CCC(C)(C)CC1]

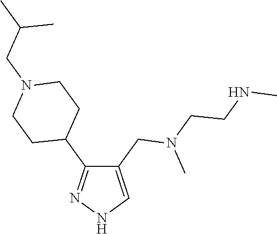 [SMILES: CNCCN(C)Cc1c[nH]nc1C1CCN(CC(C)C)CC1]

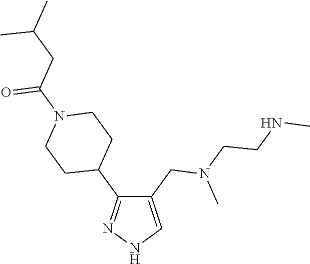 [SMILES: CNCCN(C)Cc1c[nH]nc1C1CCN(C(=O)CC(C)C)CC1]

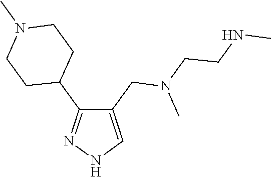

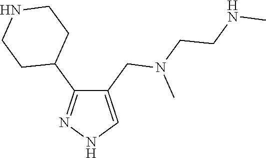 [SMILES: CNCCN(C)Cc1c[nH]nc1C1CCNCC1]

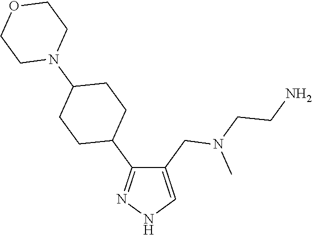 [SMILES: CN(CCN)Cc1c[nH]nc1C1CCC(N2CCOCC2)CC1]

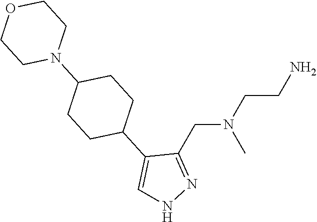 [SMILES: CN(CCN)Cc1n[nH]cc1C1CCC(N2CCOCC2)CC1]

or a pharmaceutically acceptable salt thereof.

In yet another embodiment, the Type I PRMT inhibitor is Compound B:

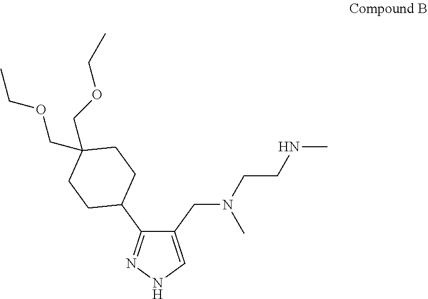 [SMILES: CCOCC1(COCC)CCC(c2n[nH]cc2CN(C)CCNC)CC1]

or a pharmaceutically acceptable salt thereof.

In still another embodiment, the PRMT inhibitor is selected from a compound disclosed in PCT/US2014/02710 (WO2014153226) or PCT/EP2020/071460 (WO2021023609), the entire contents of which are hereby incorporated by reference in their entireties.

### Combination Product

Provided herein is a combination product comprising a MAT2A inhibitor, or a pharmaceutically acceptable salt thereof, and a Type I PRMT inhibitor, or a pharmaceutically acceptable salt thereof. The combination product is useful for the treatment of a variety of cancers, including solid tumors. In another aspect, the combination product is useful for the treatment of any number of MAT2A-associated diseases. In another aspect, the combination product is useful for the treatment of a disease or disorder treatable by inhibiting MAT2A. In another aspect, the combination product is useful for the treating MTAP-deficient tumors. In another aspect, the combination product is useful for the treatment of any number of Type I PRMT-associated diseases.

In an aspect, provided herein is a combination product comprising a MAT2A inhibitor that is a compound of Formula I:

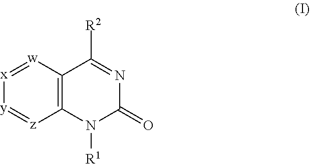

or a pharmaceutically acceptable salt thereof, and a Type I PRMT inhibitor that is a compound of Formula II:

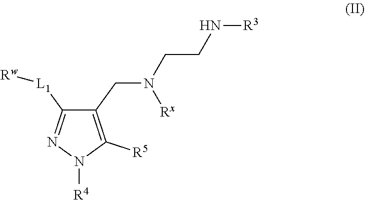

or a pharmaceutically acceptable salt thereof.

In another aspect, provided herein is a combination product comprising a MAT2A inhibitor that is a compound of Formula I, and a Type I PRMT inhibitor.

In another aspect, provided herein is a Type I PRMT inhibitor that is a compound of Formula II or a pharmaceutically acceptable salt thereof, and a MAT2A inhibitor.

In another aspect, provided herein is a combination product comprising a methionine adenosyltransferase II alpha (MAT2A) inhibitor that is Compound A:

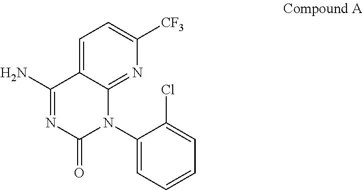 [SMILES: Nc1nc(=O)n(-c2ccccc2Cl)c2nc(C(F)(F)F)ccc12]

or a pharmaceutically acceptable salt thereof,

and a Type I protein arginine methyltransferase (Type I PRMT) inhibitor that is

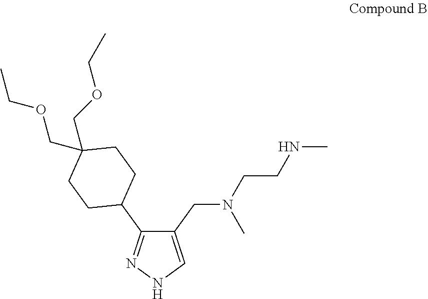 [SMILES: CCOCC1(COCC)CCC(c2n[nH]cc2CN(C)CCNC)CC1]

or a pharmaceutically acceptable salt thereof.

In yet another aspect, provided herein is a combination product comprising a first pharmaceutical composition comprising a therapeutically effective amount of a methionine adenosyltransferase II alpha (MAT2A) inhibitor and a second pharmaceutical composition comprising a therapeutically effective amount of a Type I protein arginine methyltransferase (Type I PRMT) inhibitor. In an embodiment, the MAT2A inhibitor is a compound of Formula I. In another embodiment, the Type I PRMT inhibitor is a compound of Formula II.

In another aspect, provided herein is a combination product comprising a first pharmaceutical composition comprising a therapeutically effective amount of a MAT2A inhibitor that is a compound of Formula I or a pharmaceutically acceptable salt thereof, and a second pharmaceutical composition comprising a therapeutically effective amount of a Type I PRMT inhibitor.

In another aspect, provided herein is a combination product comprising a first pharmaceutical composition comprising a therapeutically effective amount of a Type I PRMT inhibitor that is a compound of Formula II or a pharmaceutically acceptable salt thereof, and a second pharmaceutical composition comprising a therapeutically effective amount of a MAT2A inhibitor.

In an aspect, provided herein is a combination product comprising a first pharmaceutical composition comprising a therapeutically effective amount of a MAT2A inhibitor that is a compound of Formula I or a pharmaceutically acceptable salt thereof, and a second pharmaceutical composition comprising a therapeutically effective amount of a Type I PRMT inhibitor compound of Formula II or a pharmaceutically acceptable salt thereof.

In still another aspect, provided herein is a combination product comprising a first pharmaceutical composition comprising a therapeutically effective amount of Compound A, or a pharmaceutically acceptable salt thereof; and a second pharmaceutical composition comprising a therapeutically effective amount of Compound B, or a pharmaceutically acceptable salt thereof.

The administration of a pharmaceutical combination provided herein may result in a beneficial effect, e.g. a synergistic therapeutic effect, e.g., with regard to alleviating, delaying progression of or inhibiting the symptoms, and may also result in further surprising beneficial effects, e.g., fewer side-effects, an improved quality of life or a decreased morbidity, compared with a monotherapy applying only one of the pharmaceutically active ingredients used in the combination of the invention.

### Methods of Treatment

In an aspect, provided herein is a method of treating cancer in a subject in need thereof, the method comprising administering to the subject an effective amount of a methionine adenosyltransferase II alpha (MAT2A) inhibitor and administering to the subject an effective amount of Type I protein arginine methyltransferase (Type I PRMT) inhibitor.

In an embodiment, provided herein are methods of treating cancer in a subject in need thereof, the methods comprising administering to the subject a combination comprising a MAT2A inhibitor and a Type I PRMT inhibitor, together with at least a pharmaceutically acceptable carrier, thereby treating the cancer in the subject.

In an embodiment, provided herein are methods of treating cancer in a subject in need thereof, the methods comprising administering to the subject a therapeutically effective amount of a pharmaceutical composition comprising a MAT2A inhibitor and a therapeutically effective amount of a pharmaceutical composition comprising a Type I PRMT inhibitor, thereby treating the cancer in the subject.

In an embodiment, use of a combination of a MAT2A inhibitor and a Type I PRMT inhibitor for the manufacture of a medicament is provided. In one embodiment, the MAT2A inhibitor is a compound of Formula I, or a pharmaceutically acceptable salt thereof. In one embodiment, the MAT2A inhibitor is Compound A or a pharmaceutically acceptable salt thereof. In an embodiment, the Type I PRMT inhibitor is a compound of Formula II or a pharmaceutically acceptable salt thereof. In an embodiment, the Type I PRMT inhibitor is Compound B or a pharmaceutically acceptable salt thereof. In an embodiment, provided is a combination of a compound of Formula I or a pharmaceutically acceptable salt thereof, and a compound of Formula II or a pharmaceutically acceptable salt thereof, for the manufacture of a medicament. In one embodiment, provided is a combination of Compound A or a pharmaceutically acceptable salt thereof, and Compound B or a pharmaceutically acceptable salt thereof, for the manufacture of a medicament.

In another embodiment, use of a combination of a MAT2A inhibitor and a Type I PRMT inhibitor for the treatment of cancer is provided. In one embodiment, the MAT2A inhibitor is a compound of Formula I or a pharmaceutically acceptable salt thereof. In one embodiment, the MAT2A inhibitor is Compound A or a pharmaceutically acceptable salt thereof. In one embodiment, the Type I PRMT inhibitor is a compound of Formula II or a pharmaceutically acceptable salt thereof. In an embodiment, the Type I PRMT inhibitor is Compound B or a pharmaceutically acceptable salt thereof. In one embodiment, provided is use of a combination of a compound of Formula I or a pharmaceutically acceptable salt thereof, and a compound of Formula II or a pharmaceutically acceptable salt thereof, for the treatment of cancer. In one embodiment, provided is a use of a combination of Compound A or a pharmaceutically acceptable salt thereof, and Compound B or a pharmaceutically acceptable salt thereof, for the treatment of cancer.

In an embodiment, the MAT2A inhibitor is a compound of Formula I:

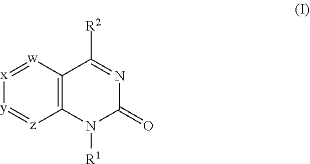

or a pharmaceutically acceptable salt thereof; wherein the variables are defined herein.

In another embodiment, the Type I PRMT inhibitor is a compound of Formula II:

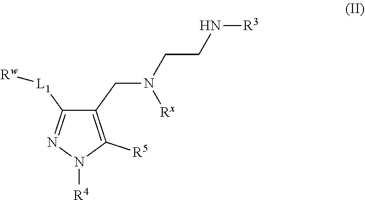

or a pharmaceutically acceptable salt thereof; wherein the variables are defined herein.

In yet another embodiment, the MAT2A inhibitor is Compound A:

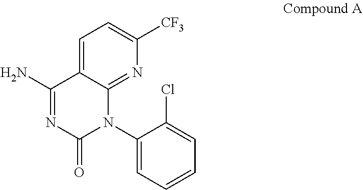 [SMILES: Nc1nc(=O)n(-c2ccccc2Cl)c2nc(C(F)(F)F)ccc12]

or a pharmaceutically acceptable salt thereof.

In still another embodiment, the PRMT1 inhibitor is Compound B:

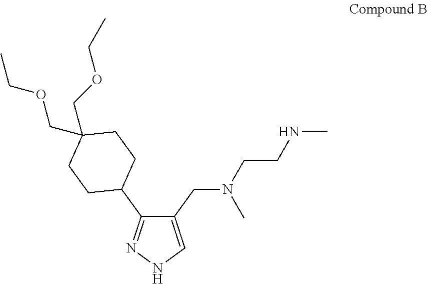 [SMILES: CCOCC1(COCC)CCC(c2n[nH]cc2CN(C)CCNC)CC1]

or a pharmaceutically acceptable salt thereof.

In an aspect, provided herein is a method of treating cancer in a subject in need thereof comprising administering to the subject a therapeutically effective amount of a MAT2A inhibitor that is a compound of Formula I, or a pharmaceutically acceptable salt thereof; and a therapeutically effective amount of a Type I PRMT inhibitor.

In an aspect, provided herein is a method of treating cancer in a subject in need thereof comprising administering to the subject a therapeutically effective amount of a Type I PRMT inhibitor that is a compound of Formula II or a pharmaceutically acceptable salt thereof, and a MAT2A inhibitor.

In an aspect, provided herein is a method of treating cancer in a subject in need thereof comprising administering to the subject a therapeutically effective amount of a MAT2A inhibitor that is a compound of Formula I, or a pharmaceutically acceptable salt thereof; and a therapeutically effective amount of a Type I PRMT inhibitor that is a compound of Formula II or a pharmaceutically acceptable salt thereof.

In an aspect, provided herein is a method of treating cancer in a subject in need thereof comprising administering to the subject a therapeutically effective amount of Compound A, or a pharmaceutically acceptable salt thereof; and a therapeutically effective amount of Compound B, or a pharmaceutically acceptable salt thereof.

In another aspect, provided herein is a method of treating cancer comprising administering to a subject in need thereof a first pharmaceutical composition comprising a therapeutically effective amount of a MAT2A inhibitor that is a compound of Formula I, or a pharmaceutically acceptable salt thereof, and a second pharmaceutical composition comprising a therapeutically effective amount of a Type I PRMT inhibitor.

In another aspect, provided herein is a method of treating cancer comprising administering to a subject in need thereof a first pharmaceutical composition comprising a therapeutically effective amount of a Type I PRMT inhibitor that is a compound of Formula II or a pharmaceutically acceptable salt thereof, and a second pharmaceutical composition comprising a therapeutically effective amount of a MAT2A inhibitor.

In another aspect, provided herein is a method of treating cancer comprising administering to a subject in need thereof a first pharmaceutical composition comprising a therapeutically effective amount of a MAT2A inhibitor that is a compound of Formula I, or a pharmaceutically acceptable salt thereof, and a second pharmaceutical composition comprising a therapeutically effective amount of a Type I PRMT inhibitor that is a compound of Formula II or a pharmaceutically acceptable salt thereof.

In another aspect, provided herein is a method of treating cancer comprising administering to a subject in need thereof a first pharmaceutical composition comprising a therapeutically effective amount of Compound A, or a pharmaceutically acceptable salt thereof; and a second pharmaceutical composition comprising a therapeutically effective amount of Compound B, or a pharmaceutically acceptable salt thereof.

In an embodiment, the cancer is characterized by a reduction or absence of MTAP gene expression, the absence of the MTAP gene, reduced level of MTAP protein, MTA accumulation, reduced function of MTAP protein, absence of MTAP protein, or a combination thereof. In another embodiment, the cancer is characterized as MTAP-null.

In yet another embodiment, the cancer is selected from the group consisting of leukemia, glioma, melanoma, pancreatic, non-small cell lung cancer, bladder cancer, astrocytoma, osteosarcoma, head and neck cancer, myxoid chondrosarcoma, ovarian cancer, endometrial cancer, breast cancer, soft tissue sarcoma, non-Hodgkin lymphoma, and mesothelioma.

In still another embodiment, the cancer is a solid tumor.

In another embodiment, the Type I PRMT inhibitor is a protein arginine methyltransferase 1 (PRMT1) inhibitor, a protein arginine methyltransferase 3 (PRMT3) inhibitor, a protein arginine methyltransferase 4 (PRMT4) inhibitor, a protein arginine methyltransferase 6 (PRMT6) inhibitor, or a protein arginine methyltransferase 8 (PRMT8) inhibitor.

In an embodiment, the methionine adenosyltransferase II alpha (MAT2A) inhibitor and PRMT Type I inhibitor are in separate dosage forms.

In another embodiment, the methionine adenosyltransferase II alpha (MAT2A) inhibitor and PRMT Type I inhibitor are in the same dosage form.

In another embodiment, the treatment comprises administering the methionine adenosyltransferase II alpha (MAT2A) inhibitor, or a pharmaceutically acceptable salt thereof, and PRMT Type I inhibitor, or a pharmaceutically acceptable salt thereof, at substantially the same time. In yet another embodiment, the treatment comprises administering methionine adenosyltransferase II alpha (MAT2A) inhibitor, or a pharmaceutically acceptable salt thereof, and PRMT Type I inhibitor, or a pharmaceutically acceptable salt thereof, at different times.

In still another embodiment, the methionine adenosyltransferase II alpha (MAT2A) inhibitor, or a pharmaceutically acceptable salt thereof, is administered to the subject, followed by administration of the PRMT Type I inhibitor, or a pharmaceutically acceptable salt thereof. In an embodiment, the PRMT Type I inhibitor, or a pharmaceutically acceptable salt thereof, is administered to the subject, followed by administration of methionine adenosyltransferase II alpha (MAT2A) inhibitor, or a pharmaceutically acceptable salt thereof.

In yet another embodiment, the method comprises administering to the subject in need thereof a methionine adenosyltransferase II alpha (MAT2A) inhibitor.

In still another embodiment, the method comprises administering to the subject in need thereof Type I protein arginine methyltransferase (Type I PRMT) inhibitor.

In an embodiment, the methionine adenosyltransferase II alpha (MAT2A) inhibitor, or a pharmaceutically acceptable salt thereof, and the PRMT Type I inhibitor, or a pharmaceutically acceptable salt thereof, are administered orally.

In another embodiment, the cancer to be treated is selected from the group consisting of lung cancer, colon and rectal cancer, breast cancer, prostate cancer, liver cancer, pancreatic cancer, brain cancer, kidney cancer, ovarian cancer, stomach cancer, skin cancer, bone cancer, gastric cancer, glioma, glioblastoma, neuroblastoma, hepatocellular carcinoma, papillary renal carcinoma, head and neck squamous cell carcinoma, leukemia, lymphomas, myelomas, retinoblastoma, cervical cancer, melanoma and/or skin cancer, bladder cancer, uterine cancer, testicular cancer, esophageal cancer, and solid tumors. In some embodiments, the cancer is lung cancer, colon cancer, breast cancer, neuroblastoma, leukemia, and lymphomas. In other embodiments, the cancer is lung cancer, colon cancer, breast cancer, neuroblastoma, leukemia, or lymphoma. In a further embodiment, the cancer is non-small cell lung cancer (NSCLC) or small cell lung cancer.

In an embodiment, the cancer is a hematologic cancer, such as leukemia or lymphoma. In a certain embodiment, lymphoma is Hodgkin's lymphoma or Non-Hodgkin's lymphoma. In certain embodiments, leukemia is myeloid, lymphocytic, myelocytic, lymphoblastic, or megakaryotic leukemia.

In yet another embodiment, the cancer is selected from the group consisting of leukemia, glioma, melanoma, pancreatic, non-small cell lung cancer, bladder cancer, astrocytoma, osteosarcoma, head and neck cancer, myxoid chondrosarcoma, ovarian cancer, endometrial cancer, breast cancer, anal cancer, stomach cancer, colon cancer, colorectal cancer, soft tissue sarcoma, non-Hodgkin lymphoma, gastric cancer, esophagogastric cancer, esophageal cancer, malignant peripheral nerve sheath tumor, and mesothelioma.

In an embodiment, the cancer is mesothelioma. In another embodiment, the cancer is nonsquamous non-small cell lung cancer. In one embodiment, the cancer is cancer of the colon or rectum. In an embodiment, the cancer is adenocarcinoma of the colon or rectum. In an embodiment, the cancer is breast cancer. In an embodiment, the cancer is adenocarcinoma of the breast. In an embodiment, the cancer is gastric cancer. In an embodiment, the cancer is gastric adenocarcinoma. In an embodiment, the cancer is pancreatic cancer. In an embodiment, the cancer is pancreatic adenocarcinoma. In an embodiment, the cancer is bladder cancer.

In an embodiment, the cancer is characterized as being MTAP-null. In an embodiment, the cancer is characterized as being MTAP-deficient.

In still another embodiment, the cancer is a solid tumor. In still another embodiment, the cancer is a MTAP-deleted solid tumor. n still another embodiment, the cancer is a metastatic MTAP-deleted solid tumor. In still another embodiment, the cancer is metastatic. In still another embodiment, the cancer is a solid malignant tumor. In still another embodiment, the cancer is MTAP-deficient lung or MTAP-deficient pancreatic cancer, including MTAP-deficient NSCLC or MTAP-deficient pancreatic ductal adenocarcinoma (PDAC) or MTAP-deficient esophageal cancer. In another embodiment, the cancer is a tumor having an MTAP gene deletion. In any one of the embodiments herein, the cancer is a solid tumor or a haematological cancer. In one embodiment, the tumor is deficient in MTAP. In another embodiment, the tumor is normal in its expression of MTAP. In still another embodiment, the cancer is NSCLC, mesothelioma, squamous carcinoma of the head and neck, salivary gland tumors, urothelial cancers, sarcomas, or ovarian cancer. In still another embodiment, the cancer is NSCLC, esophagogastric and pancreatic cancers. In still another embodiment, the cancer is characterized by a reduction or absence of MTAP gene expression, absence of MTAP gene, reduced function of MTAP protein, reduced level or absence of MTAP protein, MTA accumulation, or combination thereof. In still another embodiment, the cancer is characterized by a reduction or absence of MTAP gene expression. In still another embodiment, the cancer is characterized by reduced function of MTAP protein. In still another embodiment, the cancer is characterized reduced level or absence of MTAP protein. In still another embodiment, the cancer is characterized by MTA accumulation

In an embodiment, the cancer is pancreatic cancer. In another embodiment, the cancer is non-small cell lung cancer. In yet another embodiment, the cancer is esophageal cancer. In still another embodiment, the cancer is bladder cancer.

In an aspect, provided herein is a method of treating pancreatic cancer comprising administering to a subject in need thereof a therapeutically effective amount of Compound A, or a pharmaceutically acceptable salt thereof; and a therapeutically effective amount of Compound B, or a pharmaceutically acceptable salt thereof.

In another aspect, provided herein is a method of treating non-small cell lung cancer (NSCLC) comprising administering to a subject in need thereof a therapeutically effective amount of Compound A, or a pharmaceutically acceptable salt thereof; and a therapeutically effective amount of Compound B, or a pharmaceutically acceptable salt thereof.

In an aspect, provided herein is a method of treating esophageal cancer comprising administering to a subject in need thereof a therapeutically effective amount of Compound A, or a pharmaceutically acceptable salt thereof; and a therapeutically effective amount of Compound B, or a pharmaceutically acceptable salt thereof.

In an aspect, provided herein is a method of treating bladder cancer comprising administering to a subject in need thereof a therapeutically effective amount of Compound A, or a pharmaceutically acceptable salt thereof; and a therapeutically effective amount of Compound B, or a pharmaceutically acceptable salt thereof.

In an aspect, provided herein is a methionine adenosyltransferase II alpha (MAT2A) inhibitor, or a pharmaceutically acceptable salt thereof, and PRMT Type I inhibitor, or a pharmaceutically acceptable salt thereof, for use in therapy.

In an embodiment, the methionine adenosyltransferase II alpha (MAT2A) inhibitor, or a pharmaceutically acceptable salt thereof, and the PRMT Type I inhibitor, or a pharmaceutically acceptable salt thereof, are for use in the treatment of cancer in a subject in need thereof.

In another aspect, provided herein is a use of a methionine adenosyltransferase II alpha (MAT2A) inhibitor in the manufacture of a medicament for treating cancer, wherein the MAT2A inhibitor is to be administered simultaneously or sequentially with a Type I protein arginine methyltransferase (Type I PRMT) inhibitor.

Exemplary lengths of time associated with the course of the treatment methods disclosed herein include: about one week; two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years and about five years.

In an embodiment of the methods, the method involves the administration of a therapeutically effective amount of a combination or composition comprising compounds provided herein, or pharmaceutically acceptable salts thereof, to a subject (including, but not limited to a human or animal) in need of treatment (including a subject identified as in need).

In another embodiment of the methods, the treatment includes co-administering the amount of the methionine adenosyltransferase II alpha (MAT2A) inhibitor, or a pharmaceutically acceptable salt thereof, and the amount of the PRMT Type I inhibitor, or a pharmaceutically acceptable salt thereof. In an embodiment, the amount of the methionine adenosyltransferase II alpha (MAT2A) inhibitor or a pharmaceutically acceptable salt thereof and the amount of PRMT Type I inhibitor or a pharmaceutically acceptable salt thereof are in a single formulation or unit dosage form. In still other embodiments, the amount of methionine adenosyltransferase II alpha (MAT2A) inhibitor or a pharmaceutically acceptable salt thereof and the amount of PRMT Type I inhibitor or a pharmaceutically acceptable salt thereof are in a separate formulations or unit dosage forms.

In the foregoing methods, the treatment can include administering the amount of methionine adenosyltransferase II alpha (MAT2A) inhibitor or a pharmaceutically acceptable salt thereof and the amount of PRMT Type I inhibitor or a pharmaceutically acceptable salt thereof at substantially the same time or administering the amount of methionine adenosyltransferase II alpha (MAT2A) inhibitor or a pharmaceutically acceptable salt thereof and the amount of PRMT Type I inhibitor or a pharmaceutically acceptable salt thereof at different times. In some embodiments of the foregoing methods, the amount of methionine adenosyltransferase II alpha (MAT2A) inhibitor or a pharmaceutically acceptable salt thereof and/or the amount of PRMT Type I inhibitor or a pharmaceutically acceptable salt thereof is administered at dosages that would not be effective when one or both of methionine adenosyltransferase II alpha (MAT2A) inhibitor or a pharmaceutically acceptable salt thereof and PRMT Type I inhibitor or a pharmaceutically acceptable salt thereof is administered alone, but which amounts are effective in combination.

In another embodiment of the methods, the treatment includes co-administering the amount of the Compound A, or a pharmaceutically acceptable salt thereof, and the amount of Compound B, or a pharmaceutically acceptable salt thereof. In an embodiment, the amount of Compound A or a pharmaceutically acceptable salt thereof and the amount of Compound B or a pharmaceutically acceptable salt thereof are in a single formulation or unit dosage form. In still other embodiments, the amount of Compound A or a pharmaceutically acceptable salt thereof and the amount Compound B or a pharmaceutically acceptable salt thereof are in a separate formulations or unit dosage forms.

In the foregoing methods, the treatment can include administering the amount of Compound A or a pharmaceutically acceptable salt thereof and the amount of Compound B or a pharmaceutically acceptable salt thereof at substantially the same time or administering the amount of Compound A or a pharmaceutically acceptable salt thereof and the amount of Compound B or a pharmaceutically acceptable salt thereof at different times. In some embodiments of the foregoing methods, the amount of Compound A or a pharmaceutically acceptable salt thereof and/or the amount of Compound B or a pharmaceutically acceptable salt thereof is administered at dosages that would not be effective when one or both of Compound A or a pharmaceutically acceptable salt thereof and Compound B or a pharmaceutically acceptable salt thereof is administered alone, but which amounts are effective in combination.

### Pharmaceutical Compositions

In an aspect, provided herein is a pharmaceutical composition comprising a methionine adenosyltransferase II alpha (MAT2A) inhibitor, or a pharmaceutically acceptable salt thereof, Type I protein arginine methyltransferase (Type I PRMT) inhibitor, or a pharmaceutically acceptable salt thereof, and at least one pharmaceutically acceptable carrier.

In another aspect, provided herein is a combination product comprising a first pharmaceutical composition comprising a therapeutically effective amount of a methionine adenosyltransferase II alpha (MAT2A) inhibitor and a second pharmaceutical composition comprising a therapeutically effective amount of a Type I protein arginine methyltransferase (Type I PRMT) inhibitor.

In an embodiment, the MAT2A inhibitor is a compound of Formula I:

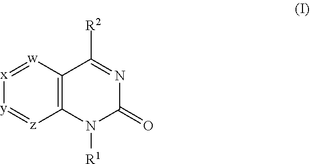

or a pharmaceutically acceptable salt thereof; wherein the variables are defined herein.

In another embodiment, the Type I PRMT inhibitor is a compound of Formula II:

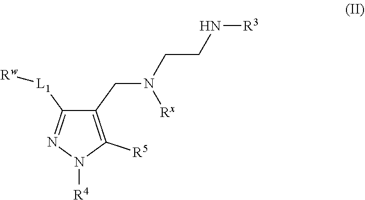

or a pharmaceutically acceptable salt thereof; wherein the variables are defined herein.

In yet another aspect, provided herein is a combination product comprising a first pharmaceutical composition comprising a therapeutically effective amount of Compound A:

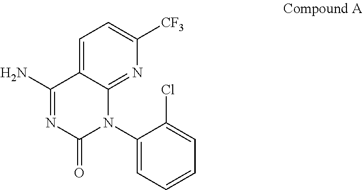 [SMILES: Nc1nc(=O)n(-c2ccccc2Cl)c2nc(C(F)(F)F)ccc12]

or a pharmaceutically acceptable salt thereof;

a second pharmaceutical composition comprising a therapeutically effective amount of Compound B:

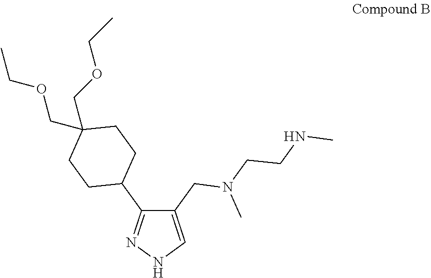 [SMILES: CCOCC1(COCC)CCC(c2n[nH]cc2CN(C)CCNC)CC1]

or a pharmaceutically acceptable salt thereof.

In still another aspect, provided herein is a pharmaceutical composition comprising a therapeutically effective amount of Compound A, or a pharmaceutically acceptable salt thereof; Compound B, or a pharmaceutically acceptable salt thereof; and a pharmaceutically acceptable carrier.

In an embodiment, the pharmaceutical composition is for use in the treatment of cancer in a patient. In an embodiment, the pharmaceutical composition is for use in the treatment of a solid tumor in a patient.

### Administration/Dosage/Formulations

In another aspect, provided herein is a pharmaceutical composition or pharmaceutical combination comprising the compounds disclosed herein, together with a pharmaceutically acceptable carrier.

In an embodiment of the combination product, methionine adenosyltransferase II alpha (MAT2A) inhibitor, or a pharmaceutically acceptable salt thereof, and PRMT Type I inhibitor, or a pharmaceutically acceptable salt thereof, are in the same formulation. In another embodiment of the combination product, methionine adenosyltransferase II alpha (MAT2A) inhibitor and PRMT Type I inhibitor, are in separate formulations. In a further embodiment of this embodiment, the formulations are for simultaneous or sequential administration.

Administration of the combination includes administration of the combination in a single formulation or unit dosage form, administration of the individual agents of the combination concurrently but separately, or administration of the individual agents of the combination sequentially by any suitable route. The dosage of the individual agents of the combination may require more frequent administration of one of the agent(s) as compared to the other agent(s) in the combination. Therefore, to permit appropriate dosing, packaged pharmaceutical products may contain one or more dosage forms that contain the combination of agents, and one or more dosage forms that contain one of the combination of agents, but not the other agent(s) of the combination.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms are dictated by and directly dependent on (a) the unique characteristics of the disclosed compound and the particular therapeutic effect to be achieved, and (b) the limitations inherent in the art of compounding/formulating such a disclosed compound for the treatment of pain, a depressive disorder, or drug addiction in a patient.

In one embodiment, the compounds provided herein are formulated using one or more pharmaceutically acceptable excipients or carriers. In one embodiment, the pharmaceutical compositions provided herein comprise a therapeutically effective amount of a disclosed compound and a pharmaceutically acceptable carrier.

The optimum ratios, individual and combined dosages, and concentrations of the drug compounds that yield efficacy without toxicity are based on the kinetics of the active ingredients' availability to target sites, and are determined using methods known to those of skill in the art.

Routes of administration of any of the compositions discussed herein include oral, nasal, rectal, intravaginal, parenteral, buccal, sublingual or topical. The compounds may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions are not limited to the particular formulations and compositions that are described herein.

For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

### Kits

In an aspect, the present disclosure provides a kit for treating cancer comprising a methionine adenosyltransferase II alpha (MAT2A) inhibitor, or a pharmaceutically acceptable salt thereof, and a PRMT Type I inhibitor, or a pharmaceutically acceptable salt thereof.

In certain embodiments, the kit comprises a pharmaceutical product comprising a pharmaceutical composition comprising methionine adenosyltransferase II alpha (MAT2A) inhibitor, or a pharmaceutically acceptable salt thereof, and a pharmaceutically acceptable carrier or diluent; and a pharmaceutical composition comprising PRMT Type I inhibitor, or a pharmaceutically acceptable salt thereof, and a pharmaceutically acceptable carrier or diluent.

In some embodiments, the kit comprises a pharmaceutical composition comprising methionine adenosyltransferase II alpha (MAT2A) inhibitor, or a pharmaceutically acceptable salt thereof; PRMT Type I inhibitor, or a pharmaceutically acceptable salt thereof; and a pharmaceutically acceptable carrier or diluent.

In additional embodiments, pharmaceutical kits are provided. The kit includes a sealed container approved for the storage of pharmaceutical compositions, the container containing one of the above-described pharmaceutical compositions. In some embodiments, the sealed container minimizes the contact of air with the ingredients, e.g. an airless bottle. In other embodiments, the sealed container is a sealed tube. An instruction for the use of the composition and the information about the composition are to be included in the kit.

In a particular embodiment, the compounds of the combination can be dosed on the same schedule, whether by administering a single formulation or unit dosage form containing all of the compounds of the combination, or by administering separate formulations or unit dosage forms of the compounds of the combination. However, some of the compounds used in the combination may be administered more frequently than once per day, or with different frequencies that other compounds in the combination. Therefore, in one embodiment, the kit contains a formulation or unit dosage form containing all of the compounds in the combination of compounds, and an additional formulation or unit dosage form that includes one of the compounds in the combination of agents, with no additional active compound, in a container, with instructions for administering the dosage forms on a fixed schedule.

The kits provided herein include comprise prescribing information, for example, to a patient or health care provider, or as a label in a packaged pharmaceutical formulation. Prescribing information may include for example efficacy, dosage and administration, contraindication and adverse reaction information pertaining to the pharmaceutical formulation.

In all of the foregoing the combination of compounds of the invention can be administered alone, as mixtures, or with additional active agents.

A kit provided herein can be designed for conditions necessary to properly maintain the components housed therein (e.g., refrigeration or freezing). A kit can contain a label or packaging insert including identifying information for the components therein and instructions for their use (e.g., dosing parameters, clinical pharmacology of the active ingredient(s), including mechanism(s) of action, pharmacokinetics and pharmacodynamics, adverse effects, contraindications, etc.).

Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial).

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings of the present disclosure as set forth.

### EXAMPLES

The compounds and methods disclosed herein are further illustrated by the following examples, which should not be construed as further limiting. The practice of the present disclosure will employ, unless otherwise indicated, conventional techniques of organic synthesis, cell biology, cell culture, and molecular biology, which are within the skill of the art.

Processes for preparing the compounds disclosed herein can be found, at least, in WO 2020/123395, WO 2014/153226, and WO 2021/023609, the contents of which are incorporated in their entirety.

### Example 1: Anti-Tumor Efficacy in Mouse Xenografts

### Efficacy of Compound A and Compound B in HCT-116 MTAP Isogenic Pair

The effect of Compound A and Compound B as single-agent anti-tumor agents and in combination was assessed in the HCT-116 human colon tumor cell line, MTAP isogenic pair (MTAP proficient (WT) or MTAP-deleted). Cells were expanded in DMEM/F12 GlutaMAX (Fisher Scientific, Catalog Number 10-565-018) with 10% fetal bovine serum. These cells were free of mycoplasma and authenticated as HCT-116 by STR profiling. Two and a half million cells in log growth phase were resuspended in Hanks Balanced Salt Solution containing 50% Matrigel and implanted subcutaneously into the flank of each recipient female CB17/lcr-Prkd^{scid}/lcrlcoCrl mouse. Mice were housed in microisolator cages with corn cob bedding with additional enrichment consisting of sterile nesting material (Innovive) and Bio-huts (Bio-Serv). Water (Innovive) and diet (Teklad Global 19% Protein Extruded Diet 2919, Irradiated) were provided ad libitum. The environment was maintained on a 12-hour light cycle at approximately 68-72° F. and 40-60% relative humidity.

Tumor Volume (TV) was calculated using the following formula: TV (mm³)=(width×width×length)/2. No dose holidays were provided for during the study and all mice were euthanized following the final dose on Day fourteen. Tumor growth inhibition (TGI) was calculated by [(TV control_{final}−TV treated_{final})/(TV control_{final}−TV controlᵢₙᵢₜᵢₐₗ)×100]. TV was analyzed for statistical significance utilizing GraphPad Prism version 9.1.0. Repeated Measures 2-Way ANOVA with Tukey's Multiple Comparisons was utilized, and P-values were presented from the final tumor measurement and were considered statistically significant if less than 0.05.

Mean tumor volume at dosing start was approximately 128-145 mm³, with seven mice randomized to each treatment group. The study design was identical for both models, with the study consisting of six treatment groups. Mice were dosed orally, once per day, with Vehicle, Compound A at 5 mg/kg, Compound B at 9.375 or 18.75 mg/kg, or Compound A at 5 mg/kg and Compound B at 9.375 mg/kg, or Compound A at 5 mg/kg and Compound B at 18.75 mg/kg. The doses were staggered with Compound A given first and Compound B administered 2 hours later. Likewise, the Vehicle B (for Compound B, Saline) was administered to animals 2 hours later than the Vehicle A (for Compound A, 0.5% 400 cps methylcellulose with 0.5% Tween-80 in sterile water).

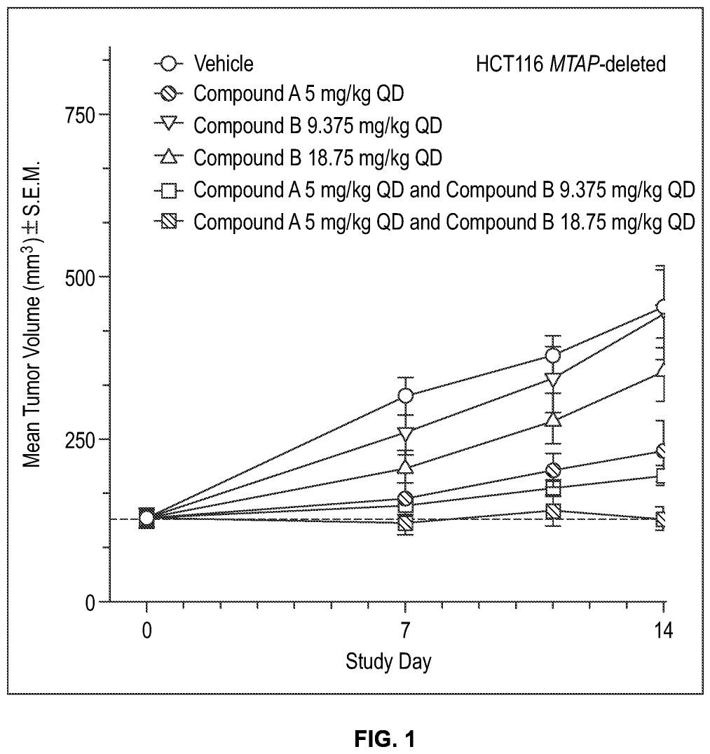

In HCT-116 MTAP-deleted, treatment with Compound A resulted in 68% TGI, while Compound B alone resulted in 4% TGI at 9.375 mg/kg and 30% TGI at 18.75 mg/kg. The combination of Compound A and Compound B at 9.375 mg/kg resulted in 80% TGI. The combination of Compound A and Compound B at 18.75 mg/kg resulted in 100% TGI, (FIG. 1). The combination of Compound A and Compound B prevented tumor growth in HCT-116 MTAP-deleted tumors. The results are shown in Table 3.

TABLE 3
HCT-116 MTAP-Deleted Model

| | | | | | p value, | p value, |
|---|---|---|---|---|---|---|
| | | Initial | Final | | treated | Combination |
| Dose A | Dose B | TV | TV | TGI | vs. | vs. |
| (mg/kg) | (mg/kg) | (mean) | (mean) | (%) | Vehicle | Compound A |
| 0 | 0 | 128 | 454 | NA | NA | NA |
| 5 | 0 | 128 | 232 | 68 | <0.0001 | NA |
| 0 | 9.375 | 128 | 441 | 4 | 0.9998 | NA |
| 0 | 18.75 | 128 | 357 | 30 | 0.2726 | NA |
| 5 | 9.375 | 128 | 194 | 80 | <0.0001 | 0.9595 |
| 5 | 18.75 | 128 | 128 | 100 | <0.0001 | 0.1953 |

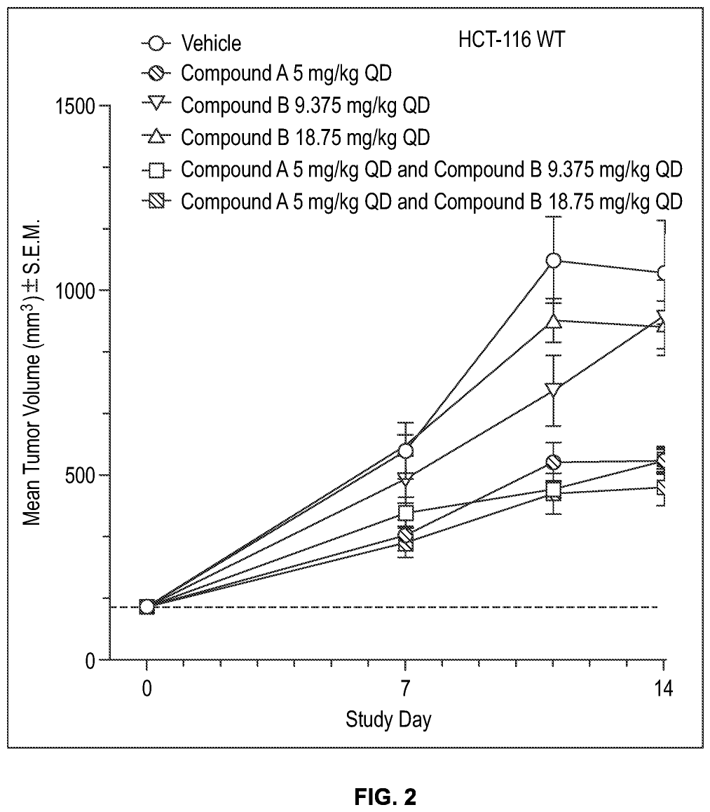

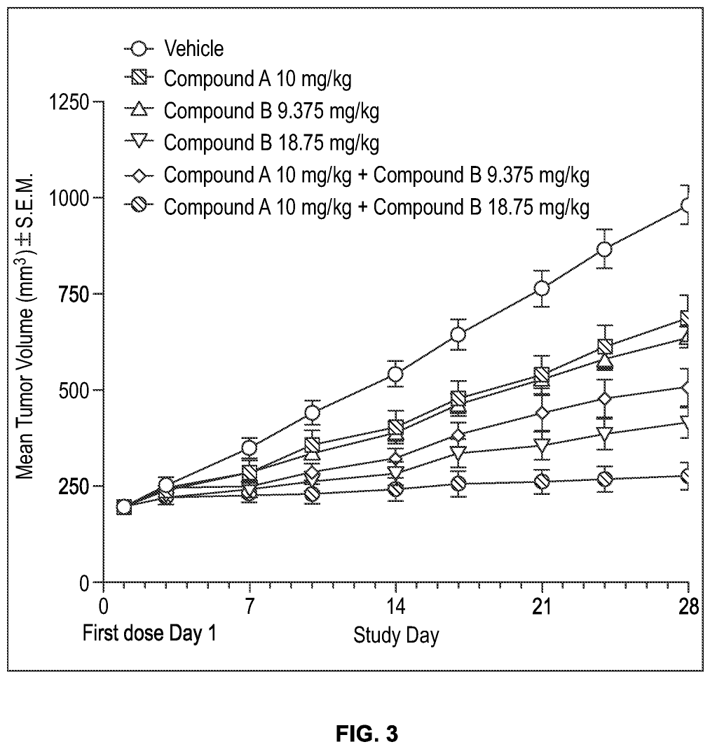

In HCT-116 MTAP WT, treatment with Compound A resulted in 56% TGI, while Compound B alone resulted in 13% TGI at 9.375 mg/kg and 16% TGI at 18.75 mg/kg. The combination of Compound A and Compound B at 9.375 mg/kg resulted in 56% TGI. The combination of Compound A and Compound B at 18.75 mg/kg resulted in 64% TGI (FIG. 2). The combination of Compound A and Compound B did not prevent tumor growth in HCT-116 WT tumors. These results are shown in Table 4.

TABLE 4
HCT-116 MTAP WT Model

| | | | | | p value, | p value, |
|---|---|---|---|---|---|---|
| | | Initial | Final | | treated | Combination |
| Dose A | Dose B | TV | TV | TGI | vs. | vs. |
| (mg/kg) | (mg/kg) | (mean) | (mean) | (%) | Vehicle | Compound A |
| 0 | 0 | 145 | 1049 | NA | NA | NA |
| 5 | 0 | 145 | 539 | 56 | <0.0001 | NA |
| 0 | 9.375 | 145 | 928 | 13 | 0.7172 | NA |
| 0 | 18.75 | 145 | 907 | 16 | 0.5604 | NA |
| 5 | 9.375 | 145 | 544 | 56 | <0.0001 | >0.9999 |
| 5 | 18.75 | 145 | 469 | 64 | <0.0001 | 0.9630 |

### Efficacy of Compound A and Compound B in Pancreatic Xenograft BxPC-3

The BxPC-3 tumor cell line was maintained in vitro as a monolayer culture in RPMI 1640 medium supplemented with 10% fetal bovine serum at 37° C. in an atmosphere of 5% CO₂ in air. The tumor cells were routinely sub-cultured, not to exceed 4-5 passages. The cells growing in an exponential growth phase were harvested and counted for tumor inoculation. Each mouse was inoculated subcutaneously on the right flank with 5 million BxPC-3 tumor cells in 0.1 mL RPMI 1640 and Matrigel mixture (1:1 ratio) for tumor development.

Tumor Volume (TV) was calculated using the following formula: TV (mm³)=(width×width×length)/2. No dose holidays were provided for during the study and all mice were euthanized following the final dose on Day 28. Tumor growth inhibition (TGI) was calculated by [(TV control_{final}−TV treated_{final})/(TV control_{final}−TV controlᵢₙᵢₜᵢₐₗ)×100]. TV was analyzed for statistical significance using GraphPad Prism version 9.1.0. Repeated Measures 2-Way ANOVA with Tukey's Multiple Comparisons was utilized, and P-values were presented from the final tumor measurement and were considered statistically significant if less than 0.05. The treatment started when the mean tumor volume reached about 197 mm³. Mice were then randomly assigned to respective groups so that the mean starting tumor volume was the same among groups. Mice were dosed orally, once per day, with Vehicle, Compound A at 10 mg/kg, Compound B at 9.375 or 18.75 mg/kg, or Compound A at 10 mg/kg and Compound B at 9.375 mg/kg, or Compound A at 10 mg/kg and Compound B at 18.75 mg/kg. The Doses were staggered with Compound A given first and Compound B administrated 2 hours later. Likewise, the Vehicle B (for Compound B, Saline) was administrated to animals 2 hours later than the Vehicle A (for Compound A, 0.5% 400 cps methylcellulose with 0.5% Tween-80 in sterile water).

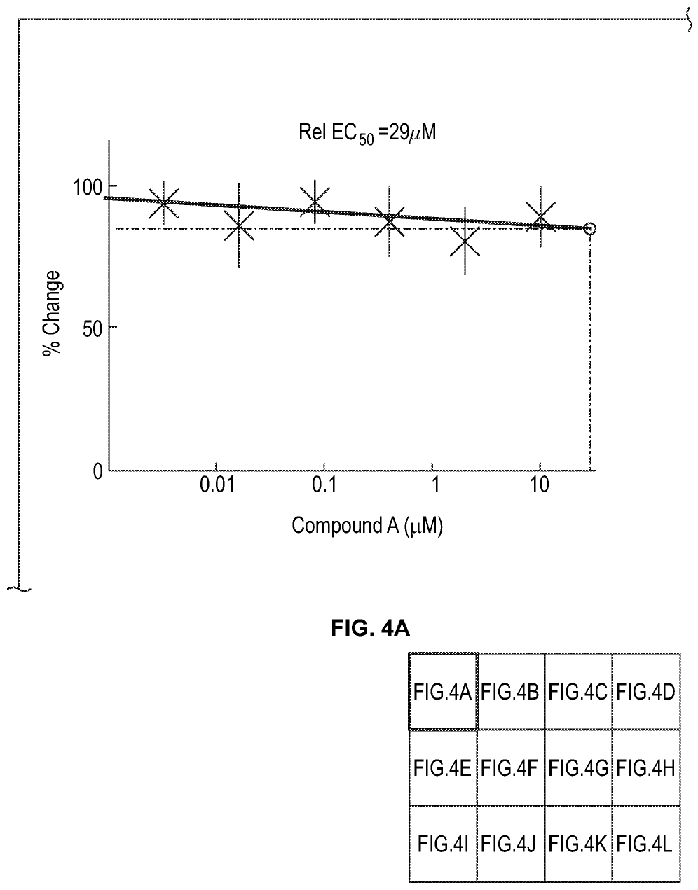

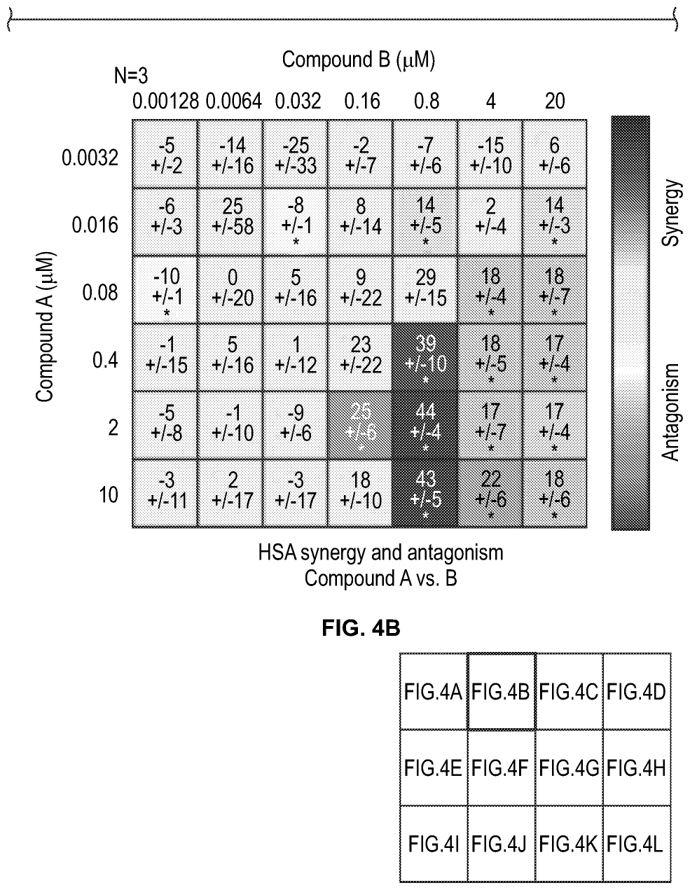

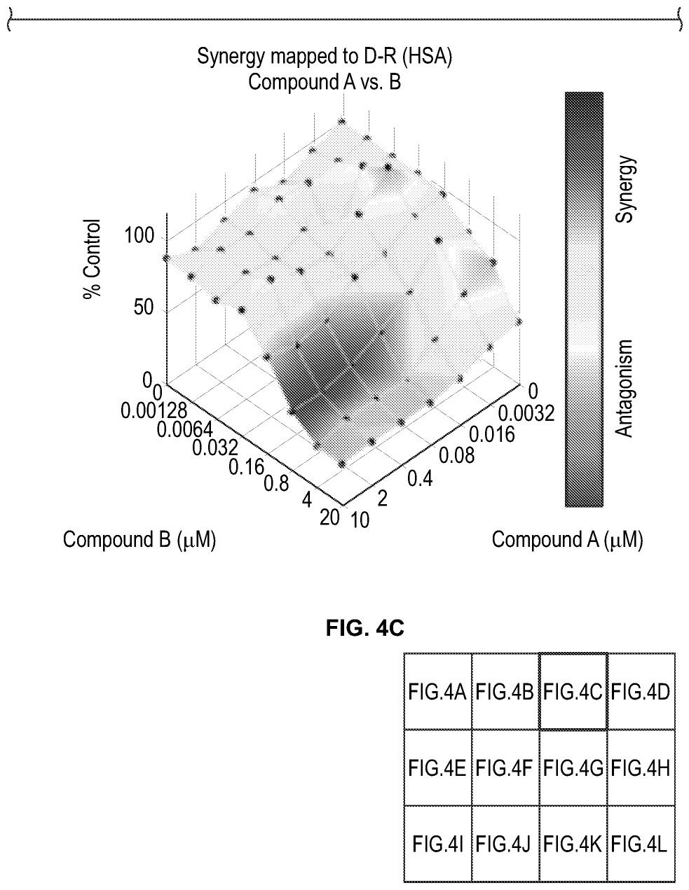

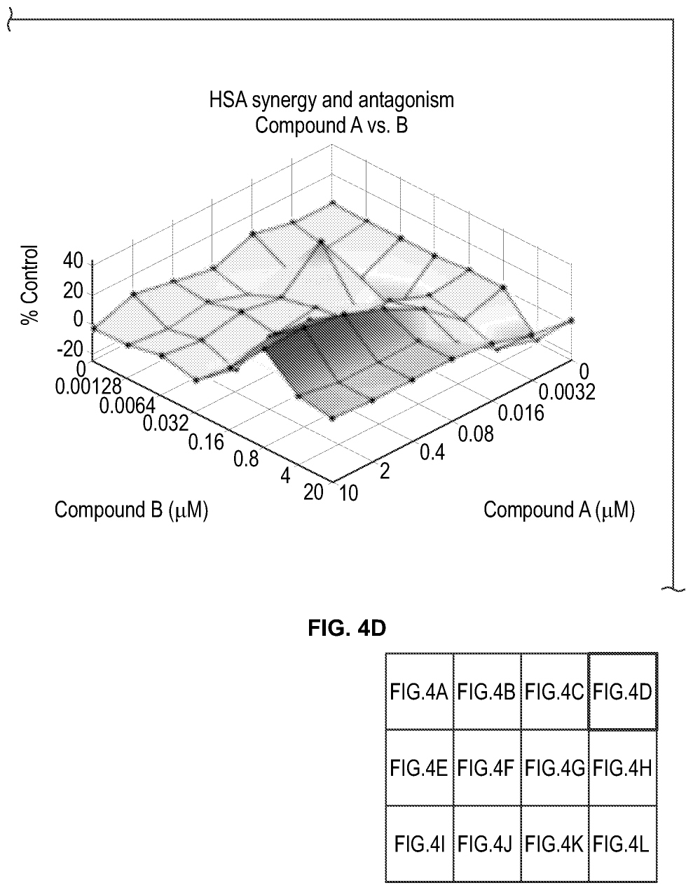

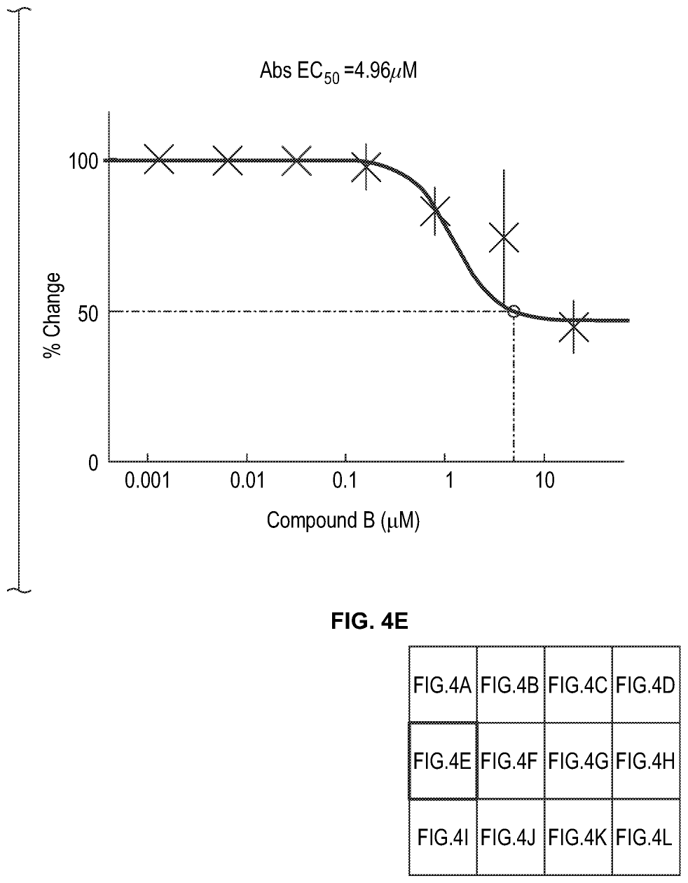

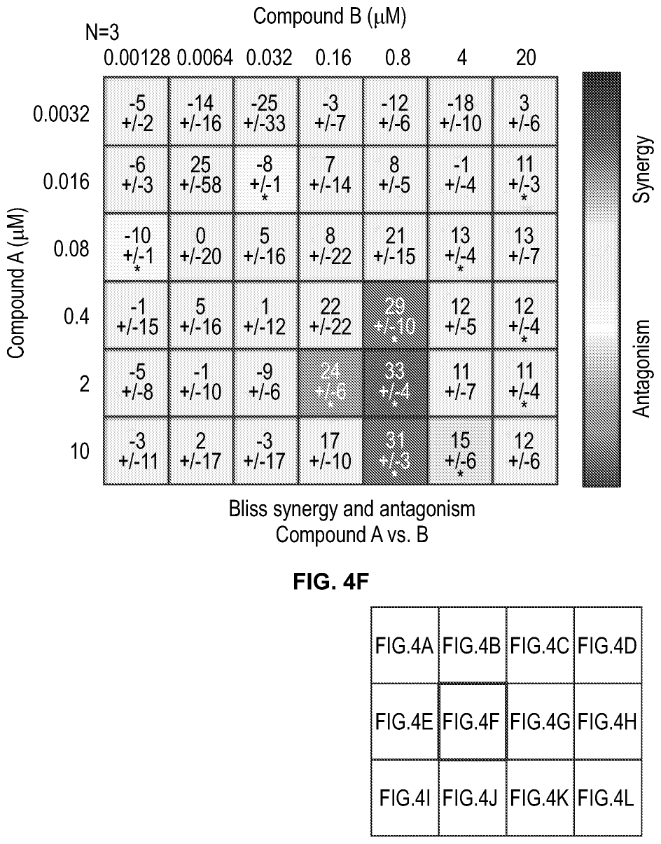

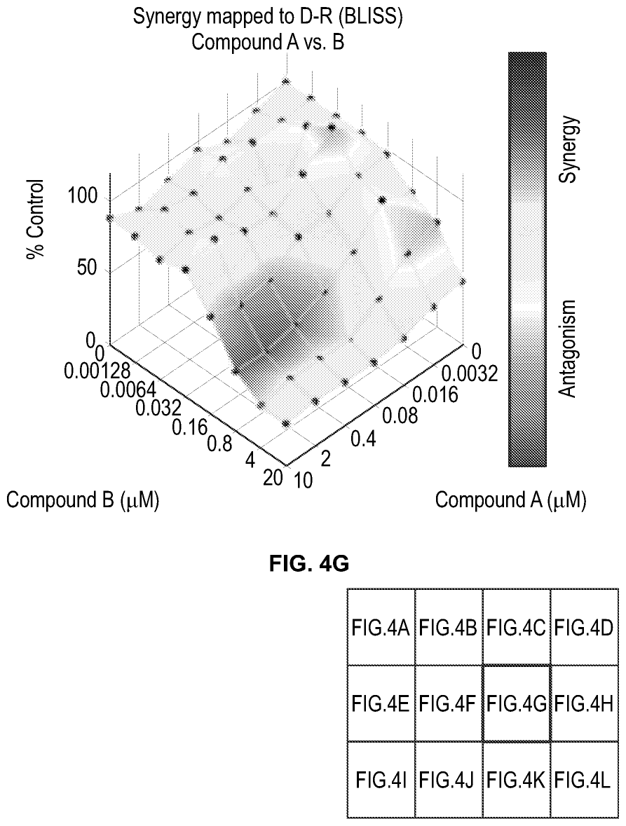

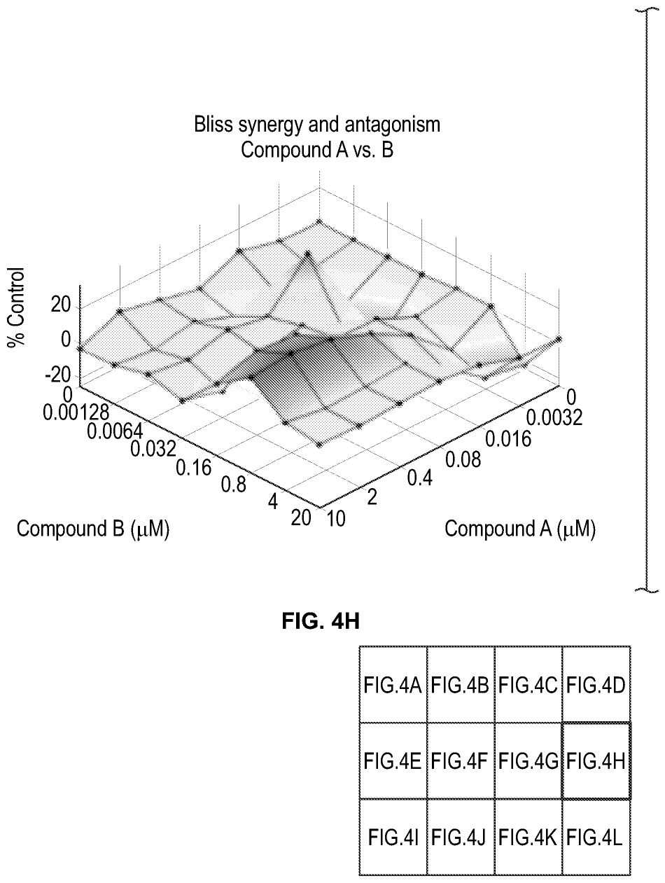

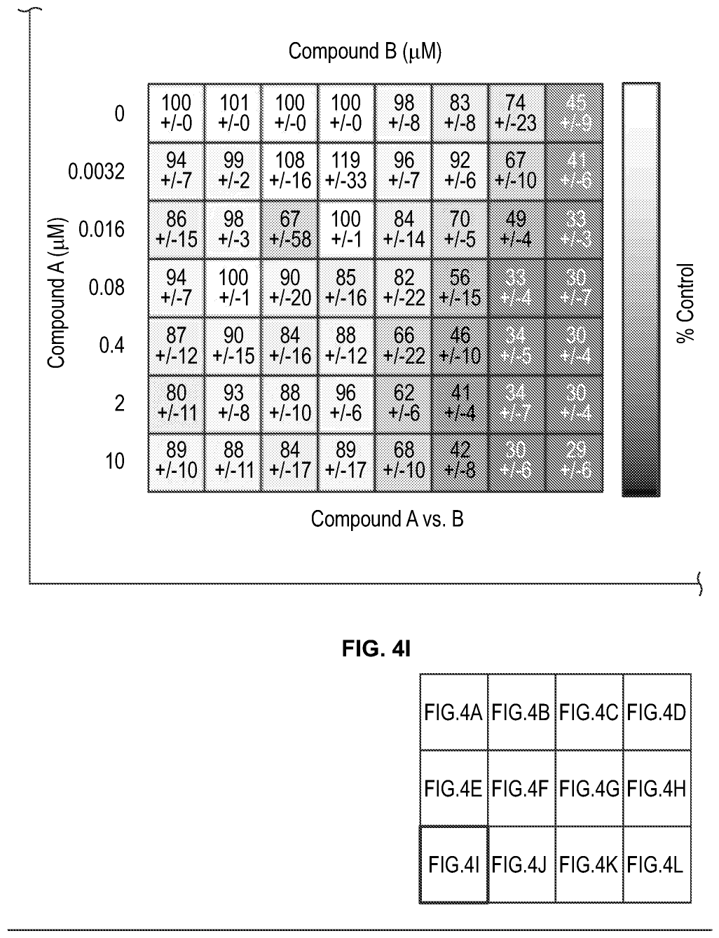

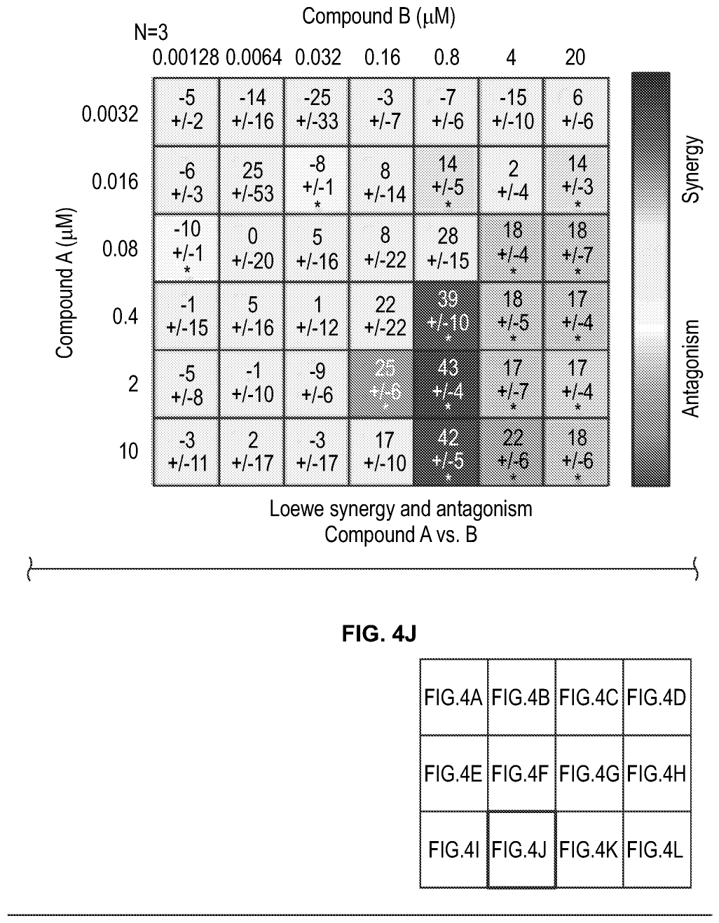

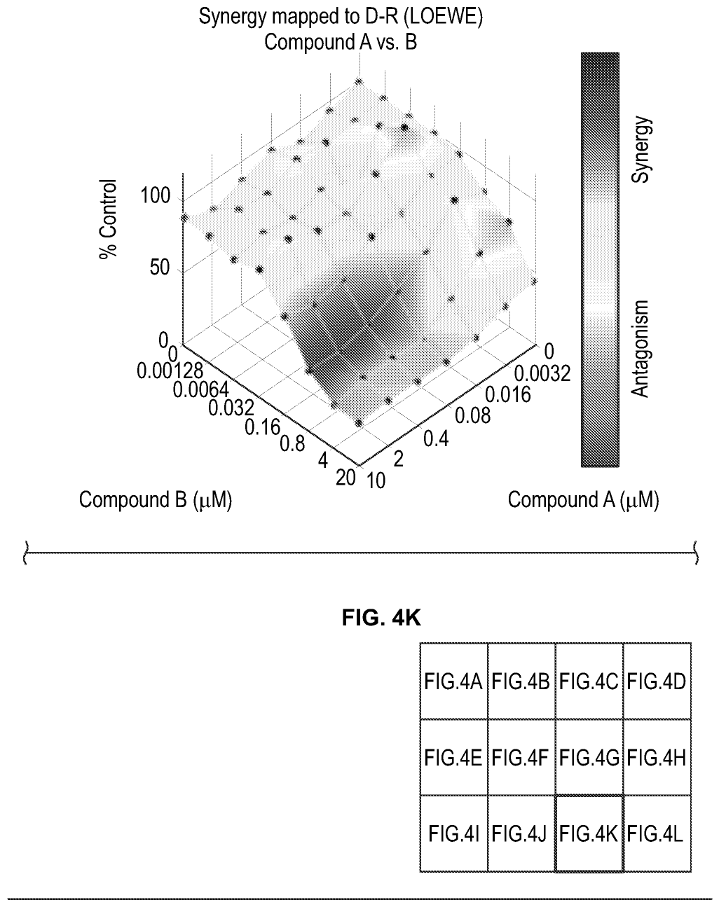

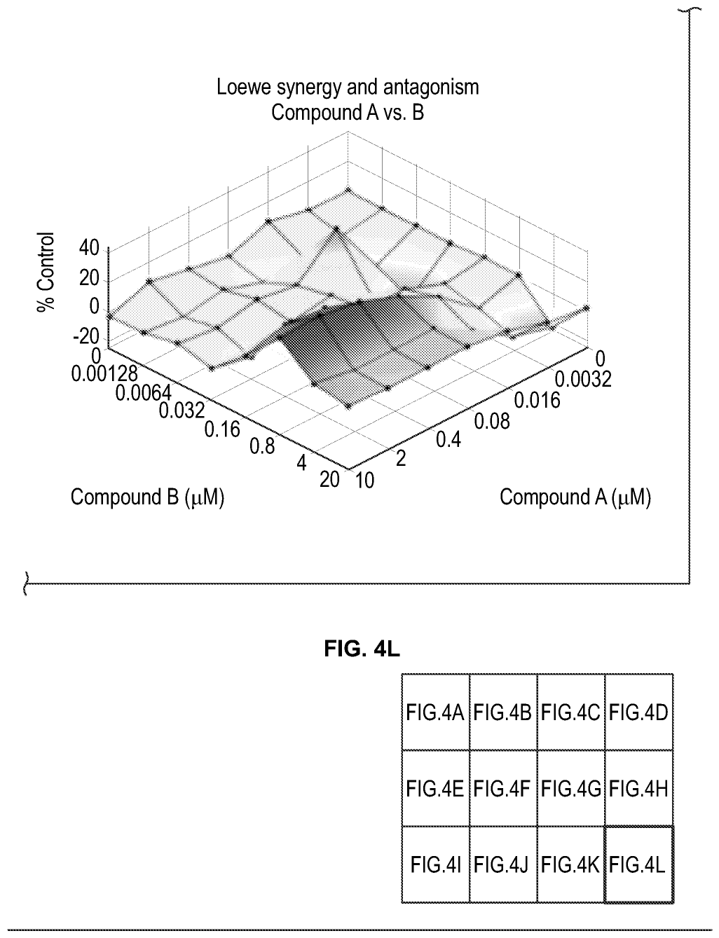

In BxPC-3, treatment with Compound A resulted in 37% TGI, 44% TGI with Compound B at 9.375 mg/kg, 72% TGI with Compound B at 18.75 mg/kg, 61% TGI with Compound A and Compound B at 9.375 mg/kg, and 90% TGI with Compound A and Compound B at 18.75 mg/kg, (FIG. 4Error! Reference source not found.). The combination of Compound A and Compound B resulted in a significant delay in tumor growth when compared to Compound A alone. The results are shown in Table 5.

TABLE 5
BxPC-3 Model

| | | | | | p value, | p value, |
|---|---|---|---|---|---|---|
| | | Initial | Final | | treated | Combination |
| Dose A | Dose B | TV | TV | TGI | vs. | vs. |
| (mg/kg) | (mg/kg) | (mean) | (mean) | (%) | Vehicle | Compound A |
| 0 | 0 | 195 | 951 | NA | NA | NA |
| 10 | 0 | 195 | 686 | 37 | <0.0001 | NA |
| 0 | 9.375 | 200 | 638 | 44 | <0.0001 | NA |
| 0 | 18.75 | 196 | 415 | 72 | <0.0001 | NA |
| 10 | 9.375 | 198 | 504 | 61 | <0.0001 | 0.0013 |
| 10 | 18.75 | 199 | 277 | 90 | <0.0001 | <0.0001 |

### Example 2: Proliferation Screening

### Materials and Methods

A 4-day proliferation screening was done in colorectal cancer lines HCT116 and HCT116 MTAP−/− (CRISPR Knock-out), and a 5-day proliferation screening was performed in pancreatic cancer cell lines BxPC3, KP4 and lung cancer line, NCI-H838. The nuclei were fluorescent tagged for all the cell lines. Combinations included a 5-fold titration from 10 uM for MAT2Ai (Compound A) and 5-fold titration from 20 uM for Type1 PRMTi (Compound B). Cells were plated at a seeding density 1000 cells/well in a 96-well format 24h before treatment and treated the following day with an 8×8 double matrix titration (five-fold dilution series) of Compound A (MAT2Ai) and Compound B (Type I PRMTi). Triplicates were run for each data point. Nuclear count was measured at the end point using the IncuCyte S3 Live-Cell Analysis System. Average fluorescence of cells treated with DMSO was set to 100% and the % viable cells were calculated accordingly. Dose response curves for single drug activity and drug-drug synergy for combinations were generated using Combenefit software. Synergy was evaluated using HSA, Loewe, and Bliss models.

### Results

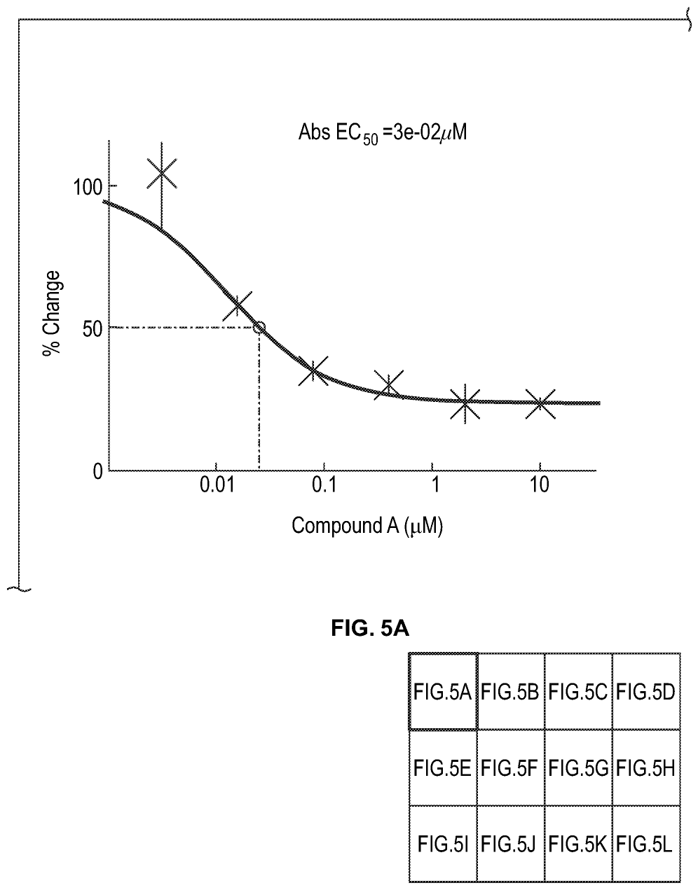

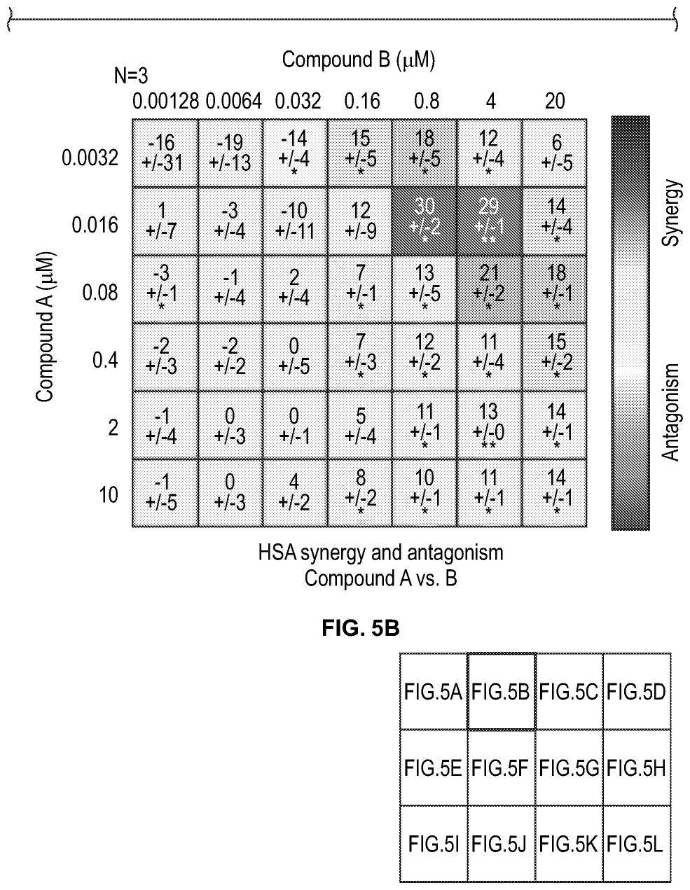

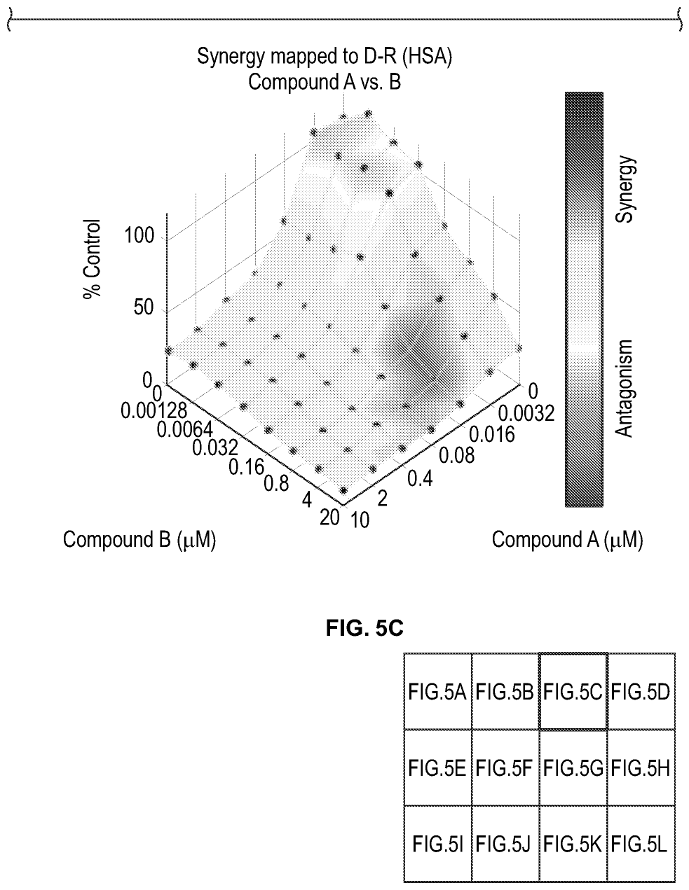

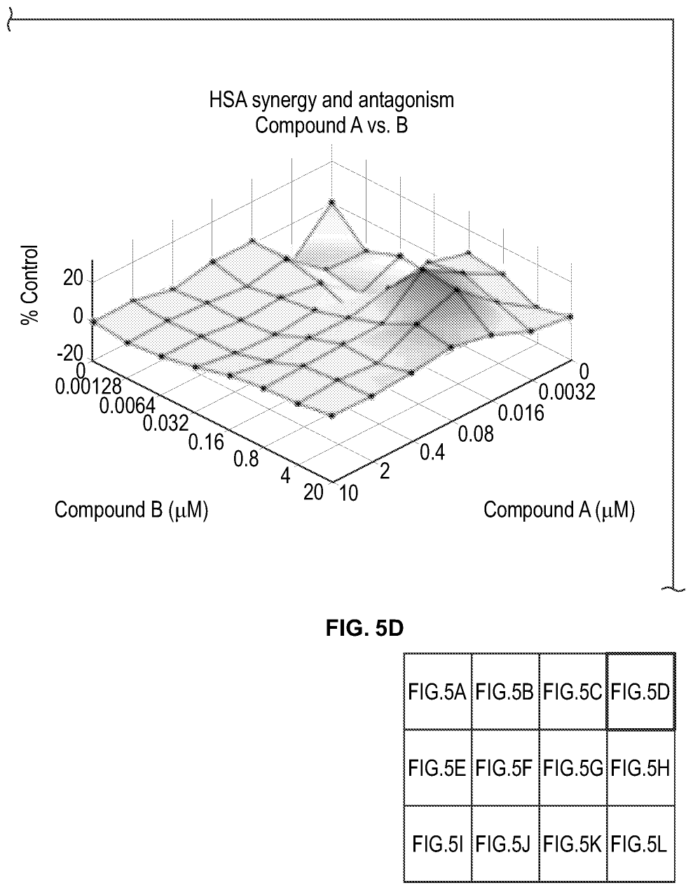

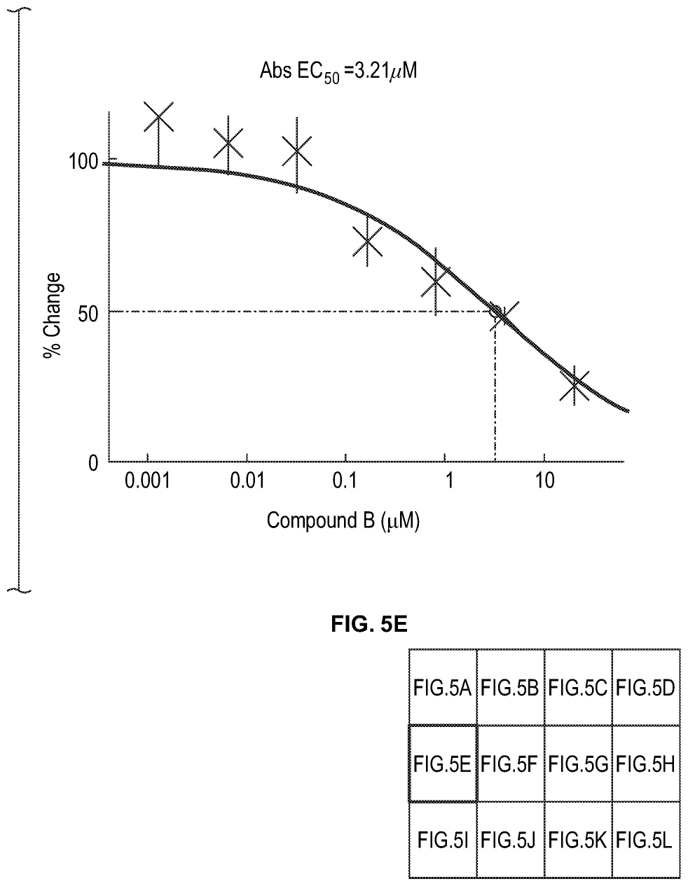

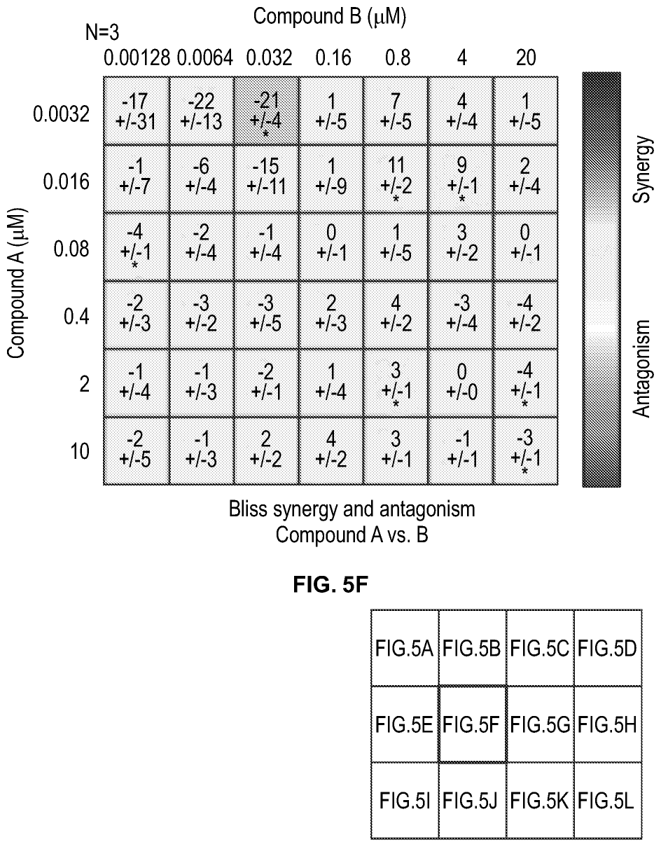

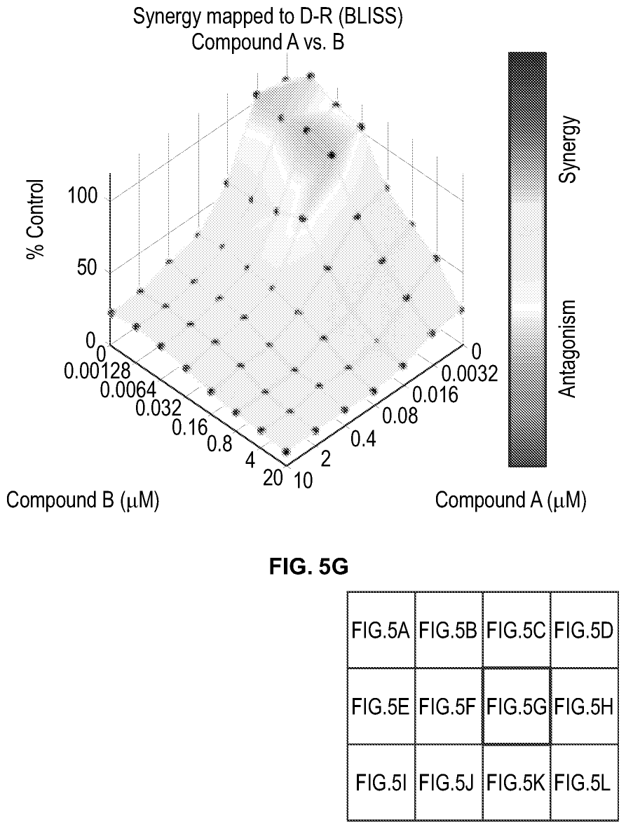

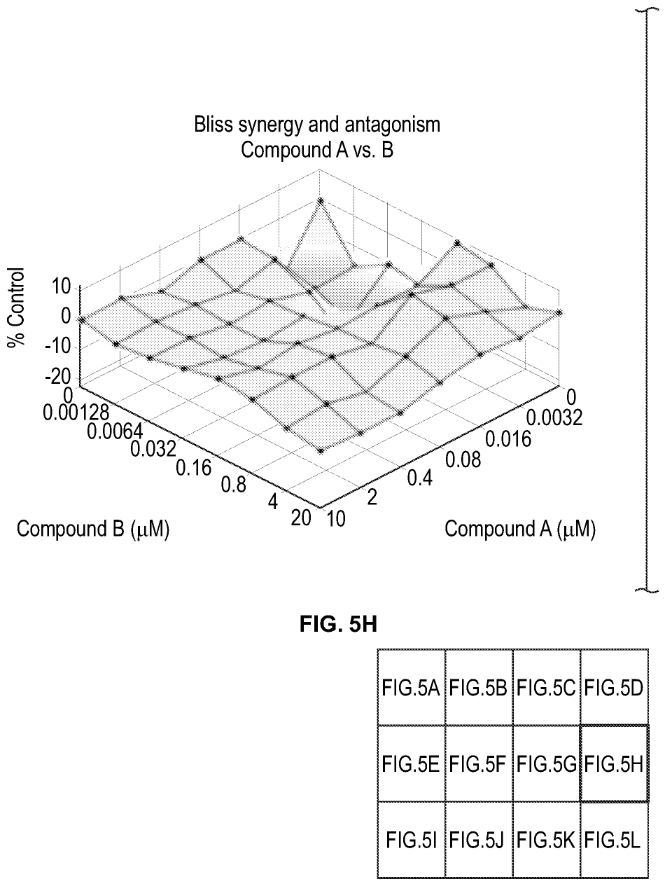

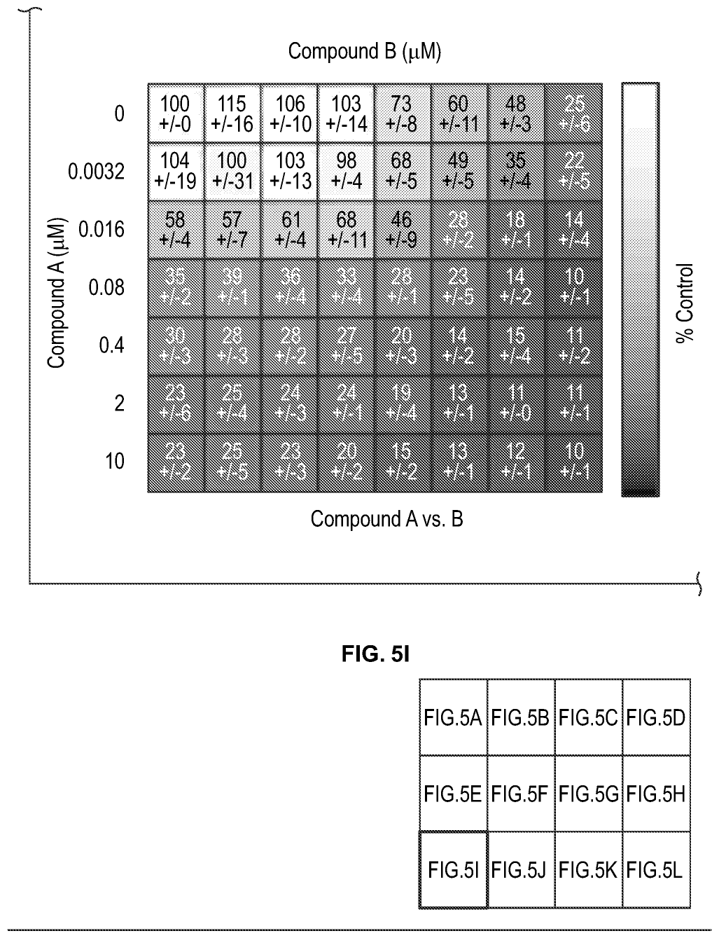

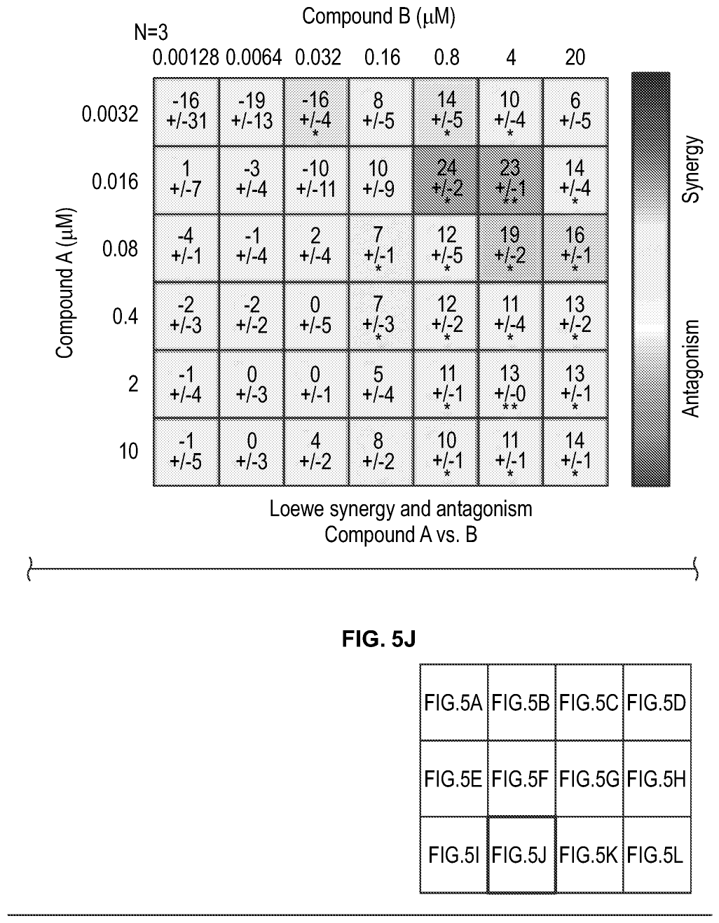

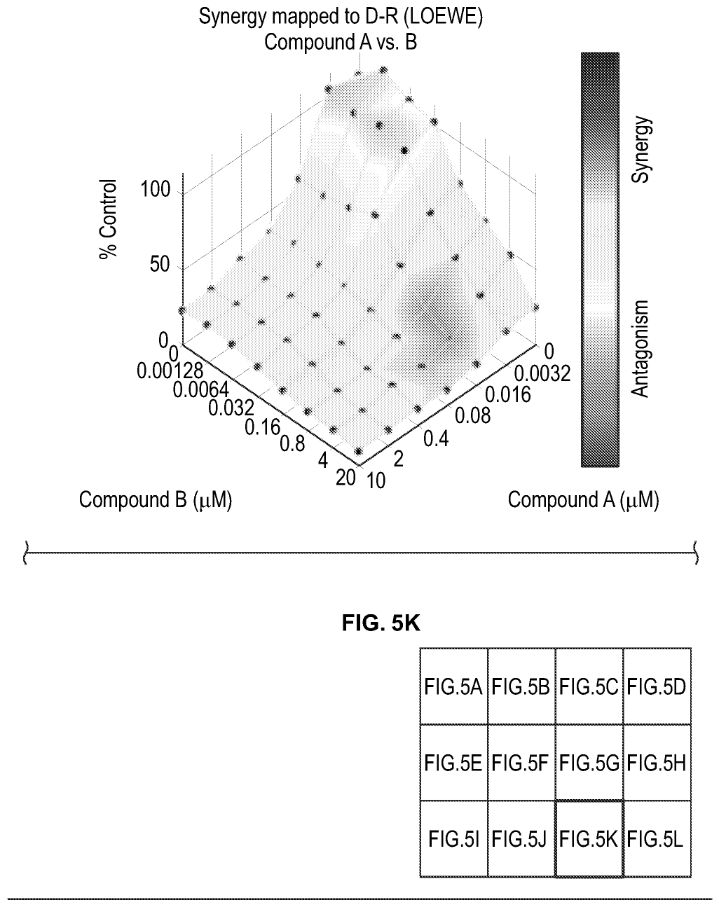

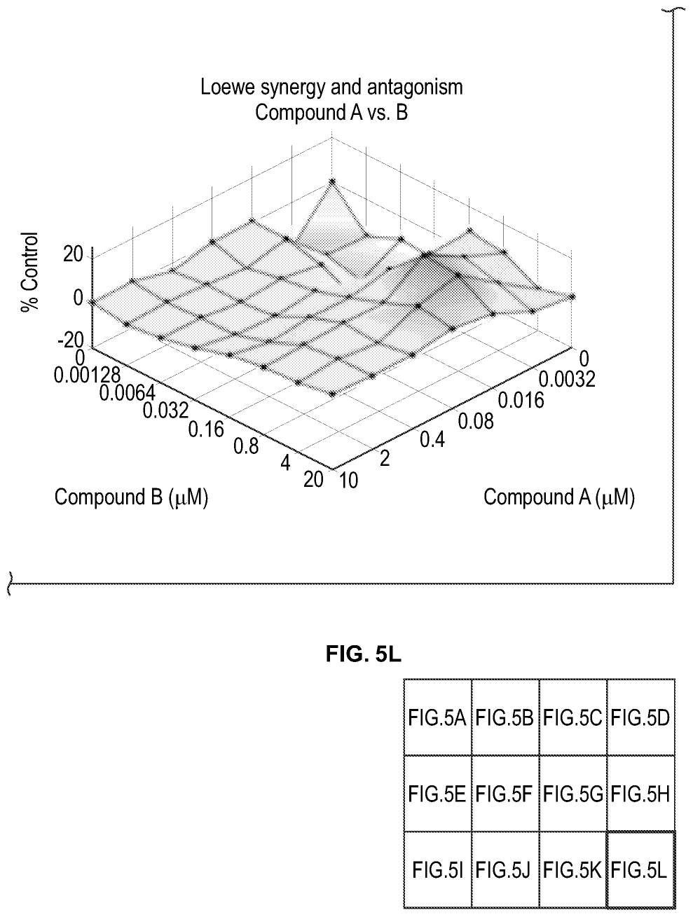

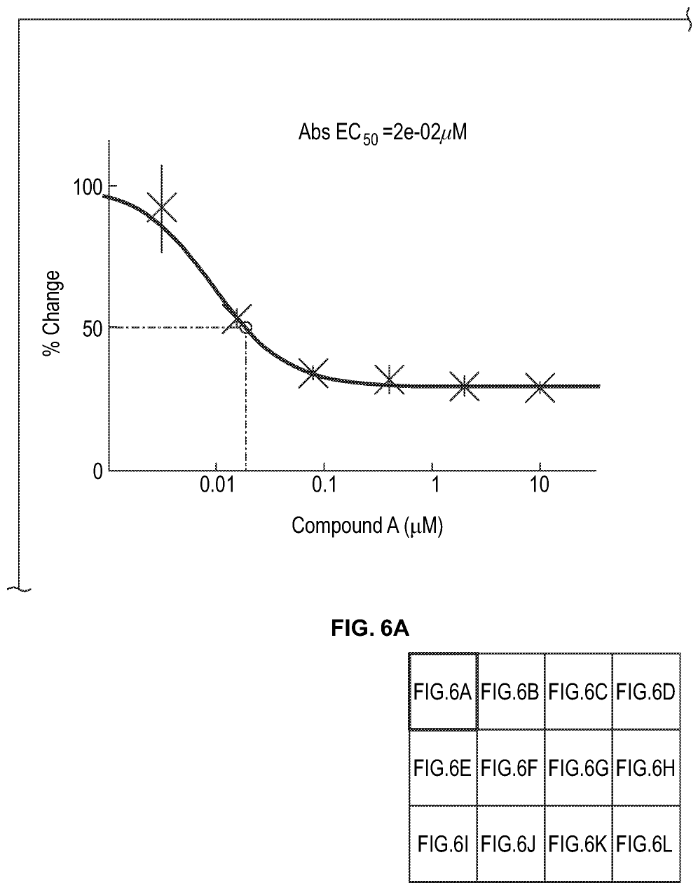

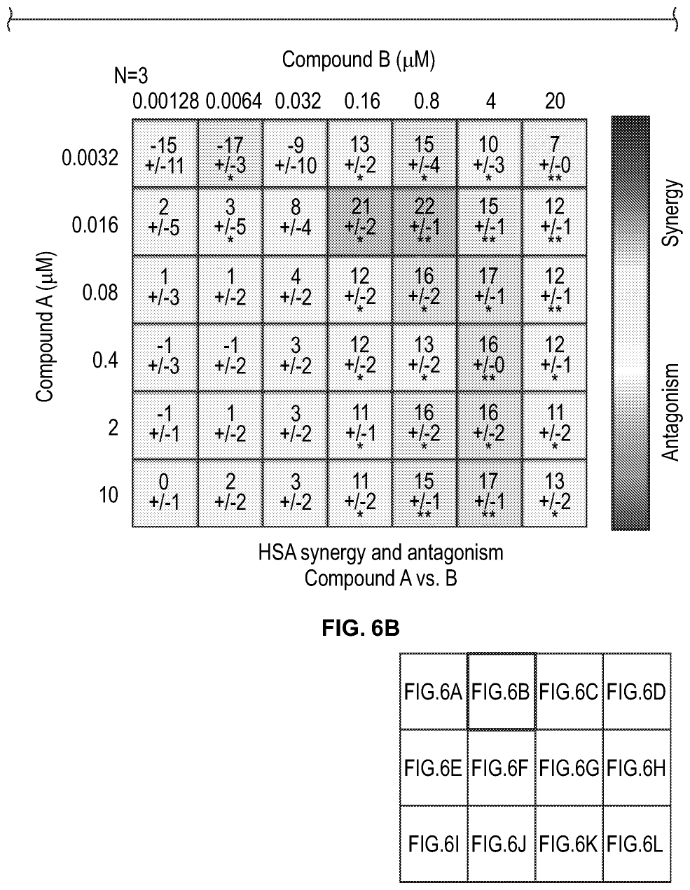

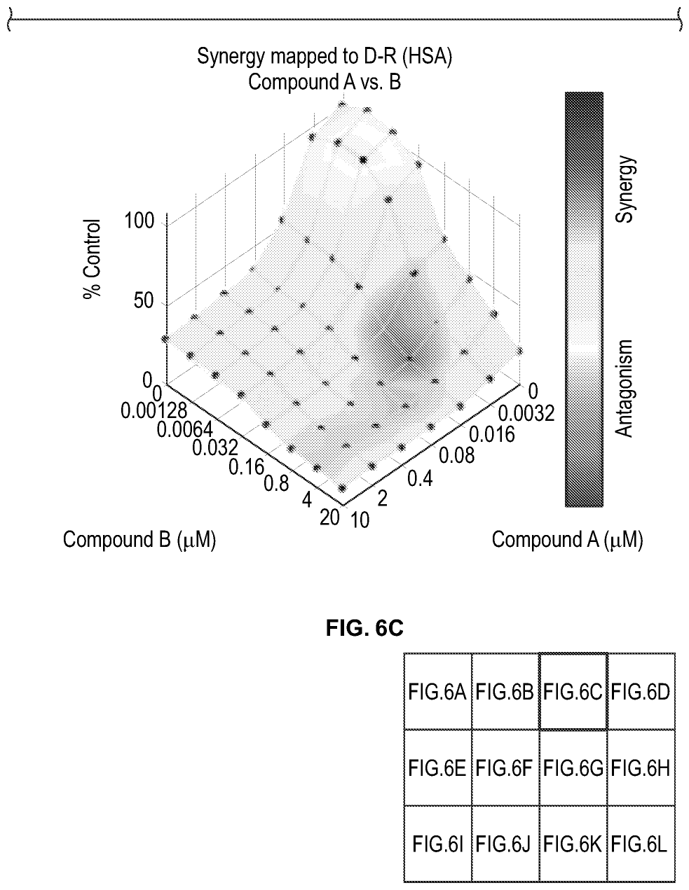

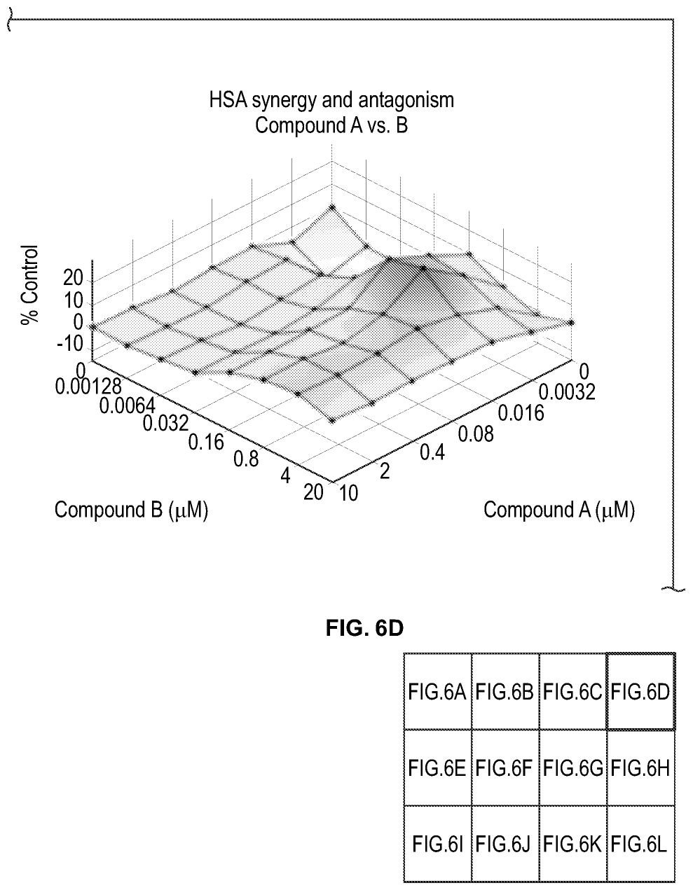

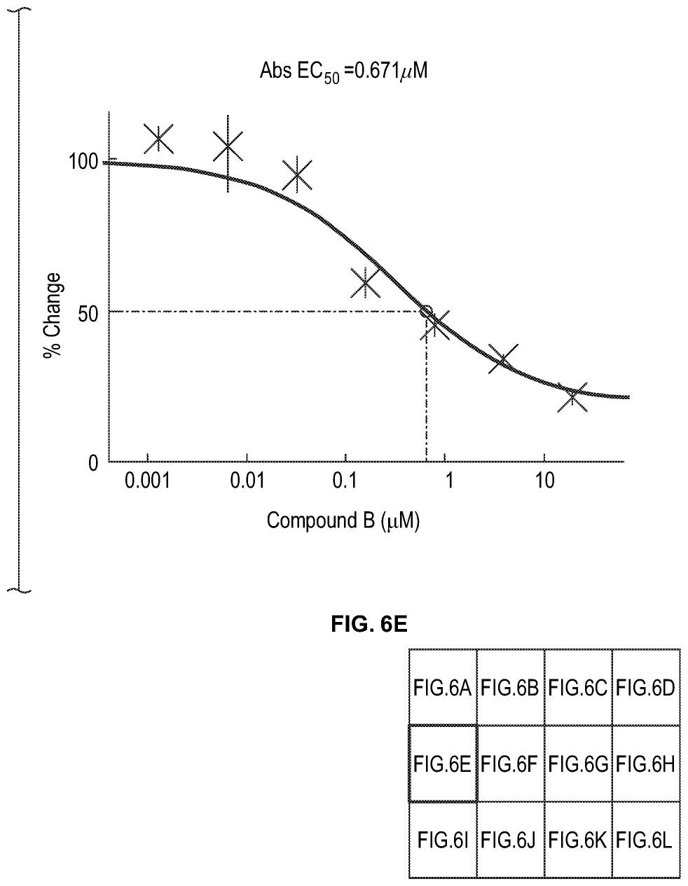

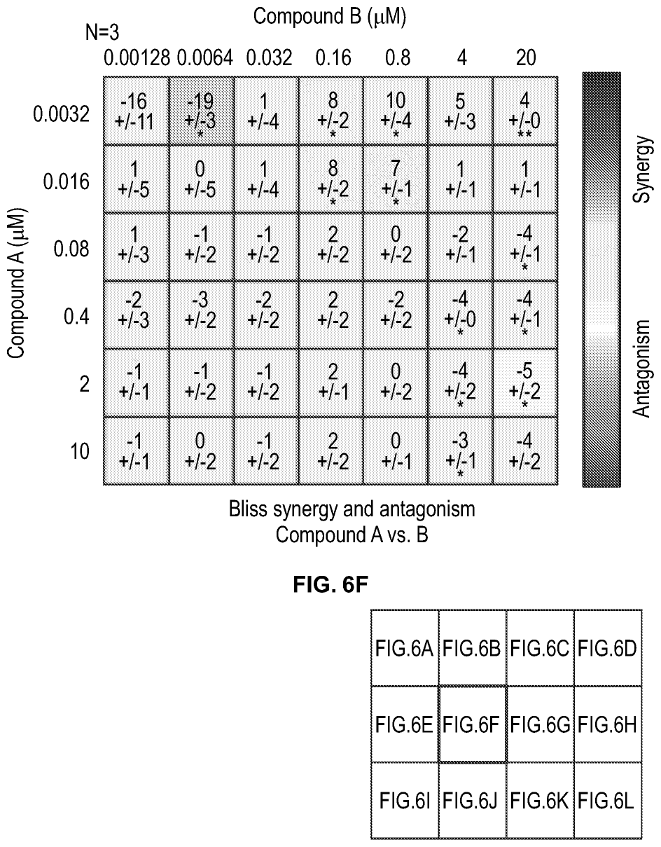

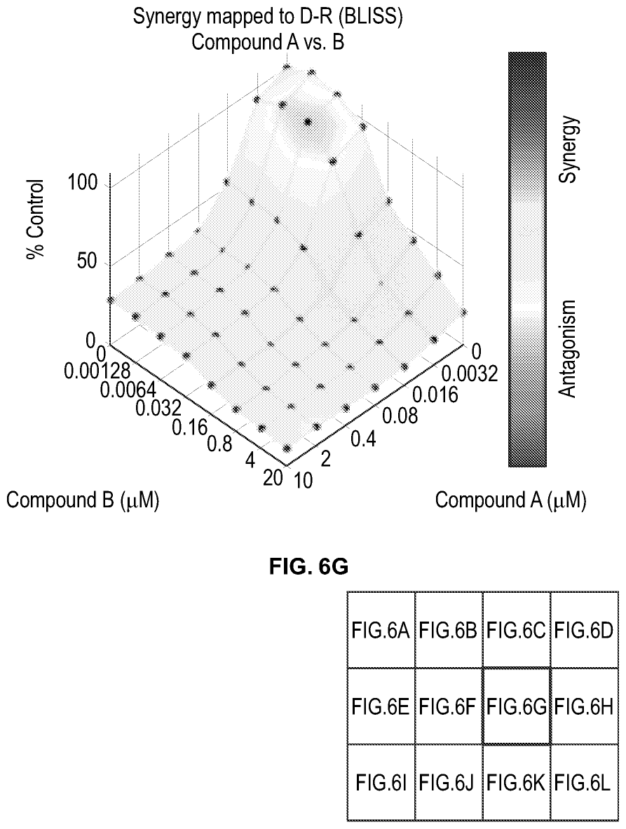

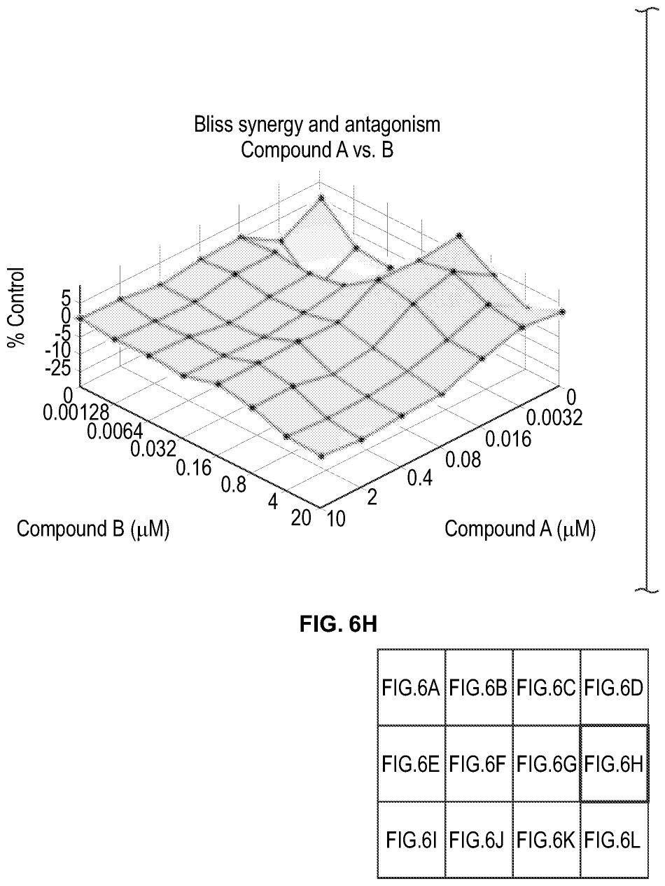

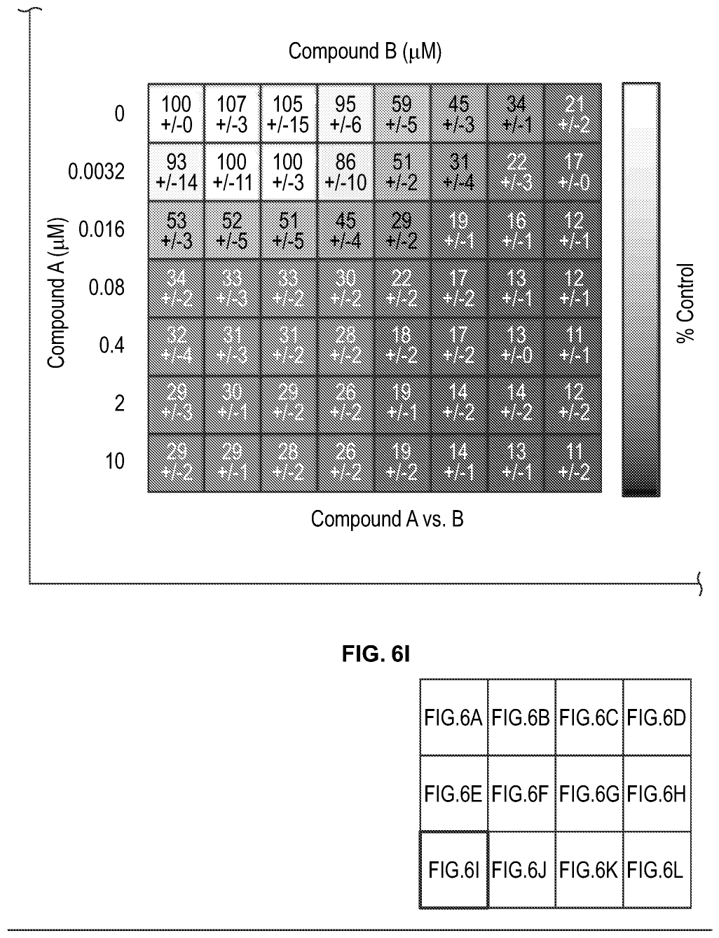

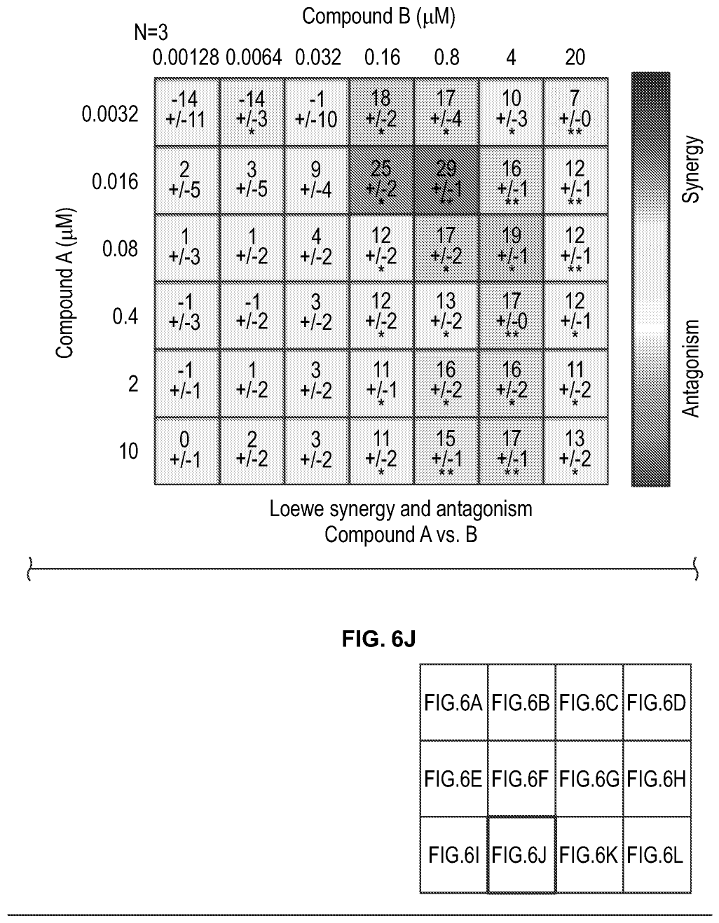

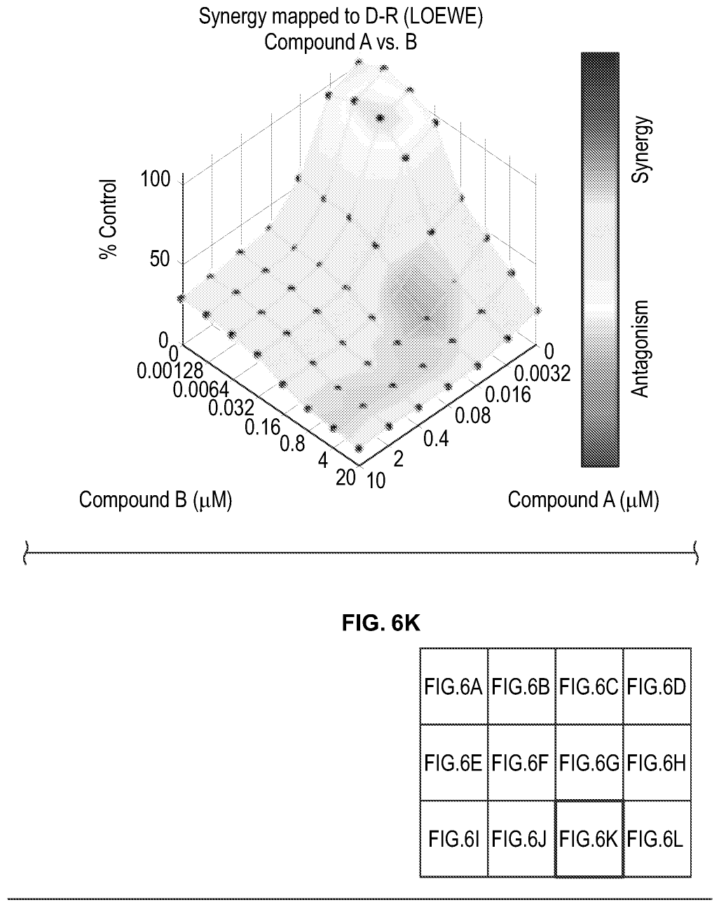

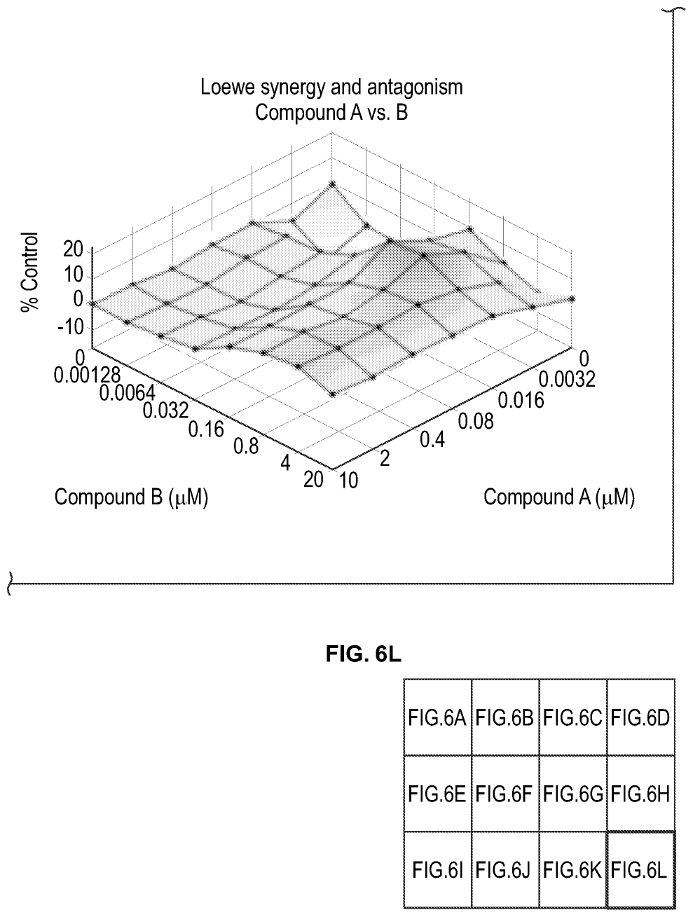

FIGS. 4-8 show that the combination effects of the MAT2A inhibitor (Compound A) with the Type I PRMT-inhibitor (Compound B) in cell lines BxPC3, KP4, NCI-H838, HCT116 and HCT116 MTAP^{-/-}. The combination resulted in modest synergistic growth inhibition in BxPC3 cell line using the HSA, Loewe and Bliss models (FIG. 4). However, no significant synergistic effect in the pancreatic cell line, KP4 using the Bliss model (FIG. 5). The modest cytotoxicity observed above 0.8 uM of Compound B in the HSA and Loewe models seems to be driven by the single agent activity of compound B. The moderate synergy observed at some dose levels using the HSA and Loewe models appears to be driven by the single agent activity of Compound B. Similarly, in the lung cell line NCI-H838, no significant synergy was observed using the Bliss model while modest synergy was observed using the HSA and Loewe models which also seems be to be driven by the single agent effect of compound B. The modest cytotoxicity observed above 0.16 uM of Compound B in the HSA and Loewe models seems to be driven by single agent activity of Compound B (FIG. 6).

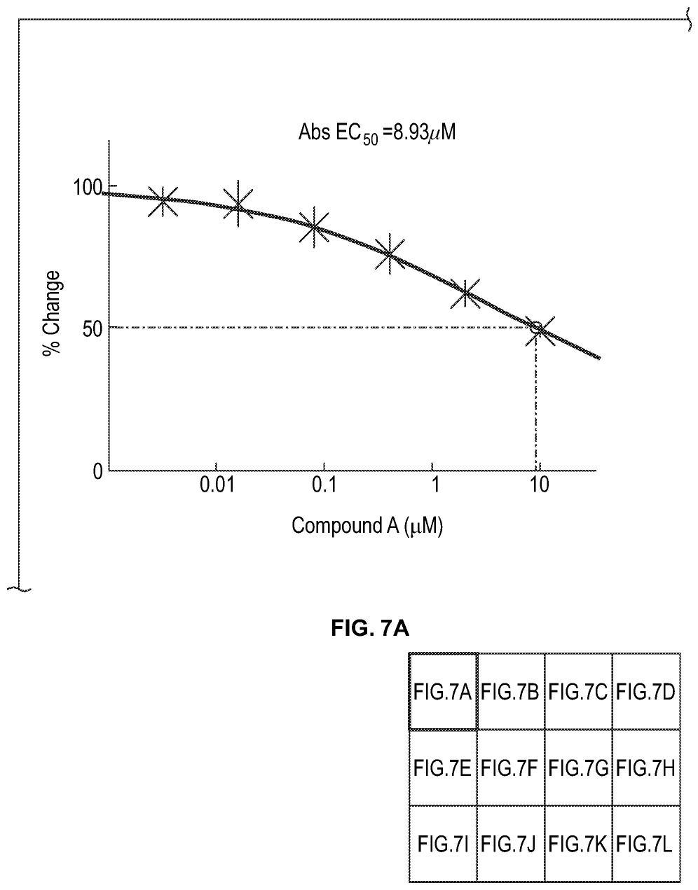

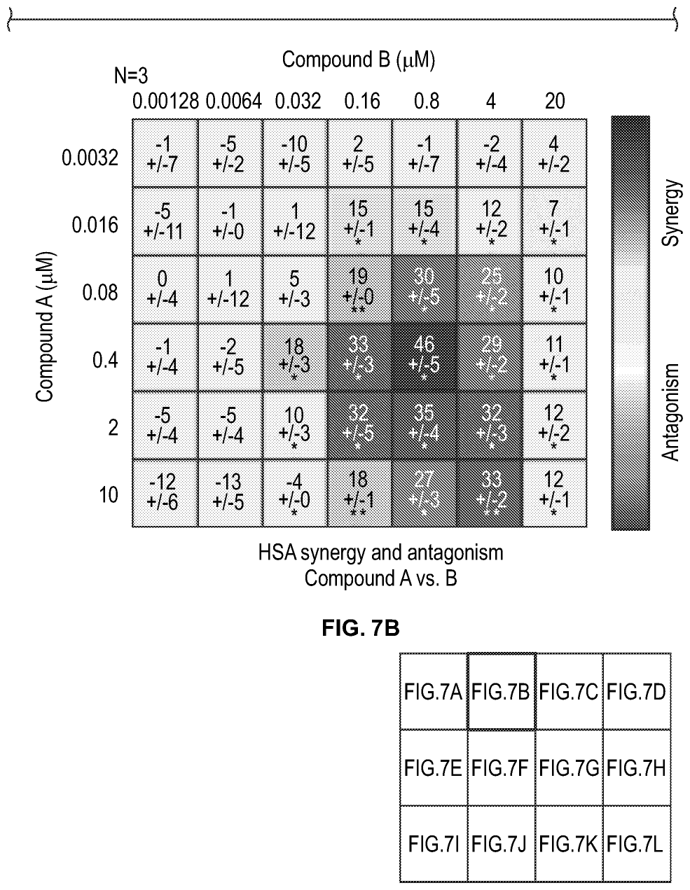

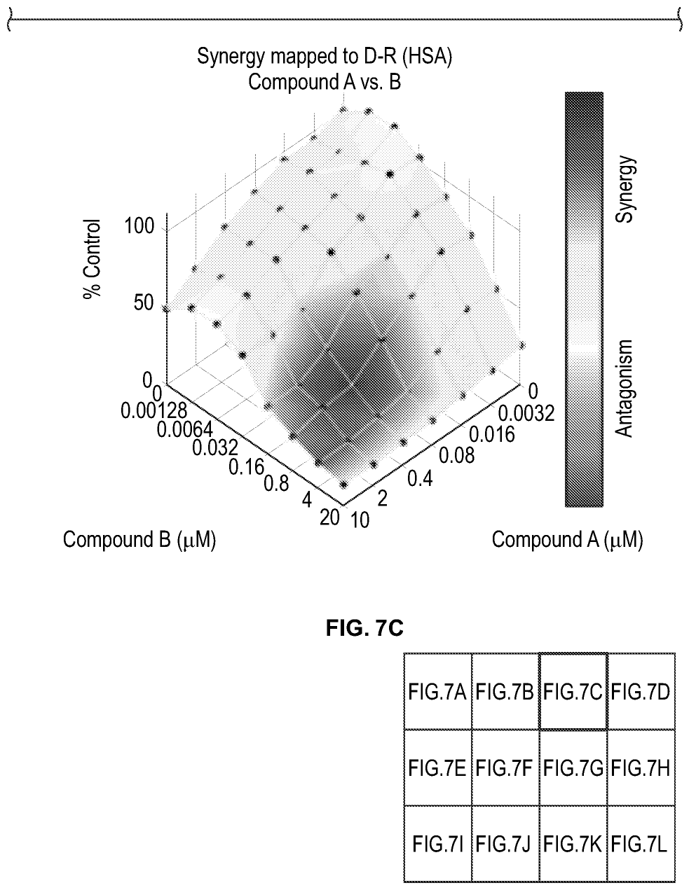

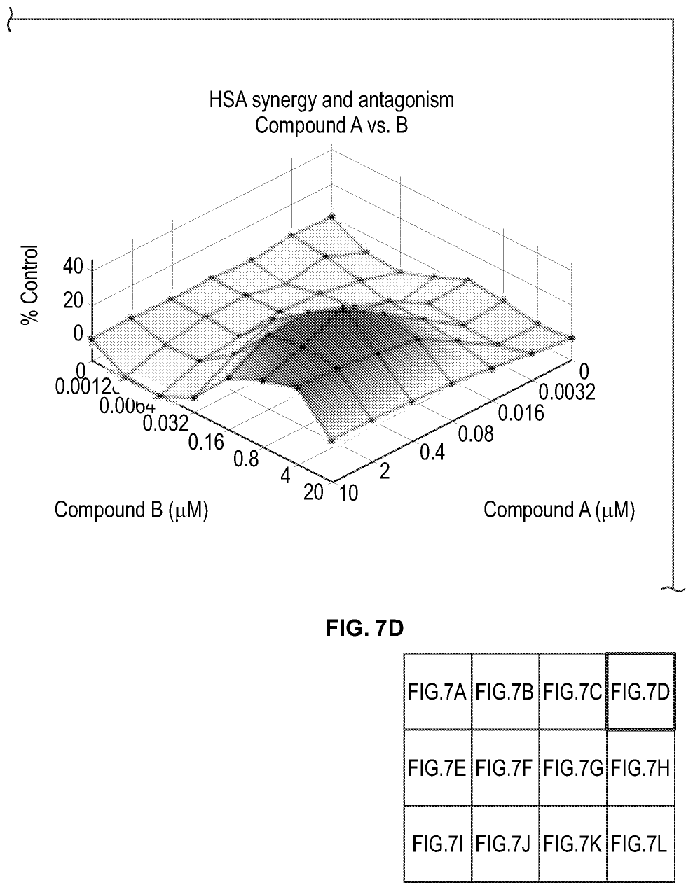

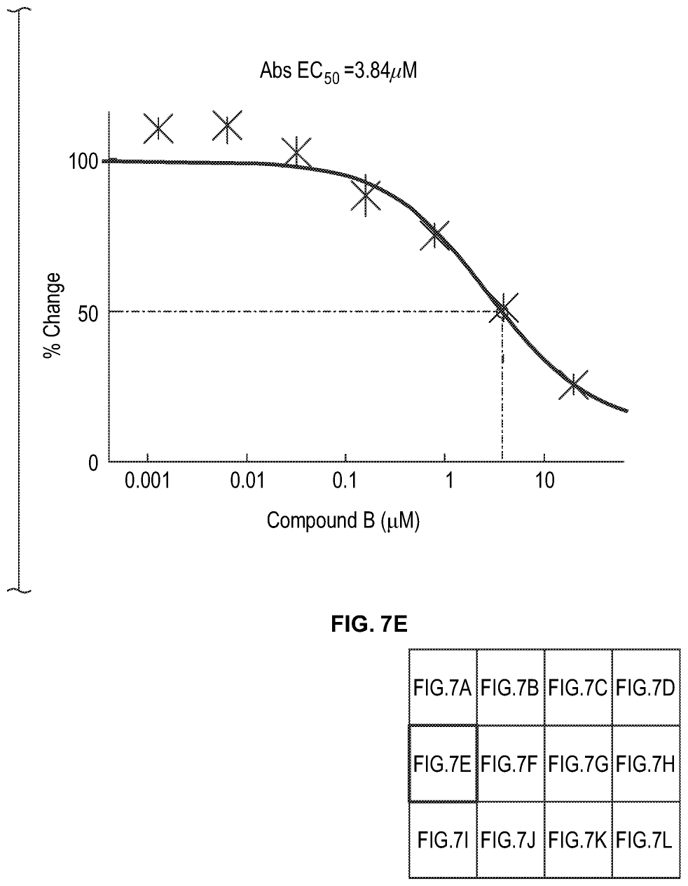

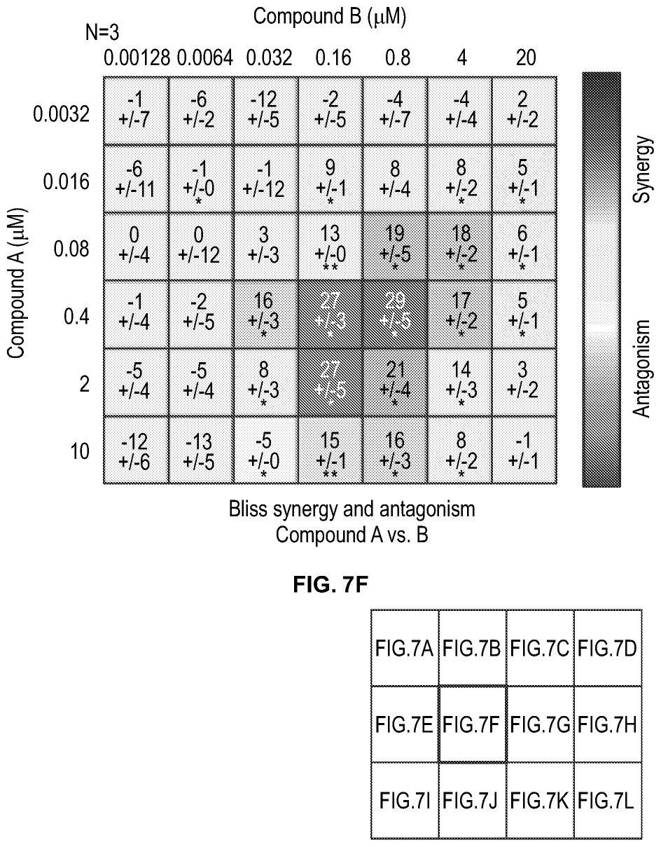

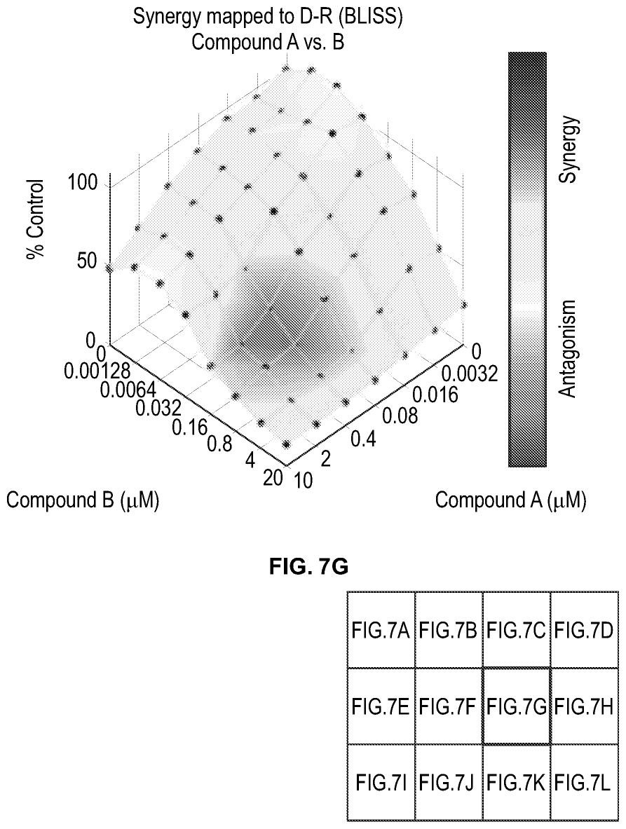

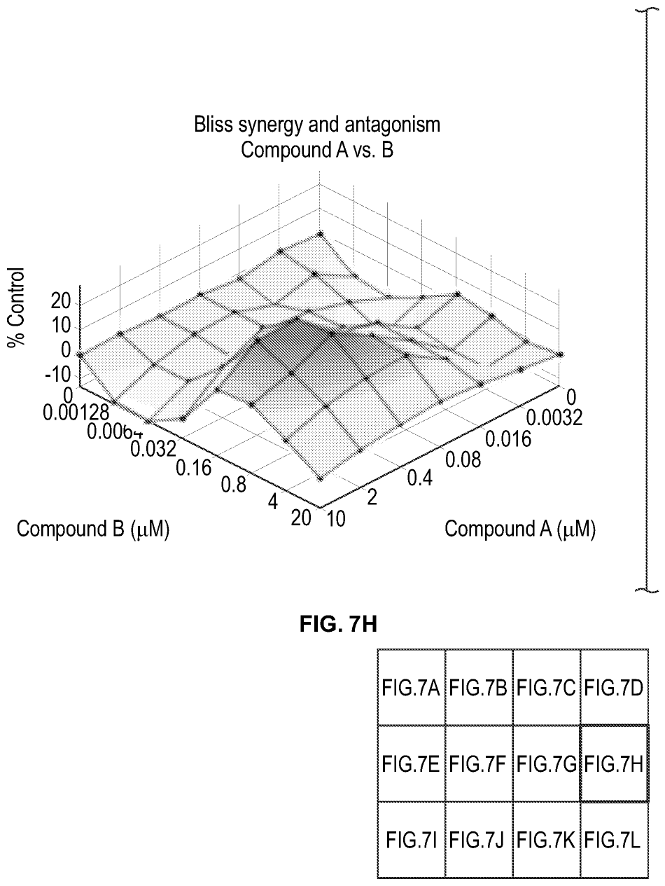

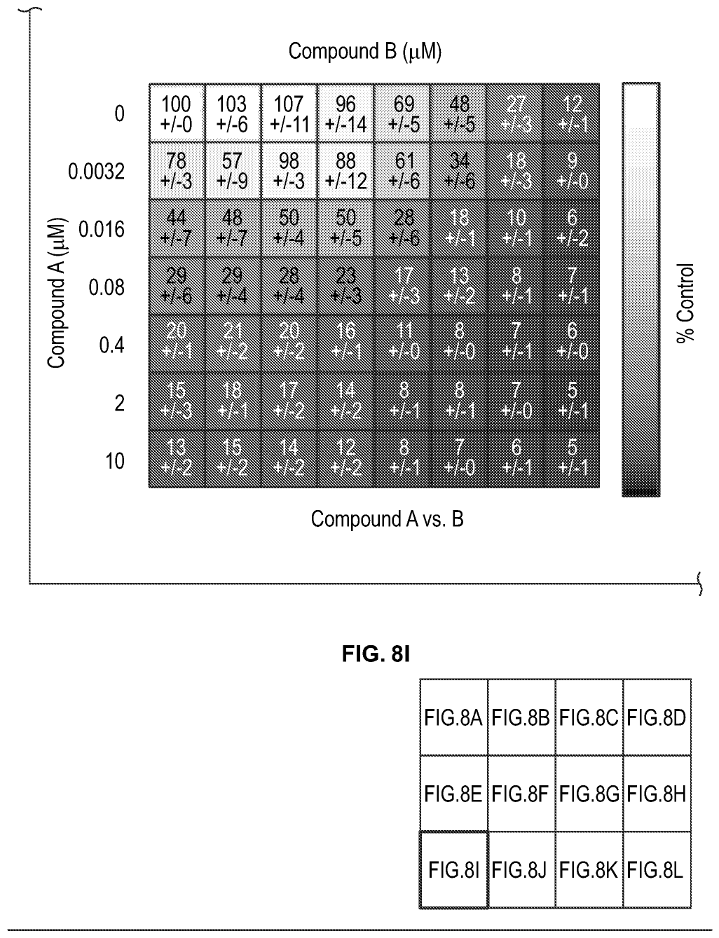

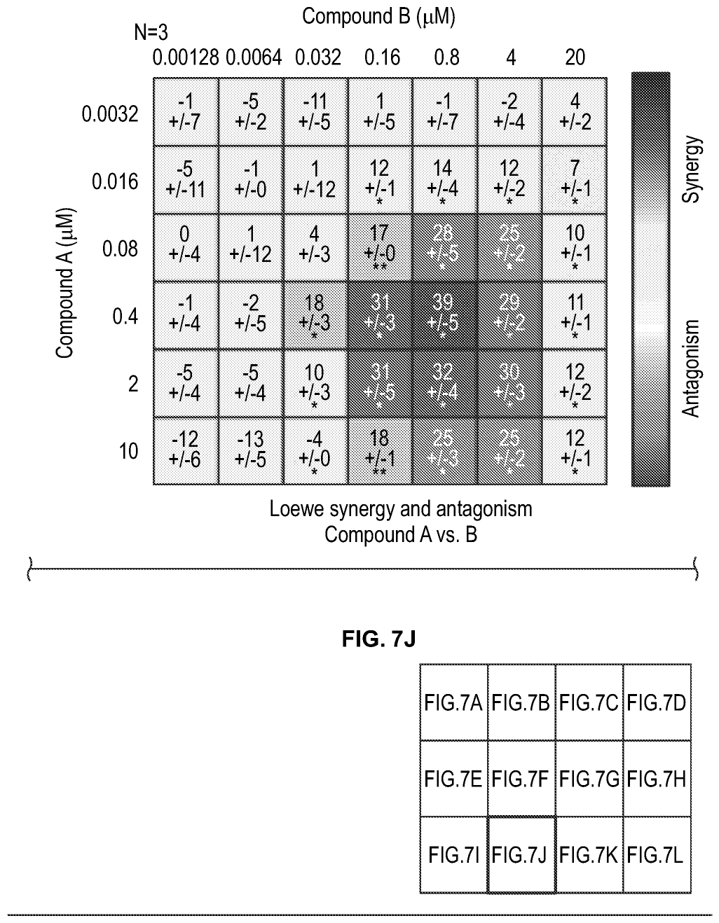

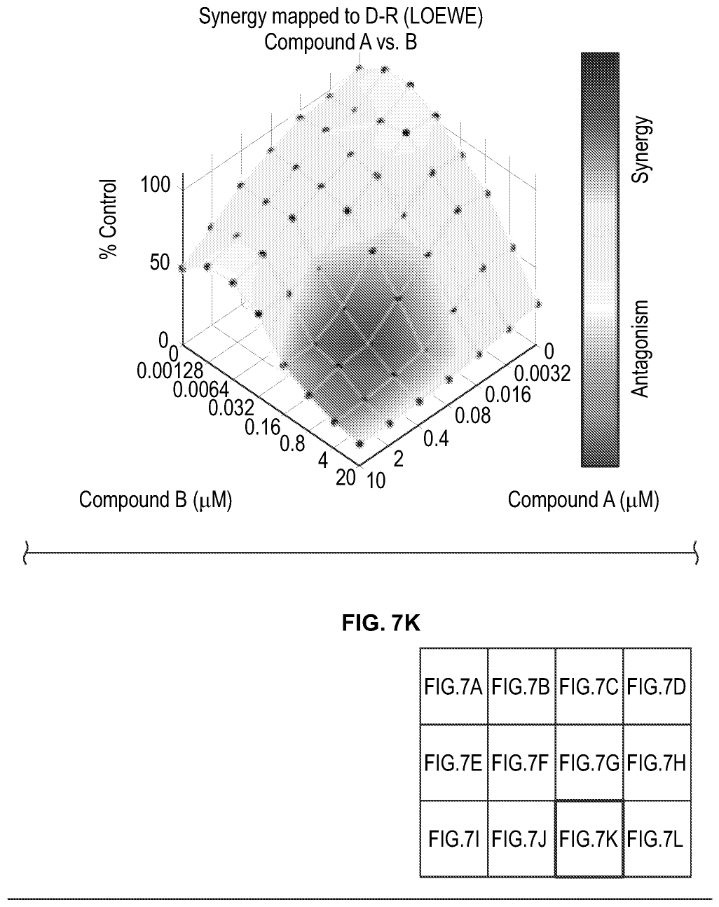

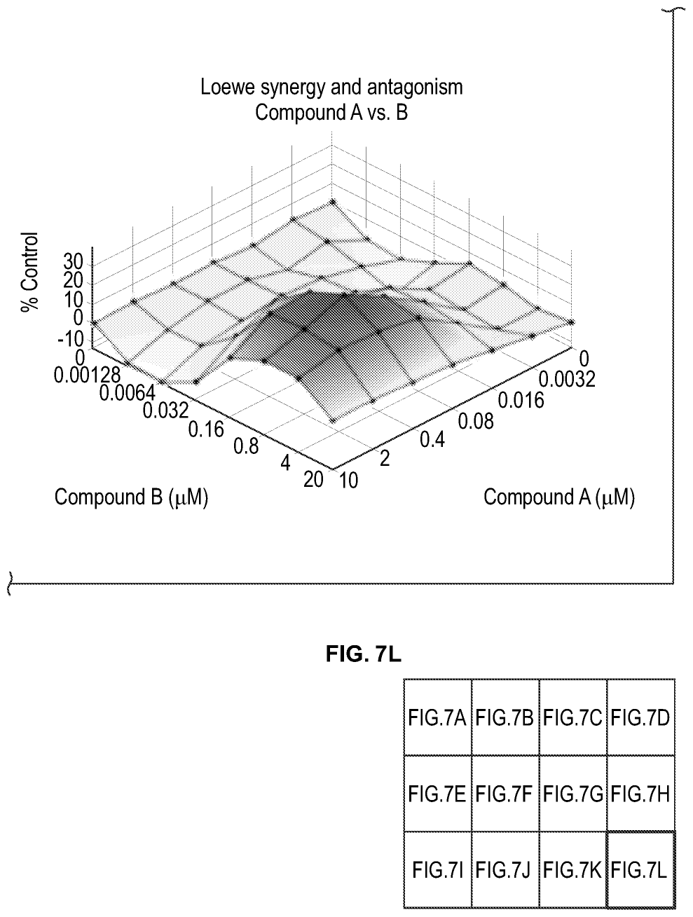

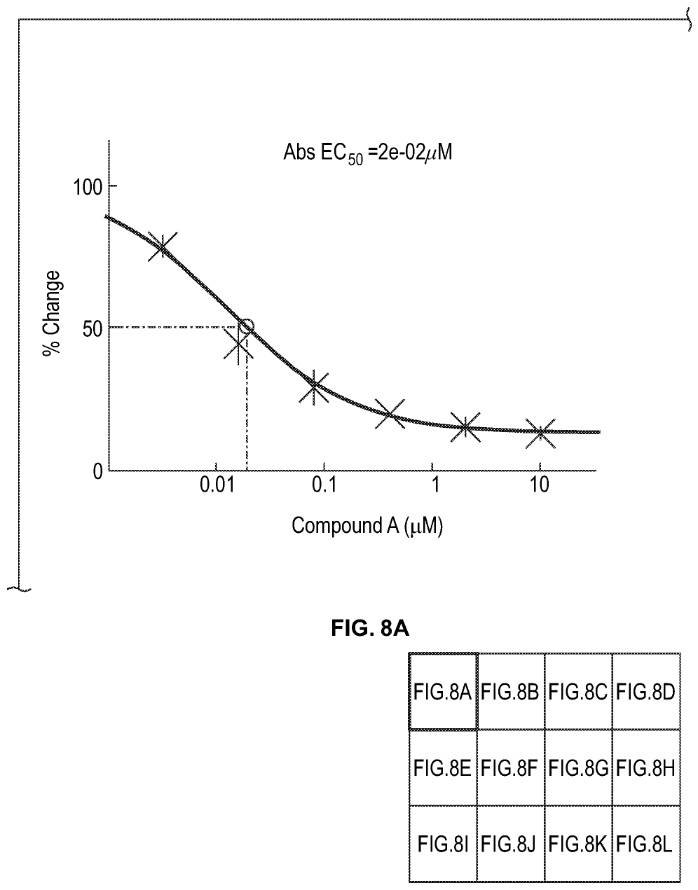

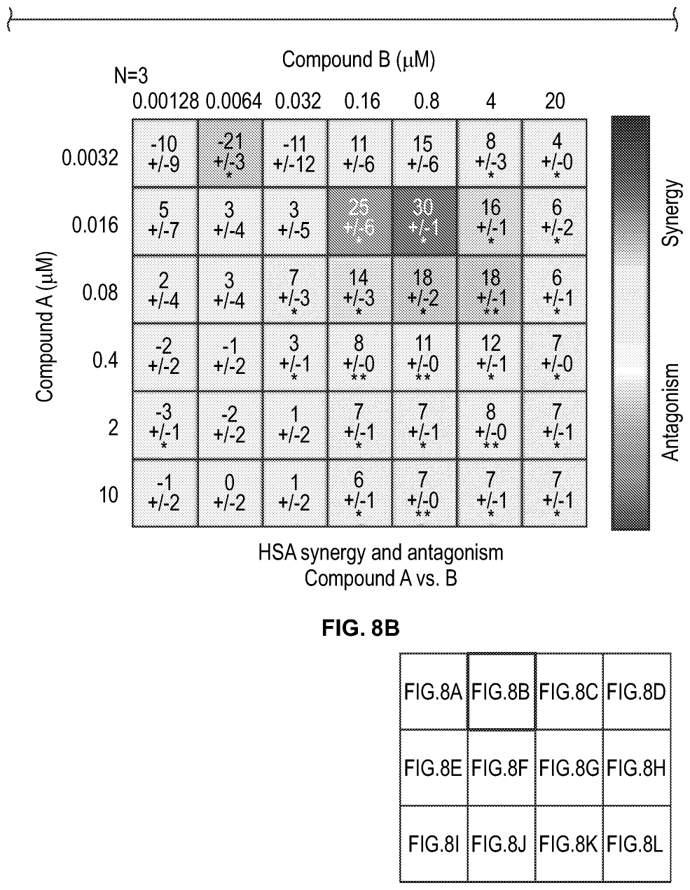

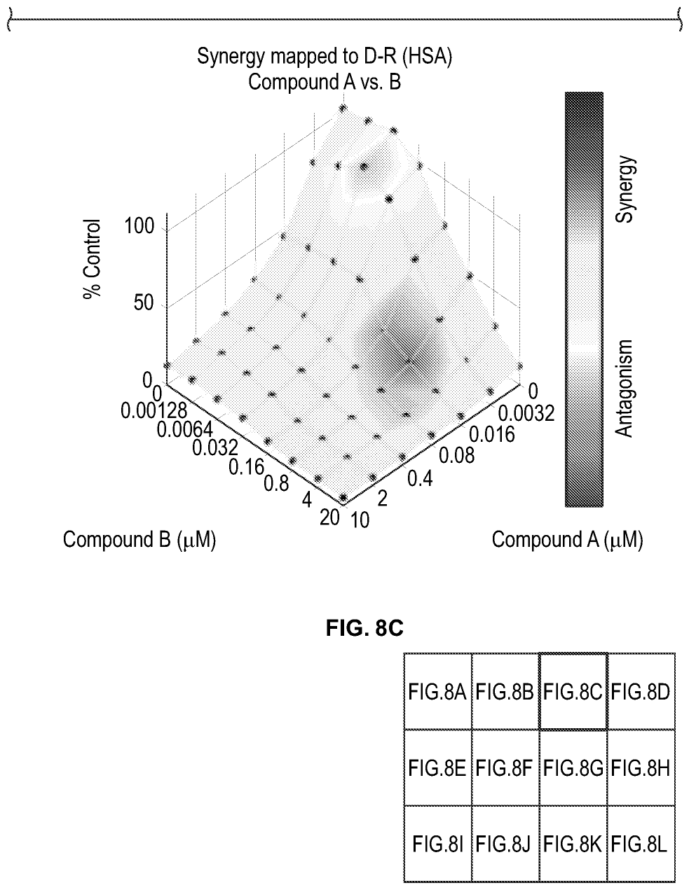

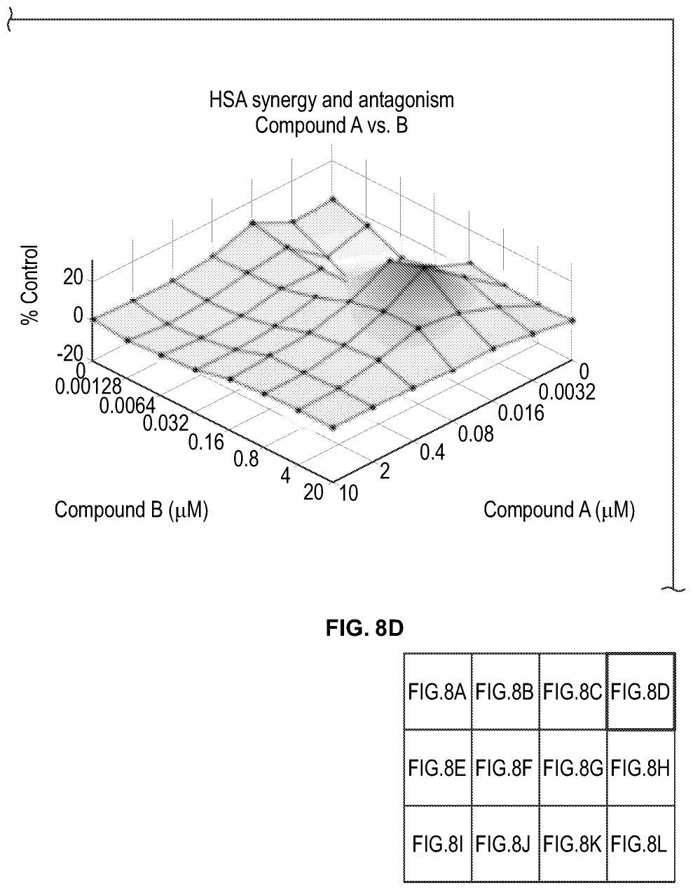

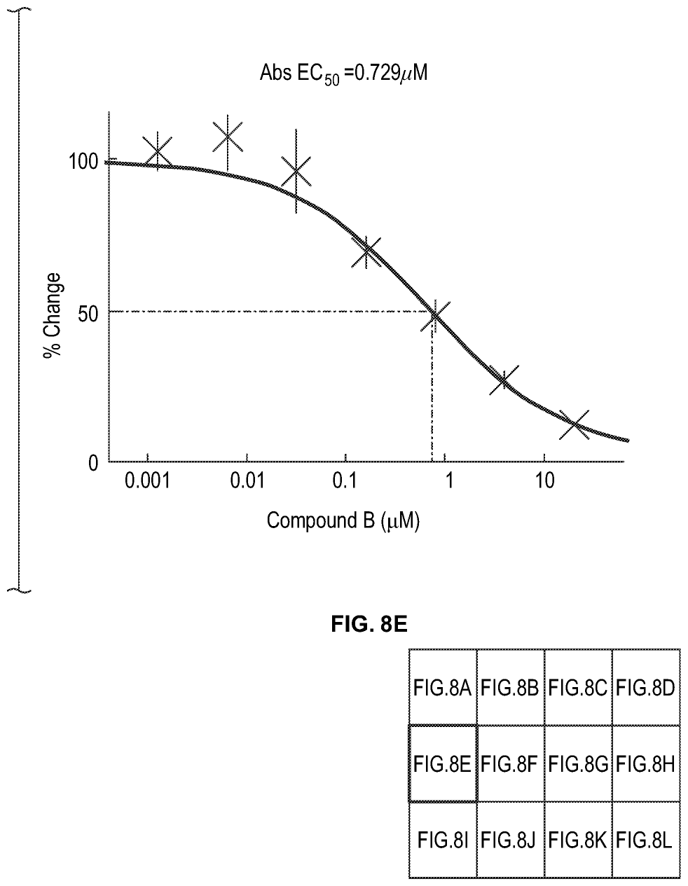

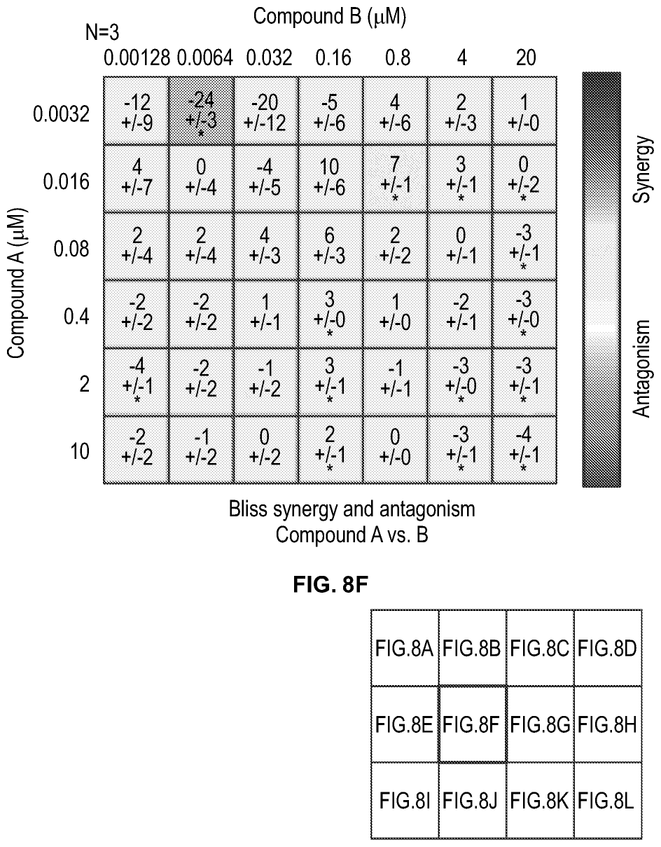

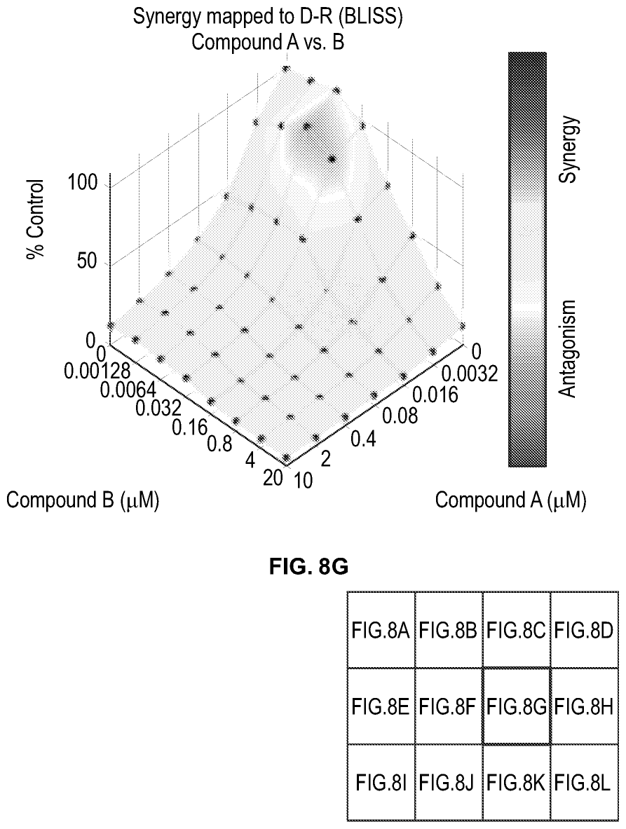

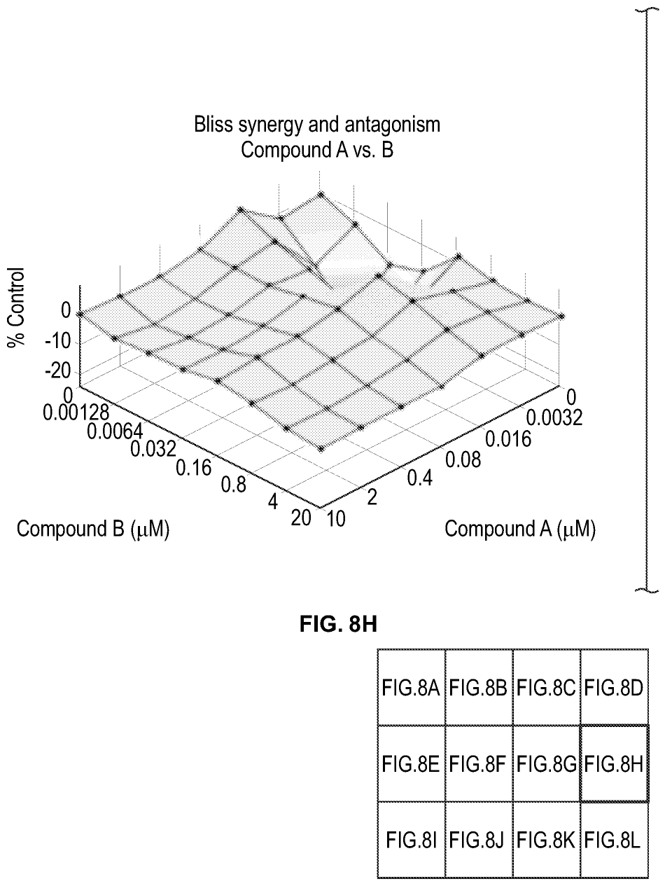

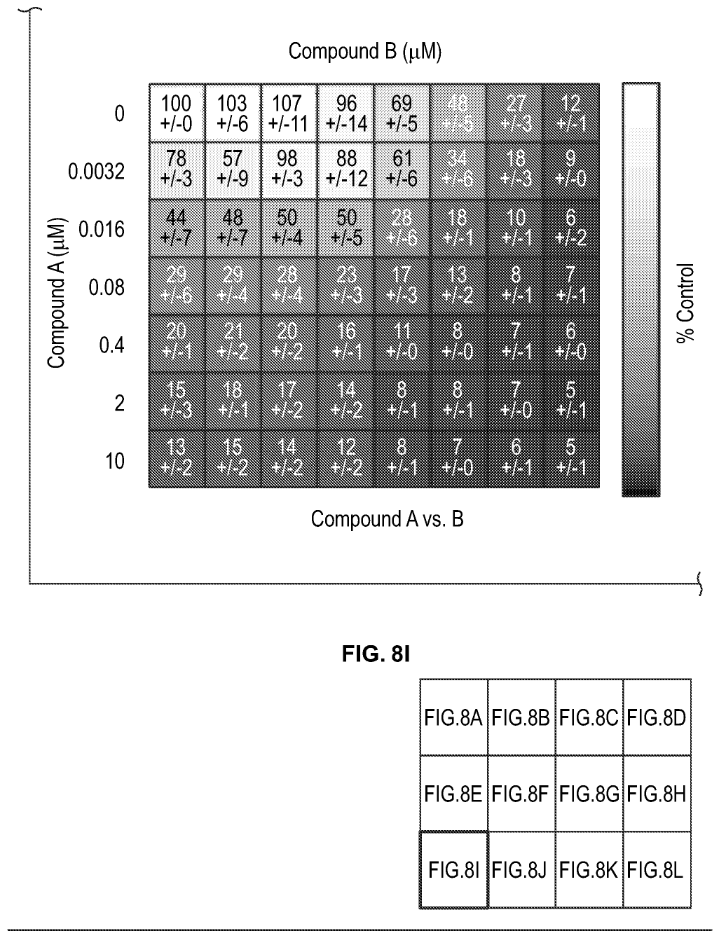

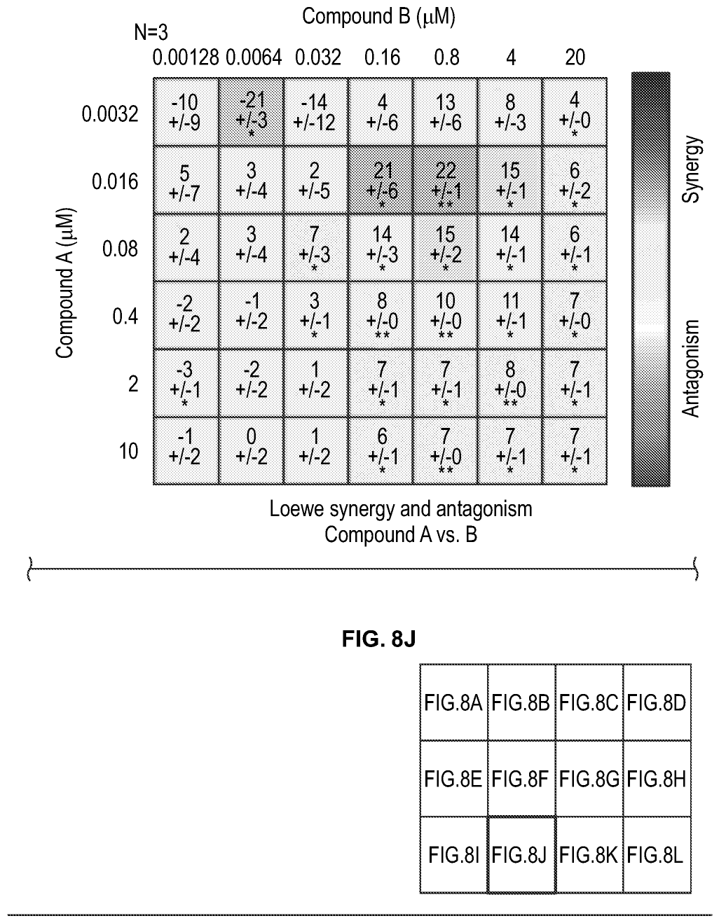

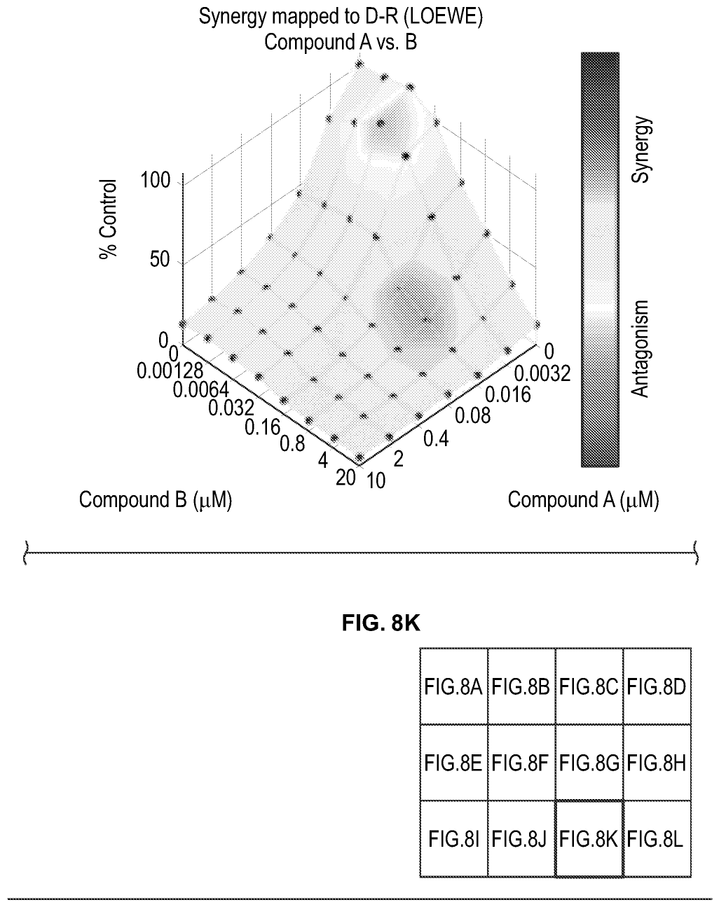

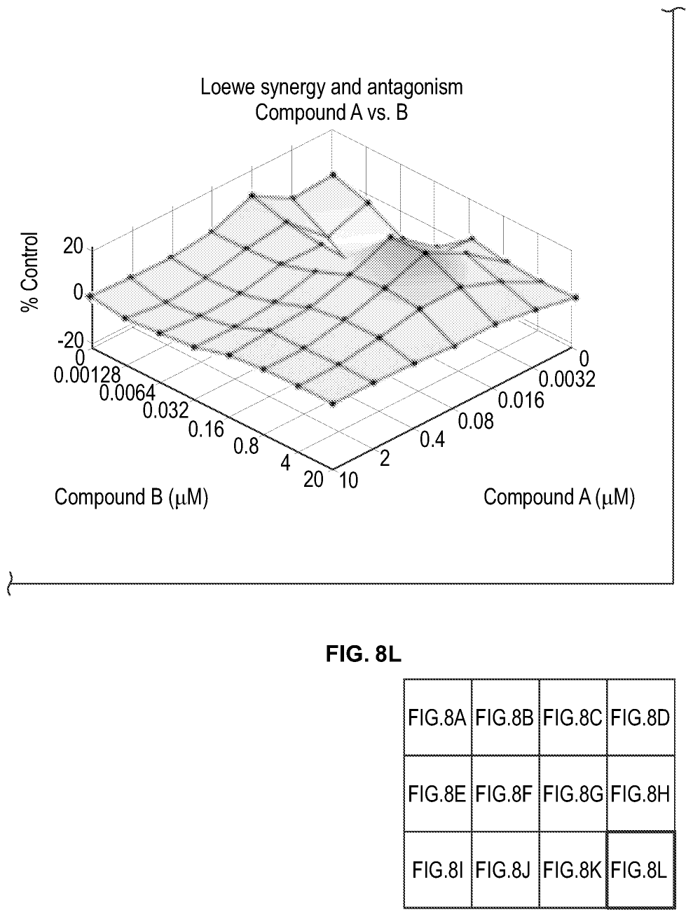

There was synergistic effects of Compound A and Compound B in HCT116 cell line (FIG. 7), however there was no significant synergistic effects in the HCT116 MTAP−/− isogenic line using the Bliss model (FIG. 8). Using the HSA and Loewe models, modest synergy was observed at 0.016 uM of compound A and 0.16 uM of compound B in the HCT116 MTAP−/− cell line. Synergy was observed at/above 0.4 uM of Compound A and 0.16 uM of Compound B in all models. In the HSA and Loewe models, modest synergy was observed at 0.016 uM of compound A and 0.16 uM of compound B.

### Example 3: MTA Measurement

### Methods

KP4, BxPC3, RT112/84, H647, H460 cells lines evaluated after 30 minutes and 48 hours of cell culture. After trypsinization and PBS wash, the cells were counted and then pelleted, snap-frozen and stored at -80 C until use. For MTA LC-MS analysis, the cells were homogenized in 30 μL of 85% acetonitrile in water with 0.1% perchloric acid with gentle shake. The homogenate sample is mixed with 100 μL of internal standard solution (D3-MTA in 85% acetonitrile in water with 0.1% perchloric acid). The mixture is vortexed on a shaker for 15 minutes and subsequently centrifuged at 4000 rpm for 15 minutes. An aliquot of 60 μL of the supernatant is mixed with 60 μL of water for the injection to the LC/MS/MS.

MTA powder is solubilized in dimethyl sulfoxide to bring the stock concentration to 1 mg/mL. 10 μL of 1 mg/mL stock solution is spiked into 990 μL of 85% acetonitrile in water with 0.1% perchloric acid. A serial dilution is performed to yield standard concentrations of 1, 2, 5, 20, 100, 200, 1000, 2000, 5000, and QC concentrations of 5, 50 and 500 ng/mL. Calibration standards and quality control samples (30 μL each) are prepared by spiking the testing compounds into 100 μL of internal standard solution (D3-MTA in 85% acetonitrile in water with 0.1% perchloric acid) and the resulting solution is processed with the unknown samples in the same batch. Then 10 μL is subjected to HPLC/MS analysis.

An Agilent 1200 binary HPLC pump with a thermo autosampler is used for all LC separations. The chromatographic separation of analytes is achieved on a Phenomenex Luna Omaga 3 μm Polar C18, 50×2.1 mm HPLC column, in conjunction with fast gradient conditions and mobile phases A (0.1% Formic acid in water) and B (0.1% Formic acid in Acetonitrile (v/v). A Sciex QTRAP 4000 (MS/MS) mass spectrometer equipped with a Turbo Ionspray interface from Applied Biosystems (Framingham, MA) is used for detection. The instrument is operated in the positive ion multiple reaction monitoring (MRM) mode employing nitrogen as a collision gas. The following MRM transitions are monitored: m/z 298.3 →250 and m/z 301.3 →250 for MTA and internal standard (D3-MTA), respectively. Data are acquired and processed by Sciex Analyst 1.7.2 software. Measured conc. (ng/mL) time with 30 (μL) to give resulted Number (pg) for each cell sample.

### Results

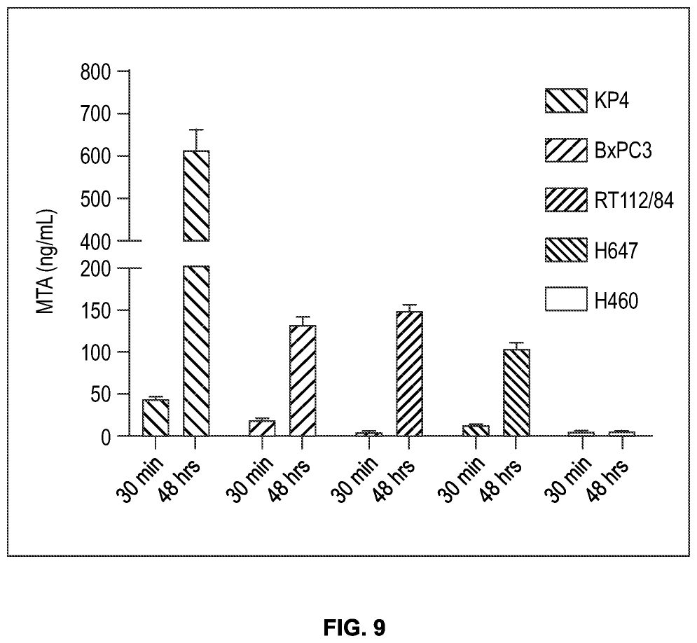

There was an increase in MTA levels after culturing for 48 hours in all MTAP deleted cell lines (FIG. 9). The pancreatic cancer cell line KP4 showed the maximum increase, followed by RT112/84 (bladder), BxPC3 (pancreas) and NCI-H647 (lung). No changes in MTA levels were observed in MTAP WT cell line NCI-H460.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

1. A method of treating cancer in a subject in need thereof, the method comprising administering to the subject an effective amount of a methionine adenosyltransferase II alpha (MAT2A) inhibitor and administering to the subject an effective amount of Type I protein arginine methyltransferase (Type I PRMT) inhibitor; wherein the MAT2A inhibitor is a compound of Formula IIId: 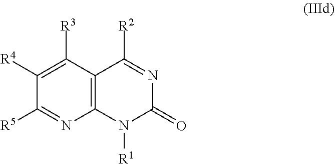
or a pharmaceutically acceptable salt thereof;
wherein
R³ is hydrogen, alkyl, alkenyl, alkynyl, alkoxy, alkylsulfonyl, halo, haloalkyl, haloalkoxy, cycloalkyl, cycloalkylalkyloxy, cyano, amino, alkylamino, dialkylamino, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroaralkyloxy, heteroarylamino, heterocyclyl, heterocyclyloxy, heterocyclylamino, heterocyclylalkyloxy, heterocyclyloxyalkoxy, or heterocyclyloxyalkylamino, wherein heterocyclyl or heteroaryl, by itself or as part of another group, is unsubstituted or substituted with R^{a}, R^{b}, or R^{c} independently selected from alkyl, cycloalkyl, haloalkyl, haloalkoxy, alkoxy, hydroxy, halo, cyano, alkoxycarbonyl, hydroxyalkyl, alkoxyalkyl, or aminoalkyl;
R⁵ is hydrogen, alkyl, alkenyl, alkynyl, alkoxy, alkylsulfonyl, halo, haloalkyl, haloalkoxy, cycloalkyl, cyano, amino, alkylamino, dialkylamino, alkoxycarbonyl, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroarylamino, heterocyclyl, heterocyclyloxy, heterocyclylamino, heterocyclyloxyalkoxy, or heterocyclyloxyalkylamino, wherein heterocyclyl or heteroaryl, by itself or as part of another group, is unsubstituted or substituted with R^{a}, R^{b}, or R^{c} independently selected from alkyl, cycloalkyl, haloalkyl, haloalkoxy, alkoxy, hydroxy, halo, cyano, hydroxyalkyl, alkoxyalkyl, or aminoalkyl;
R⁴ is hydrogen, alkyl, alkenyl, alkynyl, alkoxy, alkylthio, alkylsulfonyl, halo, haloalkyl, haloalkoxy, cycloalkyl, cyano, amino, alkylamino, dialkylamino, aminocarbonyl, alkylaminocarbonyl, or dialkylaminocarbonyl; provided that at least one of R³, R⁴, and R⁵ is other than hydrogen;
R¹ is R⁷ wherein R⁷ is cycloalkyl, bridged cycloalkyl, fused cycloalkyl, spirocycloalkyl, aryl, heteroaryl, heterocyclyl, bridged heterocyclyl, fused heterocyclyl, or spiroheterocyclyl, wherein aryl, heteroaryl, or heterocyclyl is unsubstituted or substituted with R^{d}, R^{e}, or R^{f};
R⁸ is alkyl, haloalkyl, hydroxyalkyl, alkoxyalkyl, aminoalkyl, aminocarbonylalkyl, aminosulfonylalkyl, —O—R¹, or —NR⁹R¹⁰ wherein:
R⁸ is alkyl, haloalkyl, hydroxyalkyl, alkoxyalkyl, aminoalkyl, cycloalkyl, cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heterocyclyl, heterocyclylalkyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{h}, R^{h}, or Rⁱ;
R⁹ is hydrogen, alkyl, deuteroalkyl, or cycloalkyl; and
R¹⁰ is hydrogen, alkyl, deuteroalkyl, haloalkyl, hydroxyalkyl, alkoxyalkyl, haloalkoxyalkyl, aminoalkyl, aminosulfonylalkyl, thioureidoalkyl, alkylsulfonyl, alkylsulfonylalkyl, cyanoalkyl, alkylcarbonyl, alkoxycarbonyl, alkylaminocarbonyl, dialkylaminocarbonyl, aminocarbonylalkyl, cycloalkyl, cycloalkylalkyl, substituted cycloalkyl, substituted cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heteroarylcarbonyl, heterocyclyl, heterocyclylalkyl, heterocyclylcarbonyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{j}, R^{k}, or R^{l};
and
R^{d}, R^{e}, R^{g}, R^{h}, R^{j}, R^{k}, R^{m}, and Rⁿ are independently selected from alkyl, haloalkyl, haloalkoxy, alkoxy, hydroxy, alkylsulfonyl, halo, cyano, carboxy, alkoxycarbonyl, hydroxyalkyl, alkoxyalkyl, aminoalkyl, aminosulfonyl, alkylaminosulfonyl, dialkylaminosulfonyl, sulfonylamino, aminocarbonyl, alkylaminocarbonyl, dialkylaminocarbonyl, heterocyclylcarbonyl, and ureido; and
R^{f}, Rⁱ, R^{l}, and R^{o} are independently selected from alkyl, cycloalkyl, haloalkyl, haloalkoxy, alkoxy, hydroxy, halo, amino, alkylamino, cycloalkylsulfonylamino, cyano, cyanoalkyl, alkoxycarbonylalkyl, carboxyalkyl, aminocarbonylalkyl, or —X^{c}—R¹² where X^{c} is bond, alkylene, or heteroalkylene and R¹² is optionally substituted aryl, optionally substituted heteroaryl, and optionally substituted heterocyclyl; provided that when R¹ is heterocyclyl then R^{f} is not hydroxy.

2. The method of claim 1, wherein the Type I PRMT inhibitor is a protein arginine methyltransferase 1 (PRMT1) inhibitor, a protein arginine methyltransferase 3 (PRMT3) inhibitor, a protein arginine methyltransferase 4 (PRMT4) inhibitor, a protein arginine methyltransferase 6 (PRMT6) inhibitor, or a protein arginine methyltransferase 8 (PRMT8) inhibitor.

3. The method of claim 1 or 2, wherein the Type I PRMT inhibitor is a protein arginine methyltransferase 1 (PRMT1) inhibitor.

4. The method of any one of claims 1-3, wherein
R³ is hydrogen, alkyl, alkenyl, alkynyl, alkoxy, alkylsulfonyl, halo, haloalkyl, haloalkoxy, cycloalkyl, cycloalkylalkyloxy, cyano, amino, alkylamino, dialkylamino, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroaralkyloxy, heteroarylamino, heterocyclyl, heterocyclyloxy, heterocyclylamino, heterocyclylalkyloxy, heterocyclyloxyalkoxy, or heterocyclyloxyalkylamino, wherein heterocyclyl or heteroaryl, by itself or as part of another group, is unsubstituted or substituted with R^{a}, R^{b}, or R^{c} independently selected from alkyl, cycloalkyl, haloalkyl, haloalkoxy, alkoxy, hydroxy, halo, cyano, alkoxycarbonyl, hydroxyalkyl, alkoxyalkyl, or aminoalkyl;
R⁵ is alkyl, alkenyl, alkynyl, alkoxy, alkylsulfonyl, halo, haloalkyl, haloalkoxy, cycloalkyl, cyano, amino, alkylamino, dialkylamino, alkoxycarbonyl, aminocarbonyl, alkylaminocarbonyl, dialkylaminocarbonyl, hydroxyalkyl, hydroxyalkoxy, hydroxyalkylamino, alkoxyalkyl, alkoxyalkoxy, alkoxyalkylamino, aminoalkyl, aminoalkoxy, aminoalkylamino, heteroaryl, heteroaryloxy, heteroarylamino, heterocyclyl, heterocyclyloxy, heterocyclylamino, heterocyclyloxyalkoxy, or heterocyclyloxyalkylamino, wherein heterocyclyl or heteroaryl, by itself or as part of another group, is unsubstituted or substituted with R^{a}, R^{b}, and/or R^{c} independently selected from alkyl, cycloalkyl, haloalkyl, haloalkoxy, alkoxy, hydroxy, halo, cyano, hydroxyalkyl, alkoxyalkyl, or aminoalkyl;
R⁴ is hydrogen, alkyl, alkenyl, alkynyl, alkoxy, alkylthio, alkylsulfonyl, halo, haloalkyl, haloalkoxy, cycloalkyl, cyano, amino, alkylamino, dialkylamino, aminocarbonyl, alkylaminocarbonyl, or dialkylaminocarbonyl;
R¹ is R⁷ wherein R⁷ is cycloalkyl, bridged cycloalkyl, fused cycloalkyl, spirocycloalkyl, aryl, heteroaryl, pyrrolidinyl, piperidinyl, piperazinyl, oxetanyl, morpholinyl, bridged heterocyclyl, fused heterocyclyl, or spiroheterocyclyl, wherein aryl, heteroaryl, or heterocyclyl is unsubstituted or substituted with R^{d}, R^{e}, and/or R^{f};
R² is alkyl, haloalkyl, hydroxyalkyl, alkoxyalkyl, aminoalkyl, aminocarbonylalkyl, aminosulfonylalkyl, —O—R⁸, —NR⁹R¹⁰, wherein:
R⁸ is alkyl, haloalkyl, hydroxyalkyl, alkoxyalkyl, aminoalkyl, cycloalkyl, cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heterocyclyl, heterocyclylalkyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{g}, R^{h}, or Rⁱ;
R⁹ is hydrogen, alkyl, deuteroalkyl, or cycloalkyl; and
R¹⁰ is hydrogen, alkyl, deuteroalkyl, haloalkyl, hydroxyalkyl, alkoxyalkyl, haloalkoxyalkyl, aminoalkyl, aminosulfonylalkyl, thioureidoalkyl, alkylsulfonyl, alkylsulfonylalkyl, cyanoalkyl, alkylcarbonyl, alkoxycarbonyl, alkylaminocarbonyl, dialkylaminocarbonyl, aminocarbonylalkyl, cycloalkyl, cycloalkylalkyl, substituted cycloalkyl, substituted cycloalkylalkyl, cycloalkoxyalkyl, bridged cycloalkyl, bridged cycloalkylalkyl, fused cycloalkyl, spirocycloalkyl, spirocycloalkylalkyl, aryl, aralkyl, heteroaryl, heteroaralkyl, heteroarylcarbonyl, heterocyclyl, heterocyclylalkyl, heterocyclylcarbonyl, heterocyclyloxyalkyl, fused heterocyclyl, fused heterocyclylalkyl, bridged heterocyclyl, bridged heterocyclylalkyl, spiroheterocyclyl, or spiroheterocyclylalkyl, wherein aryl, heteroaryl, or heterocyclyl, by itself or as part of another group, is unsubstituted or substituted with R^{j}, R^{k}, or R^{l};
R^{d}, R^{e}, R^{g}, R^{h}, R^{j}, R^{l}, R^{m}, and Rⁿ are independently selected from alkyl, haloalkyl, haloalkoxy, alkoxy, hydroxy, alkylsulfonyl, halo, cyano, carboxy, alkoxycarbonyl, hydroxyalkyl, alkoxyalkyl, aminoalkyl, aminosulfonyl, alkylaminosulfonyl, dialkylaminosulfonyl, sulfonylamino, aminocarbonyl, alkylaminocarbonyl, dialkylaminocarbonyl, heterocyclylcarbonyl, and ureido; and
R^{f}, Rⁱ, R^{l}, and R^{o} are independently selected from alkyl, cycloalkyl, haloalkyl, haloalkoxy, alkoxy, hydroxy, halo, amino, alkylamino, cycloalkylsulfonylamino, cyano, cyanoalkyl, alkoxycarbonylalkyl, carboxyalkyl, or —X^{c}—R¹² where X^{c} is bond, alkylene, or heteroalkylene and R¹² is optionally substituted aryl, or optionally substituted heteroaryl; provided that when R¹ is pyrrolidinyl, piperidinyl, piperazinyl, oxetanyl, or morpholinyl then R^{f} is not hydroxy; or a pharmaceutically acceptable salt thereof.

5. The method of any one of claims 1-4, wherein
R¹ is R⁷ wherein R⁷ is aryl optionally substituted with R^{d}, R^{e}, or R^{f};
R² is —NR⁹R¹⁰, wherein
R⁹ is hydrogen or alkyl; and
R¹⁰ is hydrogen or alkyl;
R³ is hydrogen;
R⁴ is hydrogen;
R⁵ is haloalkyl; and
R^{d}, R^{e}, and R^{f} are each, independently, halo.

6. The method of any one of claims 1-5, wherein the MAT2A inhibitor is Compound A: 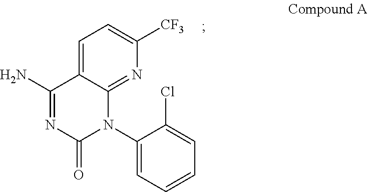 [SMILES: Nc1nc(=O)n(-c2ccccc2Cl)c2nc(C(F)(F)F)ccc12]
or a pharmaceutically acceptable salt thereof.

7. The method of any one of claims 1-6, wherein the Type I PRMT inhibitor is Compound B: 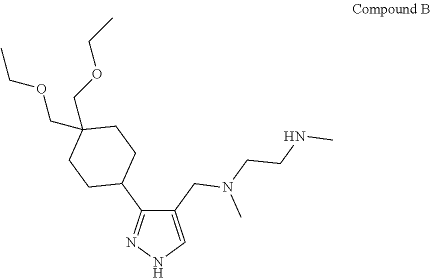 [SMILES: CCOCC1(COCC)CCC(c2n[nH]cc2CN(C)CCNC)CC1]
or a pharmaceutically acceptable salt thereof.

8. The method of any one of claims 1-6, wherein the Type I PRMT inhibitor is selected from the group consisting of a compound from Table 2, or a pharmaceutically acceptable salt thereof.

9. A method of treating cancer in a subject in need thereof, the method comprising administering to the subject an effective amount of Compound A: 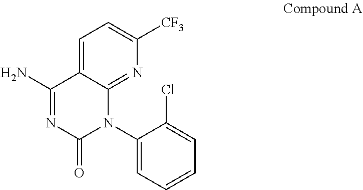 [SMILES: Nc1nc(=O)n(-c2ccccc2Cl)c2nc(C(F)(F)F)ccc12]
or a pharmaceutically acceptable salt thereof; and
administering to the subject an effective amount of Type I protein arginine methyltransferase (Type I PRMT) inhibitor, or a pharmaceutically acceptable salt thereof.

10. A method of treating cancer in a subject in need thereof, the method comprising administering to the subject an effective amount of Compound A: 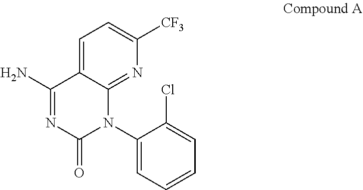 [SMILES: Nc1nc(=O)n(-c2ccccc2Cl)c2nc(C(F)(F)F)ccc12]
or a pharmaceutically acceptable salt thereof; and
administering to the subject an effective amount of Compound B: 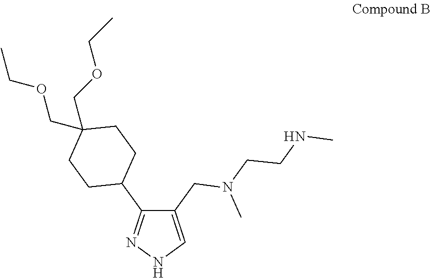 [SMILES: CCOCC1(COCC)CCC(c2n[nH]cc2CN(C)CCNC)CC1]
or a pharmaceutically acceptable salt thereof.

11. The method of any one of claims 1-10, wherein the cancer is characterized by a reduction or absence of MTAP gene expression, absence of the MTAP gene, reduced function of MTAP protein, reduced level of MTAP protein, absence of MTAP protein, MTA accumulation, or combination thereof.

12. The method of any one of claims 1-11, wherein the cancer is selected from the group consisting of leukemia, glioma, melanoma, pancreatic, non-small cell lung cancer, bladder cancer, astrocytoma, osteosarcoma, head and neck cancer, myxoid chondrosarcoma, ovarian cancer, endometrial cancer, breast cancer, soft tissue sarcoma, non-Hodgkin lymphoma, and mesothelioma.

13. The method of any of claims 1-12, wherein the cancer is a solid tumor.

14. The method of any of claims 1-12, wherein the cancer is pancreatic cancer, non-small cell lung cancer, esophageal cancer, or bladder cancer.

15. The method of any of claims 1-14, wherein the MAT2A inhibitor and PRMT type 1 inhibitor are in separate dosage forms.

16. The method of any of claims 1-14, wherein the MAT2A inhibitor and PRMT type I inhibitor are in the same dosage form.

17. A combination product comprising a methionine adenosyltransferase II alpha (MAT2A) inhibitor of Formula I, or a pharmaceutically acceptable salt thereof, and a Type I protein arginine methyltransferase (Type I PRMT) inhibitor, or a pharmaceutically acceptable salt thereof.

18. The combination product of claim 17, wherein the MAT2A inhibitor is selected from the group consisting of a compound in Table 1, or a pharmaceutically acceptable salt thereof.

19. The combination product of claim 17 or 18, wherein the MAT2A inhibitor is Compound A: 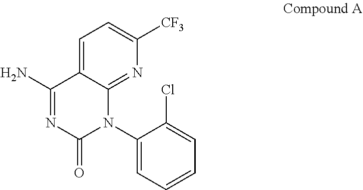 [SMILES: Nc1nc(=O)n(-c2ccccc2Cl)c2nc(C(F)(F)F)ccc12]
or a pharmaceutically acceptable salt thereof.

20. The combination product of any one of claims 17-19, wherein the Type I PRMT inhibitor is a protein arginine methyltransferase 1 (PRMT1) inhibitor.

21. The combination product of any one of claims 17-20, wherein the Type I PRMT inhibitor is selected from the group consisting of a compound in Table 2, or a pharmaceutically acceptable salt thereof.

22. The combination product of any one of claims 17-20, wherein the Type I PRMT inhibitor is Compound B: 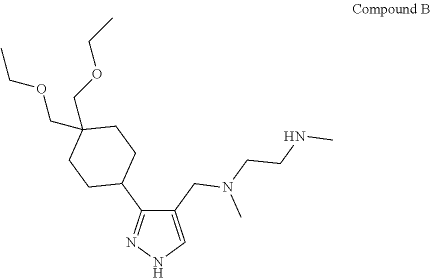 [SMILES: CCOCC1(COCC)CCC(c2n[nH]cc2CN(C)CCNC)CC1]
or a pharmaceutically acceptable salt thereof.

23. A combination product comprising Compound A: 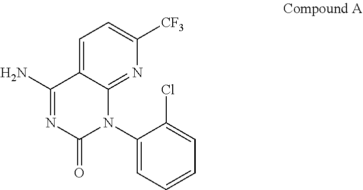 [SMILES: Nc1nc(=O)n(-c2ccccc2Cl)c2nc(C(F)(F)F)ccc12]
or a pharmaceutically acceptable salt thereof; and
a Compound B: 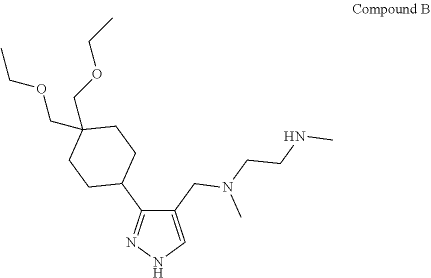 [SMILES: CCOCC1(COCC)CCC(c2n[nH]cc2CN(C)CCNC)CC1]
or a pharmaceutically acceptable salt thereof.

24. A methionine adenosyltransferase II alpha (MAT2A) inhibitor for use in treating cancer, wherein the MAT2A inhibitor is to be administered simultaneously or sequentially with a Type I protein arginine methyltransferase (Type I PRMT) inhibitor.

25. A methionine adenosyltransferase II alpha (MAT2A) inhibitor for use in treating cancer, wherein the MAT2A inhibitor is to be administered simultaneously or sequentially with a Type I protein arginine methyltransferase (Type I PRMT) inhibitor; wherein the MAT2A inhibitor is Compound A: 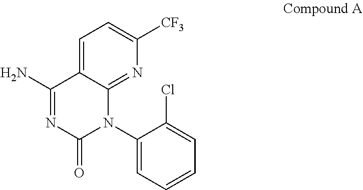 [SMILES: Nc1nc(=O)n(-c2ccccc2Cl)c2nc(C(F)(F)F)ccc12]
or a pharmaceutically acceptable salt thereof; and
the PRMT inhibitor is Compound B: 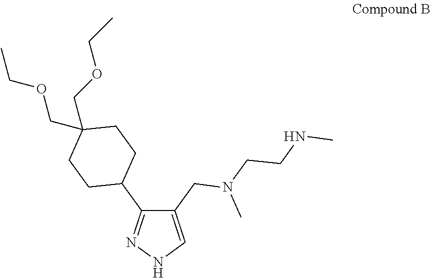 [SMILES: CCOCC1(COCC)CCC(c2n[nH]cc2CN(C)CCNC)CC1]
or a pharmaceutically acceptable salt thereof.

26. Use of a methionine adenosyltransferase II alpha (MAT2A) inhibitor in the manufacture of a medicament for treating cancer, wherein the MAT2A inhibitor is to be administered simultaneously or sequentially with a Type I protein arginine methyltransferase (Type I PRMT) inhibitor.

27. Use of a methionine adenosyltransferase II alpha (MAT2A) inhibitor in the manufacture of a medicament for treating cancer, wherein the MAT2A inhibitor is to be administered simultaneously or sequentially with a Type I protein arginine methyltransferase (Type I PRMT) inhibitor; wherein the MAT2A inhibitor is Compound A: 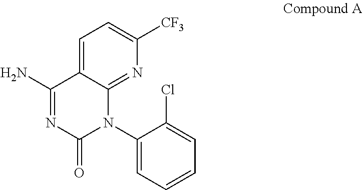 [SMILES: Nc1nc(=O)n(-c2ccccc2Cl)c2nc(C(F)(F)F)ccc12]
or a pharmaceutically acceptable salt thereof; and
the PRMT inhibitor is Compound B: 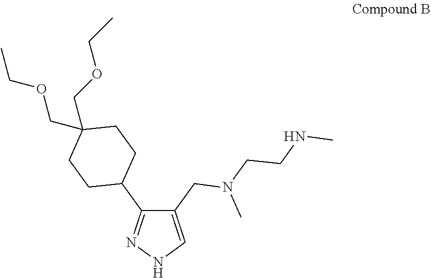 [SMILES: CCOCC1(COCC)CCC(c2n[nH]cc2CN(C)CCNC)CC1]
or a pharmaceutically acceptable salt thereof.
